(19) United States
(12) Patent Application Publication
(10) Pub. No.: US 2023/0192677 A1
(43) Pub. Date: Jun. 22, 2023

(54) CYP11A1 INHIBITORS

(71) Applicant: ORION CORPORATION, Espoo (FI)

(72) Inventors: David DIN BELLE, Espoo (FI); Esa KUMPULAINEN, Helsinki (FI); Mikko MÄKELÄ, Espoo (FI); Pekka PIETIKÄINEN, Espoo (FI); Shouming WANG, Espoo (FI); Gerd WOHLFAHRT, Berlin (DE)

(21) Appl. No.: 17/924,857

(22) PCT Filed: May 12, 2021
(86) PCT No.: PCT/FI2021/050353

§ 371 Date: Nov. 11, 2022

(30) Foreign Application Priority Data
May 14, 2020 (FI) 20205486

(51) Int. Cl. C07D 413/14; A61K 45/06; C07D 417/14; C07D 405/14; C07D 401/14; C07D 401/12; C07D 209/44; C07D 403/12; C07D 405/12

(52) U.S. Cl. CPC C07D 413/14; A61K 45/06; C07D 417/14; C07D 405/14; C07D 401/14; C07D 401/12; C07D 209/44; C07D 403/12; C07D 405/12

(57) ABSTRACT

The present invention relates to compounds of formula (I) and (II)

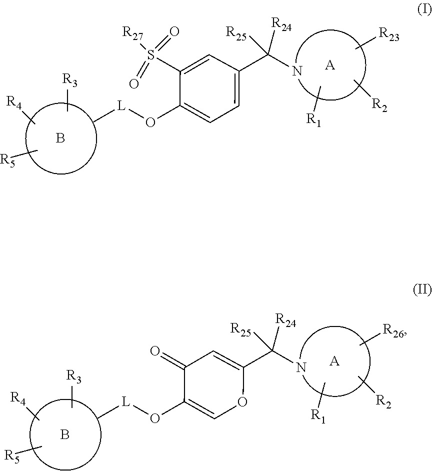

and pharmaceutically acceptable salts thereof. The compounds of formula (I) or (II) possess utility as cytochrome P450 monooxygenase 11A1 (CYP11A1) inhibitors. The compounds are useful as medicaments in the treatment of steroid receptor, particularly androgen receptor, dependent diseases and conditions, such as prostate cancer.

### TECHNICAL FIELD

The present invention relates to therapeutically active compounds useful in the treatment of a steroid receptor, such as androgen receptor (AR), dependent conditions and diseases, and to pharmaceutical compositions containing such compounds.

### BACKGROUND OF THE INVENTION

Prostate cancer is worldwide one of the most common cancers in men. Even though the 5-year survival rate of patients with localized prostate cancer is high, the prognosis for those patients, who develop castration-resistant prostate cancer (CRPC) within that 5-year follow-up period, is poor.

The androgen receptor (AR) signalling axis is critical in all stages of prostate cancer. In the CPRC stage, disease is characterized by high AR expression, AR amplification and persistent activation of the AR signalling axis by residual tissue/tumor androgens and by other steroid hormones and intermediates of steroid biosynthesis. Thus, treatment of advanced prostate cancer involves androgen deprivation therapy (ADT) such as hormonal manipulation using gonadotropin-releasing hormone (GnRH) agonists/antagonists or surgical castration, AR antagonists or CYP17A1 inhibitors (such as abiraterone acetate in combination with prednisone).

Although therapies can initially lead to disease regression, eventually majority of the patients develop a disease that is refractory to currently available therapies. Increased progesterone levels in patients treated with abiraterone acetate has been hypothesized to be one of the resistance mechanisms. Several nonclinical and clinical studies have indicated upregulation of enzymes that catalyse steroid biosynthesis at the late stage of CRPC. Very recently it has been published that 11β-OH androstenedione can be metabolized into 11-ketotestosterone (11-K-T) and 11-ketodehydrotestosterone (11-K-DHT) which can bind and activate AR as efficiently as testosterone and dihydrotestosterone. It has been shown that these steroids are found in high levels in plasma and tissue in prostate cancer patients, suggesting their role as AR agonists in CRPC. Furthermore, it has been addressed that prostate cancer resistance to CYP17A1 inhibition may still remain steroid dependent and responsive to therapies that can further suppress de novo intratumoral steroid synthesis upstream of CYP17A1, such as by CYP11A1 inhibition therapy (Cai, C. et al, Cancer Res., 71(20), 6503-6513, 2011).

Cytochrome P450 monooxygenase 11A1 (CYP11A1), also called cholesterol side chain cleavage enzyme, is a mitochondrial monooxygenase which catalyses the conversion of cholesterol to pregnenolone, the precursor of all steroid hormones. By inhibiting CYP11A1, the key enzyme of steroid biosynthesis upstream of CYP17A1, the total block of the whole steroid biosynthesis can be achieved. CYP11A1 inhibitors may therefore have a great potential for treating steroid hormone dependent cancers, such as prostate cancer, even in advanced stages of the disease, and especially in those patients who appear to be hormone refractory. It has been shown that a compound having CYP11A1 inhibitory effect significantly inhibited tumor growth in vivo in a murine CRPC xenograft model (Oksala, R. et al, Annals of Oncology, (2017) 28 (suppl. 5): Abstract/Poster 28P). CYP11A1 inhibitors have been described earlier in WO 2018/115591.

### SUMMARY OF THE INVENTION

It has been found that compounds of formula (I) or (II) are potent CYP11A1 inhibitors and are associated with lower potential of forming reactive metabolites when studied in vitro, thus having lower risk for idiosyncratic toxicity as drug candidates. The compounds of the invention are therefore particularly useful as medicaments in the treatment of steroid hormone dependent conditions and diseases where CYP11A1 inhibition is desired. Such conditions and diseases include, but are not limited to, endocrine cancers and diseases, such as prostate cancer and breast cancer. In particular, the compounds of the invention are useful in the treatment of AR dependent conditions and diseases including prostate cancer.

The present invention relates to a compound of formula (I) or (II)

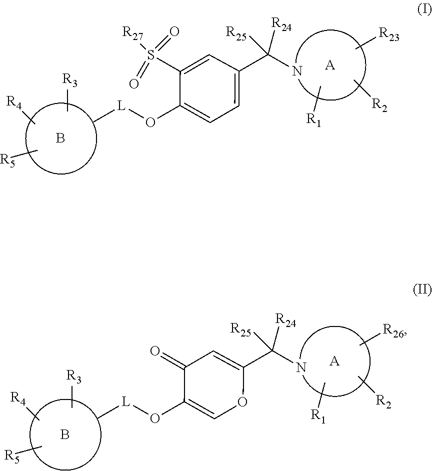

wherein

ring B is a 4-12 membered monocyclic or bicyclic ring or spiro bicyclic ring containing 0-4 heteroatoms independently selected form N, O or S;

ring A is any of the following groups

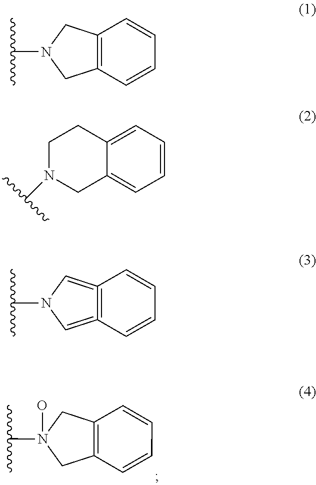

L is absent, —CH₂—, —CH(CH₃)—, —CH₂—CH₂— or —CH₂—CH₂—CH₂—;

R₁ is hydrogen, C₁₋₇ alkyl, C₁₋₇ alkoxy, halogen, cyano, nitro, halo C₁₋₇ alkyl or halo C₁₋₇ alkoxy;

R₂ is hydrogen, C₁₋₇ alkyl, halogen, hydroxy, C₁₋₇ alkoxy, halo C₁₋₇ alkyl or oxo;

or R₁ and R₂ are attached to the same carbon atom and together form, with a carbon atom to which they are attached, a C₃₋₇ cycloalkyl ring;

or R₁ and R₂ together with the carbon atoms to which they are attached form a fused C₃₋₇ cycloalkyl ring;

R₃ is hydrogen, halogen, nitro, cyano, oxo, C₁₋₇ alkyl, C₂₋₇ alkenyl, C₃₋₇ cyclo-alkyl, hydroxy C₃₋₇ cycloalkyl, C₁₋₇ alkoxy, hydroxy C₁₋₇ alkyl, halo C₁₋₇ alkyl, cyano C₁₋₇ alkyl, C₁₋₇ alkoxy C₁₋₇ alkyl, C₁₋₇ alkylthio, aminocarbonyl C₂₋₇ alkenyl, halo C₁₋₇ alkyl-thio, C₁₋₇ alkoxycarbonyl C₁₋₇ alkyl, C₁₋₇ alkoxycarbonyl C₂₋₇ alkenyl, ═NSO₂R₂₀,—S(O)—C₁₋₇ alkyl, —S(O)(NR₁₄)(R₂₂), —S(NR₁₅)(C₁₋₇ alkyl), —C(S)NR₁₈R₁₉, -D-C(O)—NR₆R₇, —C(O)R₈, -D-NR₉R₁₀, —SO₂R₁₁, an optionally substituted 3-10 membered carbocyclyl, optionally substituted 3-10 membered carbocyclyl C₁₋₇ alkyl, optionally substituted 4-10 membered heterocyclyl or optionally substituted 4-10 membered heterocyclyl C₁₋₇ alkyl;

R₄ is hydrogen, halogen, hydroxy, C₁₋₇ alkyl, halo C₁₋₇ alkyl or oxo;

R₅ is hydrogen, halogen or C₁₋₇ alkyl;

R₆ is hydrogen, C₁₋₇ alkyl, C₁₋₇ alkoxy C₁₋₇ alkyl, C₂₋₇ alkenyl, C₃₋₇ cycloalkyl, hydroxy C₁₋₇ alkyl, cyano C₁₋₇ alkyl, —C₁₋₇ alkyl-O—C(O)C₁₋₇ alkyl or optionally substituted 4-10 membered heterocyclyl;

R₈ is hydrogen, C₁₋₇ alkyl, C₂₋₇ alkenyl, C₃₋₇ cycloalkyl, C₁₋₇ alkoxy, halo C₁₋₇ alkyl, C₁₋₇ alkoxy C₁₋₇ alkyl, C₁₋₇ alkylcarbonyl, C₁₋₇ alkoxycarbonyl, —C₁₋₇ alkyl-O—C(O)—C₁₋₇ alkyl, —C₁₋₇ alkyl-SO₂(C₁₋₇ alkyl), —N═S(O)(C₁₋₇ alkyl)(C₁₋₇ alkyl) or optionally substituted 4-10 membered heterocyclyl;

R₉ is hydrogen, C₁₋₇ alkyl, C₃₋₇ cycloalkyl, C₁₋₇ alkylcarbonyl, —SO₂(C₁₋₇ alkyl) or —SO₂(C₃₋₇ cycloalkyl);

R₁₁ is C₁₋₇ alkyl, C₂₋₇ alkenyl, C₃₋₇ cycloalkyl, halo C₁₋₇ alkyl, cyano C₁₋₇ alkyl, C₁₋₇ alkoxy C₁₋₇ alkyl, —NR₁₂R₁₃, optionally substituted 3-10 membered carbocyclyl or optionally substituted 4-10 membered heterocyclyl;

R₁₂ is hydrogen, C₁₋₇ alkyl, hydroxy C₁₋₇ alkyl, cyano C₁₋₇ alkyl, C₁₋₇ alkoxy, C₁₋₇ alkoxy C₁₋₇ alkyl or C₁₋₇ alkylcarbonyl;

R₇, R₁₀, R₁₃, R₁₈, and R₁₉ are, independently, hydrogen, C₁₋₇ alkyl or C₃₋₇ cyclo-alkyl;

R₁₄ is hydrogen, C₁₋₇ alkyl, C₁₋₇ alkylcarbonyl or —SO₂R₂₁;

R₁₅ is hydrogen, C₁₋₇ alkyl, C₃₋₇ cycloalkyl, C₁₋₇ alkylcarbonyl, —SO₂R₁₇;

R₁₇ is C₁₋₇ alkyl or an optionally substituted 3-10 membered carbocyclyl;

R₂₀ and R₂₁ are, independently, C₁₋₇ alkyl, C₃₋₇ cycloalkyl or optionally substituted 3-10 membered carbocyclyl;

R₂₂ is C₁₋₇ alkyl or C₃₋₇ cycloalkyl;

R₂₃ is hydrogen, C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms or optionally substituted 4-10 membered heterocyclyl;

R₂₄ is hydrogen, C₁₋₇ alkyl or halo C₁₋₇ alkyl;

R₂₅ is hydrogen or C₁₋₇ alkyl;

R₂₆ is C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms or an optionally substituted 4-10 membered heterocyclyl, with the proviso than when ring B is a spiro bicyclic ring then R₂₆ can also be hydrogen;

R₂₇ is C₁₋₇ alkyl or C₃₋₇ cycloalkyl;

D is absent, C₁₋₇ alkyl or C₂₋₇ alkenyl;

wherein the optional substitution in each occurrence is selected from 1-3 substituents independently selected from C₁₋₇ alkyl, halogen, hydroxy, cyano, C₁₋₇ alkylcarbonyl, C₁₋₇ alkoxy, C₁₋₇ alkoxy C₁₋₇ alkyl, C₁₋₇ alkoxycarbonyl or oxo; and

wherein the heterocyclyl group in each occurrence has 1-4 heteroatoms independently selected from N, O and S;

or a pharmaceutically acceptable salt thereof.

According to one embodiment, the invention provides a method for the treatment or prevention of a steroid receptor, particularly androgen receptor (AR), dependent conditions and diseases, comprising administering to a subject in need thereof a therapeutically effective amount of a compound of formula (I) or (II)

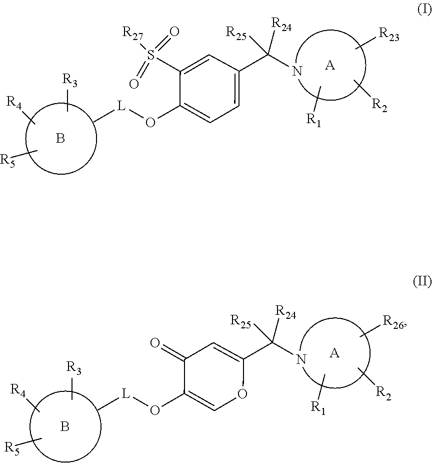

wherein

ring B is a 4-12 membered monocyclic or bicyclic ring or spiro bicyclic ring containing 0-4 heteroatoms independently selected form N, O or S;

ring A is any of the following groups

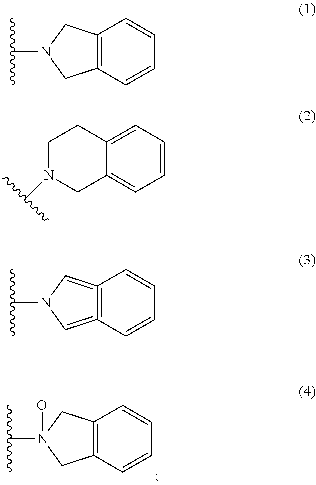

L is absent, —CH₂—, —CH(CH₃)—, —CH₂—CH₂— or —CH₂—CH₂—CH₂—;

R₁ is hydrogen, C₁₋₇ alkyl, C₁₋₇ alkoxy, halogen, cyano, nitro, halo C₁₋₇ alkyl or halo C₁₋₇ alkoxy;

R₂ is hydrogen, C₁₋₇ alkyl, halogen, hydroxy, C₁₋₇ alkoxy, halo C₁₋₇ alkyl or oxo;

or R₁ and R₂ are attached to the same carbon atom and together form, with a carbon atom to which they are attached, a C₃₋₇ cycloalkyl ring;

or R₁ and R₂ together with the carbon atoms to which they are attached form a fused C₃₋₇ cycloalkyl ring;

R₃ is hydrogen, halogen, nitro, cyano, oxo, C₁₋₇ alkyl, C₂₋₇ alkenyl, C₃₋₇ cyclo-alkyl, hydroxy C₃₋₇ cycloalkyl, C₁₋₇ alkoxy, hydroxy C₁₋₇ alkyl, halo C₁₋₇ alkyl, cyano C₁₋₇ alkyl, C₁₋₇ alkoxy C₁₋₇ alkyl, C₁₋₇ alkylthio, aminocarbonyl C₂₋₇ alkenyl, halo C₁₋₇ alkyl-thio, C₁₋₇ alkoxycarbonyl C₁₋₇ alkyl, C₁₋₇ alkoxycarbonyl C₂₋₇ alkenyl, ═NSO₂R₂₀,—S(O)—C₁₋₇ alkyl, —S(O)(NR₁₄)(R₂₂), —S(NR₁₅)(C₁₋₇ alkyl), —C(S)NR₁₈R₁₉, -D-C(O)—NR₆R₇, —C(O)R₈, -D-NR₉R₁₀, —SO₂R₁₁, an optionally substituted 3-10 membered carbocyclyl, optionally substituted 3-10 membered carbocyclyl C₁₋₇ alkyl, optionally substituted 4-10 membered heterocyclyl or optionally substituted 4-10 membered heterocyclyl C₁₋₇ alkyl;

R₄ is hydrogen, halogen, hydroxy, C₁₋₇ alkyl, halo C₁₋₇ alkyl or oxo;

R₅ is hydrogen, halogen or C₁₋₇ alkyl;

R₆ is hydrogen, C₁₋₇ alkyl, C₁₋₇ alkoxy C₁₋₇ alkyl, C₂₋₇ alkenyl, C₃₋₇ cycloalkyl, hydroxy C₁₋₇ alkyl, cyano C₁₋₇ alkyl, —C₁₋₇ alkyl-O—C(O)C₁₋₇ alkyl or optionally substituted 4-10 membered heterocyclyl;

R₈ is hydrogen, C₁₋₇ alkyl, C₂₋₇ alkenyl, C₃₋₇ cycloalkyl, C₁₋₇ alkoxy, halo C₁₋₇ alkyl, C₁₋₇ alkoxy C₁₋₇ alkyl, C₁₋₇ alkylcarbonyl, C₁₋₇ alkoxycarbonyl, —C₁₋₇ alkyl-O—C(O)—C₁₋₇ alkyl, —C₁₋₇ alkyl-SO₂(C₁₋₇ alkyl), —N═S(O)(C₁₋₇ alkyl)(C₁₋₇ alkyl) or optionally substituted 4-10 membered heterocyclyl;

R₉ is hydrogen, C₁₋₇ alkyl, C₃₋₇ cycloalkyl, C₁₋₇ alkylcarbonyl, —SO₂(C₁₋₇ alkyl) or —SO₂(C₃₋₇ cycloalkyl);

R₁₁ is C₁₋₇ alkyl, C₂₋₇ alkenyl, C₃₋₇ cycloalkyl, halo C₁₋₇ alkyl, cyano C₁₋₇ alkyl, C₁₋₇ alkoxy C₁₋₇ alkyl, —NR₁₂R₁₃, optionally substituted 3-10 membered carbocyclyl or optionally substituted 4-10 membered heterocyclyl;

R₁₂ is hydrogen, C₁₋₇ alkyl, hydroxy C₁₋₇ alkyl, cyano C₁₋₇ alkyl, C₁₋₇ alkoxy, C₁₋₇ alkoxy C₁₋₇ alkyl or C₁₋₇ alkylcarbonyl;

R₇, R₁₀, R₁₃, R₁₈, and R₁₉ are, independently, hydrogen, C₁₋₇ alkyl or C₃₋₇ cyclo-alkyl;

R₁₄ is hydrogen, C₁₋₇ alkyl, C₁₋₇ alkylcarbonyl or —SO₂R₂₁;

R₁₅ is hydrogen, C₁₋₇ alkyl, C₃₋₇ cycloalkyl, C₁₋₇ alkylcarbonyl, —SO₂R₁₇;

R₁₇ is C₁₋₇ alkyl or an optionally substituted 3-10 membered carbocyclyl;

R₂₀ and R₂₁ are, independently, C₁₋₇ alkyl, C₃₋₇ cycloalkyl or optionally substituted 3-10 membered carbocyclyl;

R₂₂ is C₁₋₇ alkyl or C₃₋₇ cycloalkyl;

R₂₃ is hydrogen, C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms or optionally substituted 4-10 membered heterocyclyl;

R₂₄ is hydrogen, C₁₋₇ alkyl or halo C₁₋₇ alkyl;

R₂₅ is hydrogen or C₁₋₇ alkyl;

R₂₆ is C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms or an optionally substituted 4-10 membered heterocyclyl, with the proviso than when ring B is a spiro bicyclic ring then R₂₆ can also be hydrogen;

R₂₇ is C₁₋₇ alkyl or C₃₋₇ cycloalkyl;

D is absent, C₁₋₇ alkyl or C₂₋₇ alkenyl;

wherein the optional substitution in each occurrence is selected from 1-3 substituents independently selected from C₁₋₇ alkyl, halogen, hydroxy, cyano, C₁₋₇ alkylcarbonyl, C₁₋₇ alkoxy, C₁₋₇ alkoxy C₁₋₇ alkyl, C₁₋₇ alkoxycarbonyl or oxo; and

wherein the heterocyclyl group in each occurrence has 1-4 heteroatoms independently selected from N, O and S;

or a pharmaceutically acceptable salt thereof.

According to one embodiment, the invention provides a pharmaceutical composition comprising a compound of formula (I) or (II) as defined in any of the above embodiments together with a pharmaceutically acceptable carrier.

According to one embodiment, the invention provides a method for the treatment or prevention of a steroid receptor, in particular androgen receptor (AR), dependent conditions and diseases. Such conditions and diseases include, but are not limited to, endocrine cancers and diseases, such as prostate cancer and breast cancer. The method comprises administering to a subject in need thereof a therapeutically effective amount of a compound of formula (I) or (II) as defined in any of the above embodiments.

### DETAILED DESCRIPTION OF THE INVENTION

The present application provides novel compounds of formula (I) or (II) or pharmaceutically acceptable salts thereof which are useful as CYP11A1 inhibitors.

One of the embodiments of the present invention provides a compound of formula (I) or (II)

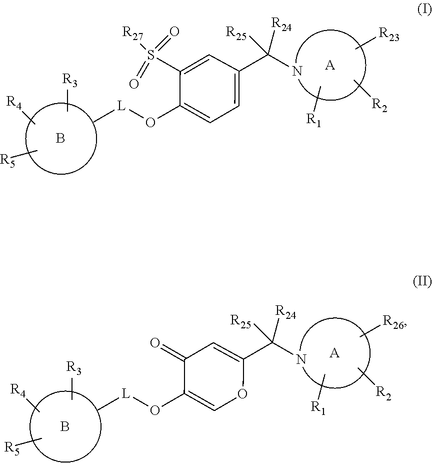

wherein

ring B is a 4-12 membered monocyclic or bicyclic ring or spiro bicyclic ring containing 0-4 heteroatoms independently selected form N, O or S;

ring A is any of the following groups

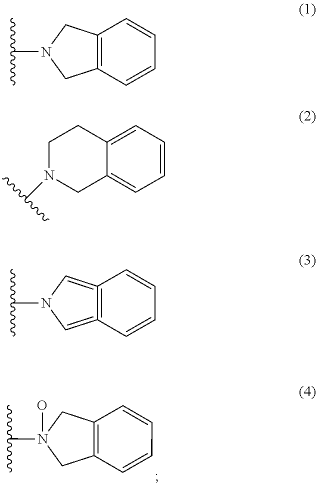

L is absent, —CH₂—, —CH(CH₃)—, —CH₂—CH₂— or —CH₂—CH₂—CH₂—;

R₁ is hydrogen, C₁₋₇ alkyl, C₁₋₇ alkoxy, halogen, cyano, nitro, halo C₁₋₇ alkyl or halo C₁₋₇ alkoxy;

R₂ is hydrogen, C₁₋₇ alkyl, halogen, hydroxy, C₁₋₇ alkoxy, halo C₁₋₇ alkyl or oxo;

or R₁ and R₂ are attached to the same carbon atom and together form, with a carbon atom to which they are attached, a C₃₋₇ cycloalkyl ring;

or R₁ and R₂ together with the carbon atoms to which they are attached form a fused C₃₋₇ cycloalkyl ring;

R₃ is hydrogen, halogen, nitro, cyano, oxo, C₁₋₇ alkyl, C₂₋₇ alkenyl, C₃₋₇ cyclo-alkyl, hydroxy C₃₋₇ cycloalkyl, C₁₋₇ alkoxy, hydroxy C₁₋₇ alkyl, halo C₁₋₇ alkyl, cyano C₁₋₇ alkyl, C₁₋₇ alkoxy C₁₋₇ alkyl, C₁₋₇ alkylthio, aminocarbonyl C₂₋₇ alkenyl, halo C₁₋₇ alkyl-thio, C₁₋₇ alkoxycarbonyl C₁₋₇ alkyl, C₁₋₇ alkoxycarbonyl C₂₋₇ alkenyl, ═NSO₂R₂₀,—S(O)—C₁₋₇ alkyl, —S(O)(NR₁₄)(R₂₂), —S(NR₁₅)(C₁₋₇ alkyl), —C(S)NR₁₈R₁₉, -D-C(O)—NR₆R₇, —C(O)R₈, -D-NR₉R₁₀, —SO₂R₁₁, an optionally substituted 3-10 membered carbocyclyl, optionally substituted 3-10 membered carbocyclyl C₁₋₇ alkyl, optionally substituted 4-10 membered heterocyclyl or optionally substituted 4-10 membered heterocyclyl C₁₋₇ alkyl;

R₄ is hydrogen, halogen, hydroxy, C₁₋₇ alkyl, halo C₁₋₇ alkyl or oxo;

R₅ is hydrogen, halogen or C₁₋₇ alkyl;

R₆ is hydrogen, C₁₋₇ alkyl, C₁₋₇ alkoxy C₁₋₇ alkyl, C₂₋₇ alkenyl, C₃₋₇ cycloalkyl, hydroxy C₁₋₇ alkyl, cyano C₁₋₇ alkyl, —C₁₋₇ alkyl-O—C(O)C₁₋₇ alkyl or optionally substituted 4-10 membered heterocyclyl;

R₈ is hydrogen, C₁₋₇ alkyl, C₂₋₇ alkenyl, C₃₋₇ cycloalkyl, C₁₋₇ alkoxy, halo C₁₋₇ alkyl, C₁₋₇ alkoxy C₁₋₇ alkyl, C₁₋₇ alkylcarbonyl, C₁₋₇ alkoxycarbonyl, —C₁₋₇ alkyl-O—C(O)—C₁₋₇ alkyl, —C₁₋₇ alkyl-SO₂(C₁₋₇ alkyl), —N═S(O)(C₁₋₇ alkyl)(C₁₋₇ alkyl) or optionally substituted 4-10 membered heterocyclyl;

R₉ is hydrogen, C₁₋₇ alkyl, C₃₋₇ cycloalkyl, C₁₋₇ alkylcarbonyl, —SO₂(C₁₋₇ alkyl) or —SO₂(C₃₋₇ cycloalkyl);

R₁₁ is C₁₋₇ alkyl, C₂₋₇ alkenyl, C₃₋₇ cycloalkyl, halo C₁₋₇ alkyl, cyano C₁₋₇ alkyl, C₁₋₇ alkoxy C₁₋₇ alkyl, —NR₁₂R₁₃, optionally substituted 3-10 membered carbocyclyl or optionally substituted 4-10 membered heterocyclyl;

R₁₂ is hydrogen, C₁₋₇ alkyl, hydroxy C₁₋₇ alkyl, cyano C₁₋₇ alkyl, C₁₋₇ alkoxy, C₁₋₇ alkoxy C₁₋₇ alkyl or C₁₋₇ alkylcarbonyl;

R₇, R₁₀, R₁₃, R₁₈, and R₁₉ are, independently, hydrogen, C₁₋₇ alkyl or C₃₋₇ cyclo-alkyl;

R₁₄ is hydrogen, C₁₋₇ alkyl, C₁₋₇ alkylcarbonyl or —SO₂R₂₁;

R₁₅ is hydrogen, C₁₋₇ alkyl, C₃₋₇ cycloalkyl, C₁₋₇ alkylcarbonyl, —SO₂R₁₇;

R₁₇ is C₁₋₇ alkyl or an optionally substituted 3-10 membered carbocyclyl;

R₂₀ and R₂₁ are, independently, C₁₋₇ alkyl, C₃₋₇ cycloalkyl or optionally substituted 3-10 membered carbocyclyl;

R₂₂ is C₁₋₇ alkyl or C₃₋₇ cycloalkyl;

R₂₃ is hydrogen, C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms or optionally substituted 4-10 membered heterocyclyl;

R₂₄ is hydrogen, C₁₋₇ alkyl or halo C₁₋₇ alkyl;

R₂₅ is hydrogen or C₁₋₇ alkyl;

R₂₆ is C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms or an optionally substituted 4-10 membered heterocyclyl, with the proviso than when ring B is a spiro bicyclic ring then R₂₆ can also be hydrogen;

R₂₇ is C₁₋₇ alkyl or C₃₋₇ cycloalkyl;

D is absent, C₁₋₇ alkyl or C₂₋₇ alkenyl;

wherein the optional substitution in each occurrence is selected from 1-3 substituents independently selected from C₁₋₇ alkyl, halogen, hydroxy, cyano, C₁₋₇ alkylcarbonyl, C₁₋₇ alkoxy, C₁₋₇ alkoxy C₁₋₇ alkyl, C₁₋₇ alkoxycarbonyl or oxo; and

wherein the heterocyclyl group in each occurrence has 1-4 heteroatoms independently selected from N, O and S;

or a pharmaceutically acceptable salt thereof.

It is to be understood that the left bond in linker L is attached to the ring B of formula (I) or (II).

According to one embodiment, specifically provided is a compound according to any of the above embodiments of formula (I) or (II) wherein L is absent, —CH₂— or —CH(CH₃)—, for example L is absent, or as another example L is —CH₂—, or as another example L is —CH(CH₃)—.

According to one embodiment, specifically provided is a compound according to any of the above embodiments of formula (I) or (II), wherein ring B is any one of the following groups

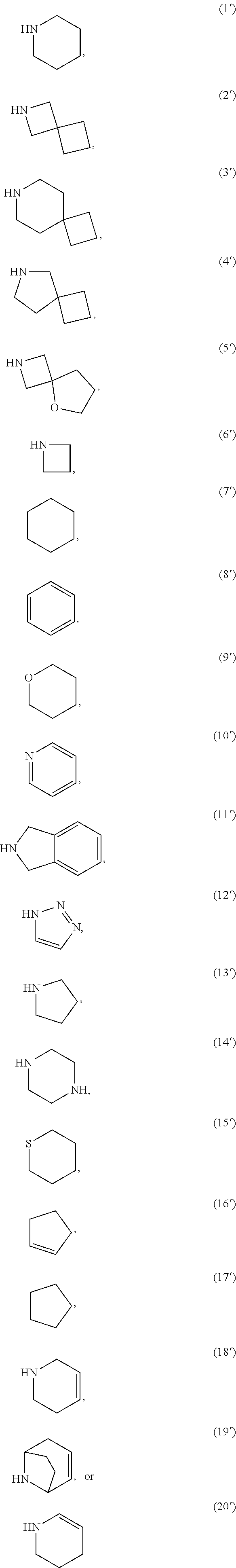

R₃, R₄ and R₅ being attached to the above B-rings.

In a subclass of the above embodiment are compounds wherein ring B is any one of the following groups

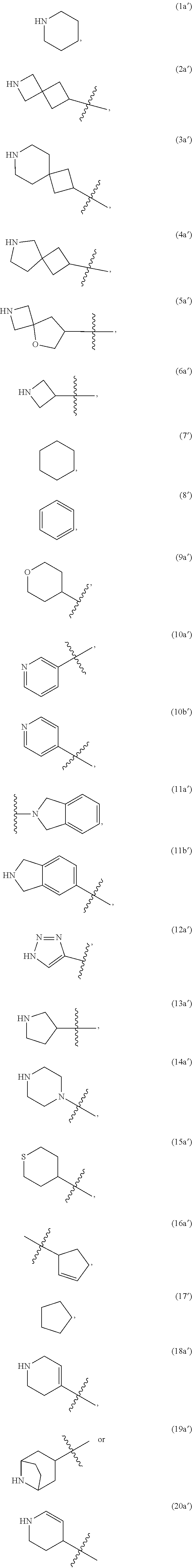

R₃, R₄ and R₅ being attached to the above B-rings and the wavy line denoting the site of attachment to L.

According to yet one embodiment, specifically provided are compounds according to any of the above embodiments wherein ring B is (1′), (2′), (3′), (4′), (5′), (6′), (7′), (8′) or (9′).

In a subclass of the above embodiment are compounds according to any of the above embodiments wherein ring B is (1a′), (2a′), (3a′), (4a′), (5a′), (6a′), (7′), (8′) or (9a′).

According to one embodiment, specifically provided are compounds according to any of the above embodiments, wherein R₃ is hydrogen, cyano, C₁₋₇ alkyl, C₂₋₇ alkenyl, hydroxy C₁₋₇ alkyl, C₁₋₇ alkylthio, C₁₋₇ alkoxycarbonyl C₁₋₇ alkyl, —S(O)(NR₁₄)(R₂₂), —S(NR₁₅)(C₁₋₇ alkyl), —C(S)NR₁₈R₁₉, -D-C(O)—NR₆R₇, —C(O)R₈, -D-NR₉R₁₀, —SO₂R₁₁, an optionally substituted 3-10 membered carbocyclyl, optionally substituted 3-10 membered carbocyclyl C₁₋₇ alkyl, optionally substituted 4-10 membered heterocyclyl or an optionally substituted 4-10 membered heterocyclyl C₁₋₇ alkyl; wherein the optional substitution in each occurrence is selected from 1-3 substituents independently selected from C₁₋₇ alkyl, halogen, hydroxy, cyano, C₁₋₇ alkylcarbonyl, C₁₋₇ alkoxy, C₁₋₇ alkoxy C₁₋₇ alkyl, C₁₋₇ alkoxycarbonyl or oxo; and wherein the heterocyclyl group in each occurrence has 1-4 heteroatoms independently selected from N, O and S.

In a subclass of the above embodiment are compounds, wherein R₃ is hydrogen, cyano, hydroxy C₁₋₇ alkyl, —S(O)(NR₁₄)(R₂₂), -D-C(O)—NR₆R₇, —C(O)R₈, —SO₂R₁₁, optionally substituted 4-10 membered heterocyclyl or an optionally substituted 4-10 membered heterocyclyl C₁₋₇ alkyl; wherein the optional substitution in each occurrence is selected from 1-3 substituents independently selected from C₁₋₇ alkyl, halogen, hydroxy, cyano, C₁₋₇ alkylcarbonyl and wherein the heterocyclyl group in each occurrence has 1-4 heteroatoms independently selected from N, O and S. In a further subclass of the above embodiment are compounds wherein R₁₄ is hydrogen, R₂₂ is C₁₋₇ alkyl, D is absent, R₆ is C₁₋₇ alkyl or C₁₋₇ alkoxy C₁₋₇ alkyl, R₇ is C₁₋₇ alkyl, R₈ is C₁₋₇ alkyl, C₁₋₇ alkoxy or optionally substituted 4-10 membered heterocyclyl and/or R₁₁ is C₁₋₇ alkyl, an optionally substituted 3-10 membered carbocyclyl or an optionally substituted 4-10 membered heterocyclyl. In the definition of R₃, R₈ and R₁₁, particular examples of 4-10 membered heterocyclyl are pyridinyl, pyrazolyl, azetidinyl and pyrimidinyl rings, and a particular example of 3-10 membered carbocyclyl is phenyl ring.

According to one embodiment, specifically provided are compounds according to any of the above embodiments, wherein R₁ is hydrogen, C₁₋₇ alkyl, halogen, cyano or halo C₁₋₇ alkyl; R₂ is hydrogen or C₁₋₇ alkyl; or R₁ and R₂ together with the carbon atom to which they are attached form a spiro C₃₋₇ cycloalkyl ring; or R₁ and R₂ together with the carbon atoms to which they are attached form a fused C₃₋₇ cycloalkyl ring.

According to one embodiment, specifically provided are compounds according to any of the above embodiments, wherein R₄ is hydrogen or halogen, and R₅ is hydrogen.

In a subclass of the above embodiment are compounds, wherein R₄ and R₅ are hydrogen.

According to one embodiment, specifically provided are compounds according to any of the above embodiments, wherein ring A is (1) or (2).

According to one embodiment, specifically provided are compounds according to any of the above embodiments, wherein R₂₄ is C₁₋₇ alkyl or halo C₁₋₇ alkyl.

According to one embodiment, the compound according to the present invention is represented by formula (IA):

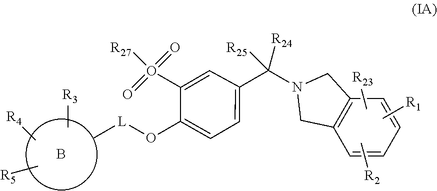

wherein R₁, R₂, R₃, R₄, R₅, R₂₃, R₂₄, R₂₅, R₂₇, L and B are as defined in any of the above embodiments for formula (I) or (II), or a pharmaceutically acceptable salt thereof.

According to one embodiment, the compound according to the present invention is represented by formula (IB):

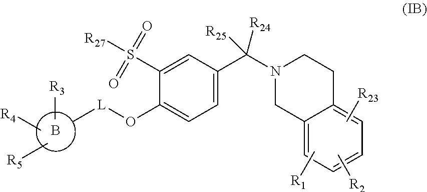

wherein R₁, R₂, R₃, R₄, R₅, R₂₃, R₂₄, R₂₅, R₂₇, L and B are as defined in any of the above embodiments for formula (I) or (II), or a pharmaceutically acceptable salt thereof.

According to one embodiment, the compound according to the present invention is represented by formula (IC):

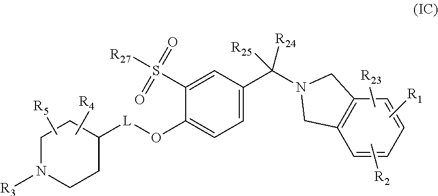

wherein R₁, R₂, R₃, R₄, R₅, R₂₃, R₂₄, R₂₅, R₂₇ and L are as defined in any of the above embodiments for formula (I) or (II), or a pharmaceutically acceptable salt thereof.

According to one embodiment, the compound according to the present invention is represented by formula (IIA):

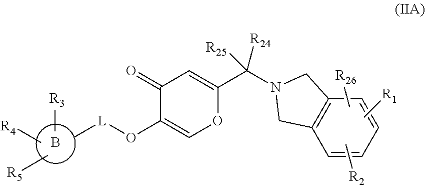

wherein R₁, R₂, R₃, R₄, R₅, R₂₄, R₂₅, R₂₆, L and B are as defined in any of the above embodiments for formula (I) or (II), or a pharmaceutically acceptable salt thereof.

According to one embodiment, the compound according to the present invention is represented by formula (IIB):

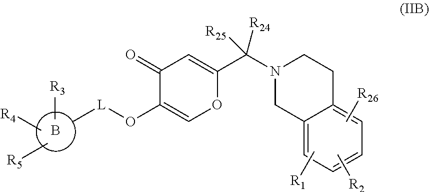

wherein R₁, R₂, R₃, R₄, R₅, R₂₄, R₂₅, R₂₆, L and B are as defined in any of the above embodiments for formula (I) or (II), or a pharmaceutically acceptable salt thereof.

According to one embodiment, the compound according to the present invention is represented by formula (IIC):

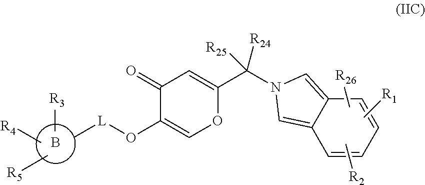

wherein R₁, R₂, R₃, R₄, R₅, R₂₄, R₂₅, R₂₆, L and B are as defined in any of the above embodiments for formula (I) or (II), or a pharmaceutically acceptable salt thereof.

According to one embodiment, the compound according to the present invention is represented by formula (IID):

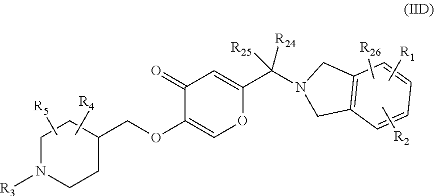

wherein R₁, R₂, R₃, R₄, R₅, R₂₄, R₂₅ and R₂₆ are as defined in any of the above embodiments for formula (I) or (II), or a pharmaceutically acceptable salt thereof.

According to one embodiment, the compound according to the present invention is represented by formula (IIE):

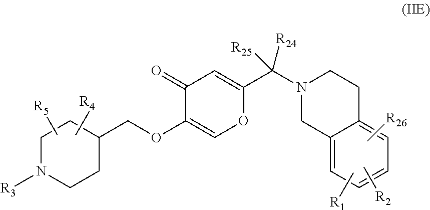

wherein R₁, R₂, R₃, R₄, R₅, R₂₄, R₂₅ and R₂₆ are as defined in any of the above embodiments for formula (I) or (II), or a pharmaceutically acceptable salt thereof.

According to one embodiment, specifically provided are compounds as defined in any of the above embodiments, wherein R₂₆ is an optionally substituted 4-10 membered heterocyclyl having 1-4 heteroatoms independently selected from N, O and S. In the definition of R₂₆, particular examples of 4-10 membered heterocyclyl are oxadiazolyl, oxazolyl, isoxazolyl, thiadiazolyl and pyrazolyl rings, and a particular example of 3-10 membered carbocyclyl is phenyl ring.

According to one embodiment, specifically provided are compounds as defined in any of the above embodiments, wherein R₃ is —SO₂R₁₁; R₁, R₂, R₄, and R₅ are hydrogen; and R₁₁ is C₁₋₇ alkyl.

According to one embodiment, specifically provided are compounds as defined in any of the above embodiments, wherein R₂₄ is C₁₋₇ alkyl or halo C₁₋₇ alkyl.

According to one embodiment, specifically provided are compounds as defined in any of the above embodiments, wherein R₃ is an optionally substituted 4-10 membered heterocyclyl C₁₋₇ alkyl; wherein the optional substitution is selected from 1-3 substituents independently selected from C₁₋₇ alkyl, halogen, hydroxy, cyano, C₁₋₇ alkylcarbonyl and wherein the heterocyclyl group has 1-4 heteroatoms independently selected from N, O and S; and R₁, R₂, R₄, and R₅ are hydrogen.

According to one embodiment, specifically provided are compounds as defined in any of the above embodiments, wherein R₂₃ is an optionally substituted 4-10 membered heterocyclyl having 1-4 heteroatoms independently selected from N, O and S. In the definition of R₂₃, particular examples of 4-10 membered heterocyclyl are oxadiazolyl, oxazolyl, isoxazolyl, thiadiazolyl, thiazolyl and pyrazolyl rings, and a particular example of 3-10 membered carbocyclyl is phenyl ring.

According to one embodiment, specifically provided are compounds as defined in any of the above embodiments, wherein ring B is (2a′), (3a′), (4a′), (5a) or (6a′), wherein R₁, R₂, R₃, R₄, R₅, R₂₄, R₂₅, R₂₆ and L are as defined in claim 1, or a pharmaceutically acceptable salt thereof.

According to one embodiment, specifically provided are compounds as defined in any of the above embodiments, wherein

R₃ is hydrogen, —C(O)R₈, —SO₂R₁₁, an optionally substituted 4-10 membered heterocyclyl, or an optionally substituted 4-10 membered heterocyclyl C₁₋₇ alkyl;

R₈ is C₁₋₇ alkoxy;

R₁₁ is C₁₋₇ alkyl, an optionally substituted 4-10 membered heterocyclyl or an optionally substituted 3-10 membered carbocyclyl;

wherein the optional substitution in each occurrence is selected from 1-3 substituents independently selected from C₁₋₇ alkyl, halogen, hydroxy, cyano, C₁₋₇ alkylcarbonyl and wherein the heterocyclyl group in each occurrence has 1-4 heteroatoms independently selected from N, O and S.

According to one embodiment, the invention provides a pharmaceutical composition comprising a compound of formula (I) or (II) as defined in any of the above embodiments together with a pharmaceutically acceptable carrier.

According to still one embodiment, the present invention provides a method for the treatment of a steroid receptor, in particular androgen receptor (AR), dependent conditions and diseases, comprising administering to a subject in need thereof a therapeutically effective amount of a compound of formula (I) or (II) as defined in any of the above embodiments.

According to one embodiment, the steroid receptor dependent disease or condition is androgen receptor dependent disease or condition including endocrine cancers and diseases, for example prostate cancer or breast cancer, particularly castration-resistant prostate cancer (CRPC). According to one embodiment of the invention, the CRPC to be treated is refractory to CYP17A1 inhibitor treatment. According to another embodiment, the androgen receptor dependent disease or condition is endocrine cancer which is dependent upon CYP11A1 activation.

The compounds of the invention can be prepared by a variety of synthetic routes analogously to the methods known in the literature using suitable starting materials. The compounds according to formula (I) or (II) can be prepared e.g. analogously or according to the following reaction Schemes. Some compounds included in the formula (I) or (II) can be obtained by converting the functional groups of the other compounds of formula (I) or (II) obtained in accordance with the following Schemes, by well known reaction steps such as oxidation, reduction, hydrolysis, acylation, alkylation, amidation, amination, sulfonation and others. It should be noted that any appropriate leaving groups, e.g. N-protecting groups, such as a t-butoxycarbonyl (t-BOC) group or a phenylsulfonyl group, can be used in well known manner during the syntheses in order to improve the selectivity of the reaction steps.

Compounds of formula (I) can be prepared according to Scheme 1, wherein R₁, R₂, R₃, R₄, R₅, R₂₃, R₂₄, R₂₅, R₂₇, L, A and B, are as defined above are as defined above, and X is a halogen. In the method of Scheme 1, the compound of formula [1] is coupled with a compound of formula [2] in a suitable solvent such as CH₃CN in the presence of a base such as DIPEA at elevated temperature to produce a compound of formula [I].

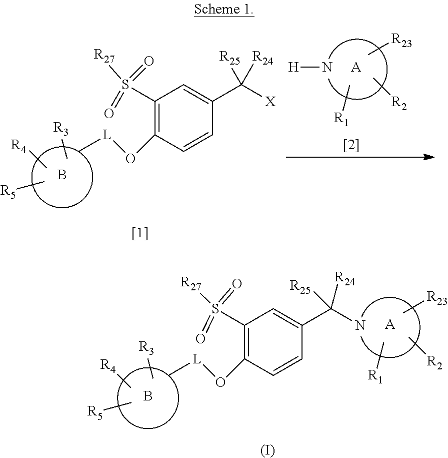

Alternatively, compounds of formula (I) can be prepared according to Scheme 2, wherein R₁, R₂, R₃, R₄, R₅, R₂₃, R₂₄, R₂₅, R₂₇, L, A and B, are as defined above are as defined above, and X is a halogen. In the method of Scheme 2, the compound of formula [3] is coupled with a NaSO₂R₂₇ in a suitable solvent such as DMSO in the presence of a base such as K₂CO₃ at elevated temperature to produce a compound of formula [I].

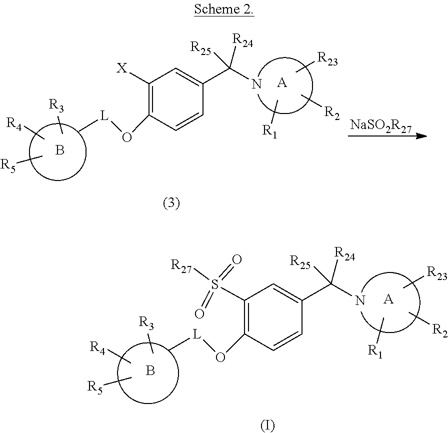

Compounds of formula (I), wherein R₂₄ and R₂₅ are hydrogen, can be prepared according to Scheme 3, wherein R₁, R₂, R₃, R₄, R₅, R₂₃, R₂₇, L, A and B, are as defined above are as defined above. In the method of Scheme 3, the compound of formula [4] is coupled by reductive amination with a compound of formula [2] in a suitable solvent such as DCM in the presence of acetic acid to produce a compound of formula [Ia].

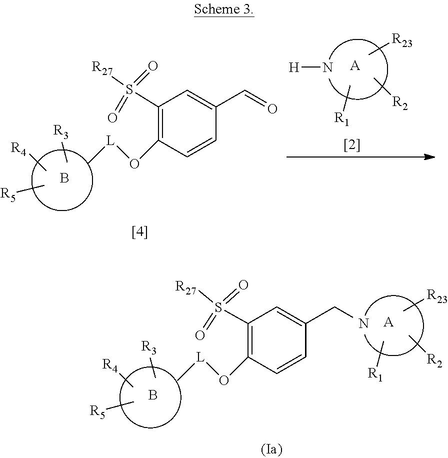

Compounds of formula (II) can be prepared according to Scheme 4, wherein R₁, R₂, R₃, R₄, R₅, R₂₄, R₂₅, R₂₆, L, A and B, are as defined above are as defined above, and X is a halogen. In the method of Scheme 4, the compound of formula [5] is coupled with a TFA or HCl salt of the compound of formula [6] in a suitable solvent such as CH₃CN in the presence of a base such as DIPEA at elevated temperature to produce a compound of formula [II].

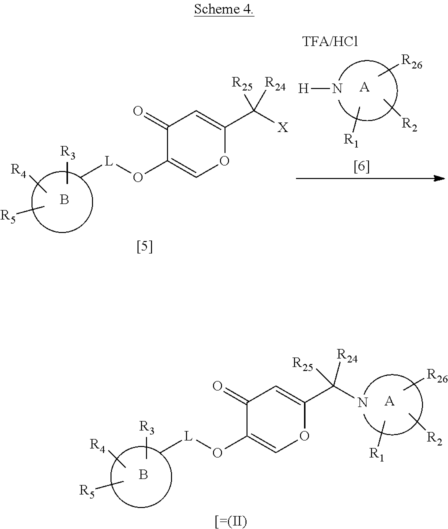

Alternatively, compounds of formula (II) can be prepared according to Scheme 5, wherein R₁, R₂, R₃, R₄, R₅, R₂₄, R₂₅, R₂₆, L, A and B are as defined above, and Z is mesyl or tosyl group. In the method of Scheme 5, the 5-hydroxy-4H-pyran-4-one derivative [7] is coupled with ring B derivative [8], where mesylate or tosylate is acting as the leaving group, in a suitable solvent in the presence of a base at elevated temperature, for example using Cs₂CO₃ or K₂CO₃ in DMSO to produce a compound of formula (II).

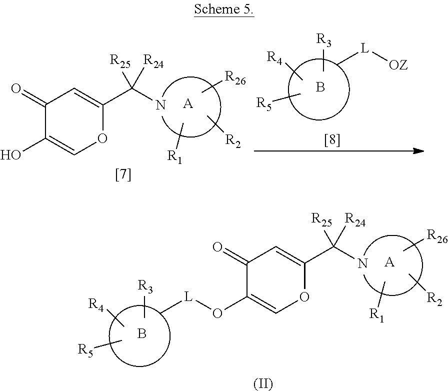

Alternatively, compounds of formula (II) can be prepared according to Scheme 6, wherein R₁, R₂, R₃, R₄, R₅, R₂₄, R₂₅, R₂₆, L, A and B are as defined above, and Z is mesyl or tosyl group. In the method of Scheme 6, the compound [9] is coupled with ring A derivative [10], where mesylate or tosylate is acting as the leaving group, in a suitable solvent in the presence of a base at elevated temperature, for example using Et₃N in THF and water to produce a compound of formula (II).

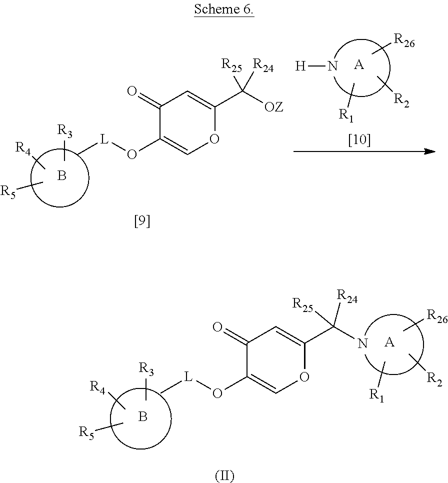

Compounds of formula (II) can be also prepared, for example, according to Scheme 7, wherein R₁, R₂, R₃, R₄, R₅, R₂₄, R₂₅, R₂₆, L, A and B, are as defined above, and X is a halogen. In the method of Scheme 7, the 5-hydroxy-4H-pyran-4-one derivative [7] is coupled with ring B derivative [11], where halogen is acting as the leaving group, in a suitable solvent in the presence of a base at elevated temperature, for example using K₂CO₃ in DMF to produce a compound of formula (II).

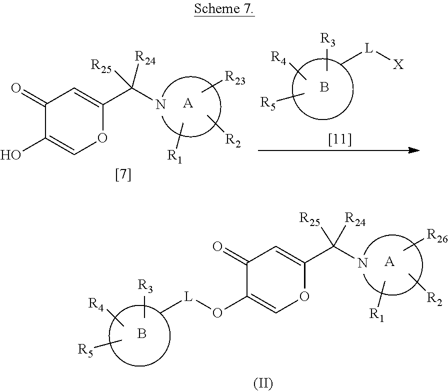

Compounds of formula (II) can be also prepared according to Scheme 8, wherein R₁, R₂, R₃, R₄, R₅, R₂₄, R₂₅, R₂₆, L, A and B are as defined above. In the method of Scheme 8, the 5-hydroxy-4H-pyran-4-one derivative [7] is coupled with ring B derivative [12], in a suitable solvent such as THF in the presence of triphenylphosphine and DIAD to produce a compound of formula (II).

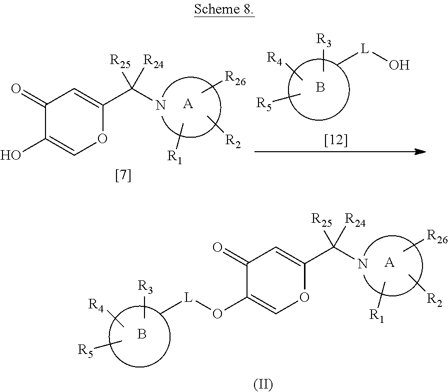

Alternatively, the compounds of formula (I) or (II) can be prepared as disclosed in the specific Examples of the present disclosure.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “subject”, as employed herein, refers to humans and animals.

The term “steroid receptor” refers to receptor which binds to and is activated by a steroid hormone. Examples of steroid receptors include, but are not limited to, androgen, glucocorticoid, and progesterone receptors.

The term “endocrine cancer” refers to partially or completely unregulated growth of one or more cellular components of the endocrine system, including, but not limited to, cancers of one or more of the adrenal glands.

The term “halo” or “halogen”, as employed herein as such or as part of another group, refers to chlorine, bromine, fluorine or iodine.

The term “C₁₋₇ alkyl”, as employed herein as such or as part of another group, refers to a straight or branched chain saturated hydrocarbon group having 1, 2, 3, 4, 5, 6 or 7 carbon atom(s). Representative examples of C₁₋₇ alkyl include, but are not limited to, methyl, ethyl, n-propyl, iso-propyl, n-butyl, iso-butyl, sec-butyl, tert-butyl, n-pentyl, iso-pentyl and n-hexyl. One preferred embodiment of “C₁₋₇ alkyl” is C₁₋₃ alkyl. The term “C₁₋₃ alkyl” refers to a preferred embodiment of “C₁₋₇ alkyl” having 1, 2 or 3 carbon atoms.

The term “C₂₋₇ alkenyl”, as employed herein as such or as part of another group, refers to an aliphatic hydrocarbon group having 2, 3, 4, 5, 6 or 7 carbon atoms and containing one or several double bonds. Representative examples include, but are not limited to, ethenyl, propenyl and cyclohexenyl.

The term “C₃₋₇ cycloalkyl”, as employed herein as such or as part of another group, refers to a saturated cyclic hydrocarbon group containing 3, 4, 5, 6 or 7 carbon atoms. Representative examples of cycloalkyl include, but are not limited to, cyclo-propyl, cyclobutyl, cyclopentyl and cyclohexyl.

The term “C₃₋₇ cycloalkyl C₁₋₇ alkyl”, as employed herein refers to a C₃₋₇ cyclo-alkyl group, as defined herein, appended to the parent molecular moiety through a C₁₋₇ alkyl group, as defined herein.

The term “hydroxy”, as employed herein as such or as part of another group, refers to an —OH group.

The term “cyano”, as employed herein as such or as part of another group, refers to a —CN group.

The term “carboxy”, as employed herein as such or as part of another group, refers to —COOH group.

The term “carbonyl”, as employed herein as such or as part of another group, refers to a carbon atom double-bonded to an oxygen atom (C═O).

The term “oxo”, as employed herein as such or as part of another group, refers to oxygen atom linked to another atom by a double bond (═O).

The term “C₁₋₇ alkoxy”, as employed herein as such or as part of another group, refers to C₁₋₇ alkyl, as defined herein, appended to the parent molecular moiety through an oxygen atom. Representative examples of C₁₋₇ alkoxy include, but are not limited to methoxy, ethoxy, propoxy, butoxy, isobutoxy, sec-butoxy and tert-butoxy.

The term “hydroxy C₁₋₇ alkyl”, as employed herein, refers to at least one hydroxy group, as defined herein, appended to the parent molecular moiety through a C₁₋₇ alkyl group, as defined herein. Representative examples of hydroxy C₁₋₇ alkyl include, but are not limited to, hydroxymethyl, 2,2-dihydroxyethyl, 1-hydroxyethyl, 3-hydroxypropyl, 1-hydroxypropyl, 1-methyl-1-hydroxyethyl and 1-methyl-1-hydroxypropyl.

The term “hydroxy C₃₋₇ cycloalkyl”, as employed herein, refers to at least one hydroxy group, as defined herein, appended to the parent molecular moiety through a C₃₋₇ cycloalkyl group, as defined herein. Representative examples of hydroxy C₁₋₇ alkyl include, but are not limited to, hydroxycyclopropyl, hydroxycyclobutyl, hydroxycyclo-pentyl, hydroxycyclohexyl and 2,2-dihydroxycyclopropyl.

The term “halo C₁₋₇ alkyl”, as employed herein, refers to at least one halogen, as defined herein, appended to the parent molecular moiety through a C₁₋₇ alkyl group, as defined herein. Representative examples of halo C₁₋₇ alkyl include, but are not limited to, fluoromethyl, difluoromethyl, trifluoromethyl, 2-chloroethyl and 3-bromopropyl.

The term “cyano C₁₋₇ alkyl”, as employed herein, refers to a cyano group, as defined herein, appended to the parent molecular moiety through a C₁₋₇ alkyl group, as defined herein. Representative examples of cyano C₁₋₇ alkyl include, but are not limited to, cyanomethyl, 1-cyanoethyl, 1-cyanopropyl and 2-cyanopropyl.

The term “halo C₁₋₇ alkoxy”, as employed herein, refers to at least one halogen, as defined herein, appended to the parent molecular moiety through a C₁₋₇ alkoxy group, as defined herein.

The term “phenyl C₁₋₇ alkyl”, as employed herein, refers to at least one phenyl group appended to the parent molecular moiety through a C₁₋₇ alkyl group, as defined herein.

The term “C₁₋₇ alkyl carbonyl”, as employed herein as such or as part of another group, refers to a C₁₋₇ alkyl group, as defined herein, appended to the parent molecular moiety through a carbonyl group, as defined herein.

The term “C₁₋₇ alkoxy C₁₋₇ alkyl”, as employed herein as such or as part of another group, refers to at least one C₁₋₇ alkoxy group, as defined herein, appended to the parent molecular moiety through a C₁₋₇ alkyl group, as defined herein.

The term “hydroxy C₁₋₇ alkoxy”, as employed herein such or as part of another group, refers to at least one hydroxy group, as defined herein, appended to the parent molecular moiety through an C₁₋₇ alkoxy group, as defined herein.

The term “hydroxy C₁₋₇ alkoxy C₁₋₇ alkyl”, as employed herein, refers to a hydroxy C₁₋₇ alkoxy group, as defined herein, appended to the parent molecular moiety through a C₁₋₇ alkyl group, as defined herein.

The term “4-10 membered heterocyclyl” as employed herein, refers to a saturated, partially saturated or aromatic ring with 4-10 ring atoms, of which 1-4 atoms are heteroatoms selected from a group consisting of N, O and S. One embodiment of a “4-10 membered heterocyclyl” is a “4-6 membered heterocyclyl” which refers to a saturated, partially saturated or aromatic ring with 4-6 ring atoms, of which 1-4 atoms are heteroatoms selected from a group consisting of N, O and S. Representative examples of a 4-10 membered heterocyclic ring include, but are not limited to, oxetanyl, azetidinyl, pyrazolyl, 1,2,4-triazol-1-yl, 1,2,3-triazol-1-yl, pyrimidinyl, pyridinyl, piperidinyl, tetrazolyl, piperazinyl, furanyl, morpholinyl, piperidinyl, pyrrolidinyl, thiazolyl, isoxazolyl, pyrazinyl tetrahydropyranyl, 1,2,4-oxadiazolyl, oxazolyl, imidazolyl, indolyl and 4,5-dihydroimidazolyl rings.

The term “3-10 membered carbocyclyl”, as employed herein, refers to a saturated, partially saturated or aromatic ring with 3 to 10 ring atoms consisting of carbon atoms only. One embodiment of a “3-10 membered carbocyclyl” is a “3-6 membered carbocyclyl” which refers to a saturated, partially saturated or aromatic ring with 3 to 6 ring atoms consisting of carbon atoms only. Representative examples of a 3-10 membered carbocyclic ring include, but are not limited to, phenyl, cyclohexyl, cyclohexenyl, cyclopentyl, cyclopentenyl and cyclobutyl rings.

The term “4-12 membered monocyclic, bicyclic or spiro bicyclic ring”, as employed herein, refers to a saturated, partially saturated or aromatic monocyclic or bicyclic (fused, bridged or in spiro configuration) ring system with 4 to 12 ring atoms.

The term “spiro bicyclic ring”, as employed herein, refers to a bicyclic ring where the two rings are both joined at the same carbon.

The term “4-12 membered bicyclic ring”, as employed herein, refers to a saturated, partially saturated or aromatic ring system where the two rings have two common ring atoms.

The term “4-10 membered heterocyclyl C₁₋₇ alkyl” as employed herein, refers to a “4-10 membered heterocyclyl” group, as defined herein, appended to the parent molecular moiety through a C₁₋₇ alkyl group, as defined herein.

The term “3-10 membered carbocyclyl C₁₋₇ alkyl” as employed herein, refers to a “3-10 membered carbocyclyl” group, as defined herein, appended to the parent molecular moiety through a C₁₋₇ alkyl group, as defined herein.

The term “substituted” as used herein in connection with various residues refers to, if not otherwise defined, to halogen substituents, such as fluorine, chlorine, bromine, iodine, or C₁₋₇ alkyl, C₃₋₇ cycloalkyl, hydroxy, amino, nitro, cyano, thiol C₁₋₇ alkyl, methylsulfonyl, C₁₋₇ alkoxy, halo C₁₋₇ alkyl, hydroxy C₁₋₇ alkyl or amino C₁₋₇ alkyl substituents. Preferred are halogen, C₁₋₇ alkyl, hydroxy, amino, halo C₁₋₇ alkyl, C₁₋₇ alkoxy and methylsulfonyl substituents. In one group of preferred substituents are 1-2 substituents selected from C₁₋₇ alkyl or halogen substituents, particularly C₁₋₃ alkyl or halogen substituents, particularly methyl, ethyl, chloro, fluoro or bromo substituents.

The “substituted” groups may contain 1 to 3, preferably 1 or 2, of the above mentioned substituents, if not otherwise defined.

Optically active enantiomers or diastereomers of compounds of formula (I) or (II) can be prepared e.g. by resolution of the racemic end product by known methods or by using suitable optically active starting materials. Similarly, racemic compounds of formula (I) or (II) can be prepared by using racemic starting materials. Resolution of racemic compounds of formula (I) or (II) or a racemic starting material thereof can be carried out, for example, by converting the racemic compound into its diastereromeric salt mixture by reaction with an optically active acid and subsequent separation of the diastereomers by crystallization. Representative examples of said optically active acids include, but are not limited to, D-tartaric acid and dibenzoyl-D-tartaric acid. Alternatively, preparative chiral chromatography may be used for resolution of the racemic mixture.

Pharmaceutically acceptable salts are well known in the field of pharmaceuticals. Non-limiting examples of suitable salts include metal salts, ammonium salts, salts with an organic base, salts with an inorganic acid, salts with organic acid, and salts with basic or acidic amino acid. Non-limiting examples of metal salts include alkali metal salts such as sodium salt and potassium salt; alkaline earth metal salts such as calcium salt, and magnesium salt. Non-limiting examples of salts with inorganic or organic acids include chlorides, bromides, sulfates, nitrates, phosphates, sulfonates, methane sulfonates, formates, tartrates, maleates, citrates, benzoates, salicylates, ascorbates, acetates, oxalates, fumarates, hemifumarates, and succinates. Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols, e.g. methyl, ethyl, propyl, isopropyl, butyl, isobutyl, sec-butyl, tert-butyl esters. Phosphate esters and carbonate esters, are also within the scope of the invention.

The definition of formula (I) or (II) above is inclusive of all the possible isotopes and isomers, such as stereoisomers, of the compounds, including geometric isomers, for example Z and E isomers (cis and trans isomers), and optical isomers, e.g. diastereomers and enantiomers, and prodrug esters, e.g. phosphate esters and carbonate esters.

It will be appreciated by those skilled in the art that the present compounds may contain at least one chiral center. Accordingly, the compounds may exist in optically active or racemic forms. It is to be understood that the formula (I) includes any racemic or optically active form, or mixtures thereof. In one embodiment, the compounds are the pure (R)-isomers. In another embodiment, the compounds are the pure (S)-isomers. In another embodiment, the compounds are a mixture of the (R) and the (S) isomers. In another embodiment, the compounds are a racemic mixture comprising an equal amount of the (R) and the (S) isomers. The compounds may contain two chiral centers. In such case, according to one embodiment, the compounds are a mixture of diasteromers. According to another embodiment, the compounds of the invention are a mixture of enantiomers. According to still another embodiment, the compounds are pure enantiomers. The individual isomers may be obtained using the corresponding isomeric forms of the starting material or they may be separated after the preparation of the end compound according to conventional separation methods. For the separation of optical isomers, e.g. enantiomers or diastereomers, from the mixture thereof the conventional resolution methods, e.g. fractional crystallisation, may be used.

The present compounds may also exist as tautomers or equilibrium mixtures thereof wherein a proton of a compound shifts from one atom to another. Examples of tautomerism include, but are not limited to, amido-imido, keto-enol, phenol-keto, oxime-nitroso, nitro-aci, imine-enamine, annular tautomerism of heterocyclic rings, and the like. Tautomeric forms are intended to be encompassed by compounds of formula (I) or (II), even though only one tautomeric form may be depicted.

Examples of preferred compounds of one group of formula (I) or (II) include

2-((5-(1,3,4-Oxadiazol-2-yl)isoindolin-2-yl)methyl)-5-(((methylsulfonyl)piperidin-4-yl)methoxy)-4H-pyran-4-one (Compound 1);

5-((1-(Methylsulfonyl)piperidin-4-yl)methoxy)-2-((5-(oxazol-5-yl)isoindolin-2-yl)methyl)-4H-pyran-4-one (Compound 2);

5-((1-(Methylsulfonyl)piperidin-4-yl)methoxy)-2-((5-(oxazol-2-yl)isoindolin-2-yl)methyl)-4H-pyran-4-one (Compound 3);

2-((5-(Isoxazol-5-yl)isoindolin-2-yl)methyl)-5-((1-(methylsulfonyl)piperidin-4-yl)methoxy)-4H-pyran-4-one (Compound 4);

2-((5-(Isoxazol-3-yl)isoindolin-2-yl)methyl)-5-((1-(methylsulfonyl)piperidin-4-yl)methoxy)-4H-pyran-4-one (Compound 5);

2-((5-(1,3,4-Thiadiazol-2-yl)isoindolin-2-yl)methyl)-5-((1-(methylsulfonyl)-piperidin-4-yl)methoxy)-4H-pyran-4-one (Compound 6);

2-((5-(1H-Pyrazol-1-yl)isoindolin-2-yl)methyl)-5-((1-(methylsulfonyl)piperidin-4-yl)methoxy)-4H-pyran-4-one (Compound 7);

2-((5-(1,2,4-Oxadiazol-3-yl)isoindolin-2-yl)methyl)-5-((1-(methylsulfonyl)-piperidin-4-yl)methoxy)-4H-pyran-4-one (Compound 8);

5-((1-(Methylsulfonyl)piperidin-4-yl)methoxy)-2-((5-(2,2,2trifluorohydroxyethyl) isoindolin-2-yl)methyl)-4H-pyran-4-one (Compound 9);

2-(2-(3-(Methylsulfonyl)-4-((1-(methylsulfonyl)piperidin-4-yl)methoxy)benzyl)-isoindolin-5-yl)-1,3,4-oxadiazole (Compound 10);

3-(2-(3-(Methylsulfonyl)-4-((1-(methylsulfonyl)piperidin-4-yl)methoxy)benzyl)-isoindolin-5-yl)isoxazole (Compound 11);

5-Bromo-2-(3-(methylsulfonyl)-4-((1-(methylsulfonyl)piperidin-4-yl)methoxy)-benzyl)isoindoline (Compound 12a);

2-(3-(Methylsulfonyl)-4-((1-(methylsulfonyl)piperidin-4-yl)methoxy)benzyl)-5-(1H-pyrazol-1-yl)isoindoline (Compounds 12b);

5-(2-(3-(Methylsulfonyl)-4-((1-(methylsulfonyl)piperidin-4-yl)methoxy)-benzyl)isoindolin-5-yl)oxazole (Compound 13a);

2-(2-(3-(Methylsulfonyl)-4-((1-(methylsulfonyl)piperidin-4-yl)methoxy)-benzyl)isoindolin-5-yl)oxazole (Compound 13b);

5-(2-(3-(Methylsulfonyl)-4-((1-(methylsulfonyl)piperidin-4-yl)methoxy)benzyl)-isoindolin-5-yl)thiazole (Compound 14);

1-(2-(3-(Methylsulfonyl)-4-((1-(methylsulfonyl)piperidin-4-yl)methoxy)-benzyl)isoindolin-5-yl)ethan-1-one (Compound 15a);

3-(Dimethylamino)-1-(2-(3-(methylsulfonyl)-4-((1-(methylsulfonyl)piperidin-4-yl)methoxy)benzyl)isoindolin-5-yl)prop-2-en-1-one (Compound 15b);

5-(2-(3-(Methylsulfonyl)-4-((1-(methylsulfonyl)-piperidin-4-yl)methoxy)benzyl)isoindolin-5-yl)isoxazole (Compound 15c);

2-(3-(Methylsulfonyl)-4-((1-(methylsulfonyl)piperidin-4-yl)methoxy)benzyl)-isoindoline-5-carbonitrile (Compound 16a);

N-Hydroxy-2-(3-(methylsulfonyl)-4-((1-(methylsulfonyl)piperidin-4-yl)-methoxy)benzyl)isoindoline-5-carboximidamide (Compound 16b);

3-(2-(3-(Methylsulfonyl)-4-((1-(methylsulfonyl)piperidin-4-yl)methoxy)benzyl)-isoindolin-5-yl)-1,2,4-oxadiazole (Compound 16c);

2-(1-(5-Bromoisoindolin-2-yl)ethyl)-5-((1-(methylsulfonyl)piperidin-4-yl)-methoxy)-4H-pyran-4-one (Compound 17a);

5-((1-(Methylsulfonyl)piperidin-4-yl)methoxy)-2-(1-(5-(oxazol-2-yl)isoindolin-2-yl)ethyl)-4H-pyran-4-one (Compound 17b);

2-(1-(5-((1-(Methylsulfonyl)piperidin-4-yl)methoxy)-4-oxo-4H-pyran-2-yl)ethyl)-isoindoline-5-carbonitrile (Compound 18a);

N-Hydroxy-2-(1-(5-((1-(methylsulfonyl)piperidin-4-yl)methoxy)-4-oxo-4H-pyran-2-yl)ethyl)isoindoline-5-carboximidamide (Compound 18b);

2-(1-(5-(1,2,4-Oxadiazol-3-yl)isoindolin-2-yl)ethyl)-5-((1-(methylsulfonyl)-piperidin-4-yl)methoxy)-4H-pyran-4-one (Compound 18c);

2-(1-(3,4-Dihydroisoquinolin-2(1H)-yl)-2,2,2-trifluoroethyl)-5-((1-(methyl-sulfonyl)piperidin-4-yl)methoxy)-4H-pyran-4-one (Compound 19);

5-((1-(Methylsulfonyl)piperidin-4-yl)methoxy)-2-(2,2,2-trifluoro-1-(isoindolin-2-yl)ethyl)-4H-pyran-4-one (Compound 20);

2-(2,2,2-Trifluoro-1-(3-(methylsulfonyl)-4-((1-(methylsulfonyl)piperidin-4-yl)methoxy)phenyl)ethyl)isoindoline (Compound 21);

2-(2,2-Difluoro-1-(3-(methylsulfonyl)-4-((1-(pyrimidin-2-ylmethyl)piperidin-4-yl)methoxy)phenyl)ethyl)isoindoline (Compound 22);

2-((5-(1,3,4-Oxadiazol-2-yl)isoindolin-2-yl)methyl)-5-(piperidin-4-ylmethoxy)-4H-pyran-4-one (Compound 23a);

2-((5-(1,3,4-Oxadiazol-2-yl)isoindolin-2-yl)methyl)-5-((1-(pyrimidin-2-yl-methyl) piperidin-4-yl)methoxy)-4H-pyran-4-one (Compound 23b);

2-((5-(1,3,4-Oxadiazol-2-yl)-2H-isoindol-2-yl)methyl)-5-((1-(pyrimidin-2-ylmethyl) piperidin-4-yl)methoxy)-4H-pyran-4-one (Compound 23c);

5-((1-(Methylsulfonyl)piperidin-4-yl)methoxy)-2-((S*)-1-((1aR*,7bR*)-1,1a,3,7b-tetrahydro-2H-cyclopropa[c]isoquinolin-2-yl)ethyl)-4H-pyran-4-one

(Compound 24a);

5-((1-(Methylsulfonyl)piperidin-4-yl)methoxy)-2-((R*)-1-((1aR*,7bR*)-1,1a,3,7b-tetrahydro-2H-cyclopropa[c]isoquinolin-2-yl)ethyl)-4H-pyran-4-one (Compound 24b);

2-(3-(Ethylsulfonyl)-4-((1-(methylsulfonyl)piperidin-4-yl)methoxy)benzyl)iso-indoline (Compound 25);

2-(3-(Cyclopropylsulfonyl)-4-((1-(methylsulfonyl)piperidin-4-yl)methoxy)-benzyl)isoindoline (Compound 26);

2-(3-(Ethylsulfonyl)-4-((1-(methylsulfonyl)piperidin-4-yl)methoxy)benzyl)-5-(trifluoromethyl)isoindoline (Compound 27);

2-(3-(Butylsulfonyl)-4-((1-(methylsulfonyl)piperidin-4-yl)methoxy)benzyl)iso-indoline (Compound 28);

2-((1′H-Spiro[cyclopropane-1,4′-isoquinolin]-2′(3′H)-yl)methyl)-5-((1-(methyl-sulfonyl)piperidin-4-yl)methoxy)-4H-pyran-4-one (Compound 29);

tert-Butyl 6-(((6-((3,4-dihydroisoquinolin-2(1H)-yl)methyl)-4-oxo-4H-pyran-3-yl)oxy)methyl)-2-azaspiro[3.3]heptane-2-carboxylate (Compound 30);

tert-Butyl 6-((6-((3,4-dihydroisoquinolin-2(1H)-yl)methyl)-4-oxo-4H-pyran-3-yl)oxy)-2-azaspiro[3.3]heptane-2-carboxylate (Compound 31);

tert-Butyl 2-((6-((3,4-dihydroisoquinolin-2(1H)-yl)methyl)-4-oxo-4H-pyran-3-yl)oxy)-7-azaspiro[3.5]nonane-7-carboxylate (Compound 32);

tert-Butyl 2-((6-((3,4-dihydroisoquinolin-2(1H)-yl)methyl)-4-oxo-4H-pyran-3-yl)oxy)-6-azaspiro[3.4]octane-6-carboxylate (Compound 33);

tert-Butyl 7-((6-((3,4-dihydroisoquinolin-2(1H)-yl)methyl)-4-oxo-4H-pyran-3-yl)oxy)-5-oxa-2-azaspiro[3.4]octane-2-carboxylate (Compound 34);

tert-Butyl 2-(((6-((3,4-dihydroisoquinolin-2(1H)-yl)methyl)-4-oxo-4H-pyran-3-yl)oxy)methyl)-7-azaspiro[3.5]nonane-7-carboxylate (Compound 35);

tert-Butyl 6-(1-((6-((3,4-dihydroisoquinolin-2(1H)-yl)methyl)-4-oxo-4H-pyran-3-yl)oxy)ethyl)-2-azaspiro[3.3]heptane-2-carboxylate (Compound 36);

tert-Butyl 2-(1-((6-((3,4-dihydroisoquinolin-2(1H)-yl)methyl)-4-oxo-4H-pyran-3-yl)oxy)ethyl)-7-azaspiro[3.5]nonane-7-carboxylate (Compound 37);

5-((2-Azaspiro[3.3]heptan-6-yl)oxy)-2-((3,4-dihydroisoquinolin-2(1H)-yl)-methyl)-4H-pyran-4-one bis-trifluoroacetate (Compound 38);

5-((7-Azaspiro[3.5]nonan-2-yl)oxy)-2-((3,4-dihydroisoquinolin-2(1H)-yl)methyl)-4H-pyran-4-one (Compound 39);

5-((7-Azaspiro[3.5]nonan-2-yl)methoxy)-2-((3,4-dihydroisoquinolin-2(1H)-yl)methyl)-4H-pyran-4-one (Compound 40);

5-((6-Azaspiro[3.4]octan-2-yl)oxy)-2-((3,4-dihydroisoquinolin-2(1H)-yl)methyl)-4H-pyran-4-one (Compound 41);

5-((2-Azaspiro[3.3]heptan-6-yl)methoxy)-2-((3,4-dihydroisoquinolin-2(1H)-yl)methyl)-4H-pyran-4-one (Compound 42);

5-(1-(2-Azaspiro[3.3]heptan-6-yl)ethoxy)-2-((3,4-dihydroisoquinolin-2(1H)-yl)methyl)-4H-pyran-4-one (Compound 43);

5-(1-(7-Azaspiro[3.5]nonan-2-yl)ethoxy)-2-((3,4-dihydroisoquinolin-2(1H)-yl)methyl)-4H-pyran-4-one (Compound 44);

2-((3,4-Dihydroisoquinolin-2(1H)-yl)methyl)-5-((2-(pyrimidin-2-yl)-2-azaspiro-[3.3]heptan-6-yl)oxy)-4H-pyran-4-one (Compound 45);

2-((3,4-Dihydroisoquinolin-2(1H)-yl)methyl)-5-((7-(pyrimidin-2-yl)-7-azaspiro-[3.5]nonan-2-yl)oxy)-4H-pyran-4-one (Compound 46);

2-((3,4-Dihydroisoquinolin-2(1H)-yl)methyl)-5-((7-(pyrimidin-2-ylmethyl)-7-azaspiro[3.5]nonan-2-yl)oxy)-4H-pyran-4-one (Compound 47);

2-((3,4-Dihydroisoquinolin-2(1H)-yl)methyl)-5-((7-(pyrimidin-2-yl)-7-azaspiro-[3.5]nonan-2-yl)methoxy)-4H-pyran-4-one (Compound 48);

2-((3,4-Dihydroisoquinolin-2(1H)-yl)methyl)-5-((6-(pyrimidin-2-yl)-6-azaspiro-[3.4]octan-2-yl)oxy)-4H-pyran-4-one (Compound 49);

2-((3,4-Dihydroisoquinolin-2(1H)-yl)methyl)-5-((2-(pyrimidin-2-yl)-2-azaspiro-[3.3]heptan-6-yl)methoxy)-4H-pyran-4-one (Compound 50);

6-(6-(((6-((3,4-Dihydroisoquinolin-2(1H)-yl)methyl)-4-oxo-4H-pyran-3-yl)oxy)-methyl)-2-azaspiro[3.3]heptan-2-yl)nicotinonitrile (Compound 51);

6-(3-(((6-((3,4-Dihydroisoquinolin-2(1H)-yl)methyl)-4-oxo-4H-pyran-3-yl)oxy)-methyl)azetidin-1-yl)nicotinonitrile (Compound 52);

2-((3,4-Dihydroisoquinolin-2(1H)-yl)methyl)-5-((7-((1-methyl-1H-pyrazol-5-yl)sulfonyl)-7-azaspiro[3.5]nonan-2-yl)oxy)-4H-pyran-4-one (Compound 53);

2-((3,4-Dihydroisoquinolin-2(1H)-yl)methyl)-5-((7-(pyridin-3-ylsulfonyl)-7-azaspiro[3.5]nonan-2-yl)oxy)-4H-pyran-4-one (Compound 54);

2-((3,4-Dihydroisoquinolin-2(1H)-yl)methyl)-5-((7-((1-methyl-1H-pyrazol-4-yl)sulfonyl)-7-azaspiro[3.5]nonan-2-yl)oxy)-4H-pyran-4-one (Compound 55);

2-((3,4-Dihydroisoquinolin-2(1H)-yl)methyl)-5-((2-(pyridin-3-ylsulfonyl)-2-azaspiro[3.3]heptan-6-yl)oxy)-4H-pyran-4-one (Compound 56);

2-((3,4-Dihydroisoquinolin-2(1H)-yl)methyl)-5-((2-((1-methyl-1H-pyrazol-4-yl)sulfonyl)-2-azaspiro[3.3]heptan-6-yl)methoxy)-4H-pyran-4-one (Compound 57);

2-((3,4-Dihydroisoquinolin-2(1H)-yl)methyl)-5-((2-((4-fluorophenyl)sulfonyl)-2-azaspiro[3.3]heptan-6-yl)methoxy)-4H-pyran-4-one (Compound 58);

2-((3,4-Dihydroisoquinolin-2(1H)-yl)methyl)-5-(1-(2-(methylsulfonyl)-2-azaspiro[3.3]heptan-6-yl)ethoxy)-4H-pyran-4-one (Compound 59);

2-((3,4-Dihydroisoquinolin-2(1H)-yl)methyl)-5-(1-(2-(pyridin-3-ylsulfonyl)-2-azaspiro[3.3]heptan-6-yl)ethoxy)-4H-pyran-4-one (Compound 60);

2-((3,4-Dihydroisoquinolin-2(1H)-yl)methyl)-5-(1-(7-(methylsulfonyl)-7-azaspiro[3.5]nonan-2-yl)ethoxy)-4H-pyran-4-one (Compound 61);

2-((3,4-Dihydroisoquinolin-2(1H)-yl)methyl)-5-((2-(methylsulfonyl)-2-azaspiro[3.3]heptan-6-yl)methoxy)-4H-pyran-4-one (Compound 62);

2-((3,4-Dihydroisoquinolin-2(1H)-yl)methyl)-5-((7-(methylsulfonyl)-7-azaspiro[3.5]nonan-2-yl)oxy)-4H-pyran-4-one (Compound 63);

2-(1-(5-Acetylisoindolin-2-yl)ethyl)-5-((1-(methylsulfonyl)piperidin-4-yl)-methoxy)-4H-pyran-4-one (Compound 64);

2-(3-(Methylsulfonyl)-4-((1-(methylsulfonyl)piperidin-4-yl)methoxy)benzyl)-isoindoline (Compound 65);

2-(3-(Methylsulfonyl)-4-((1-(methylsulfonyl)piperidin-4-yl)methoxy)benzyl)-isoindoline (Compound 66);

1-((1r,4r)-4-((4-(Isoindolin-2-ylmethyl)-2-(methylsulfonyl)phenoxy)methyl)-cyclohexyl)ethan-1-one (Compound 67);

4-((4-(Isoindolin-2-ylmethyl)-2-(methylsulfonyl)phenoxy)methyl)-N,N-dimethylbenzamide (Compound 68);

4-((4-(Isoindolin-2-ylmethyl)-2-(methylsulfonyl)phenoxy)methyl)-N-(2-methoxyethyl)-N-methylbenzamide (Compound 69);

1-(4-((4-Isoindolin-2-ylmethyl)-2-(methylsulfonyl)phenoxy)methyl)piperidin-1-yl)propan-1-one (Compound 70);

Ethyl(imino)(4-((4-(isoindolin-2-ylmethyl)-2-(methylsulfonyl)phenoxy)methyl)-phenyl)-λ⁶-sulfanone (Compound 71);

Imino(4-((4-(isoindolin-2-ylmethyl)-2-(methylsulfonyl)phenoxy)methyl)phenyl)(methyl)-λ⁶-sulfanone (Compound 72);

1-(4-((4-(Isoindolin-2-ylmethyl)-2-(methylsulfonyl)phenoxy)methyl)piperidin-1-yl)-2-methylpropan-1-one (Compound 73);

4-((4-(Isoindolin-2-ylmethyl)-2-(methylsulfonyl)phenoxy)methyl)-N,N-dimethylcyclohexane-1-carboxamide (Compound 74);

2-(3-(Methylsulfonyl)-4-((1-(oxetan-3-ylsulfonyl)piperidin-4-yl)methoxy)-benzyl)isoindoline (Compound 75);

2-(4-((4-(Ethylsulfonyl)benzyl)oxy)-3-(methylsulfonyl)benzyl)isoindoline (Compound 76);

1-((4-((4-(Isoindolin-2-ylmethyl)-2-(methylsulfonyl)phenoxy)methyl)phenyl)-sulfonyl)azetidin-3-ol (Compound 77);

N-(Dimethyl(oxo)-λ⁶-sulfaneylidene)-4-((4-(isoindolin-2-ylmethyl)-2-(methyl-sulfonyl)phenoxy)methyl)benzamide (Compound 78);

(1r,4r)-4-((4-(Isoindolin-2-ylmethyl)-2-(methylsulfonyl)phenoxy)methyl)-N,N-dimethylcyclohexane-1-carboxamide (Compound 79);

Azetidin-1-yl(4-((4-(isoindolin-2-ylmethyl)-2-(methylsulfonyl)phenoxy)-methyl)cyclohexyl)methanone (Compound 80);

2-(4-((3-Fluoro-4-(methylsulfonyl)benzyl)oxy)-3-(methylsulfonyl)benzyl)-isoindoline (Compound 81);

2-(3-(Methylsulfonyl)-4-((4-(methylsulfonyl)benzyl)oxy)benzyl)isoindoline (Compound 82);

2-(3-(Methylsulfonyl)-4-((tetrahydro-2H-pyran-4-yl)methoxy)benzyl)isoindoline (Compound 83);

(4-((4-(Isoindolin-2-ylmethyl)-2-(methylsulfonyl)phenoxy)methyl)phenyl)-(methyl)-λ⁴-sulfanimine (Compound 84);

Imino(methyl)(4-((2-(methylsulfonyl)-4-((5-(trifluoromethyl)isoindolin-2-yl)methyl)phenoxy)methyl)phenyl)-λ⁶-sulfanone (Compound 85);

2-(4-((4-(Isoindolin-2-ylmethyl)-2-(methylsulfonyl)phenoxy)methyl)phenyl)-propan-2-ol (Compound 86);

1-(4-((4-(Isoindolin-2-ylmethyl)-2-(methylsulfonyl)phenoxy)methyl)piperidin-1-yl)ethan-1-one (Compound 87);

2-((1′H-Spiro[cyclopropane-1,4′-isoquinolin]-2′(3′H)-yl)methyl)-5-((1-tosyl-piperidin-4-yl)methoxy)-4H-pyran-4-one (Compound 88);

2-((5-Acetylisoindolin-2-yl)methyl)-5-((1-(methylsulfonyl)piperidin-4-yl)-methoxy)-4H-pyran-4-one (Compound 89);

(E)-2-((5-(1-(Hydroxyimino)ethyl)isoindolin-2-yl)methyl)-5-((1-(methylsulfonyl)piperidin-4-yl)methoxy)-4H-pyran-4-one (Compound 90);

(E)-2-((5-(1-(Methoxyimino)ethyl)isoindolin-2-yl)methyl)-5-((1-(methylsulfonyl)piperidin-4-yl)methoxy)-4H-pyran-4-one (Compound 91);

1-(4-((4-(Isoindolin-2-ylmethyl)-2-(methylsulfonyl)phenoxy)methyl)phenyl)-ethanone (Compound 92a);

1-(4-((4-(Isoindolin-2-ylmethyl)-2-(methylsulfonyl)phenoxy)methyl)phenyl)-ethanol (Compound 92b);

2-((5-(1-Hydroxyethyl)isoindolin-2-yl)methyl)-5-((1-(methylsulfonyl)piperidin-4-yl)methoxy)-4H-pyran-4-one (Compound 93);

1-(4-((4-(Isoindolin-2-ylmethyl)-2-(methylsulfonyl)phenoxy)methyl)cyclohexyl)ethan-1-ol (Compound 94);

(R)-Imino(4-((4-(isoindolin-2-ylmethyl)-2-(methylsulfonyl)phenoxy)methyl)-phenyl)(methyl)-λ⁶-sulfanone (Compound 95);

(S)-Imino(4-((4-(isoindolin-2-ylmethyl)-2-(methylsulfonyl)phenoxy)methyl)-phenyl)(methyl)-λ⁶-sulfanone (Compound 96);

(S)-Ethyl(imino)(4-((4-(isoindolin-2-ylmethyl)-2-(methylsulfonyl)phenoxy)-methyl)phenyl)-λ⁶-sulfanone (Compound 97);

(R)-Ethyl(imino)(4-((4-(isoindolin-2-ylmethyl)-2-(methylsulfonyl)phenoxy)-methyl)phenyl)-λ⁶-sulfanone (Compound 98);

(R)-2-(1-(3,4-dihydroisoquinolin-2(1H)-yl)-2,2,2-trifluoroethyl)-5-((1-(methylsulfonyl)piperidin-4-yl)methoxy)-4H-pyran-4-one (Compound 99);

(S)-2-(1-(3,4-dihydroisoquinolin-2(1H)-yl)-2,2,2-trifluoroethyl)-5-((1-(methylsulfonyl)piperidin-4-yl)methoxy)-4H-pyran-4-one (Compound 100);

2-((6-(Isoxazol-4-yl)-3,4-dihydroisoquinolin-2(1H)-yl)methyl)-5-((1-(methyl-sulfonyl)piperidin-4-yl)methoxy)-4H-pyran-4-one (Compound 101);

2-((6-(1H-Pyrazol-1-yl)-3,4-dihydroisoquinolin-2(1H)-yl)methyl)-5-((1-(methyl-sulfonyl)piperidin-4-yl)methoxy)-4H-pyran-4-one (Compound 102);

2-((1′H-Spiro[cyclopropane-1,4′-isoquinolin]-2′(3′H)-yl)methyl)-5-((1-(methyl-sulfonyl)piperidin-4-yl)methoxy)-4H-pyran-4-one (Compound 103);

2-((1′H-Spiro[cyclopentane-1,4′-isoquinolin]-2′(3′H)-yl)methyl)-5-((4-(2-hydroxypropan-2-yl)benzyl)oxy)-4H-pyran-4-one (Compound 104);

4-Fluoro-2-(3-(methylsulfonyl)-4-((1-(methylsulfonyl)piperidin-4-yl)methoxy)-benzyl)isoindoline (Compound 105);

and tautomers and pharmaceutically acceptable salts thereof.

Compounds of the invention may be administered to a patient in therapeutically effective amounts which range usually from about 0.5 to about 2000 mg, more typically form about 1 to about 500 mg, daily depending on the age, sex, weight, ethnic group, condition of the patient, condition to be treated, administration route and the active ingredient used. The compounds of the invention can be formulated into dosage forms using the principles known in the art. The compound can be given to a patient as such or in combination with suitable pharmaceutical excipients in the form of tablets, granules, capsules, suppositories, emulsions, suspensions or solutions. Choosing suitable ingredients for the composition is a routine for those of ordinary skill in the art. Suitable carriers, solvents, gel forming ingredients, dispersion forming ingredients, antioxidants, colours, sweeteners, wetting compounds and other ingredients normally used in this field of technology may also be used. The compositions containing the active compound can be given enterally or parenterally, the oral route being the preferred way. The contents of the active compound in the composition is from about 0.5 to 100%, preferably from about 0.5 to about 20%, per weight of the total composition.

The compounds of the invention can be given to the subject as the sole active ingredient or in combination with one of more other active ingredients for treatment of a particular disease.

In the treatment of a steroid receptor dependent disease or condition, such as endocrine cancers and disorders including prostate cancer and breast cancer, a combination of therapeutic agents and/or other treatments (e.g., radiation therapy) is often advantageous. The second (or third) agent to be administered may have the same or different mechanism of action than the primary therapeutic agent.

Accordingly, a compound of the invention may be administered in combination with other anti-cancer treatments useful in the treatment of cancers such as prostate cancer or breast cancer. For example, a compound of the invention can be packaged together with instructions that the compound is to be used in combination with other anti-cancer agents and treatments for the treatment of cancer. The present invention further comprises combinations of a compound of the invention and one or more additional agents in kit form, for example, where they are packaged together or placed in separate packages to be sold together as a kit, or where they are packaged to be formulated together.

According to one embodiment of the invention, the therapeutically effective amount of a compound of formula (I) or (II) is co-administered with a glucocorticoid and/or a mineralocorticoid and, optionally, with one or more anti-cancer agents.

Examples of suitable glucocorticoids include, but are not limited to, hydrocortisone, prednisone, prednisolone, methylprednisolone and dexamethasone. Examples of suitable mineralocorticoids include, but are not limited to, fludrocortisone, deoxycorticosterone, 11-desoxycortisone and deoxycorticosterone acetate.

The optional other anti-cancer agents which can be administered in addition to a compound of formula (I) or (II) include, but are not limited to,
non-steroidal androgen receptor antagonists (e.g. enzalutamide, apalutamide and darolutamide);
steroidogenesis inhibitors (e.g. CYP17A1 inhibitors such as abiraterone acetate and seviteronel);
chemotherapeutic agents (e.g. docetaxel and paclitaxel);
antiestrogens (e.g. tamoxifen and fulvestrant);
epigenetic modulators (e.g. BET inhibitors and HDAC inhibitors);
mTOR inhibitors (e.g. everolimus);
AKT inhibitors (e.g. AZ5363);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib).
   According to one embodiment of the invention, the therapeutically effective amount of a compound of formula (I) or (II) is administered to a subject in need thereof in addition to a therapeutically effective amount of one or more anti-cancer agents selected from the list consisting of
non-steroidal androgen receptor antagonists (e.g. enzalutamide, apalutamide and darolutamide);
steroidogenesis inhibitors (e.g. CYP17A1 inhibitors such as abiraterone acetate and seviteronel);
chemotherapeutic agents (e.g. docetaxel and paclitaxel);
antiestrogens (e.g. tamoxifen and fulvestrant);
epigenetic modulators (e.g. BET inhibitors and HDAC inhibitors);
mTOR inhibitors (e.g. everolimus);
AKT inhibitors (e.g. AZ5363);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib).

According to one embodiment of the invention, the therapeutically effective amount of a compound of formula (I) or (II) is administered to a subject in need thereof in addition to a therapeutically effective amount of a steroidogenesis inhibitor (e.g. a CYP17A1 inhibitor). Examples of suitable CYP17A1 inhibitors include, but are not limited to, abiraterone acetate and seviteronel.

According to another embodiment of the invention, the therapeutically effective amount of a compound of formula (I) or (II) is administered to a subject in need thereof in addition to a therapeutically effective amount of a non-steroidal androgen receptor antagonist. Examples of suitable non-steroidal androgen receptor (AR) antagonists include, but are not limited to, enzalutamide, apalutamide and darolutamide.

According to still another embodiment, the present invention provides a pharmaceutical combination comprising a compound of formula (I) or (II) and at least one additional active ingredient selected from the list consisting of
a glucocorticoid,
a mineralocorticoid,
a steroidogenesis inhibitor (e.g. a CYP17A1 inhibitor),
a non-steroidal androgen receptor antagonist,
chemotherapeutic agents (e.g. docetaxel and paclitaxel),
antiestrogens (e.g. tamoxifen and fulvestrant),
epigenetic modulators (e.g. BET inhibitors and HDAC inhibitors),
mTOR inhibitors (e.g. everolimus);
AKT inhibitors (e.g. AZ5363);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib)
   for simultaneous, separate or sequential administration.

The above other therapeutic agents, when employed in combination with a compound of the invention can be used, for example, in those amounts indicated in the Physicians' Desk Reference (PDR) or as otherwise determined by one of ordinary skill in the art.

The compounds of the invention can be prepared by a variety of synthetic routes analogously to the methods known in the literature using suitable starting materials. The present invention will be explained in more detail by the following experiments and examples. The experiments and examples are meant only for illustrating purposes and do not limit the scope of the invention defined in claims.

### EXAMPLES

### Intermediate 2a: 2-(Isoindolin-5-yl)-1,3,4-oxadiazole trifluoroacetate

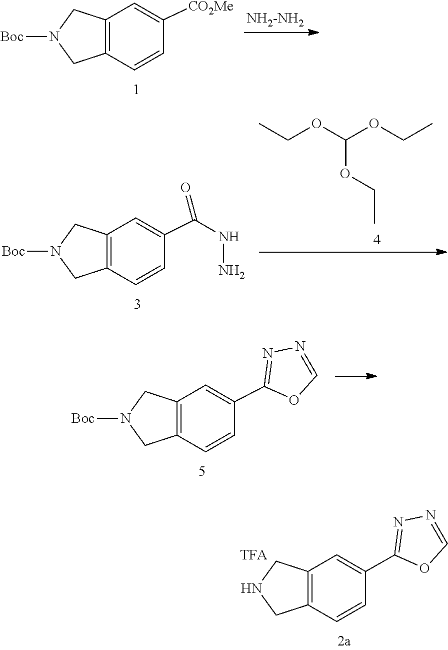

a) tert-Butyl 5-(hydrazinecarbonyl)isoindoline-2-carboxylate (3)

To a solution of 2-(tert-butyl)5-methyl isoindoline-2,5-dicarboxylate (3.50 g, 12.6 mmol, 1.0 eq.) in 25 mL of EtOH, was added hydrazine hydrate (0.40 g, 0.40 mL, 12.6 mmol, 1.0 eq.) at 25° C. The resulting mixture was heated at 80° C. for 24 h. After completion of the reaction, solvent was evaporated under reduced pressure and the resulting crude product was forwarded to the next step without further purification. R_{f} (EtOAc/heptane: 8/2)=0.2. Yield 4.5 g (crude). MS (ESI) m/z [M+1]⁺: 278.09.

b) tert-Butyl 5-(1,3,4-oxadiazol-2-yl)isoindoline-2-carboxylate (5)

tert-Butyl 5-(hydrazinecarbonyl)isoindoline-2-carboxylate (4.5 g, 16.2 mmol, 1.0 eq.) and triethylorthoformate (50 mL) was taken in a 100 mL seal tube. Then the mixture was heated at 120° C. for 16 h. After completion of the reaction, the reaction mixture was quenched with ice cold water and extracted (3 times) with EtOAc. The combined organic layer was washed with brine, dried over anhydrous sodium sulphate and concentrated under reduced pressure. This crude product was purified by combi-flash chromatography using 0-50% ethyl acetate in heptane as an eluent. R_{f} (EtOAc/heptane: 5/5)=0.3. Yield 0.6 g, 13%. MS (ESI) m/z [M+1]⁺: 288.03.

¹H-NMR (400 MHz, DMSO-d₆): δ 9.34 (s, 1H), 7.84-8.01 (m, 2H), 7.53-7.60 (m, 1H), 4.31-4.70 (m, 4H), 1.46 (s, 9H).

c) 2-(Isoindolin-5-yl)-1,3,4-oxadiazole trifluoroacetate (2a)

To a solution of tert-butyl 5-(1,3,4-oxadiazol-2-yl)isoindoline-2-carboxylate (0.24 g, 2.09 mmol, 1.0 eq.) in 10 mL DCM was added 2,2,2-trifluoroacetic acid (1.5 mL) at 0° C. Then the resulting mixture was stirred at 0° C. for 1 h. After completion of the reaction, solvent was evaporated under reduced pressure and the resulting crude product was forwarded to the next step without further purification. R_{f} (EtOAc/Heptane: 3/7)=0.2. Yield 0.6 g (Crude). MS (ESI) m/z [M+1]⁺:188,08.

¹H-NMR (400 MHz, DMSO-d₆): δ 9.55 (bs, 2H), 9.43 (s, 1H), 8.13 (s, 1H), 8.03 (d, 1H), 7.64 (d, 1H), 4.62 (s, 4H).

### Intermediates 2b and 2c: 5-(Isoindolin-5-yl)oxazole Hydrogen Chloride (2b) and 2-(isoindolin-5-yl)oxazole Hydrogen Chloride (2c)

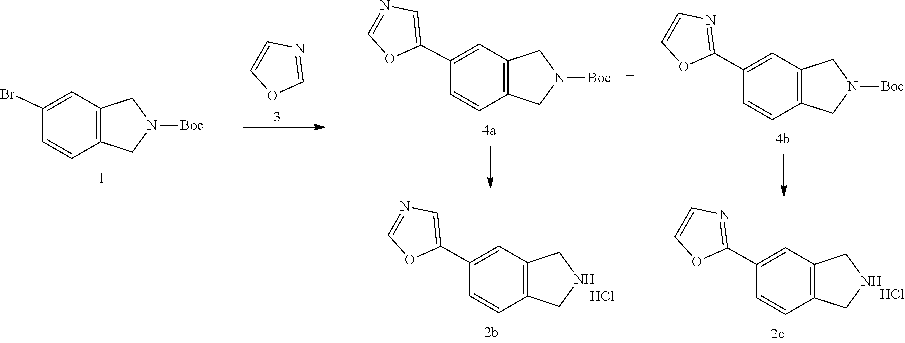

a) tert-Butyl 5-(oxazol-5-yl)isoindoline-2-carboxylate (4a) and tert-butyl 5-(oxazol-2-yl)isoindoline-2-carboxylate (4b)

To a solution of tert-butyl 5-bromoisoindoline-2-carboxylate (2.0 g, 6.73 mmol, 1.0 eq.) in 20 mL of DMA, were added oxazole (1.85 g, 26.9 mmol, 4 eq.), cesium carbonate (5.47 g, 16.8 mmol, 2.5 eq.) and pivalic acid (0.28 g, 2.69 mmol, 0.4 eq.) at 25° C. This mixture was degassed by argon for 15 min. Subsequently palladium(II) acetate (0.83 g, 0.34 mmol, 0.05 eq.) and di(1-adamantyl)-N-butylphosphine (0.12 g, 0.34 mmol 0.05 eq) were added to the reaction mixture and the resulting reaction mixture was heated at 110° C. for 16 h. After completion of the reaction, the reaction mixture was quenched with water and extracted (3 times) with EtOAc. The combined organic layer was washed with brine, dried over anhydrous sodium sulphate and concentrated under reduced pressure. This crude product was purified by combi-flash chromatography using 0-20% EtOAc in heptane as an eluent. The corresponding two regioisomers were obtained separately. R_{f} (EtOAc/Heptane: 3/7)=0.2. Yields 0.42 g, 22% and 0.72 g, 37%. MS(ESI) m/z [M+1]⁺: 287.01.

Intermediate (4a): ¹H-NMR (400 MHz, DMSO-d₆): δ 8.44 (s, 1H), 7.63-7.90 (m, 3H), 7.35-7.47 (m, 1H), 4.57-4.65 (m, 4H), 1.53 (s, 9H).

Intermediate (4b): ¹H-NMR (400 MHz, DMSO-d₆): δ 8.21 (s, 1H), 7.89-7.94 (m, 2H), 7.46-7.49 (m, 1H), 7.38 (s, 1H), 4.60-4.68 (m, 4H), 1.46 (s, 9H).

b) 5-(Isoindolin-5-yl)oxazole Hydrogen Chloride (2b)

Treatment of tert-Butyl 5-(oxazol-5-yl)isoindoline-2-carboxylate (0.25 g, 0.87 mmol, 1.0 eq.) with HCl in EtOAc (12.0 mL, 1.0 M solution) in EtOAc (30 mL) and MeOH (2 mL) for 12 h afforded the title product as white solid. Yield 0.20 g (crude). MS(ESI) m/z [M+1]⁺:187.08. ¹H-NMR (400 MHz, DMSO-d₆): δ 9.80 (bs, 2H), 8.48 (s, 1H), 7.75-7.77 (m, 1H), 7.73 (s, 1H), 7.51 (d, 1H), 4.53-4.62 (m, 4H).

c) 2-(Isoindolin-5-yl)oxazole Hydrogen Chloride (2c)

Treatment of tert-butyl 5-(oxazol-2-yl)isoindoline-2-carboxylate (0.25 g, 0.87 mmol, 1.0 eq.) with HCl in EtOAc (12.0 mL, 1.0 M solution) in EtOAc (30 mL) and MeOH (2 mL) for 12 h afforded the title product as white solid. Yield 0.20 g (crude). MS (ESI) m/z [M+1]⁺:187.08. ¹H-NMR (400 MHz, /DMSO-d₆): δ 10.01 (s, 1H), 8.25 (s, 1H), 8.03 (s, 1H), 7.97 (d, 1H), 7.56 (d, 1H), 7.40 (s, 1H), 4.50-4.59 (m, 4H).

### Intermediate 2d: 5-(Isoindolin-5-yl)isoxazole Hydrogen Chloride

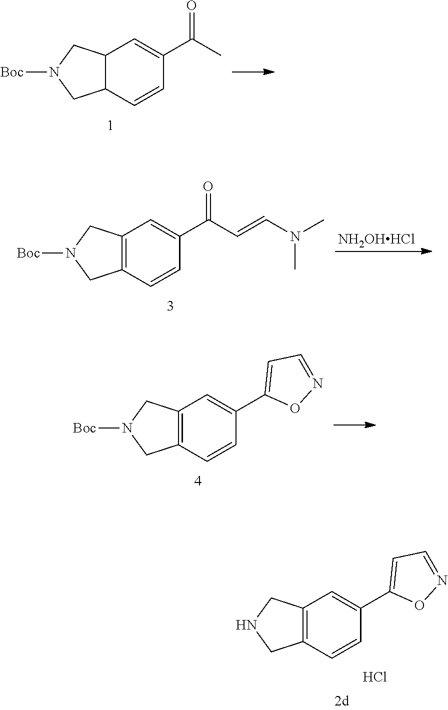

a) tert-Butyl (E)-5-(3-(dimethylamino)acryloyl)isoindoline-2-carboxylate (3)

To a solution of tert-butyl 5-acetylisoindoline-2-carboxylate (0.9 g, 3.40 mmol, 1.0 eq.) in 12 mL DMF was added DMF-DMA (40 mL) at 25° C. The resulting mixture was stirred at 90° C. for 16 h. After completion of the reaction, solvent was evaporated under reduced pressure and resulting crude product was forwarded to the next step without purification. R_{f} (MeOH/DCM: 0.5/9.5)=0.3. Yield 0.89 g (crude). MS (ESI) m/z [M+1]⁺:317.20.

b) tert-Butyl 5-(isoxazol-5-yl)isoindoline-2-carboxylate (4)

A mixture of tert-butyl (E)-5-(3-(dimethylamino)acryloyl)isoindoline-2-carboxylate (0.85 g, 2.68 mmol, 1.0 eq.) and hydroxyl amine hydrochloride (0.22 g, 3.22 mmol, 1.2 eq.) in 40 mL EtOH was refluxed for 16 h. After completion of the reaction, solvent was evaporated under reduced pressure and resulting crude product was forwarded to the next step without further purification. R_{f} (MeOH/DCM: 0.5/9.5)=0.5. Yield 0.55 g (crude). MS (ESI) m/z [M−1]⁺: 285.01. ¹H-NMR (400 MHz, DMSO-d₆) δ 8.65 (s, 1H), 7.85 (d, 1H), 7.81 (d, 1H), 7.48-7.53 (m, 1H), 7.01 (d, 1H), 4.60-4.68 (m, 4H), 1.36 (s, 9H).

c) 5-(Isoindolin-5-yl)isoxazole Hydrogen Chloride (2d)

Treatment of tert-butyl 5-(isoxazol-5-yl)isoindoline-2-carboxylate (0.35 g, 1.22 mmol, 1.0 eq.) with HCl in EtOAc (20.0 mL, 1.0 M solution) in MeOH (2 mL) for 12 h afforded the title product as a white solid. R_{f} (EtOAc/Heptan: 8/2)=0.2. Yield 0.27 g (crude). MS (ESI) m/z [M+1]⁺:187.03. ¹H-NMR (400 MHz, DMSO-d₆): δ 9.87 (bs, 1H), 8.68 (s, 1H), 7.93-7.98 (m, 1H), 7.82-7.90 (m, 1H), 7.57 (d, 1H), 7.06 (s, 1H), 4.57 (s, 4H).

### Intermediate 2e: 3-(Isoindolin-5-yl)isoxazole trifluoroacetate

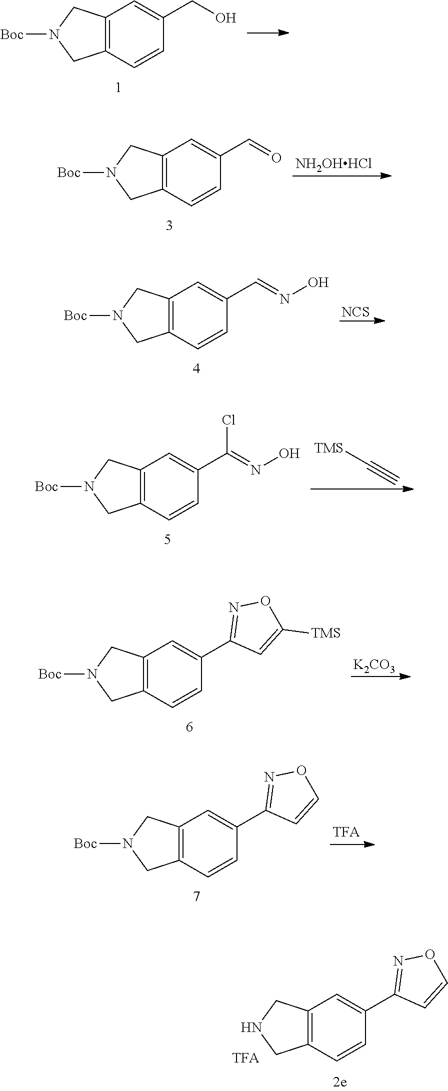

a) tert-Butyl 5-formylisoindoline-2-carboxylate (3)

To a solution of tert-butyl 5-(hydroxymethyl)isoindoline-2-carboxylate (1.00 g, 4.01 mmol, 1.00 eq.) in mixture of DCM:DMSO (5:1, 24 mL) were added Et₃N (2.86 g, 3.9 mL, 28.1 mmol, 7.0 eq.) and sulphur trioxide pyridine (3.18 g, 20.0 mmol, 5.0 eq.) at 0° C. The resulting mixture was stirred at 25° C. for 12 h. After completion of the reaction, the reaction mixture was quenched with water and extracted (3 times) with EtOAc. The combined organic layer was washed with brine, dried over anhydrous sodium sulphate and concentrated under reduced pressure to get crude product. This crude product was purified by combi-flash chromatography using 0-30% ethyl acetate in heptane as an eluent. R_{f} (EtOAc/Heptane: 5/5)=0.4. Yield 0.96 g, 96%. MS (ESI) m/z [M+1]⁺: 248.10. ¹H-NMR (400 MHz, DMSO-d₆): δ 10.0 (s, 1H), 7.84-7.87 (m, 2H), 7.54-7.58 (m, 1H), 4.63-4.68 (m, 4H), 1.46 (s, 9H).

b) tert-Butyl (E)-5-((hydroxyimino)methyl)isoindoline-2-carboxylate (4)

To a solution of tert-butyl 5-formylisoindoline-2-carboxylate (0.96 g, 3.87 mmol, 1.00 eq.) in EtOH (90 mL) were added Et₃N (0.60 g, 0.82 mL, 5.80 mmol, 1.5 eq.) and hydroxylamine hydrochloride (0.41 g, 5.80 mmol, 1.5 eq.) at 25° C. The resulting mixture was stirred at 90° C. for 16 h. After completion of the reaction, the reaction mixture was quenched with water and extracted (3 times) with EtOAc. The combined organic layer was washed with brine, dried over anhydrous sodium sulphate and concentrated under reduced pressure. The resulting crude product was forwarded to the next step without further purification. R_{f} (EtOAc/heptane: 3/7)=0.4. Yield 0.96 g (crude). MS (ESI) [M+1]⁺: 263.04. ¹H-NMR (400 MHz, DMSO-d₆): δ 11.21 (s, 1H), 8.13 (s, 1H), 7.48-7.55 (m, 2H), 7.33-7.36 (m, 1H), 4.54-4.61 (m, 4H), 1.45 (s, 9H).

c) tert-Butyl (Z)-5-(chloro(hydroxyimino)methyl)isoindoline-2-carboxylate (5)

To a solution of tert-butyl (E)-5-((hydroxyimino)methyl)isoindoline-2-carboxylate (0.96 g, 3.66 mmol, 1.00 eq.) in 60 mL DMF was added N-chlorosuccin-imide (0.49 g, 3.66 mmol, 1.0 eq.) at 0° C. The resulting mixture was stirred at 25° C. for 16 h. After completion of the reaction, the reaction mixture was quenched with water and extracted (3 times) with EtOAc. The combined organic layer was washed with brine, dried over anhydrous sodium sulphate and concentrated under reduced pressure. The resulting crude product was forwarded to the next step without purification. R_{f} (EtOAc/Heptane: 5/5)=0.4. Yield 1.10 g (crude). MS (ESI) unionized.

¹H-NMR (400 MHz, DMSO-d₆): δ 12.37 (s, 1H), 7.70-7.75 (m, 2H), 7.40-7.43 (m, 1H), 4.62 (s, 4H), 1.46 (s, 9H).

d) tert-Butyl 5-(5-(trimethylsilyl)isoxazol-3-yl)isoindoline-2-carboxylate (6)

To a solution of tert-butyl (Z)-5-(chloro(hydroxyimino)methyl)isoindoline-2-carboxylate (1.10 g, 3.7 mmol, 1.00 eq.) in 20 mL DCM, were added triethylamine (1.48 g, 2.01 mL, 13.0 mmol, 3.5 eq.) and trimethylsilylacetylene (1.20 g, 1.6 mL, 11.1 mmol, 3.0 eq.) at 0° C. The resulting mixture was stirred at 25° C. for 16 h. After completion of the reaction, solvent was evaporated under reduced pressure to get the crude product. This crude product was purified by combi-flash chromatography using 0-30% ethyl acetate in heptane as an eluent R_{f} (EtOAc/heptane: 3/7)=0.4. Yield 1.2 g, 90%. MS (ESI) m/z [M+1]⁺: 359.11. ¹H-NMR (400 MHz, CDCl₃): δ 7.69-7.80 (m, 2H), 7.31-7.41 (m, 1H), 6.71-6.79 (m, 1H), 4.60-4.81 (m, 4H), 1.53 (s, 9H), 0.38 (s, 9H).

e) tert-Butyl 5-(isoxazol-3-yl)isoindoline-2-carboxylate (7)

To a solution of tert-butyl 5-(5-(trimethylsilyl)isoxazol-3-yl)isoindoline-2-carboxylate (1.20 g, 3.3 mmol, 1.00 eq.) in 25 mL MeOH, was added potassium carbonate (0.60 g, 5.0 mmol, 1.5 eq.) at 25° C. The resulting mixture was stirred at 60° C. for 2 h. After completion of the reaction, the reaction mixture was quenched with water and extracted (3 times) with EtOAc. The combined organic layer was washed with brine, dried over anhydrous sodium sulphate and concentrated under reduced pressure. The resulting crude product was forwarded to the next step without further purification. R_{f} (EtOAc/heptane: 5/5)=0.4. Yield: 1.1 g (crude). MS (ESI) m/z [M+1−56]⁺: 231.04. ¹H-NMR (400 MHz, CDCl₃): δ 8.46 (s, 1H), 7.70-7.75 (m, 2H), 7.34 (dd, 1H), 6.61-6.69 (m, 1H), 4.61-4.82 (m, 4H), 1.45 (s, 9H).

f) 3-(Isoindolin-5-yl)isoxazole trifluoroacetate (2e)

Treatment of tert-butyl 5-(isoxazol-3-yl)isoindoline-2-carboxylate (0.3 g, 1.05 mmol) with trifluoroacetic acid (2.0 mL) in in DCM (20 mL) afforded the title product as black gummy mass. R_{f} (MeOH/DCM: 0.5/9.5)=0.3. Yield 0.3 g (crude). MS (ESI) m/z [M+1]+:187.05. ¹H-NMR (400 MHz, CDCl₃): δ 9.10 (bs, 2H), 8.52 (d, 1H), 7.83 (d, 1H), 7.81 (s, 1H), 7.48 (d, 1H), 6.71 (d, 1H), 4.78 (s, 4H).

### Intermediate 2f: 2-(Isoindolin-5-yl)-1,3,4-thiadiazole Hydrochloride

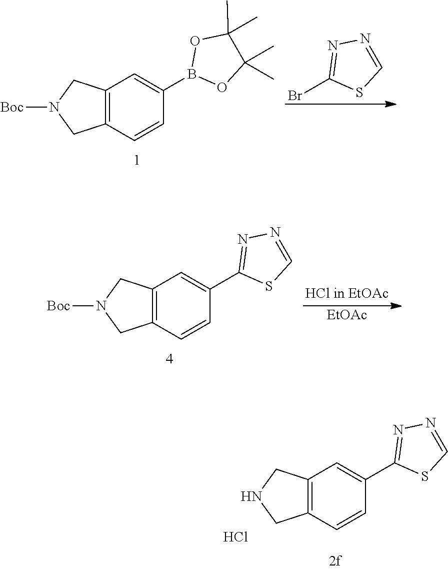

a) tert-Butyl 5-(1,3,4-thiadiazol-2-yl)isoindoline-2-carboxylate (4)

To a solution of 2-bromo-1,3,4-thiadiazole (0.50 g, 3.05 mmol, 1.00 eq.) in mixture of dioxane and water (2:1, 15 mL), were added tert-butyl 5-(4,4,5,5-tetramethyl-1,3,2-dioxaborolan-2-yl)isoindoline-2-carboxylate (1.88 g, 5.49 mmol, 1.8 eq.) and cesium carbonate (1.97 g, 6.10 mmol, 2.0 eq.) at 25° C. The mixture was then degassed by argon for 15 min. Then tetrakis(triphenylphosphine)palladium(0) (0.35 g, 0.30 mmol, 0.1 eq.) was added and the resulting mixture was heated at 80° C. for 16 h. After completion of the reaction, the mixture was quenched with water and extracted (3 times) with EtOAc. The combined organic layer was washed with brine, dried over anhydrous sodium sulphate, filtered and concentrated under reduced pressure. This crude product was purified by combi-flash chromatography using 0-20% ethyl acetate in heptane as an eluent. R_{f} (EtOAc/Heptane: 3/7)=0.4. Yield 0.43 g, 91%. MS(ESI) m/z [M+1]⁺: 304.05.

b) 2-(Isoindolin-5-yl)-1,3,4-thiadiazole Hydrochloride (2f)

Treatment of tert-butyl 5-(1,3,4-thiadiazol-2-yl)isoindoline-2-carboxylate (0.40 g, 1.32 mmol) with HCl in EtOAc (10.0 mL, 1.0 M solution) in EtOAc (10 mL) for 12 h afforded the title product as white solid. Yield 0.24 g (crude). MS(ESI) m/z [M+1]⁺: 203.97. ¹H NMR (DMSO-d₆) δ: 9.66 (s, 1H), 9.63 (bs, 1H), 8.10 (s, 1H), 8.02 (d, 1H), 7.59 (d, 1H), 4.56-4.60 (m, 4H).

### Intermediate 2g: 5-(1H-Pyrazol-1-yl)isoindoline trifluoroacetate

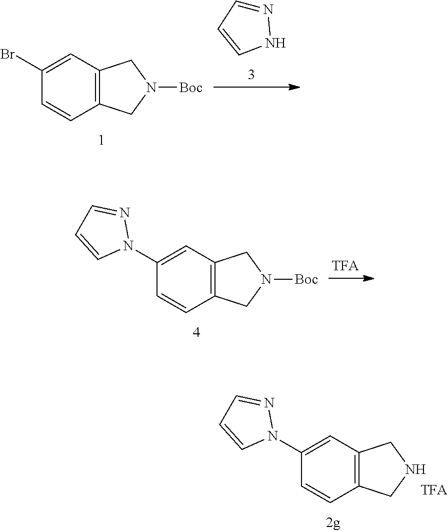 [SMILES: CC(C)(C)OC(=O)N1Cc2ccc(Br)cc2C1 ; c1cn[nH]c1 ; CC(C)(C)OC(=O)N1Cc2ccc(-n3cccn3)cc2C1 ; c1cnn(-c2ccc3c(c2)CNC3)c1]

a) tert-Butyl 5-(1H-pyrazol-1-yl)isoindoline-2-carboxylate (4)

To a solution of tert-butyl 5-bromoisoindoline-2-carboxylate (2.0 g, 6.73 mmol, 1.00 eq.) in 20 mL of DMA were added 1H-pyrazole (0.91 g, 13.4 mmol, 2.0 eq.) and potassium carbonate (2.7 g, 20.2 mmol, 3.0 eq.) at 25° C. The mixture was then degassed with argon for 15 min. Thereafter DMEDA (0.29 g, 3.36 mmol, 0.5 eq.) and CuI (0.63 g, 3.3 mmol, 0.5 eq.) were added and the resulting reaction mixture was heated at 150° C. for 16 h. After completion of the reaction, the reaction mixture was quenched with water and extracted (3 times) with EtOAc. The combined organic layer was washed with brine, dried over anhydrous sodium sulphate, filtered and concentrated under reduced pressure. This crude residue was purified by combi-flash chromatography using 0-20% ethyl acetate in heptane as an eluent. R_{f} (EtOAc/Heptane: 3/7)=0.6. Yield 0.5 g, 26%. MS (ESI) m/z [M+1]⁺: 286.14. ¹H-NMR (400 MHz, DMSO-d₆): δ 8.46 (d, 1H), 7.72-7.82 (m, 3H), 7.41-7.46 (m, 1H), 6.54 (s, 1H), 4.56-4.66 (m, 4H), 1.46 (s, 9H).

b) 5-(1H-Pyrazol-1-yl)isoindoline trifluoroacetate (2g)

Treatment of tert-butyl 5-(1H-pyrazol-1-yl)isoindoline-2-carboxylate (0.4 g, 1.40 mmol, 1.0 eq.) with trifluoroacetic acid (2 mL) in DCM (20 mL) at 25° C. for 16 h afforded the title product. R_{f} (MeOH/DCM: 0.5/9.5)=0.5. Yield 0.35 g (crude). MS (ESI) m/z [M+1]⁺:186.02. ¹H-NMR (400 MHz, DMSO-d₆): δ 9.50-9.80 (bs, 2H), 8.50 (s, 1H), 7.90 (s, 1H), 7.84 (d, 1H), 7.76 (s, 1H), 7.51 (d, 1H), 6.56 (s, 1H), 4.50-4.60 (m, 4H).

### Intermediate 2h: 3-(Isoindolin-5-yl)-1,2,4-oxadiazole trifluoroactete

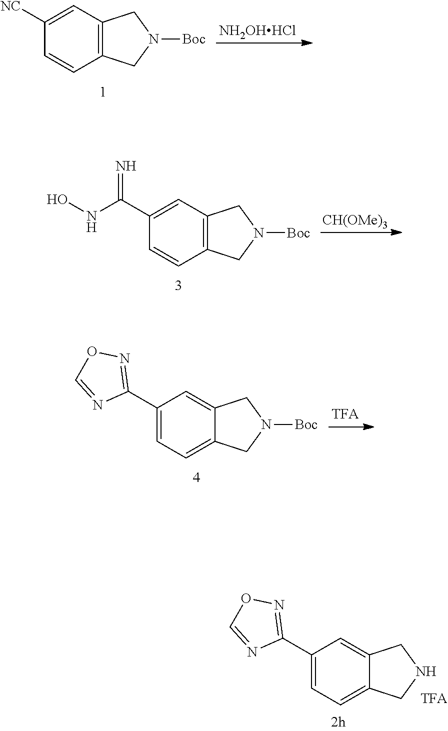

a) tert-Butyl 5-(N-hydroxycarbamimidoyl)isoindoline-2-carboxylate (3)

In a 100 mL seal tube tert-butyl 5-cyanoisoindoline-2-carboxylate (1.3 g, 5.32 mmol, 1.0 eq.) was dissolved in 30 mL of EtOH under nitrogenous atmosphere. Et₃N (4.5 g, 6.1 mL, 42.6 mmol, 8.0 eq.) and NH₂OH·HCl (1.1 g, 15.9 mmol, 3.0 eq.) were then added and the mixture was stirred at 90° C. for 12 h. After completion of the reaction, the reaction mixture was filtered and the resulting solid was triturated with EtOH and dried under vacuum to afford the title product. R_{f} (EtOAc/heptane: 3/7)=0.6. Yield 1.4 g (crude). MS (ESI) m/z [M+1]⁺: 278.13. ¹H-NMR (400 MHz, DMSO-d₆): δ 9.61 (s, 1H), 7.55-7.65 (m, 2H), 7.25-7.38 (m, 1H), 5.72-5.89 (m, 2H), 4.51-4.61 (m, 4H), 1.45 (s, 9H).

b) tert-Butyl 5-(1,2,4-oxadiazol-3-yl)isoindoline-2-carboxylate (4)

In a 100 mL seal tube tert-butyl 5-(N-hydroxycarbamimidoyl)isoindoline-2-carboxylate (0.85 g, 3.06 mmol, 1.0 eq.) was dissolved in 15 mL of triethylorthoformate under nitrogen atmosphere. TFA (0.2 mL) was then added and the mixture was stirred at 70° C. for 12 h. After completion of the reaction, the reaction mixture was quenched with water and extracted (3 times) with EtOAc. The combined organic layer was washed with brine, dried over anhydrous sodium sulphate, filtered and concentrated under reduced pressure. This crude product was forwarded to the next step without further purification. R_{f} (EtOAc/heptane: 3/7)=0.5. Yield 0.88 g (crude). MS (ESI) m/z [M+1−100]⁺: 188.04. ¹H-NMR (400 MHz, DMSO-d₆): δ 9.71 (s, 1H), 8.00 (d, 1H), 7.96 (d, 1H), 7.49-7.55 (m, 1H), 4.62-4.70 (m, 4H), 1.47 (s, 9H).

c) 3-(Isoindolin-5-yl)-1,2,4-oxadiazole trifluoroactete (2h)

Treatment of tert-butyl 5-(1,2,4-oxadiazol-3-yl)isoindoline-2-carboxylate (0.4 g, 1.39 mmol, 1.0 eq.) with trifluoroacetic acid (2 mL) in DCM (12 mL) at 25° C. for 2 h afforded the title product as black gummy liquid. R_{f} (MeOH/DCM: 0.5/9.5)=0.3. Yield 0.41 g (crude). MS (ESI) m/z [M+1]⁺:188.04. ¹H-NMR (400 MHz, DMSO-d₆): δ 9.75 (s, 1H), 9.58 (bs, 2H), 8.10 (s, 1H), 8.05 (d, 1H), 7.62 (d, 1H), 4.58-4.63 (m, 4H).

### Intermediate 2i: 2,2,2-Trifluoro-1-(isoindolin-5-yl)ethan-1-ol hydrogen Chloride

a) tert-Butyl 5-(2,2,2-trifluoro-1-hydroxyethyl)isoindoline-2-carboxylate (3)

To a solution of tert-butyl 5-formylisoindoline-2-carboxylate (2.5 g, 10.1 mmol, 1.0 eq.) in 10 mL of DMF were added trimethyl(trifluoromethyl)silane (3.5 g, 25.3 mmol, 2.5 eq.) and potassium carbonate (1.39 g, 10.1 mmol, 1.0 eq.) at 0° C. The mixture was stirred at 0° C. for 0.5 h. After 0.5 h the starting material was consumed and a nonpolar spot was observed in the TLC. The mixture was quenched with ice cold water and extracted (3 times) with EtOAc. The combined organic layer was washed with brine, dried over anhydrous sodium sulphate and concentrated under reduced pressure to get the crude product. The crude residue was dissolved in 10 mL MeOH and K₂CO₃ (0.7 g, 5.06, 0.5 eq.) was added followed by stirring for 0.25 h at 50° C. After completion of the reaction, the reaction mixture was quenched with ice cold water and extracted (3 times) with EtOAc. The combined organic layer was washed as dried as above and concentrated under reduced pressure to get the crude product. This crude residue was purified by combi-flash chromatography using 0-50% ethyl acetate in heptane as an eluent. R_{f} (EtOAc/Heptane: 8/2)=0.6. Yield 1.5 g, 47%. MS (ESI) m/z [M+1]+: 318.13. ¹H-NMR (400 MHz, DMSO-d₆) δ: 7.31-7.47 (m, 3H), 6.90 (d, 1H), 5.12-5.24 (m, 1H), 4.39-4.80 (m, 4H), 1.52 (s, 9H).

b) 2,2,2-Trifluoro-1-(isoindolin-5-yl)ethan-1-ol hydrogen chloride (2i)

Treatment of tert-butyl 5-(2,2,2-trifluoro-1-hydroxyethyl)isoindoline-2-carboxylate (0.40 g, 1.26 mmol, 1.0 eq.) with HCl in EtOAc (10.0 mL, 1.0 M solution) in EtOAc (10 mL) for 12 h afforded the title product as a white solid. R_{f} (MeOH/DCM: 0.5/9.5)=0.3. Yield 0.25 g (crude). MS (ESI) m/z [M+1]⁺: 218.04. ¹H-NMR (400 MHz, DMSO-d₆): δ 9.88 (bs, 2H), 7.53 (s, 1H), 7.41-7.47 (m, 2H), 6.96 (d, 1H), 5.16-5.27 (m, 1H), 4.50 (s, 4H).

### Intermediate 3: tert-Butyl 6-(1-hydroxyethyl)-2-azaspiro[3.3]heptane-2-carboxylate

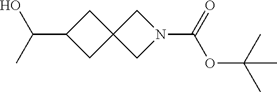 [SMILES: CC(O)C1CC2(C1)CN(C(=O)OC(C)(C)C)C2]

a) tert-Butyl 6-(methoxy(methyl)carbamoyl)-2-azaspiro[3.3]heptane-2-carboxylate

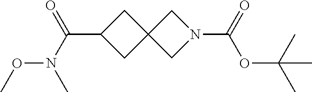 [SMILES: CON(C)C(=O)C1CC2(C1)CN(C(=O)OC(C)(C)C)C2]

To a cooled (0-5° C.) mixture of 2-(tert-butoxycarbonyl)-2-azaspiro[3.3]heptane-6-carboxylic acid (0.241 g, 1.0 mmol), N,O-dimethylhydroxylamine hydrochloride (0.122 g, 1.25 mmol) and triethylamine (0.80 ml, 5.74 mmol) in dry DMF (2.0 ml) was added 1-propanephosphonic acid cyclic anhydride (50% in EtOAc, 0.80 ml, 1.358 mmol). The mixture was stirred at RT overnight followed by diluting with water and extracting with EtOAc. Organic phase was washed with saturated NaHCO₃ solution, water and brine, followed by drying and evaporating to afford the title compound. Yield 0.23 g. ¹H NMR (400 MHz, CDCl₃): δ 3.96 (s, 2H), 3.85 (s, 2H), 3.65 (s, 2H), 3.26-3.43 (m, 1H), 3.17 (s, 3H), 2.41-2.52 (m, 2H), 2.28-2.38 (m, 2H), 1.43 (s, 9H).

b) tert-Butyl 6-acetyl-2-azaspiro[3.3]heptane-2-carboxylate

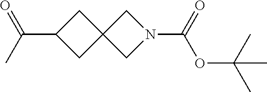 [SMILES: CC(=O)C1CC2(C1)CN(C(=O)OC(C)(C)C)C2]

To a cooled (0-5° C.) solution of tert-butyl 6-(methoxy(methyl)carbamoyl)-2-azaspiro[3.3]heptane-2-carboxylate (0.23 g, 0.809 mmol) in dry THF (3.0 ml) was added methylmagnesium bromide (3M in ether, 0.40 ml, 1.20 mmol). The mixture was stirred 1 h at 0-5° C. and then overnight at RT. Reaction was quenched by adding saturated NH₄Cl solution and water followed by extracting with DCM. Organic phase was dried and evaporated to afford the title compound. Yield 0.19 g. ¹H NMR (400 MHz, CDCl₃): δ 3.94 (s, 2H), 3.81 (s, 2H), 3.12 (quint, 1H), 2.27-2.42 (m, 4H), 2.09 (s, 3H), 1.43 (s, 9H).

c) tert-Butyl 6-(1-hydroxyethyl)-2-azaspiro[3.3]heptane-2-carboxylate

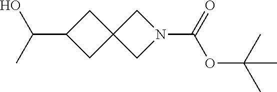 [SMILES: CC(O)C1CC2(C1)CN(C(=O)OC(C)(C)C)C2]

To a solution of tert-butyl 6-acetyl-2-azaspiro[3.3]heptane-2-carboxylate (0.19 g, 0.794 mmol) in dry methanol (3.0 ml) was added sodium borohydride (0.045 g, 1.191 mmol) in small portions. The mixture was stirred at RT until the reaction was completed. Methanol was evaporated, water and EtOAc were added and the phases separated. Aqueous phase was extracted with EtOAc. Combined organic phases were washed with brine, dried and evaporated to afford the title compound. Yield 0.19 g. ¹H NMR (400 MHz, CDCl₃): δ 3.92 (s, 2H), 3.76-3.84 (m, 2H), 3.60-3.69 (m, 1H), 2.06-2.25 (m, 3H), 1.98-2.05 (m, 1H), 1.86-1.94 (m, 1H), 1.43 (s, 9H), 1.32 (d, 1H), 1.09 (d, 3H).

The following intermediates were prepared according to the procedure described in Example 10(a) from the starting materials indicated on the table.

| No. | Structure | LC-MS | Starting material |
|---|---|---|---|
| Int-4 | 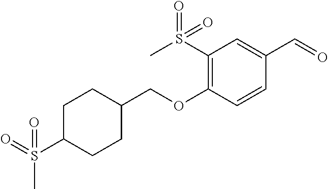 [SMILES: CS(=O)(=O)c1cc(C=O)ccc1OCC1CCC(S(C)(=O)=O)CC1] | m/z 376.2 [M + H]⁺ | 4-Hydroxy-3-(methylsulfonyl)- benzaldehyde and 4-(Methane- sulphonyloxymethyl)-1- methanesulphonylpiperidine |
| Int-5 | 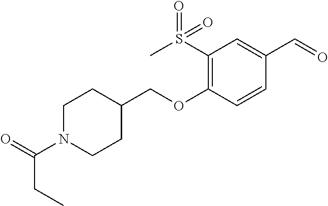 [SMILES: CCC(=O)N1CCC(COc2ccc(C=O)cc2S(C)(=O)=O)CC1] | m/z 354.6 [M + H]⁺ | 4-Hydroxy-3-(methylsulfonyl)- benzaldehyde and (1-Propionyl- piperidin-4-yl)methyl 4-methyl- benzenesulfonate |
| Int-6 | 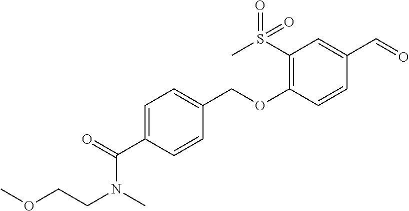 [SMILES: COCCN(C)C(=O)c1ccc(COc2ccc(C=O)cc2S(C)(=O)=O)cc1] | m/z 406.3 [M + H]⁺ | 4-Hydroxy-3-(methylsulfonyl)- benzaldehyde and 4-(Chloro- methyl)-N-(2-methoxyethyl)-N- methylbenzamide |
| Int-7 | 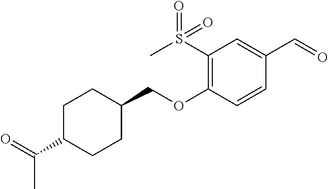 | m/z 339.3 [M + H]⁺ | 4-Hydroxy-3-(methylsulfonyl)- benzaldehyde and ((1r,4r)-4- Acetylcyclohexyl)methyl 4- methylbenzenesulfonate |
| Int-8 | 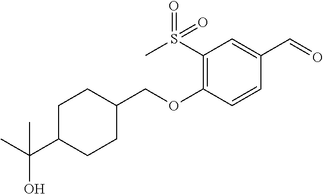 | m/z 349.4 [M + H]⁺ | 4-Hydroxy-3-(methylsulfonyl)- benzaldehyde and 2-(4-(Bromo- methyl)phenyl)propan-2-ol |
| Int-9 | 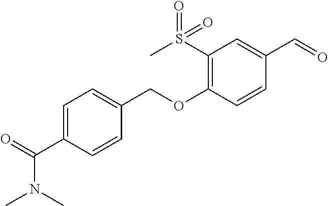 [SMILES: CN(C)C(=O)c1ccc(COc2ccc(C=O)cc2S(C)(=O)=O)cc1] | m/z 362.3 [M + H]⁺ | 4-Hydroxy-3-(methylsulfonyl)- benzaldehyde and Benzamide,4- (chloromethyl)-N,N-dimethyl |
| Int-10 | 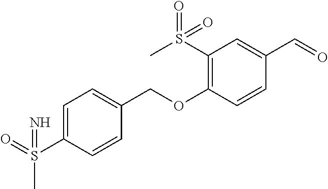 [SMILES: CS(=N)(=O)c1ccc(COc2ccc(C=O)cc2S(C)(=O)=O)cc1] | m/z 368.4 [M + H]⁺ | 4-Hydroxy-3-(methylsulfonyl)- benzaldehyde and 1-(chloro- methyl)-4-(S-methylsulfon- imidoyl)benzene |
| Int-11 | 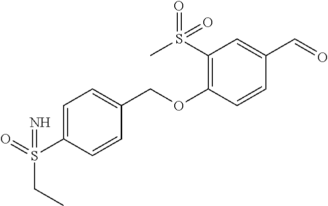 [SMILES: CCS(=N)(=O)c1ccc(COc2ccc(C=O)cc2S(C)(=O)=O)cc1] | m/z 382.3 [M + H]⁺ | 4-Hydroxy-3-(methylsulfonyl)- benzaldehyde and (4-(Chloro- methyl)phenyl)(ethyl)(imino)- λ⁶-sulfanone |
| Int-12 | 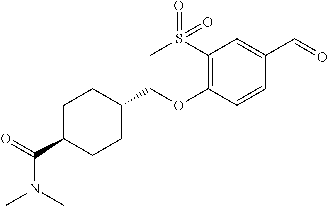 [SMILES: CN(C)C(=O)[C@H]1CC[C@H](COc2ccc(C=O)cc2S(C)(=O)=O)CC1] | m/z 368.5 [M + H]⁺ | 4-Hydroxy-3-(methylsulfonyl)- benzaldehyde and ((1r,4r)-4- (Dimethylcarbamoyl)cyclohexyl) methyl 4-methylbenzenesulfonate |

In the following synthesis examples reference is made to the preparative HPLC methods A, B or C.

Preparative HPLC Methods:

Method A:

Instruments: Agilent technologies 1260 infinity Column: SUNFIRE C-18, 10 micron, 19×250 mm; Gradient [time (min)/solvent B in A (%)]: 0.01/10, 3.00/10, 13.00/35; Solvents: solvent A=0.1% TFA in water; solvent B=acetonitrile; Detection wavelength 214 nm; Flow rate 20 mL/min.

Method B:

Instruments: Agilent technologies 1260 infinity Column: X select hexyl phenyl, 5 micron, 19×250 mm; Gradient [time (min)/solvent B in A (%)]: 0.01/15, 3.00/15, 12.00/35, 16.00/35; Solvents: solvent A=0.1% TFA in water; solvent B=acetonitrile; Detection wavelength 214 nm; Flow rate 15 mL/min.

Method C:

Instruments: Agilent technologies 1260 infinity Column: X bridge shield, 10 micron, 19×250 mm; Gradient [time (min)/solvent B in A (%)]: 0.01/45, 3.00/45, 13.00/65, 14.00/65; Solvents: solvent A=5 mM ammonium acetate in water; solvent B=acetonitrile; Detection wavelength 214 nm; Flow rate 18 mL/min.

### Example 1

2-((5-(1,3,4-Oxadiazol-2-yl)isoindolin-2-yl)methyl)-5-(((methylsulfonyl)piperidin-4-yl)methoxy)-4H-pyran-4-one (Compound 1)

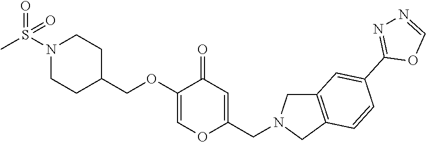 [SMILES: CS(=O)(=O)N1CCC(COc2coc(CN3Cc4ccc(-c5nnco5)cc4C3)cc2=O)CC1]

To a solution of 2-(chloromethyl)-5-((1-(methylsulfonyl)piperidin-4-yl)methoxy)-4H-pyran-4-one (0.29 g, 0.86, 1.0 eq.) in CH₃CN (30 mL) was added DIPEA (0.55 g, 4.3 mmol, 5.0 eq.) and 2-(isoindolin-5-yl)-1,3,4-oxadiazole, trifluoroacetate salt (0.25 g, 0.86 mmol, 1.0 eq.). The resulting mixture was stirred at 90° C. for 16 h. After completion of the reaction as indicated by TLC, the reaction mixture was quenched with ice cold water and extracted (3 times) with EtOAc. The combined organic layer was washed with brine, dried over anhydrous sodium sulphate and concentrated under reduced pressure to get the crude product. This crude product was purified by preparative HPLC to afford the pure product as a white solid, R_{f} (MeOH/DCM: 0.5/9.5)=0.3. Yield 0.070 g. ¹H NMR (400 MHz, DMSO-d₆) δ: 9.33 (s, 1H), 8.16 (s, 1H), 7.92 (s, 1H), 7.89 (d, 1H), 7.48 (d, 1H), 6.41 (s, 1H), 4.04 (s, 4H), 3.82 (s, 2H), 3.72 (d, 2H), 3.58 (d, 2H), 2.85 (s, 3H), 2.72 (t, 2H), 1.75-1.91 (m, 3H), 1.21-1.36 (m, 2H). MS (ESI) m/z [M+1]⁺: 487.4.

### Example 2

5-((1-(Methylsulfonyl)piperidin-4-yl)methoxy)-2-((5-(oxazol-5-yl)isoindolin-2-yl)methyl)-4H-pyran-4-one (Compound 2)

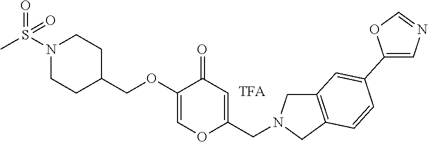 [SMILES: CS(=O)(=O)N1CCC(COc2coc(CN3Cc4ccc(-c5cnco5)cc4C3)cc2=O)CC1]

Following the procedure of Example 1, treatment of 2-(isoindolin-5-yl)oxazole hydrogen chloride (0.2 g, 0.90 mmol, 1.0 eq.) with 2-(chloromethyl)-5-((1-(methyl-sulfonyl)piperidin-4-yl)methoxy)-4H-pyran-4-one (0.30 g, 0.90 mmol, 1.0 eq.) and DIPEA (0.46 g, 3.60 mmol, 4.0 eq.) in CH₃CN (10 mL) at 70° C. for 16 h followed by preparative HPLC purification using method A (rt: 12.03 min) afforded the title product as TFA salt. R_{f} (MeOH/DCM: 0.5/9.5)=0.6. Yield 0.021 g. ¹H NMR (400 MHz, DMSO-d₆) δ: 8.46 (s, 1H), 8.21 (s, 1H), 7.70-7.71 (m, 3H), 7.45 (d, 1H), 6.57 (s, 1H), 4.42-4.53 (m, 6H), 3.73 (d, 2H), 3.59 (d, 2H), 2.85 (s, 3H), 2.72 (t, 2H), 1.83-1.86 (m, 3H), 1.25-1.34 (m, 2H). MS (ESI) m/z (M+1)⁺ 486.3.

### Example 3

5-((1-(Methylsulfonyl)piperidin-4-yl)methoxy)-2-((5-(oxazol-2-yl)isoindolin-2-yl)methyl)-4H-pyran-4-one (Compound 3)

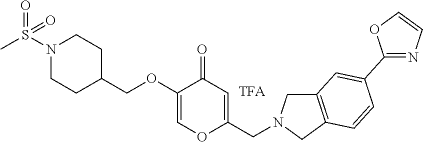 [SMILES: CS(=O)(=O)N1CCC(COc2coc(CN3Cc4ccc(-c5ncco5)cc4C3)cc2=O)CC1]

Following the procedure of Example 1, treatment of 2-(isoindolin-5-yl)oxazole hydrogen chloride (0.2 g, 0.90 mmol, 1.0 eq.) with 2-(chloromethyl)-5-((1-(methyl-sulfonyl)piperidin-4-yl)methoxy)-4H-pyran-4-one (0.30 g, 0.90 mmol, 1.0 eq) and DIPEA (0.46 g, 3.60 mmol, 4.0 eq.) in CH₃CN (10 mL) at 70° C. for 16 h followed by preparative HPLC purification using method A (rt: 12.46 min) afforded the title product as TFA salt. R_{f} (MeOH/DCM: 0.5/9.5)=0.6. Yield 0.011 g. ¹H NMR (400 MHz, DMSO-d₆) δ: 8.22 (d, 2H), 7.93-7.97 (m, 2H), 7.50 (d, 1H), 7.40 (s, 1H), 6.49-6.58 (m, 1H), 4.42-4.85 (m, 6H), 3.73 (d, 2H), 3.59 (d, 2H), 2.86 (s, 3H), 2.72 (t, 2H), 1.83-1.86 (m, 3H), 1.25-1.34 (m, 2H). MS (ESI) m/z [M+1]⁺: 486.3.

### Example 4

2-((5-(Isoxazol-5-yl)isoindolin-2-yl)methyl)-5-((1-(methylsulfonyl)piperidin-4-yl)methoxy)-4H-pyran-4-one (Compound 4)

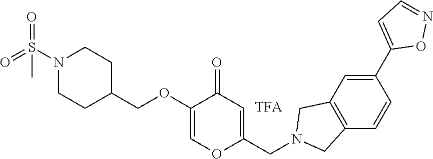 [SMILES: CS(=O)(=O)N1CCC(COc2coc(CN3Cc4ccc(-c5ccno5)cc4C3)cc2=O)CC1]

Following the procedure of Example 1, treatment of 5-(isoindolin-5-yl)isoxazole hydrogen chloride (0.27 g, 1.22 mmol, 1.0 eq.) with 2-(chloromethyl)-5-((1-(methyl-sulfonyl)piperidin-4-yl)methoxy)-4H-pyran-4-one (0.41 g, 1.22 mmol, 1.0 eq.) and DIPEA (0.79 g, 1.1 mL, 6.10 mmol, 5.0 eq.) in CH₃CN (15 mL) at 80° C. for 16 h followed by preparative HPLC purification using method A afforded the title product as a brown solid. R_{f} (MeOH/DCM: 0.5/9.5)=0.2. Yield 0.048 g. ¹H NMR (400 MHz, DMSO-d₆) δ: 8.67 (d, 1H), 8.21 (s, 1H), 7.84-7.87 (m, 2H), 7.51 (d, 1H), 7.03 (s, 1H), 6.56 (s, 1H), 4.30-4.48 (m, 4H), 4.03-4.20 (m, 2H), 3.72 (d, 2H), 3.58 (d, 2H), 2.86 (s, 3H), 2.72 (t, 2H), 1.83-1.85 (m, 3H), 1.25-1.33 (m, 2H). MS (ESI) m/z [M+1]⁺: 486.3.

### Example 5

2-((5-(Isoxazol-3-yl)isoindolin-2-yl)methyl)-5-((1-(methylsulfonyl)piperidin-4-yl)methoxy)-4H-pyran-4-one (Compound 5)

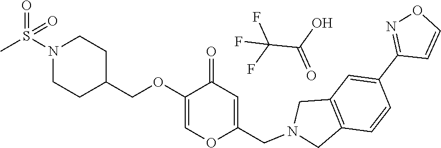

Following the procedure of Example 1, treatment of 3-(isoindolin-5-yl)isoxazole trifluoroacetate (0.3 g, 1.06 mmol, 1.0 eq.) with 2-(chloromethyl)-5-((1-(methylsulfo-nyl)piperidin-4-yl)methoxy)-4H-pyran-4-one (0.35 g, 1.06 mmol, 1.0 eq.) and DIPEA (0.68 g, 0.98 mL, 5.30 mmol, 5.0 eq.) in CH₃CN (20 mL) at 80° C. for 16 h followed by preparative HPLC purification using method A (rt: 11.78 min) afforded the title product as TFA salt. R_{f} (MeOH/DCM: 0.5/9.5)=0.3. Yield 0.019 g. ¹H NMR (400 MHz, DMSO-d₆) δ: 9.02 (s,1H), 8.21 (s,1H), 7.80-7.90 (m, 2H), 7.45-7.55 (m, 1H), 7.14 (d, 1H), 6.49-6.60 (m, 1H), 4.10-4.80 (m, 6H), 3.73 (d, 2H), 3.51-3.59 (m, 2H), 2.85 (s, 3H), 2.71 (q, 2H), 1.84 (d, 3H), 1.21-1.33 (m, 2H). MS (ESI) m/z [M+1]⁺: 486.2.

### Example 6

2-((5-(1,3,4-Thiadiazol-2-yl)isoindolin-2-yl)methyl)-5-((1-(methylsulfonyl)-piperidin-4-yl)methoxy)-4H-pyran-4-one (Compound 6)

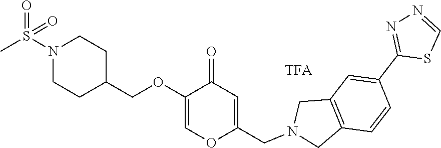 [SMILES: CS(=O)(=O)N1CCC(COc2coc(CN3Cc4ccc(-c5nncs5)cc4C3)cc2=O)CC1]

Following the procedure of Example 1, treatment of 2-(isoindolin-5-yl)-1,3,4-thiadiazole hydrochloride (0.20 g, 0.83 mmol) with 2-(chloromethyl)-5-((1-(methyl-sulfonyl)piperidin-4-yl)methoxy)-4H-pyran-4-one (0.28 g, 0.83 mmol, 1.0 eq.) and DIPEA (0.54 g, 0.76 mL, 4.15 mmol, 5.0 eq.) in CH₃CN (10 mL) at 80° C. for 16 h followed by preparative HPLC purification using method A (rt: 11.43 min) afforded the title product as TFA salt. R_{f} (MeOH/DCM: 1/9)=0.3. Yield 0.044 g. ¹H NMR (400 MHz, DMSO-d₆) δ: 9.64 (d, 1H), 8.22 (s, 1H), 8.03 (s, 1H), 7.96 (d, 1H), 7.53 (d, 1H), 6.57 (s, 1H), 4.48 (s, 6H), 3.73 (d, 2H), 3.59 (d, 2H), 2.86 (s, 3H), 2.72 (t, 2H), 1.83-1.85 (m, 3H), 1.25-1.34 (m, 2H). MS (ESI) m/z [M+1]⁺: 503.2.

### Example 7

2-((5-(1H-Pyrazol-1-yl)isoindolin-2-yl)methyl)-5-((1-(methylsulfonyl)piperidin-4-yl)methoxy)-4H-pyran-4-one (Compound 7)

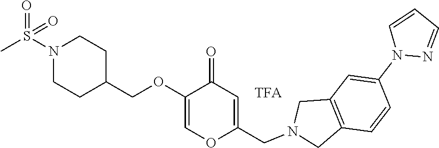 [SMILES: CS(=O)(=O)N1CCC(COc2coc(CN3Cc4ccc(-n5cccn5)cc4C3)cc2=O)CC1]

Following the procedure of Example 1, treatment of 5-(1H-pyrazol-1-yl)isoindo-line trifluoroacetate (0.35 g, 1.24 mmol, 1.0 eq.) with 2-(chloromethyl)-5-((1-(methyl-sulfonyl)piperidin-4-yl)methoxy)-4H-pyran-4-one (0.41 g, 1.24 mmol, 1.0 eq.) and DIPEA (0.80 g, 1.14 mL, 6.2 mmol, 5.0 eq.) in CH₃CN (10 mL) at 90° C. for 16 h followed by preparative HPLC purification using method A (rt: 11.33 min) afforded the title product as TFA salt. R_{f} (MeOH/DCM: 0.5/9.5)=0.3. Yield 0.060 g. ¹H NMR (400 MHz, DMSO-d₆) δ: 8.47 (d, 1H), 8.22 (s, 1H), 7.86 (s, 1H), 7.80 (d, 1H), 7.75 (s, 1H), 7.46 (d, 1H), 6.59 (s, 1H), 6.56 (s, 1H), 4.43-4.54 (m, 6H), 3.73 (d, 2H), 3.59 (d, 2H), 2.86 (s, 3H), 2.73 (t, 2H), 1.83-1.86 (m, 3H), 1.28-1.34 (m, 2H). MS (ESI) m/z [M+1]⁺: 485.3.

### Example 8

2-((5-(1,2,4-Oxadiazol-3-yl)isoindolin-2-yl)methyl)-5-((1-(methylsulfonyl)-piperidin-4-yl)methoxy)-4H-pyran-4-one (Compound 8)

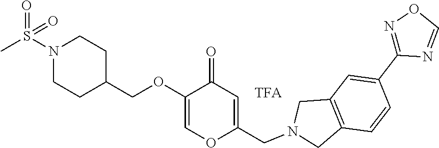 [SMILES: CS(=O)(=O)N1CCC(COc2coc(CN3Cc4ccc(-c5ncon5)cc4C3)cc2=O)CC1]

Following the procedure of Example 1, treatment of 3-(isoindolin-5-yl)-1,2,4-oxadiazole trifluoroactete (0.4 g, 1.40 mmol, 1.0 eq.) with 2-(chloromethyl)-5-((1-(methylsulfonyl)piperidin-4-yl)methoxy)-4H-pyran-4-one (0.47 g, 1.40 mmol, 1.0 eq.) and DIPEA (0.90 g, 1.3 mL, 7.0 mmol, 5.0 eq.) in CH₃CN (50 mL) at 90° C. for 12 h followed by column chromatography afforded the title product as a white solid. TFA salt of this compound was synthesized by stirring the title product with 0.1% aqueous TFA in CH₃CN (5 mL) for 15 min followed by lyophilization. R_{f} (MeOH/DCM: 0.5/9.5)=0.5. Yield 0.34 g. ¹H NMR (400 MHz, DMSO-d₆) δ: 9.69 (s, 1H), 8.16 (s, 1H), 7.90-7.92 (m, 2H), 7.45 (d, 1H), 6.41 (s, 1H), 4.04 (s, 2H), 4.03 (s, 2H), 4.82 (d, 2H), 3.72 (d, 2H), 3.58 (d, 2H), 2.85 (s, 3H), 2.72 (t, 2H), 1.83-1.85 (m, 3H), 1.24-1.33 (m, 2H). MS (ESI) m/z [M+1]⁺: m/z 487.2.

### Example 9

5-((1-(Methylsulfonyl)piperidin-4-yl)methoxy)-2-((5-(2,2,2trifluorohydroxyethyl) isoindolin-2-yl)methyl)-4H-pyran-4-one (Compound 9)

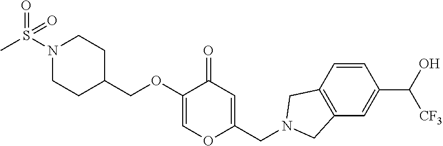 [SMILES: CS(=O)(=O)N1CCC(COc2coc(CN3Cc4ccc(C(O)C(F)(F)F)cc4C3)cc2=O)CC1]

Following the procedure of Example 1, treatment of 2-(chloromethyl)-5-((1-(methylsulfonyl)piperidin-4-yl)methoxy)-4H-pyran-4-one (0.25 g, 0.75 mmol, 1.0) with 2,2,2-trifluoro-1-(isoindolin-5-yl)ethan-1-ol hydrogen chloride (0.28 g, 1.12 mmol, 1.5 eq.) and DIPEA (0.48 g, 0.69 mL, 3.75 mmol, 5.0 eq.) in CH₃CN (10 mL) at 65° C. for 12 h followed by column chromatography afforded the desire product as brown solid. R_{f} (MeOH/DCM: 0.5/9.5)=0.5. Yield 0.25 g. ¹H NMR (400 MHz, DMSO-d₆) δ: 8.15 (s, 1H), 7.22-7.38 (m, 3H), 6.79 (d, 1H), 6.39 (s, 1H), 5.08-5.15 (m, 1H), 3.96 (s, 4H), 3.79 (s, 2H), 3.72 (d, 2H), 3.57 (d, 2H), 2.85 (s, 3H), 2.65-2.77 (m, 2H), 1.81-1.88 (m, 3H), 1.21-1.35 (m, 2H). MS (ESI) m/z [M+1]⁺: 517.3.

### Example 10

2-(2-(3-(Methylsulfonyl)-4-((1-(methylsulfonyl)piperidin-4-yl)methoxy)benzyl)-isoindolin-5-yl)-1,3,4-oxadiazole (Compound 10)

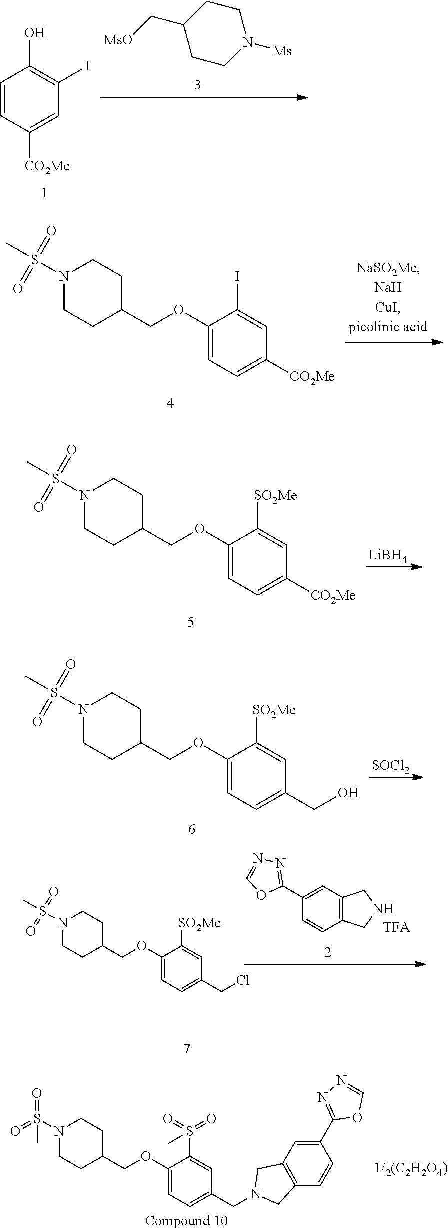

a) Methyl 3-iodo-4-((1-(methylsulfonyl)piperidin-4-yl)methoxy)benzoate (4)

To a solution of methyl 4-hydroxy-3-iodobenzoate (20.0 g, 71.9 mmol, 1.0 eq.) in 400 mL of DMF were added (1-(methylsulfonyl)piperidin-4-yl)methyl methane-sulfonate (19.5 g, 71.9 mmol, 1.0 eq.) and potassium carbonate (14.5 g, 107.91 mmol, 1.5 eq.) at 25° C. The resulting mixture was stirred at 110° C. for 16 h. After completion of the reaction, the mixture was cooled to 0° C. and quenched with ice cold water to get the precipitate. The mixture was filtered and the resulting solid was washed with water and dried in vacuo. This product was forwarded to the next step without further purification. R_{f} (EtOAc/Heptane: 6/4)=0.5. Yield 28.1 g. MS (ESI) m/z [M+1]+: 454.10. ¹H-NMR (400 MHz, DMSO-d₆) δ: 8.28 (s, 1H), 7.95 (d, 1H), 7.09 (d, 1H), 4.04 (d, 2H), 3.82 (s, 3H), 3.61 (d, 2H), 2.86 (s, 3H), 2.76 (t, 2H), 1.87-1.94 (m, 3H), 1.40-1.53 (m, 2H).

b) Methyl 3-(methylsulfonyl)-4-((1-(methylsulfonyl)piperidin-4-yl)methoxy)-benzoate (5)

To a solution of methyl 3-iodo-4-((1-(methylsulfonyl)piperidin-4-yl)methoxy)-benzoate (20.0 g, 44.2 mmol, 1.00 eq.) in 200 mL of DMSO were added sodium methanesulfinate (9.0 g, 88.3 mmol, 2.0 eq.) and sodium hydride (1.76 g, 44.2 mmol, 1.0 eq.) at 25° C. The mixture was then degassed by argon for 15 min. Then copper iodide (II) (0.8.38 g, 44.2 mmol, 1.0 eq.) and picolinic acid (5.43 g, 44.15 mmol, 1.0 eq.) were added followed by heating at 100° C. for 16 h. After completion of the reaction, the mixture was quenched with water and extracted (3 times) with EtOAc. The combined organic layer was washed with brine, dried over anhydrous sodium sulphate, filtered and concentrated under reduced pressure. This crude residue was purified by column chromatography using 100-200 mesh silica gel and 0-60% ethyl acetate in heptane as an eluent. R_{f} (EtOAc/heptane: 6/4)=0.2. Yield 12.0 g, 67%. MS (ESI) m/z [M+1]⁺: 406.20. ¹H-NMR (400 MHz, DMSO-d₆) δ: 8.35 (d, 1H), 8.24 (dd, 1H), 7.43 (d, 1H), 4.19 (d, 2H), 3.87 (s, 3H), 3.60 (d, 2H), 3.29 (s, 3H), 2.82 (s, 3H), 2.76 (t, 2H), 1.94-2.02 (m, 1H), 1.91 (d, 2H), 1.35-1.49 (m, 2H).

c) (3-(Methylsulfonyl)-4-((1-(methylsulfonyl)piperidin-4-yl)methoxy)phenyl)-methanol (6)

To a solution of methyl 3-(methylsulfonyl)-4-((1-(methylsulfonyl)piperidin-4-yl)methoxy)benzoate (6.0 g, 14.8 mmol, 1.0 eq.) in 20 mL of THF were added lithium tetrahydroborate (24.6 mL, 73.98 mmol, 5.0 eq) at 0° C. The resulting mixture was heated at 50° C. for 24 h. After completion of the reaction, the mixture was cooled to 0° C., quenched with NH₄Cl solution and extracted (3 times) with EtOAc. The combined organic layer was washed with brine, dried over anhydrous sodium sulphate and concentrated under reduced pressure. This crude was forwarded to the next step without further purification. R_{f} (MeOH/DCM: 0.5/9.5)=0.2. Yield 3.2 g (crude). MS (ESI) m/z [M+1]⁺: 378.18. ¹H-NMR (400 MHz, DMSO-d₆) δ: 7.78 (s, 1H), 7.57 (d, 1H), 7.24 (d, 1H), 5.30 (t, 1H), 4.48 (d, 2H), 4.07 (d, 2H), 3.60 (d, 2H), 3.23 (s, 3H), 2.86 (s, 3H), 2.75 (t, 2H), 1.87-2.01 (m, 3H), 1.33-1.47 (m, 2H).

d) 4-((4-(Chloromethyl)-2-(methylsulfonyl)phenoxy)methyl) (methylsulfonyl)piperidine (7)

To a solution of (3-(methylsulfonyl)-4-((1-(methylsulfonyl)piperidin-4-yl)-methoxy)phenyl)methanol (1.70 g, 4.50 mmol, 1.00 eq.) in 50 mL of CH₃CN and DMF (2-3 drops) was added thionyl chloride (8.04 g, 4.9 mL, 67.6 mmol, 15.0 eq.) at 0° C. The mixture was stirred at 25° C. for 1 h. After completion of the reaction, solvent was evaporated under reduced pressure to get the crude product. R_{f} (MeOH/EtOAc: 0.5/9.5)=0.6. Yield 1.6 g (crude). MS (ESI) m/z [M+1]⁺: 396.17. ¹H-NMR (400 MHz, DMSO-d₆) δ: 7.89 (d, 1H), 7.74 (d, 1H), 7.34 (d, 1H), 4.84 (s, 2H), 4.10 (d, 2H), 3.60 (d, 2H), 3.27 (s, 3H), 2.86 (s, 3H), 2.75 (t, 2H), 1.85-2.02 (m, 3H), 1.41-1.49 (m, 2H).

e) 2-(2-(3-(Methylsulfonyl)-4-((1-(methylsulfonyl)piperidin-4-yl)methoxy)-benzyl)isoindolin-5-yl)-1,3,4-oxadiazole (Compound 10)

Treatment of 2-(isoindolin-5-yl)-1,3,4-oxadiazole trifluoroacetate (0.4 g, 1.32 mmol, 1.0 eq.) with 4-((4-(chloromethyl)-2-(methylsulfonyl)phenoxy)methyl)-1-(methylsulfonyl)piperidine (0.52 g, 1.3 mmol, 1.0 eq.) in the presence of DIPEA (0.85 g, 1.2 mL, 6.6 mmol, 5.0 eq.) in CH₃CN (20 mL) at 90° C. for 16 h followed by column chromatography afforded the title product as light pink solid. Oxalic salt of the product was synthesized by employing oxalic acid (0.5 eq.) in MeOH at 0° C. for 3 h followed by filtration of the product. R_{f} (MeOH/DCM: 0.5/9.5)=0.6. Yield 0.34 g. ¹H NMR(400 MHz, DMSO-d₆) : δ 9.32 (s, 1H), 7.88-7.94 (m, 3H), 7.72 (d, 1H), 7.50 (d, 1H), 7.31 (d, 1H), 3.90-4.30 (m, 8H), 3.61 (d, 2H), 3.26 (s, 3H), 2.86 (s, 3H), 2.76 (t, 2H), 1.95-2.05 (m, 1H), 1.92 (d, 2H), 1.38-1.46 (m, 2H).

### Example 11

3-(2-(3-(Methylsulfonyl)-4-((1-(methylsulfonyl)piperidin-4-yl)methoxy)benzyl)-isoindolin-5-yl)isoxazole (Compound 11)

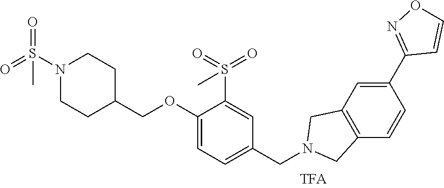 [SMILES: CS(=O)(=O)c1cc(CN2Cc3ccc(-c4ccon4)cc3C2)ccc1OCC1CCN(S(C)(=O)=O)CC1]

Following the procedure of Example 10, treatment of 3-(isoindolin-5-yl)isoxazole hydrochloride (0.19 g, 0.63 mmol, 1.0 eq.) with 4-((4-(chloromethyl)-2-(methylsulfonyl)phenoxy)methyl)-1-(methylsulfonyl)piperidine (0.25 g, 0.63 mmol, 1.0 eq.) in the presence of DIPEA (0.40 g, 0.6 mL, 3.16 mmol, 5.0 eq.) in CH₃CN (10 mL) at 90° C. for 16 h followed by column chromatography afforded the title product as white solid. TFA salt of the product was synthesized by treating the title product with 0.1% of TFA in CH₃CN at 25° C. for 5 min followed by lyophilization. R_{f} (MeOH/DCM: 0.5/9.5)=0.6. Yield 0.055 g. MS (ESI) m/z [M+1]⁺: 546.3 (M+H)⁺. ¹H NMR (400 MHz, DMSO-d₆): δ 10.90 (bs, 1H), 9.04 (s, 1H), 8.10-8.15 (m, 1H), 7.94-7.99 (s, 1H), 7.80-7.90 (m, 2H), 7.55 (d, 1H), 7.43 (d, 1H), 7.14 (s, 1H), 4.60-4.78 (s, 6H), 4.14 (d, 2H), 3.62 (d, 2H), 3.28 (s, 3H), 2.87 (s, 3H), 2.76 (t, 2H), 1.95-2.05 (m, 1H), 1.92 (d, 2H), 1.38-1.47 (m, 2H).

### Example 12

5-Bromo-2-(3-(methylsulfonyl)-4-((1-(methylsulfonyl)piperidin-4-yl)methoxy)-benzyl)isoindoline (Compound 12a) and

2-(3-(Methylsulfonyl)-4-((1-(methylsulfonyl)piperidin-4-yl)methoxy)benzyl)-5-(1H-pyrazol-1-yl)isoindoline (Compound 12b)

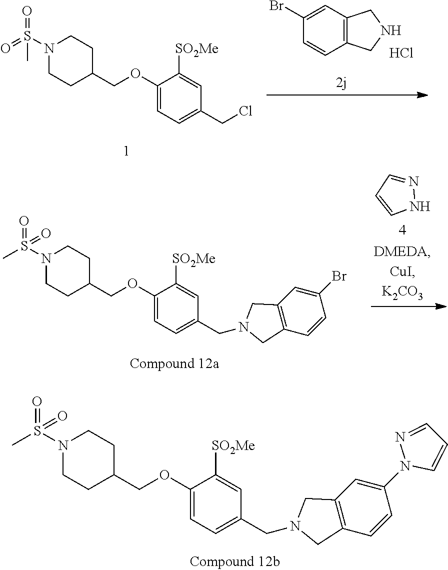 [SMILES: CS(=O)(=O)c1cc(CCl)ccc1OCC1CCN(S(C)(=O)=O)CC1 ; Brc1ccc2c(c1)CNC2 ; Cl ; c1cn[nH]c1 ; CS(=O)(=O)c1cc(CN2Cc3ccc(Br)cc3C2)ccc1OCC1CCN(S(C)(=O)=O)CC1 ; CS(=O)(=O)c1cc(CN2Cc3ccc(-n4cccn4)cc3C2)ccc1OCC1CCN(S(C)(=O)=O)CC1]

a) 5-Bromo-2-(3-(methylsulfonyl)-4-((1-(methylsulfonyl)piperidin-4-yl)methoxy)-benzyl)isoindoline (Compound 12a)

Treatment of 5-bromoisoindoline hydrogen chloride (0.5 g, 2.11 mmol, 1.0 eq.) with 4-((4-(chloromethyl)-2-(methylsulfonyl)phenoxy)methyl)-1-(methylsulfonyl)-piperidine (1.02 g, 2.59 mmol, 1.2 eq.) in the presence of DIPEA (1.0 g, 1.5 mL, 8.44 mmol, 4.0 eq.) in 10 mL of CH₃CN at 100° C. for 3 h followed by column chromatography afforded the title product. R_{f} (MeOH/EtOAc: 0.5/9.5)=0.2. Yield 0.66 g, 94%. MS (ESI) m/z [M+1]⁺: 557.11. ¹H-NMR (400 MHz, DMSO-d₆): δ 7.80 (d, 1H), 7.65 (dd, 1H), 7.43-7.47 (m, 1H), 7.37 (d, 1H), 7.27 (d, 1H), 7.19 (d, 1H), 4.08 (d, 2H), 3.86 (s, 2H), 3.82 (s, 2H), 3.79 (s, 2H), 3.60 (d, 2H), 3.25 (s, 3H), 2.87 (s, 3H), 2.75 (t, 2H), 1.88-2.01 (m, 3H), 1.36-1.51 (m, 2H).

b) 2-(3-(Methylsulfonyl)-4-((1-(methylsulfonyl)piperidin-4-yl)methoxy)benzyl)-5-(1H-pyrazol-1-yl)isoindoline (Compound 12b)

Treatment of 5-bromo-2-(3-(methylsulfonyl)-4-((1-(methylsulfonyl)piperidin-4-yl)methoxy)benzyl)isoindoline (0.36 g, 0.64 mmol, 1.00 eq.) with 1H-pyrazole (0.88 g, 1.29 mmol, 2.0 eq.) in the presence of potassium carbonate (0.27 g, 1.92 mmol, 3.0 eq.), DMEDA (0.028 g, 0.32 mmol, 0.5 eq.) and CuI (0.061 g, 0.32 mmol, 0.5 eq.) in 20 mL of DMA at 150° C. for 20 h followed by preparative HPLC purification using method A (rt: 12.32 min) afforded the title product as a white solid. R_{f} (MeOH/DCM: 0.5/9.5)=0.3. Yield 0.072 g. MS (ESI) m/z [M+1]⁺: 545.4. ¹H NMR (400 MHz, DMSO-d₆): δ 8.48 (d, 1H), 8.11 (s, 1H), 7.83-7.91 (m, 3H), 7.76 (s, 1H), 7.51 (d, 1H), 7.43 (d, 1H), 6.57 (s, 1H), 4.62-4.68 (m, 6H), 4.14 (d, 2H), 3.62 (d, 2H), 3.28 (s, 3H), 2.87 (s, 3H), 2.76 (t, 2H), 1.95-2.05 (m, 1H), 1.93 (d, 2H), 1.39-1.47 (m, 2H).

### Example 13

5-(2-(3-(Methylsulfonyl)-4-((1-(methylsulfonyl)piperidin-4-yl)methoxy)-benzyl)isoindolin-5-yl)oxazole (Compound 13a) and

2-(2-(3-(Methylsulfonyl)-4-((1-(methylsulfonyl)piperidin-4-yl)methoxy)-benzyl)isoindolin-5-yl)oxazole (Compound 13b)

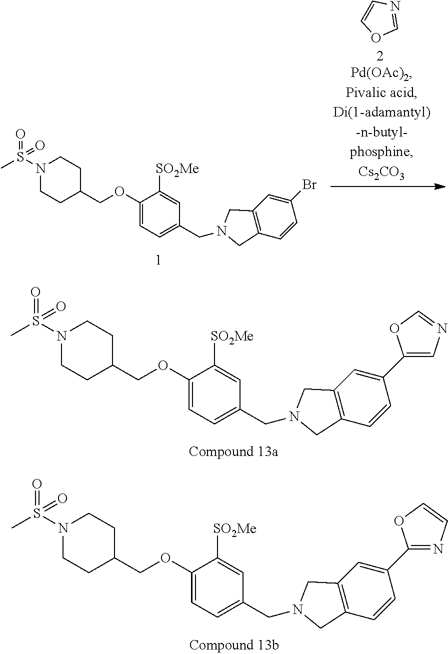 [SMILES: c1cocn1 ; CS(=O)(=O)c1cc(CN2Cc3ccc(Br)cc3C2)ccc1OCC1CCN(S(C)(=O)=O)CC1 ; CS(=O)(=O)c1cc(CN2Cc3ccc(-c4cnco4)cc3C2)ccc1OCC1CCN(S(C)(=O)=O)CC1 ; CS(=O)(=O)c1cc(CN2Cc3ccc(-c4ncco4)cc3C2)ccc1OCC1CCN(S(C)(=O)=O)CC1]

Treatment of 5-bromo-2-(3-(methylsulfonyl)-4-((1-(methylsulfonyl)piperidin-4-yl)methoxy)benzyl)isoindoline (0.25 g, 0.45 mmol, 1.0 eq.) with oxazole (0.046 g, 0.67 mmol, 1.5 eq.) in the presence of cesium carbonate (0.36 g, 1.12 mmol, 2.5 eq.), pivalic acid (0.018 g, 0.18 mmol, 0.4 eq.), palladium (II) acetate (0.006 g, 0.022 mmol, 0.05 eq) and di(1-adamantyl)-n-butylphosphine (0.008 g, 0.022 mmol 0.05 eq) in 20 mL of DMF at 110° C. for 16 h followed by preparative HPLC purification using method C (rt: 10.43 min and 11.41 min, column: X Sunfire C18) afforded the two regioisomers as white solids. R_{f} (MeOH/DCM: 0.5/9.5)=0.4. Yield 0.008 g (Compound 13a) and 0.005 g (Compound 13b).

Compound 13a: MS (ESI) m/z [M+1]⁺: 546.4. ¹H NMR (400 MHz, DMSO-d₆): δ 8.41 (s, 1H), 7.80-7.87 (m, 1H), 7.40-7.70 (m, 4H), 7.31 (dd, 2H), 4.09 (d, 2H), 3.80-3.95 (m, 5H), 3.61 (d, 2H), 3.27 (s, 3H), 2.86 (s, 3H), 2.76 (t, 2H), 2.40-2.60 (m, 1H), 1.95-2.05 (m, 1H), 1.92 (d, 2H), 1.38-1.46 (m, 2H).

Compound 13b: MS (ESI) m/z [M+1]⁺: 546.4. ¹H NMR (400 MHz, DMSO-d₆): δ 8.19 (s, 1H), 7.81-7.85 (m, 3H), 7.66-7.68 (m, 1H), 7.38 (d, 1H), 7.35 (s, 1H), 7.28 (d, 1H), 4.09 (d, 2H), 3.85-3.95 (m, 6H), 3.61 (d, 2H), 3.26 (s, 3H), 2.86 (s, 3H), 2.76 (t, 2H), 1.95-2.05 (m, 1H), 1.92 (d, 2H), 1.38-1.46 (m, 2H).

### Example 14

5-(2-(3-(Methylsulfonyl)-4-((1-(methylsulfonyl)piperidin-4-yl)methoxy)benzyl)-isoindolin-5-yl)thiazole (Compound 14)

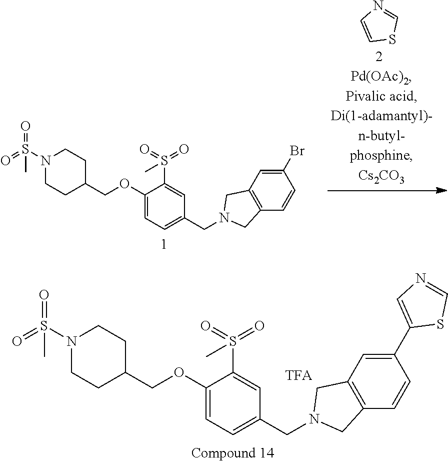 [SMILES: c1cscn1 ; CS(=O)(=O)c1cc(CN2Cc3ccc(Br)cc3C2)ccc1OCC1CCN(S(C)(=O)=O)CC1 ; CS(=O)(=O)c1cc(CN2Cc3ccc(-c4cncs4)cc3C2)ccc1OCC1CCN(S(C)(=O)=O)CC1]

Treatment of 5-bromo-2-(3-(methylsulfonyl)-4-((1-(methylsulfonyl)piperidin-4-yl)methoxy)benzyl)isoindoline (0.5 g, 0.90 mmol, 1.0 eq.) with thiazole (0.098 g, 1.16 mmol, 1.3 eq.) in the presence of cesium carbonate (0.73 g, 2.24 mmol, 2.5 eq.), pivalic acid (0.04 g, 0.35 mmol, 0.4 eq.), palladium (II) acetate (0.01 g, 0.04 mmol, 0.05 eq.) and di(1-adamantyl)-n-butylphosphine (0.02 g, 0.04 mmol 0.05 eq) in 20 mL of DMF at 110° C. for 16 h followed by preparative HPLC purification using method B (rt: 10.36 min) afforded the title product as a white solid. R_{f} (MeOH/DCM: 0.5/9.5)=0.5. Yield 0.050 g. MS (ESI) m/z [M+1]⁺: 562.4. ¹H NMR (400 MHz, DMSO-d₆): δ 10.84 (bs, 1H), 9.11 (s, 1H), 8.33 (s, 1H), 8.11 (s, 1H), 7.87 (d, 1H), 7.70-7.80 (m, 2H), 7.48 (d, 1H), 7.43 (d, 1H), 4.58-4.75 (m, 6H), 4.14 (d, 2H), 3.62 (d, 2H), 3.28 (s, 3H), 2.87 (s, 3H), 2.76 (t, 2H), 1.95-2.05 (m, 1H), 1.92 (d, 2H), 1.38-1.48 (m, 2H).

### Example 15

1-(2-(3-(Methylsulfonyl)-4-((1-(methylsulfonyl)piperidin-4-yl)methoxy)-benzyl)isoindolin-5-yl)ethan-1-one (Compound 15a),

3-(Dimethylamino)-1-(2-(3-(methylsulfonyl)-4-((1-(methylsulfonyl)piperidin-4-yl)methoxy)benzyl)isoindolin-5-yl)prop-2-en-1-one (Compound 15b) and

5-(2-(3-(Methylsulfonyl)-4-((1-(methylsulfonyl)-piperidin-4-yl)methoxy)benzyl)isoindolin-5-yl)isoxazole (Compound 15c)

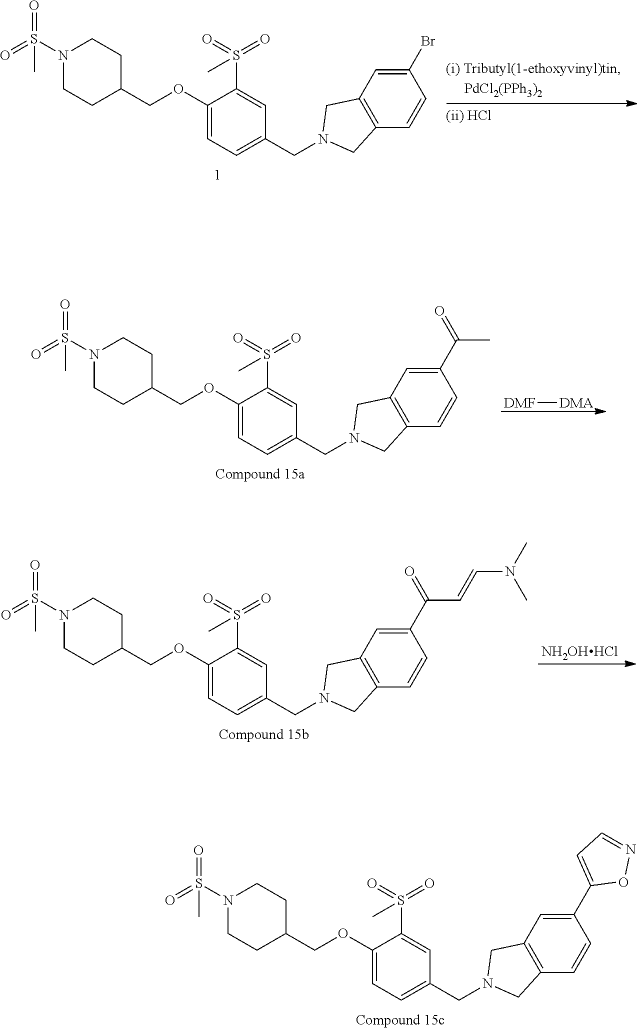

a) 1-(2-(3-(Methylsulfonyl)-4-((1-(methylsulfonyl)piperidin-4-yl)methoxy)-benzyl)isoindolin-5-yl)ethan-1-one (Compound 15a)

To a solution of 5-bromo-2-(3-(methylsulfonyl)-4-((1-(methylsulfonyl)piperidin-4-yl)methoxy)benzyl)isoindoline (2.0 g, 3.59 mmol, 1.0 eq.) in 20 mL DMA, was added tributyl(1-ethoxyvinyl)tin (2.0 g, 5.53 mmol, 1.4 eq.) at 25° C. The mixture was degassed by argon for 15 min. Bis(triphenylphosphine)palladium (II) dichloride (0.13 g, 0.17 mmol, 0.05 eq.) was then added and the resulting mixture was heated at 100° C. for 16 h. After completion of the reaction, the reaction mixture was quenched with water and extracted (3 times) with EtOAc. The combined organic layer was washed with brine, dried over anhydrous sodium sulphate and concentrated under reduced pressure to get crude product. This crude product was dissolved in 2 N HCl (10 mL) and stirred at 25° C. for 2 h. The mixture was then extracted (3 times) with EtOAc and combined organic layer was washed with brine, dried over anhydrous sodium sulphate and concentrated under reduced pressure to get crude product. This crude residue was purified by combi-flash chromatography using 0-25% ethyl acetate in heptane as an eluent. R_{f} (MeOH/EtOAc: 0.5/9.5)=0.6. Yield 0.8 g, 43%. MS (ESI) m/z [M+1]⁺: 521.38.

b) 3-(Dimethylamino)-1-(2-(3-(methylsulfonyl)-4-((1-(methylsulfonyl)piperidin-4-yl)methoxy)benzyl)isoindolin-5-yl)prop-2-en-1-one (Compound 15b)

Treatment of 1-(2-(3-(methylsulfonyl)-4-((1-(methylsulfonyl)piperidin-4-yl)-methoxy)benzyl)isoindolin-5-yl)ethan-1-one (1.0 g, 1.92 mmol, 1.0 eq.) with DMF-DMA (40 mL) in DMF at 90° C. for 16 h afforded the title product as yellow gummy mass. R_{f} (MeOH/DCM: 0.5/9.5)=0.6. Yield 0.9 g (crude). MS (ESI) m/z [M+1]⁺: 576.14. ¹H-NMR (400 MHz, DMSO-d₆): δ 7.82 (s, 1H), 7.75 (s, 1H), 7.64-7.71 (m, 2H), 7.52-7.59 (m, 1H), 7.28 (d, 1H), 7.22 (d, 1H), 6.66 (s, 1H), 4.08 (d, 2H), 3.81-3.90 (m, 6H), 3.61 (d, 2H), 3.31 (s, 6H), 3.26 (s, 3H), 2.87 (s, 3H), 2.75 (t, 2H), 1.90-1.95 (m, 3H), 1.40-1.45 (m, 2H).

c) 5-(2-(3-(Methylsulfonyl)-4-((1-(methylsulfonyl)piperidin-4-yl)methoxy)-benzyl)isoindolin-5-yl)isoxazole (Compound 15c)

Treatment of 3-(dimethylamino)-1-(2-(3-(methylsulfonyl)-4-((1-(methylsulfonyl)piperidin-4-yl)methoxy)benzyl)isoindolin-5-yl)prop-2-en-1-one (0.9 g, 1.56 mmol, 1.0 eq.) with hydroxyl amine hydrochloride (0.32 g, 4.6 mmol, 3.0 eq.) in 40 mL of EtOH at 80° C. for 3 h followed by preparative HPLC purification using method B (rt: 14.01 min) afforded the title product as brown solid. R_{f} (MeOH/DCM: 0.5/9.5)=0.6. Yield 0.10 g. ¹H NMR (400 MHz, DMSO-d₆): δ 8.68 (d, 1H), 8.11 (s, 1H), 7.86-7.93 (m, 3H), 7.57 (d, 1H), 7.43 (d, 1H), 7.04 (d, 1H), 4.67 (bs, 6H), 4.14 (d, 2H), 3.62 (d, 2H), 3.28 (s, 3H), 2.87 (s, 3H), 2.76 (t, 2H), 1.95-2.05 (m, 1H), 1.93 (d, 2H), 1.39-1.47 (m, 2H). MS (ESI) m/z [M+1]⁺: 546.4.

### Example 16

2-(3-(Methylsulfonyl)-4-((1-(methylsulfonyl)piperidin-4-yl)methoxy)benzyl)-isoindoline-5-carbonitrile (Compound 16a),

N-Hydroxy-2-(3-(methylsulfonyl)-4-((1-(methylsulfonyl)piperidin-4-yl)-methoxy)benzyl)isoindoline-5-carboximidamide (Compound 16b) and

3-(2-(3-(Methylsulfonyl)-4-((1-(methylsulfonyl)piperidin-4-yl)methoxy)benzyl)-isoindolin-5-yl)-1,2,4-oxadiazole (Compound 16c)

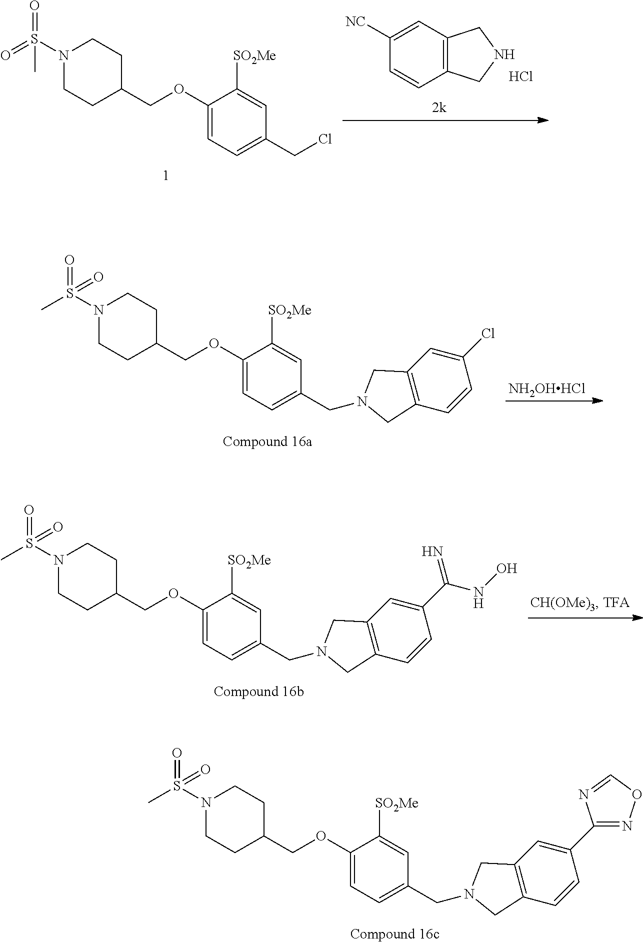

a) 2-(3-(Methylsulfonyl)-4-((1-(methylsulfonyl)piperidin-4-yl)methoxy)benzyl)-isoindoline-5-carbonitrile (Compound 16a)

Treatment of isoindoline-5-carbonitrile hydrochloride (0.9 g, 5.0 mmol, 1.0 eq.) with 4-((4-(chloromethyl)-2-(methylsulfonyl)phenoxy)methyl)-1-(methylsulfonyl)-piperidine (1.97 g, 5.0 mmol, 1.0 eq.) in the presence of DIPEA (3.88 g, 5.2 mL, 30.0 mmol, 6.0 eq.) in CH₃CN (15 mL) at 80° C. for 4 h followed by column chromatography afforded the title product as a white solid. R_{f} (MeOH/DCM: 0.5/9.5)=0.6. Yield 0.7 g, 61%. MS (ESI) m/z [M+1]⁺: 504.23.

b) N-Hydroxy-2-(3-(methylsulfonyl)-4-((1-(methylsulfonyl)piperidin-4-yl)-methoxy)benzyl)isoindoline-5-carboximidamide (Compound 16b)

Treatment of 2-(3-(methylsulfonyl)-4-((1-(methylsulfonyl)piperidin-4-yl)-methoxy)benzyl)isoindoline-5-carbonitrile (0.3 g, 0.6 mmol, 1.0 eq.) with NH₂OH·HCl (0.04 g, 0.6 mmol, 1.0 eq.) in the presence of Et₃N (0.32 g, 0.4 mL, 3.0 mmol, 5.0 eq.) in EtOH (10 mL) at 90° C. for 12 h afforded the title product as a white solid. R_{f} (MeOH/DCM: 0.5/9.5)=0.4. Yield 0.28 g (crude). MS (ESI) m/z [M+1]⁺: 537.15. ¹H-NMR (400 MHz, DMSO-d₆): δ 9.53 (s, 1H), 7.81 (s, 1H), 7.69 (d, 1H), 7.47-7.67 (m, 2H), 7.28 (d, 1H), 7.20 (d, 1H), 5.73 (s, 2H), 4.08 (d, 2H), 3.81-3.90 (m, 6H), 3.60 (d, 2H), 3.26 (s, 3H), 2.86 (s, 3H), 2.76 (t, 2H), 1.88-1.95 (m, 3H), 1.35-1.48 (m, 2H).

c) 3-(2-(3-(Methylsulfonyl)-4-((1-(methylsulfonyl)piperidin-4-yl)methoxy)-benzyl)isoindolin-5-yl)-1,2,4-oxadiazole (Compound 16c)

Treatment of N-hydroxy-2-(3-(methylsulfonyl)-4-((1-(methylsulfonyl)piperidin-4-yl)methoxy)benzyl)isoindoline-5-carboximidamide (0.28 g, 0.52 mmol, 1.0 eq.), with trimethylorthoformate (10 mL) in the presence of TFA (0.1 mL) at 70° C. for 2 h followed by preparative HPLC purification using method A afforded the title product as a white solid. R_{f} (MeOH/DCM: 0.5/9.5)=0.4. Yield 0.14 g. MS (ESI) m/z [M+1]⁺: 547.3. ¹H NMR (400 MHz, DMSO-d₆): δ 11.00 (bs, 1H), 9.74 (s, 1H), 8.10 (bs, 2H), 8.05 (d, 1H), 7.88 (d, 1H), 7.61 (d, 1H), 7.43 (d, 1H), 4.60-4.80 (m, 6H), 4.14 (d, 2H), 3.62 (d, 2H), 3.28 (s, 3H), 2.87 (s, 3H), 2.76 (t, 2H), 1.95-2.05 (m, 1H), 1.92 (d, 2H), 1.40-1.50 (m, 2H).

### Example 17

2-(1-(5-Bromoisoindolin-2-yl)ethyl)-5-((1-(methylsulfonyl)piperidin-4-yl)-methoxy)-4H-pyran-4-one (Compound 17a) and

5-((1-(Methylsulfonyl)piperidin-4-yl)methoxy)-2-(1-(5-(oxazol-2-yl)isoindolin-2-yl)ethyl)-4H-pyran-4-one (Compound 17b)

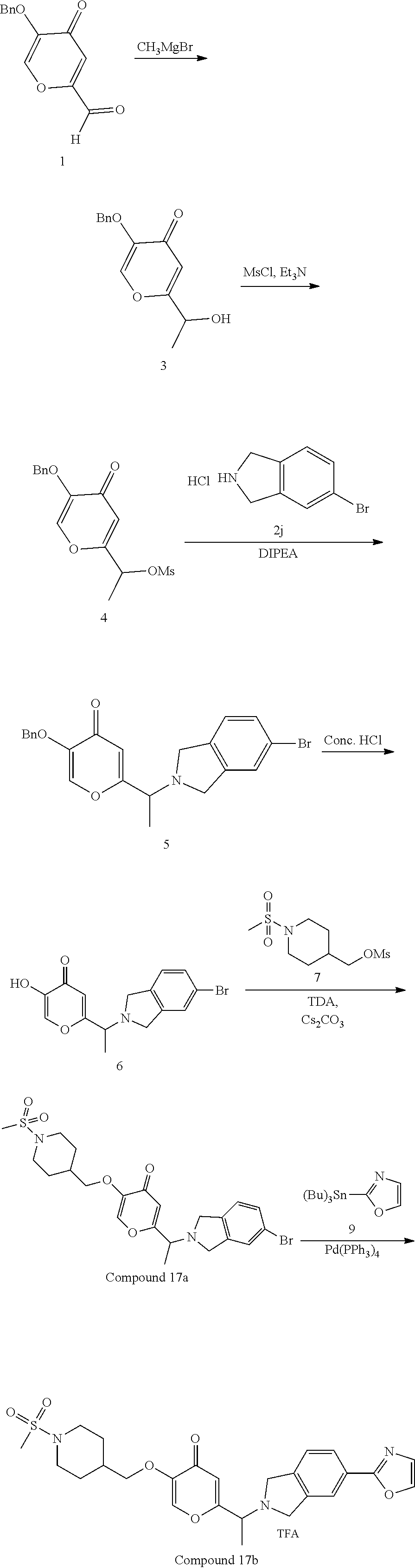

a) 5-(Benzyloxy)-2-(1-hydroxyethyl)-4H-pyran-4-one

To a solution of 5-(benzyloxy)-4-oxo-4H-pyran-2-carbaldehyde (10.0 g, 43.4 mmol, 1.0 eq.) in 300 mL of THF was added methylmagnesium bromide (3.0 M solution) (23 mL, 69.5 mmol, 1.6 eq.) dropwise at 0° C. The mixture was stirred at 0° C. for 0.5 h. After the completion of the reaction, the reaction mixture was quenched with NH₄Cl solution and extracted (3 times) with EtOAc. The combined organic layer was washed with brine, dried over anhydrous sodium sulphate and concentrated under reduced pressure to get the crude product. This crude residue was purified by combi-flash chromatography using 0-50% ethyl acetate in heptane as an eluent. R_{f} (EtOAc/heptane: 8/2)=0.2. Yield 6.0 g, 56%. MS (ESI) m/z [M+1]⁺: 247.07.

b) 1-(5-(Benzyloxy)-4-oxo-4H-pyran-2-yl)ethyl methanesulfonate

To a solution of 5-(benzyloxy)-2-(1-hydroxyethyl)-4H-pyran-4-one (4.0 g, 16.3 mmol, 1.0 eq.) in 50 mL of THF were added Et₃N (4.9 g, 6.6 mL, 48.8 mmol, 3.0 eq.) and methanesulphonyl chloride (2.8 g, 1.8 mL, 24.4 mmol, 1.5 eq.) dropwise at 0° C. The mixture was stirred at 25° C. for 1.5 h. After completion of the reaction, the mixture was diluted with water and extracted (3 times) with EtOAc. The combined organic layer was washed with brine, dried over anhydrous sodium sulphate and concentrated under reduced pressure. This crude product was forwarded to the next step without further purification. R_{f} (EtOAc/heptane: 4/6)=0.3. Yield 4.0 g (crude). MS (ESI) m/z [M+1]⁺: 325.14. ¹H-NMR (400 MHz, DMSO-d₆): δ 8.30 (s, 1H), 7.34-7.44 (m, 5H), 6.56 (s, 1H), 5.61 (q, 1H), 4.95 (s, 2H), 3.29 (s, 3H), 1.58 (d, 3H).

c) 5-(Benzyloxy)-2-(1-(5-bromoisoindolin-2-yl)ethyl)-4H-pyran-4-one

Treatment of 1-(5-(benzyloxy)-4-oxo-4H-pyran-2-yl)ethyl methanesulfonate (2.2 g, 6.8 mmol, 1.0 eq.) with 5-bromoisoindoline (2.0 g, 6.8 mmol, 1.0 eq.) in the presence of DIPEA (2.6 g, 3.6 mL, 20.3 mmol, 3.0 eq.) in 20 mL of CH₃CN at 70° C. for 16 h followed by column chromatography afforded the title compound. R_{f} (MeOH/DCM: 0.5/9.5)=0.8. Yield 1.5 g, 52%. MS (ESI) m/z [M+1]⁺: 426.14.

d) 2-(1-(5-Bromoisoindolin-2-yl)ethyl)-5-hydroxy-4H-pyran-4-one

5-(Benzyloxy)-2-(1-(5-bromoisoindolin-2-yl)ethyl)-4H-pyran-4-one (1.4 g, 3.3 mmol, 1.0 eq.) and 10 mL of concentrated HCl were taken in a 30 mL reaction vial. The mixture was heated at 60° C. for 16 h. After completion of the reaction, the reaction mixture was quenched with saturated aqueous NaHCO₃ and extracted (3 times) with EtOAc. The combined organic layer was washed with brine, dried over anhydrous sodium sulphate, filtered and concentrated under reduced pressure to get the crude product which was forwarded to the next step without purification. R_{f} (MeOH/DCM: 0.5/9.5)=0.1. Yield 1.05 g (crude). MS (ESI) m/z [M+1]⁺: 336.11.

e) 2-(1-(5-Bromoisoindolin-2-yl)ethyl)-5-((1-(methylsulfonyl)piperidin-4-yl)-methoxy)-4H-pyran-4-one (Compound 17a)

To a solution of 2-(1-(5-bromoisoindolin-2-yl)ethyl)-5-hydroxy-4H-pyran-4-one (1.05 g, 3.12 mmol, 1.0 eq.) in 10 mL of DMSO were added (1-(methylsulfonyl)-piperidin-4-yl)methyl methanesulfonate (0.84 g, 3.13 mmol, 1.0 eq.), cesium carbonate (2.04 g, 6.25 mmol, 2.0 eq.) and tris(2-(2-methoxyethoxy)ethyl)amine (0.10 g, 0.31 mmol, 0.1 eq.) at 25° C. The resulting mixture was stirred at 60° C. for 16 h. After completion of the reaction, the reaction mixture was quenched with ice cold water and extracted (3 times) with EtOAc. The combined organic layer was washed with brine, dried over anhydrous sodium sulphate and concentrated under reduced pressure to get the white gummy mass as crude product. This crude product was purified by combi-flash chromatography using 0-50% ethyl acetate in heptane as an eluent. R_{f} (MeOH/DCM: 0.5/9.5)=0.5. Yield 0.25 g, 16%. MS (ESI) m/z [M+1]⁺: 511.11. ¹H-NMR (400 MHz, DMSO-d₆): δ 8.16 (s, 1H), 7.45 (s, 1H), 7.37 (d, 1H), 7.20 (d, 1H), 6.37 (s, 1H), 3.80-3.94 (m, 5H), 3.65-3.80 (m, 2H), 3.57 (d, 2H), 2.85 (s, 3H), 2.66-2.80 (m, 2H), 1.79-1.90 (m, 3H), 1.38 (d, 3H), 1.21-1.35 (m, 2H).

f) 5-((1-(Methylsulfonyl)piperidin-4-yl)methoxy)-2-(1-(5-(oxazol-2-yl)isoindolin-2-yl)ethyl)-4H-pyran-4-one (Compound 17b)

The solution of 2-(1-(5-bromoisoindolin-2-yl)ethyl)-5-((1-(methylsulfonyl)-piperidin-4-yl)methoxy)-4H-pyran-4-one (0.10 g, 0.19 mmol, 1.0 eq.) in 5 mL of dioxane was degassed using argon for 10 min. Then 2-(tributylstannyl)oxazole (0.084 g, 0.23 mmol, 1.2 eq.) and tetrakis(triphenylphosphine)palladium(0) (0.022 g, 0.02 mmol , 0.1 eq.) were added to the reaction mixture followed by again purging with argon for 10 min. The reaction vial was sealed and heated at 110° C. for 16 h. After completion of the reaction, the reaction mixture was quenched with cold water and extracted (3 times) with EtOAc. The combined organic layer was washed with brine, dried over anhydrous sodium sulphate, filtered and concentrated under reduced pressure to get the crude product. The crude product was purified by preparative HPLC using method C (rt 13.17 min). The TFA salt of this compound was prepared by passing the compound through reverse phase column with 0.1% TFA as buffer to afford the title product as TFA salt. R_{f} (MeOH/DCM: 0.5/9.5)=0.4. Yield 0.02 g. MS (ESI) m/z [M+1]⁺: 500.2. ¹H NMR (400 MHz, DMSO-d₆): δ 8.22 (d, 2H), 7.90-7.99 (m, 2H), 7.49 (d, 1H), 7.39 (s, 1H), 6.52-6.61 (m, 1H), 4.35-4.71 (m, 2H), 3.50-3.76 (m, 7H), 2.86 (s, 3H), 2.66-2.75 (m, 2H), 1.83-1.85 (m, 3H), 1.51-1.65 (m, 3H), 1.24-1.33 (m, 2H).

### Example 18

2-(1-(5-((1-(Methylsulfonyl)piperidin-4-yl)methoxy)-4-oxo-4H-pyran-2-yl)ethyl)-isoindoline-5-carbonitrile (Compound 18a),

N-Hydroxy-2-(1-(5-((1-(methylsulfonyl)piperidin-4-yl)methoxy)-4-oxo-4H-pyran-2-yl)ethyl)isoindoline-5-carboximidamide (Compound 18b) and

2-(1-(5-(1,2,4-Oxadiazol-3-yl)isoindolin-2-yl)ethyl)-5-((1-(methylsulfonyl)-piperidin-4-yl)methoxy)-4H-pyran-4-one (Compound 18c)

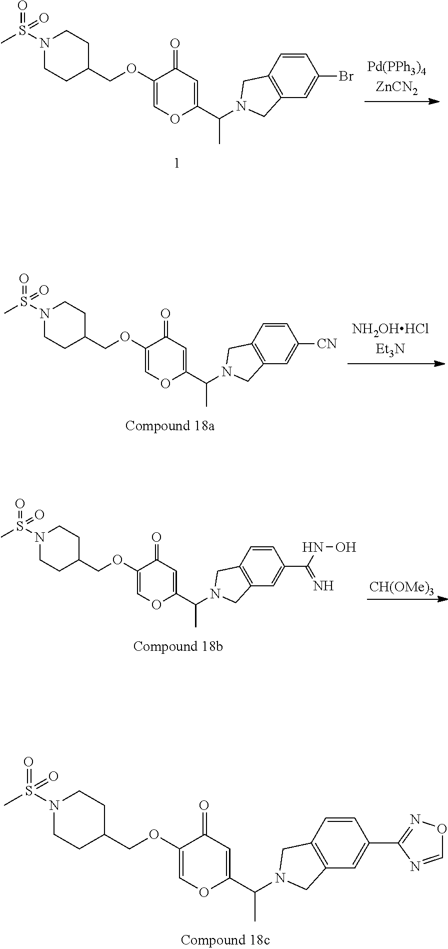

a) 2-(1-(5-((1-(Methylsulfonyl)piperidin-4-yl)methoxy)-4-oxo-4H-pyran-2-yl)ethyl)-isoindoline-5-carbonitrile (Compound 18a)

A solution of 2-(1-(5-bromoisoindolin-2-yl)ethyl)-5-((1-(methylsulfonyl)-piperidin-4-yl)methoxy)-4H-pyran-4-one (0.65 g, 1.27 mmol, 1.0 eq.) in 1,4-dioxane (15 mL), were added zinc (II) cyanide (0.44 g, 3.8 mmol, 3.0 eq.). The mixture was degassed by argon for 15 min. Then tetrakis(triphenylphosphine)palladium(0) (0.29 g, 0.25 mmol, 0.2 eq.) was added and the mixture was again degassed with argon for 10 min. The reaction vial was sealed and heated at 110° C. for 16 h. After completion of the reaction, the mixture was quenched with water and extracted (3 times) with EtOAc. The combined organic layer was washed with brine, dried over anhydrous sodium sulphate, filtered and concentrated under reduced pressure. This crude was purified by combi-flash chromatography using 0-80% ethyl acetate in heptane as an eluent. R_{f} (EtOAc/heptane: 8/2)=0.3. Yield 0.25 g, 43%. MS (ESI) m/z [M+1]⁺: 458.36.

b) N-Hydroxy-2-(1-(5-((1-(methylsulfonyl)piperidin-4-yl)methoxy)-4-oxo-4H-pyran-2-yl)ethyl)isoindoline-5-carboximidamide (Compound 18b)

Treatment of 2-(1-(5-((1-(methylsulfonyl)piperidin-4-yl)methoxy)-4-oxo-4H-pyran-2-yl)ethyl)isoindoline-5-carbonitrile (0.25 g, 0.546 mmol, 1.0 eq.) with hydroxyl-amine hydrochloride (0.075 g, 1.09 mmol, 2.0 eq.) in the presence of Et₃N (0.22 g, 0.30 mL, 2.18 mmol, 4.0 eq.) in 10 mL of EtOH at 70° C. for 18 h followed by column chromatography afforded the title product. R_{f} (MeOH/DCM: 0.5/9.5)=0.5. Yield 0.26 g, 97%. MS (ESI) m/z [M+1]⁺: 491.37. ¹H-NMR (400 MHz, DMSO-d₆): δ 9.91-10.19 (m, 4H), 8.17 (s, 1H), 7.51-7.54 (m, 2H), 7.26 (s, 1H), 3.84-3.99 (m, 4H), 3.70-3.74 (m, 3H), 3.56 (d, 2H), 2.85 (s, 3H), 2.65-2.75 (m, 2H), 1.82-1.84 (m, 3H), 1.74 (d, 3H), 1.39-49 (m, 2H).

c) 2-(1-(5-(1,2,4-Oxadiazol-3-yl)isoindolin-2-yl)ethyl)-5-((1-(methylsulfonyl)-piperidin-4-yl)methoxy)-4H-pyran-4-one (Compound 18c)

Treatment of N-hydroxy-2-(1-(5-((1-(methylsulfonyl)piperidin-4-yl)methoxy)-4-oxo-4H-pyran-2-yl)ethyl)isoindoline-5-carboximidamide (0.4 g, 0.85 mmol, 1.0 eq.) with trimethyl orthoformate (1.7 g, 1.8 mL, 16.3 mmol, 20.0 eq.) at 60° C. for 18 h followed by preparative HPLC purification using method C (rt: 13.24 min, column: X bridge C18) afforded the title compound. R_{f} (MeOH/DCM: 0.5/9.5)=0.5. Yield 0.011 g. MS (ESI) m/z [M+1]⁺: 501.2. ¹H NMR (400 MHz, DMSO-d₆): δ 9.67 (s,1H), 8.16 (s,1H), 7.89 (d, 2H), 7.44 (d, 1H), 6.40 (s, 1H), 3.92-4.05 (m, 4H), 3.71-3.77 (m, 3H), 3.57 (d, 2H), 2.85 (s, 3H), 2.69-2.75 (m, 2H), 1.82-1.89 (m, 3H), 1.42 (d, 3H), 1.23-1.33 (m, 2H).

### Example 19

2-(1-(3,4-Dihydroisoquinolin-2(1H)-yl)-2,2,2-trifluoroethyl)-5-((1-(methyl-sulfonyl)piperidin-4-yl)methoxy)-4H-pyran-4-one (Compound 19)

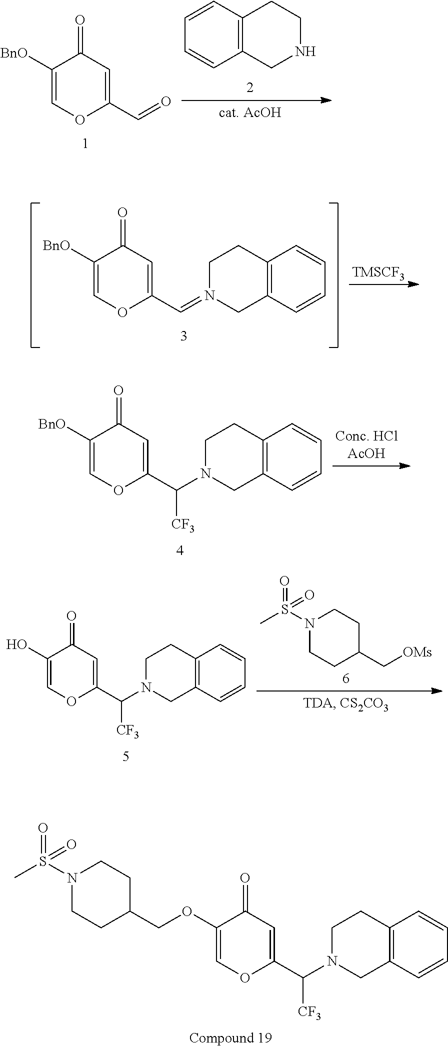

a) 2-((5-(Benzyloxy)-4-oxo-4H-pyran-2-yl)methylene)-1,2,3,4-tetrahydroiso-quinolin-2-ium

To a solution of 5-(benzyloxy)-4-oxo-4H-pyran-2-carbaldehyde (1.0 g, 4.3 mmol, 1.0 eq.) in 10 mL DCE were added tetrahydroisoquinoline (0.57 g, 4.3 mmol, 1.0 eq.) and acetic acid (0.3 mL) at 25° C. The mixture was heated at 50° C. for 16 h. After completion of the reaction, solvent was evaporated under reduced pressure and the resulting crude product (stored under nitrogenous condition at −40° C.) was forwarded to the next step without further purification. R_{f} (EtOAc/Heptane: 5/5)=0.3. Yield 1.5 g (crude). MS(ESI) m/z [M+1]⁺: 347.25.

b) 5-(Benzyloxy)-2-(1-(3,4-dihydroisoquinolin-2(1H)-yl)-2,2,2-trifluoroethyl)-4H-pyran-4-one

In a 100 mL sealed tube 2-((5-(benzyloxy)-4-oxo-4H-pyran-2-yl)methylene)-1,2,3,4-tetrahydroisoquinolin-2-ium (1.5 g, 4.3 mmol, 1.0 eq.) was dissolved in 60 mL of CH₃CN. Then trimethyl(trifluoromethyl)silane (6.8 g, 47.5 mmol, 11.0 eq.) was added at 25° C. The resulting mixture was heated at 120° C. for 16 h. After completion of the reaction, the reaction mixture was quenched with ice cold water and extracted (3 times) with EtOAc. The combined organic layer was washed with brine, dried over anhydrous sodium sulphate and concentrated under reduced pressure. The crude product was purified by column chromatography using 100-200 mesh silica gel and 0-50% ethyl acetate in heptane as an eluent. R_{f} (EtOAc/heptane: 5/5)=0.3. Yield 0.4 g, 22%. MS (ESI) m/z [M+1]⁺: 416.26. ¹H NMR (400 MHz, DMSO-d₆): δ 8.19 (s, 1H), 7.14-7.43 (m, 9H), 6.43 (s, 1H), 4.93 (s, 2H), 4.69-4.78 (m, 1H), 3.81-3.93 (m, 2H), 3.17-3.22 (m, 1H), 2.79-2.86 (m, 1H), 2.65-2.75 (m, 2H).

c) 2-(1-(3,4-Dihydroisoquinolin-2(1H)-yl)-2,2,2-trifluoroethyl)-5-hydroxy-4H-pyran-4-one

Treatment of 5-(benzyloxy)-2-(1-(3,4-dihydroisoquinolin-2(1H)-yl)-2,2,2-tri-fluoroethyl)-4H-pyran-4-one (0.30 g, 0.72 mmol, 1.0 eq.) with concentrated HCl (3 mL) and AcOH (0.6 mL) at 65° C. for 16 h afforded the title product as a brown solid. R_{f} (EtOAc/heptane: 4/6)=0.6. Yield 0.13 g (crude). MS (ESI) m/z [M+1]⁺: 326.05. ¹H NMR (400 MHz, DMSO-d₆): δ 9.10 (s, 1H), 8.03 (s, 1H), 7.21-7.35 (m, 4H), 6.43 (s, 1H), 4.73 (q, 1H), 3.79-3.93 (m, 2H), 3.13-3.28 (m, 1H), 2.79-2.89 (m, 1H), 2.65-2.78 (m, 2H).

d) 2-(1-(3,4-Dihydroisoquinolin-2(1H)-yl)-2,2,2-trifluoroethyl)-5-((1-(methyl-sulfonyl) piperidin-4-yl)methoxy)-4H-pyran-4-one (Compound 19)

Treatment of 2-(1-(3,4-dihydroisoquinolin-2(1H)-yl)-2,2,2-trifluoroethyl)-5-hydroxy-4H-pyran-4-one (0.13 g, 0.39 mmol, 1.0 eq.) with (1-(methylsulfonyl)piperidin-4-yl)methyl methanesulfonate (0.11 g, 0.44 mmol, 1.1 eq.) in the presence of tris(2-(2-methoxyethoxy)ethyl)amine (0.077 g, 0.24 mmol, 0.6 eq.) and cesium carbonate (0.26 g, 0.80 mmol, 2.0 eq.) in DMSO (10 mL) at 60° C. for 2 h followed by preparative HPLC purification using method A (rt: 13.53 min) afforded the title compound as a white solid. R_{f} (MeOH/DCM: 1/9)=0.3. Yield 0.095 g. MS (ESI) m/z [M+1]⁺: 501.4. ¹H NMR (400 MHz, DMSO-d₆): δ 8.13 (s, 1H), 7.20-7.32 (m, 4H), 6.40 (s, 1H), 4.73 (q, 1H), 3.82-3.91 (m, 2H), 3.70 (d, 2H), 3.57 (d, 2H), 3.12-3.20 (m, 1H), 2.70-2.80 (m, 4H), 2.68-2.74 (m, 4H), 1.81-1.84 (m, 3H), 1.23-1.32 (m, 2H).

### Example 20

5-((1-(Methylsulfonyl)piperidin-4-yl)methoxy)-2-(2,2,2-trifluoro-1-(isoindolin-2-yl)ethyl)-4H-pyran-4-one (Compound 20)

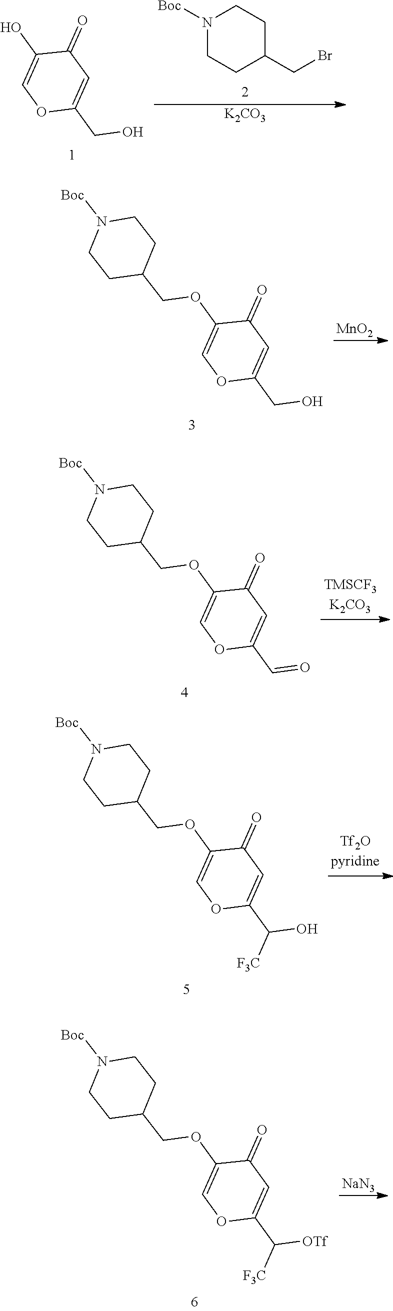

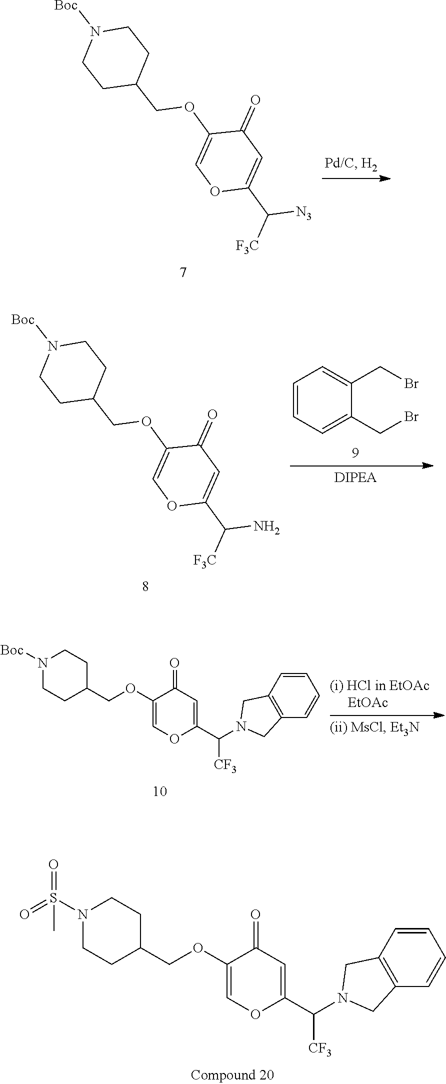 [SMILES: CC(C)(C)OC(=O)N1CCC(COc2coc(C(N=[N+]=[N-])C(F)(F)F)cc2=O)CC1 ; CC(C)(C)OC(=O)N1CCC(COc2coc(C(N)C(F)(F)F)cc2=O)CC1 ; BrCc1ccccc1CBr ; CC(C)(C)OC(=O)N1CCC(COc2coc(C(N3Cc4ccccc4C3)C(F)(F)F)cc2=O)CC1 ; CS(=O)(=O)N1CCC(COc2coc(C(N3Cc4ccccc4C3)C(F)(F)F)cc2=O)CC1]

a) tert-Butyl 4-(((6-(hydroxymethyl)-4-oxo-4H-pyran-3-yl)oxy)methyl)piperi-dine-1-carboxylate

Treatment of 5-hydroxy-2-(hydroxymethyl)-4H-pyran-4-one (10.0 g, 70.4 mmol, 1.0 eq.) with tert-butyl 4-(bromomethyl)piperidine-1-carboxylate (29.0 g, 105.6 mmol, 1.5 eq.) in the presence of potassium carbonate (29.1 g, 211.1 mmol, 3.0 eq.) in 120 mL of DMF at 70° C. for 16 h afforded the title compound as a white solid. R_{f} (EtOAc/heptane: 10/0)=0.4. Yield 8.1 g, 34%. MS (ESI) m/z [M+1]⁺: 340.31. ¹H-NMR (400 MHz, DMSO-d₆): δ 8.10 (s, 1H), 6.29 (s, 1H), 5.67 (t, 1H), 4.28 (d, 2H), 3.95 (d, 2H), 3.67 (d, 2H), 2.65-2.75 (m, 2H), 1.79-1.91 (m, 1H), 1.70 (d, 2H), 1.39 (s, 9H), 1.04-1.16 (m, 2H).

b) tert-Butyl 4-(((6-formyl-4-oxo-4H-pyran-3-yl)oxy)methyl)piperidine-1-carboxylate

In a 250 mL seal tube 4-(((6-(hydroxymethyl)-4-oxo-4H-pyran-3-yl)oxy)methyl)-piperidine-1-carboxylate (8.1 g, 23.9 mmol, 1.0 eq.) was dissolved in 100 mL of MeOH. Activated manganese dioxide (10.63 g, 10.63 mmol, 5.0 eq.) was then added to the solution at 25° C. The mixture was heated at 90° C. for 16 h. After completion of the reaction, the mixture was filtered and washed with MeOH and the resulting solution was concentrated under reduced pressure to get the crude product which was purified by combi-flash chromatography using ethyl acetate as an eluent. R_{f} (EtOAc/heptane: 8/2)=0.4. Yield 5.2 g, 65%. MS (ESI) m/z [M+1]⁺: 337.9.

c) tert-Butyl 4-(((4-oxo-6-(2,2,2-trifluoro-1-hydroxyethyl)-4H-pyran-3-yl)oxy)-methyl)piperidine-1-carboxylate

To a solution of tert-butyl 4-(((6-formyl-4-oxo-4H-pyran-3-yl)oxy)methyl)-piperidine-1-carboxylate (5.2 g, 15.40 mmol, 1.0 eq.) in 100 mL of DMF were added trimethyl-(trifluoromethyl)silane (3.42 mL, 23.1 mmol, 1.5 eq.) and potassium carbonate (0.21 g, 1.54 mmol, 0.1 eq.) at 0° C. followed by stirring at 25° C. for 4 h. After completion of the reaction, the mixture was quenched with ice-cold water and extracted (3 times) with EtOAc. The combined organic layer was washed with brine, dried over anhydrous sodium sulphate and concentrated under reduced pressure to get the crude product which was purified by combi-flash chromatography using 0-50% ethyl acetate in hexane as an eluent. R_{f} (EtOAc/heptane: 8/2)=0.4. Yield 3.0 g, 47%. MS (ESI) m/z [M+1]⁺: 408.05.

d) tert-Butyl 4-(((4-oxo-6-(2,2,2-trifluoro-1-(((trifluoromethyl)sulfonyl)oxy)-ethyl)-4H-pyran-3-yl)oxy)methyl)piperidine-1-carboxylate

To a solution of tert-butyl 4-(((4-oxo-6-(2,2,2-trifluoro-1-hydroxyethyl)-4H-pyran-3-yl)oxy)methyl)piperidine-1-carboxylate (1.0 g, 2.45 mmol, 1.0 eq.) in 20 mL of DCM were added pyridine (0.58 g, 0.58 mL, 7.35 mmol, 3.0 eq) and trifluoromethane-sulphonic anhydride (1.72 g, 1.03 mL, 6.14 mmol, 2.5 eq) at 0° C. The mixture was stirred at 0° C. for 0.5 h. After completion of the reaction, the mixture was concentrated under reduced pressure. The crude product was forwarded to the next step without further purification. R_{f} (EtOAc/Hexane: 8/2)=0.7. Yield 1.2 g (crude). MS (ESI) m/z [M+1]⁺: 540.05.

e) tert-Butyl 4-(((6-(1-azido-2,2,2-trifluoroethyl)-4-oxo-4H-pyran-3-yl)oxy)-methyl)piperidine-1-carboxylate

To a solution of tert-butyl 4-(((4-oxo-6-(2,2,2-trifluoro-1-(((trifluoromethyl)-sulfonyl)-oxy)ethyl)-4H-pyran-3-yl)oxy)methyl)piperidine-1-carboxylate (1.2 g, 2.23 mmol, 1.0 eq.) in 20 mL of DMF was added sodium azide (0.25 g, 5.56 mmol, 2.5 eq.) at 25° C. followed by stirring at 25° C. for 16 h. After completion of the reaction, the reaction mixture was quenched with water and extracted (3 times) with EtOAc. The combined organic layer was washed with brine, dried over anhydrous sodium sulphate, filtered and concentrated under reduced pressure. This crude was forwarded to the next step without further purification. R_{f} (EtOAc/Hexane: 8/2)=0.4. Yield 1.2 g (Crude). MS (ESI) m/z [M+1]⁺: 433.09.

f) tert-Butyl 4-(((6-(1-amino-2,2,2-trifluoroethyl)-4-oxo-4H-pyran-3-yl)oxy)-methyl)piperidine-1-carboxylate

To a solution of tert-butyl 4-(((6-(1-azido-2,2,2-trifluoroethyl)-4-oxo-4H-pyran-3-yl)oxy)methyl)piperidine-1-carboxylate (1.2 g, 2.78 mmol, 1.0 eq.) in 10 mL MeOH was added 10% Pd/C (50% wet, 0.6 g, 0.28 mmol) at 25° C. in nitrogenous atmosphere. H₂ gas was then purged into the reaction mixture via hydrogen balloon and the resulting mixture was stirred at 25° C. for 2 h. After completion of the reaction, the mixture was filtered through celite and washed with MeOH. The organic fraction was concentrated under reduced pressure. This crude product was purified by column chromatography using 100-200 mesh silica gel and 0-10% MeOH in EtOAc as an eluent. R_{f} (EtOAc/heptane: 8/2)=0.2. Yield 0.2 g, 22%. MS (ESI) m/z [M+1]⁺: 407.09.

g) tert-Butyl 4-(((4-oxo-6-(2,2,2-trifluoro-1-(isoindolin-2-yl)ethyl)-4H-pyran-3-yl)oxy)methyl)piperidine-1-carboxylate

To a solution of tert-butyl 4-(((6-(1-amino-2,2,2-trifluoroethyl)-4-oxo-4H-pyran-3-yl)oxy)methyl)piperidine-1-carboxylate (0.2 g, 0.49 mmol, 1.0 eq.) in 10 mL of CH₃CN, were added 1,2-bis(bromomethyl)benzene (0.38 g, 1.47 mmol, 3.0 eq.) and N-ethyl-N-isopropylpropan-2-amine (0.19 g, 0.27 mL, 1.42 mmol, 3.0 eq.) at 25° C. followed by stirring at 65° C. for 12 h. After completion of the reaction, the reaction mixture was quenched with water and extracted (3 times) with EtOAc. The combined organic layer was washed with brine, dried over anhydrous sodium sulphate and concentrated under reduced pressure. This crude residue was purified by column chromatography using 100-200 mesh silica gel and 0-10% MeOH in EtOAc as an eluent. R_{f} (EtOAc/Heptane: 8/2)=0.2. Yield 0.05 g, 20%. MS (ESI) m/z[M+1]⁺: 509.09. ¹H-NMR (400 MHz, DMSO-d₆): δ 8.25 (s, 1H), 7.19-7.30 (m, 4H), 6.52 (s, 1H), 5.01-5.10 (m, 1H), 4.12-4.20 (m, 4H), 3.92 (d, 2H), 3.67 (d, 2H), 2.65-2.80 (m, 2H), 1.81-1.92 (m, 1H), 1.69 (d, 2H), 1.38 (s, 9H), 1.01-1.15 (m, 2H).

h) 5-((1-(Methylsulfonyl)piperidin-4-yl)methoxy)-2-(2,2,2-trifluoro-1-(isoindolin-2-yl)ethyl)-4H-pyran-4-one (Compound 20)

To a solution of tert-butyl 4-(((4-oxo-6-(2,2,2-trifluoro-1-(isoindolin-2-yl)ethyl)-4H-pyran-3-yl)oxy)methyl)piperidine-1-carboxylate (0.35 g, 0.68 mmol, 1.0 eq.) in EtOAc, HCl in EtOAc (5.0 mL, 1.0 M solution) was added followed by stirring for 12 h. The reaction mass was concentrated to get the crude product as brown gummy mass. The crude product was dissolved in 50 mL of THF and Et₃N (0.21 g, 0.29 mL, 2.06 mmol, 3.0 eq.) was added followed by methane sulphonyl chloride (0.12 g, 0.08 mL, 1.03 mmol, 1.5 eq.) dropwise at 0° C. The mixture was stirred at 25° C. for 1.5 h. After completion of the reaction, the reaction mixture was concentrated under reduced pressure to get the crude product which was purified by preparative HPLC using method C (rt: 11.98, column: Sunfire C-18) to afford the title compound as a white solid. R_{f} (MeOH/DCM: 0.5/9.5)=0.3. Yield 0.006 g. MS (ESI) m/z [M+1]⁺: 487.2. ¹H NMR (400 MHz, DMSO-d₆): δ 8.27 (s, 1H), 7.20-7.27 (m, 4H), 6.60 (s, 1H), 5.05 (q, 1H), 4.11-4.16 (m, 4H), 3.70-3.76 (m, 2H), 3.56 (d, 2H), 2.85 (s, 3H), 2.67-2.74 (m, 2H), 1.84-1.90 (m, 3H), 1.26-1.29 (m, 2H).

### Example 21

2-(2,2,2-Trifluoro-1-(3-(methylsulfonyl)-4-((1-(methylsulfonyl)piperidin-4-yl)methoxy)phenyl)ethyl)isoindoline (Compound 21)

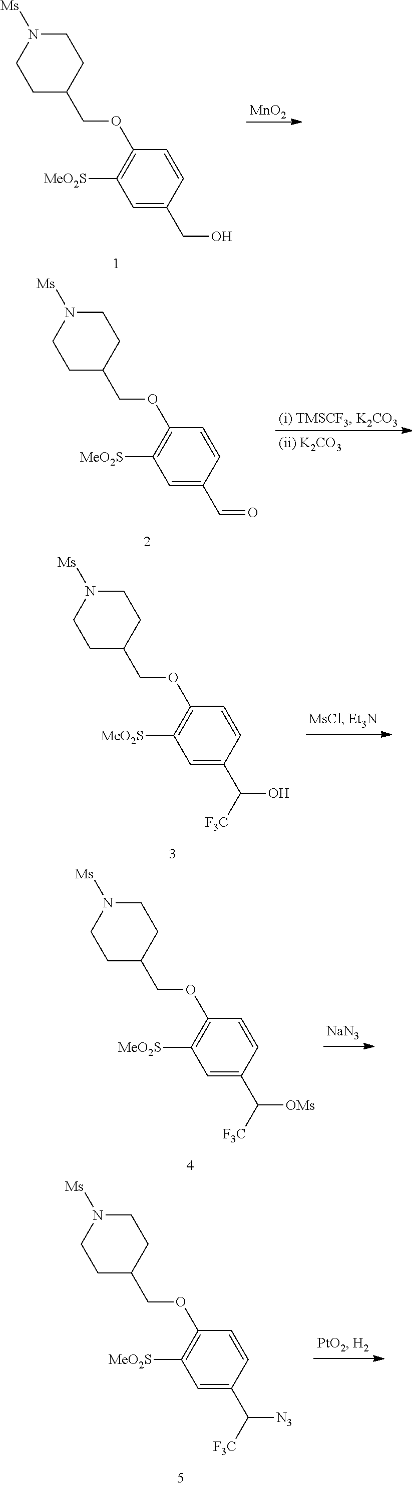

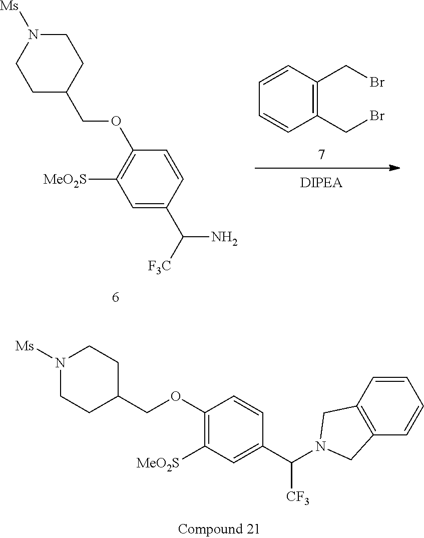 [SMILES: CS(=O)(=O)c1cc(C(N)C(F)(F)F)ccc1OCC1CCN(S(C)(=O)=O)CC1 ; BrCc1ccccc1CBr ; CS(=O)(=O)c1cc(C(N2Cc3ccccc3C2)C(F)(F)F)ccc1OCC1CCN(S(C)(=O)=O)CC1]

a) 3-(Methylsulfonyl)-4-((1-(methylsulfonyl)piperidin-4-yl)methoxy)benzaldehyde

Treatment of (3-(methylsulfonyl)-4-((1-(methylsulfonyl)piperidin-4-yl)methoxy)phenyl)methanol (3.0 g, 7.94 mmol, 1.0 eq.) with activated manganese dioxide (3.4 g, 39.7 mmol, 5.0 eq.) in 40 mL of MeOH at 60° C. for 12 h afforded the title compound as a white solid. R_{f} (EtOAc/heptane: 8/2)=0.4. Yield 2.0 g, 67%. MS (ESI) m/z [M+1]⁺: 376.13. ¹H-NMR (400 MHz, DMSO-d₆): δ 9.98 (s, 1H), 8.34 (s, 1H), 8.22 (d, 1H), 7.52 (d, 1H), 4.23 (d, 2H), 3.61 (d, 2H), 3.34 (s, 3H), 2.87 (s, 3H), 2.76 (t, 2H), 1.97-2.04 (m, 1H), 1.92 (d, 2H), 1.37-1.49 (m, 2H).

b) 2,2,2-Trifluoro-1-(3-(methylsulfonyl)-4-((1-(methylsulfonyl)piperidin-4-yl)-methoxy)phenyl)ethan-1-ol

Treatment of (methylsulfonyl)piperidin-4-yl)methoxy)benzaldehyde (2.0 g, 5.327 mmol, 1.0 eq.) with trimethyl(trifluoromethyl)silane (3.0 g, 21.31 mmol, 4.0 eq.) in the presence of potassium carbonate (0.36 g, 2.6 mmol, 0.5 eq.) in 20 mL of DMF at 0° C. for 4 h followed by column chromatography the title compound as a white solid. R_{f} (EtOAc/heptane: 8/2)=0.4. Yield 1.1 g, 46%. MS (ESI) m/z [M+1]⁺: 445.99. ¹H-NMR (400 MHz, DMSO-d₆): δ 7.96 (s, 1H), 7.80 (d, 1H), 7.34 (d, 1H), 6.96 (d, 1H), 5.26-5.31 (m, 1H), 4.11 (d, 2H), 3.61 (d, 2H), 3.27 (s, 3H), 2.86 (s, 3H), 2.76 (t, 2H), 1.95-2.01 (m, 1H), 1.91 (d, 2H), 1.35-1.47 (m, 2H).

c) 2,2,2-Trifluoro-1-(3-(methylsulfonyl)-4-((1-(methylsulfonyl)piperidin-4-yl)-methoxy)phenyl)ethyl methanesulfonate

Treatment of 2,2,2-trifluoro-1-(3-(methylsulfonyl)-4-((1-(methylsulfonyl)-piperidin-4-yl)methoxy)phenyl)ethan-1-ol (1.1 g, 3.7 mmol, 1.0 eq.) with methane-sulfonyl chloride (0.42 g, 3.69 mmol, 1.5 eq.) in the presence of Et₃N (0.37 g, 3.7 mmol, 1.5 eq) in 20 mL of DCM at 0° C. for 2 h afforded the title compound as a yellow liquid. R_{f} (EtOAc/Heptane: 8/2)=0.6. Yield 1.7 g (crude). MS (ESI) m/z [M+1]⁺: 523.89.

d) 4-((4-(1-Azido-2,2,2-trifluoroethyl)-2-(methylsulfonyl)phenoxy)methyl)-1-(methylsulfonyl)piperidine

Treatment of 2,2,2-trifluoro-1-(3-(methylsulfonyl)-4-((1-(methylsulfonyl)-piperidin-4-yl)methoxy)phenyl)ethyl methanesulfonate (1.3 g, 2.48 mmol, 1.0 eq.) with sodium azide (1.6 g, 24.8 mmol, 10.0 eq.) in 20 mL of DMF at 90° C. for 12 h afforded the title compound as a yellow liquid. R_{f} (EtOAc/heptane: 8/2)=0.5. Yield 1.2 g (crude). MS (ESI) m/z [M+1]⁺: 471.07.

e) 2,2,2-Trifluoro-1-(3-(methylsulfonyl)-4-((1-(methylsulfonyl)piperidin-4-yl)-methoxy)phenyl)ethan-1-amine

Treatment of 4-((4-(1-azido-2,2,2-trifluoroethyl)-2-(methylsulfonyl)phenoxy)-methyl)-1-(methylsulfonyl)piperidine (1.2 g, 2.55 mmol, 1.0 eq.) with platinum oxide (2.90 g, 12.8 mmol, 5.0 eq.) in 20 mL of MeOH at 25° C. for 2 h followed by column chromatography afforded the title compound as a yellow gummy mass. R_{f} (EtOAc/Heptane: 8/2)=0.2 (ninhydrin). Yield 0.35 g, 31%. MS (ESI) m/z [M+1]⁺: 445.11. ¹H-NMR (400 MHz, DMSO-d₆): δ 7.93 (s, 1H), 7.80 (d, 1H), 7.32 (d, 1H), 4.60-4.63 (m, 1H), 4.10 (d, 2H), 3.59 (d, 2H), 3.26 (s, 3H), 2.86 (s, 3H), 2.76 (t, 2H), 2.60 (bs, 2H), 1.90-1.99 (m, 3H), 1.32-1.49 (m, 2H).

f) 2-(2,2,2-Trifluoro-1-(3-(methylsulfonyl)-4-((1-(methylsulfonyl)piperidin-4-yl) methoxy)phenyl)ethyl)isoindoline (Compound 21)

Treatment of 2,2,2-trifluoro-1-(3-(methylsulfonyl)-4-((1-(methylsulfonyl)-piperidin-4-yl)methoxy)phenyl)ethan-1-amine (0.2 g, 0.45 mmol, 1.0 eq.) with 1,2-bis(bromomethyl)benzene (0.35 g, 1.35 mmol, 3.0 eq.) in the presence of N-ethyl-N-isopropylpropan-2-amine (0.17 g, 1.35 mmol, 3.0 eq.) in 10 mL of CH₃CN at 65° C. for 12 h followed by preparative HPLC purification using method C (rt: 13.28 min) afforded the title compound as a white solid. R_{f} (EtOAc/heptane: 8/2)=0.5. Yield 0.035 g. MS (ESI) m/z (M+H)⁺: 547.3. ¹H NMR (400 MHz, DMSO-d₆): δ 7.96 (d, 1H), 7.83 (dd, 1H), 7.39 (d, 1H), 7.18-7.24 (m, 4H), 4.77-4.83 (m, 1H), 4.13 (d, 2H), 3.94 (s, 2H), 3.90 (s, 2H), 3.59-3.62 (m, 2H), 3.30 (s, 3H), 2.86 (s, 3H), 2.74-2.79 (m, 2H), 1.92-2.07 (m, 1H), 1.93 (d, 2H), 1.37-1.47 (m, 2H).

### Example 22

2-(2,2-Difluoro-1-(3-(methylsulfonyl)-4-((1-(pyrimidin-2-ylmethyl)piperidin-4-yl)methoxy)phenyl)ethyl)isoindoline (Compound 22)

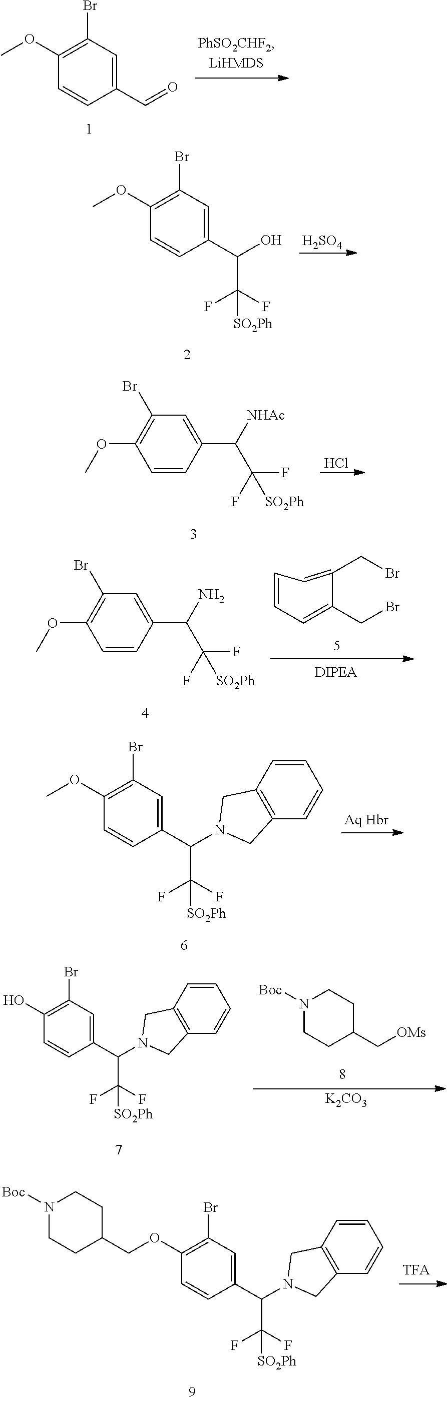

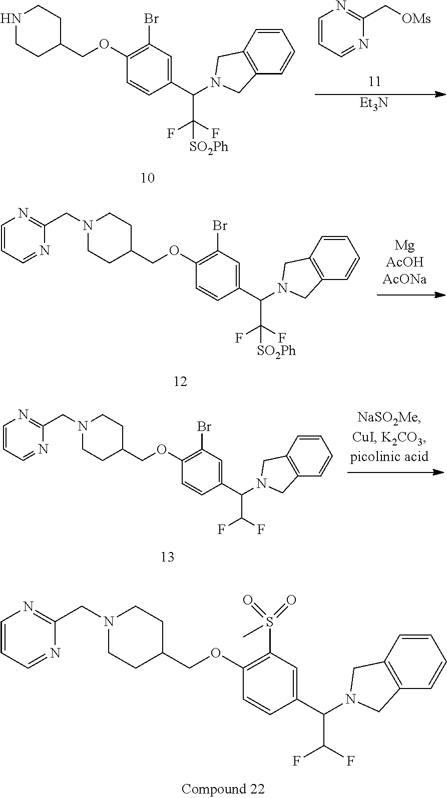

a) 1-(3-Bromo-4-methoxyphenyl)-2,2-difluoro-2-(phenylsulfonyl)ethan-1-ol

To a solution of 3-bromo-4-methoxybenzaldehyde (20.0 g, 93.5 mmol, 1.0 eq.) in THF (200 mL) and HMPA (3.0 mL), ((difluoromethyl)sulfonyl)benzene (18.0 g, 93.5 mmol, 1.0 eq.) was added at 0° C. and then lithium bis(trimethylsilyl)amide solution (93.5 mL, 1.0 M in THF, 93.5 mmol, 1.0 eq.) was added slowly at 0° C. and stirred for 3 days at 25° C. The reaction mixture was quenched with ice cold water and extracted (3 times) with ethyl acetate. The combined organic layer was washed with brine, dried over anhydrous sodium sulphate, filtered and concentrated under reduced pressure. This crude product was purified by combi-flash chromatography using 0-10% ethyl acetate in hexane as an eluent. The desired fractions were concentrated under reduced pressure to afford the title compound as an off white sticky solid. Yield 10 g, 26%. MS (ESI) m/z [M+1]⁺: 407.01. ¹H NMR (400 MHz, DMSO-d₆): δ 7.99 (d, 2H), 7.72-7.80 (m, 1H), 7.58-7.68 (m, 3H), 7.39 (d, 1H), 6.89 (d, 1H), 5.49 (d, 1H), 3.89 (s, 3H).

b) N-(1-(3-Bromo-4-methoxyphenyl)-2,2-difluoro-2-(phenylsulfonyl)ethyl)-acetamide

To a stirred solution of 1-(3-bromo-4-methoxyphenyl)-2,2-difluoro-2-(phenyl-sulfonyl) ethan-1-ol (10 g, 25 mmol, 1.0 eq.) in ACN (10 mL) at 0° C., H₂SO₄ (5 mL) was added and the mixture was heated at 70° C. for 16 h. After completion of the reaction, the reaction mixture was quenched with ice cold water and extracted (3 times) with ethyl acetate. The combined organic layer was washed with brine, dried over anhydrous sodium sulphate, filtered and concentrated under reduced pressure to afford the title compound as a brown sticky solid. Yield 11.0 g (crude). MS (ESI) m/z [M+1]⁺: 448.04. ¹H NMR (400 MHz, DMSO-d₆): δ 9.02 (d, 1H), 7.88-7.92 (m, 3H), 7.71-7.75 (m, 2H), 7.67 (s, 1H), 7.41-7.43 (m, 1H), 7.10 (d, 1H), 5.85-5.95 (m, 1H), 3.84 (s, 3H), 1.84 (s, 3H).

c) 1-(3-Bromo-4-methoxyphenyl)-2,2-difluoro-2-(phenylsulfonyl)ethan-1-amine hydrochloride

A solution of N-(1-(3-bromo-4-methoxyphenyl)-2,2-difluoro-2-(phenylsulfonyl)ethyl) acetamide (11.0 g, 24.5 mmol, 1.0 eq.) in MeOH (100.0 mL), and concentrated HCl (100.0 mL) was added and the mixture was heated at 100° C. for 2 days. After completion of the reaction, the mixture was concentrated under reduced pressure to afford the title compound as a white solid. Yield 10.0 g (crude). MS (ESI) m/z [M+1]⁺: 405.99. ¹H NMR (400 MHz, DMSO-d₆): δ 9.27 (bs, 3H), 7.88-7.92 (m, 1H), 7.80-7.82 (m, 2H), 7.76 (s, 1H), 7.68-7.71 (m, 2H), 7.57 (d, 1H), 7.18 (d, 1H), 5.58-5.71 (m, 1H), 3.89 (s, 3H).

d) 2-(1-(3-Bromo-4-methoxyphenyl)-2,2-difluoro-2-(phenylsulfonyl)ethyl)iso-indoline

Treatment of 1-(3-bromo-4-methoxyphenyl)-2,2-difluoro-2-(phenylsulfonyl)-ethan-1-amine (10.0 g, 24.6 mmol, 1.0 eq.) and 1,2-bis(bromomethyl)benzene (13.0 g, 49.2 mmol, 2.0 eq.) in the presence of N-ethyl-N-isopropylpropan-2-amine (15.9 g, 21 mL, 123 mmol, 5.0 eq.) in CH₃CN (100 mL) at 70° C. for 36 h followed by Combi-flash column chromatography afforded the title compound as a white solid. Yield 8.0 g, 64%. MS (ESI) m/z [M+1]⁺: 507.97. ¹H NMR (400 MHz, DMSO-d₆): δ 7.86 (d, 2H), 7.70-7.75 (m, 2H), 7.48-7.53 (m, 3H), 7.10-7.20 (m, 5H), 5.12-5.21 (m, 1H), 3.97 (d, 2H), 3.84 (s, 3H), 3.77 (d, 2H).

e) 2-Bromo-4-(2,2-difluoro-1-(isoindolin-2-yl)-2-(phenylsulfonyl)ethyl)phenol

2-(1-(3-Bromo-4-methoxyphenyl)-2,2-difluoro-2-(phenylsulfonyl)ethyl)iso-indoline (8.0 g, 15.7 mmol, 1.0 eq.) was taken in aqueous HBr (200 mL) at 25° C. The mixture was heated at 110° C. for 5 days. After completion of the reaction, the mixture was concentrated under reduced pressure to afford the title compound as a brown solid. Yield 7.5 g (crude). MS (ESI) m/z [M+1]⁺ 494.01. ¹H NMR (400 MHz, DMSO-d₆): δ 7.85 (d, 2H), 7.70-7.75 (m, 1H), 7.61-7.66 (m, 1H), 7.49-7.53 (m, 2H), 7.31 (d, 1H), 7.10-7.20 (m, 4H), 6.96 (d, 1H), 5.10-5.25 (m, 1H), 3.95-4.05 (m, 2H), 3.79-3.84 (m, 2H).

f) tert-Butyl 4-((2-bromo-4-(2,2-difluoro-1-(isoindolin-2-yl)-2-(phenylsulfonyl)-ethyl)phenoxy)methyl)piperidine-1-carboxylate

Treatment of 2-bromo-4-(2,2-difluoro-1-(isoindolin-2-yl)-2-(phenylsulfonyl)-ethyl)phenol (3.0 g, 6.1 mmol, 1.0 eq.) with tert-butyl 4-(((methylsulfonyl)oxy)methyl)-piperidine-1-carboxylate (3.2 g, 11.0 mmol, 1.8 eq.) in the presence of potassium carbonate (4.2 g, 30.5 mmol, 5.0 eq.) in DMF (50 mL) at 60° C. for 16 h followed by column chromatography afforded the title compound as an off white solid. Yield 3.0 g, 72%. MS (ESI) m/z [M+1]⁺: 691.25. ¹H NMR (400 MHz, DMSO-d₆): δ 8.03 (d, 2H), 7.68-7.75 (m, 2H), 7.44-7.52 (m, 3H), 7.05-7.15 (m, 5H), 5.11-5.20 (m, 1H), 3.90-4.00 (m, 4H), 3.70-3.82 (m, 2H), 2.60-2.72 (m, 2H), 1.92-2.00 (m, 2H), 1.70-1.80 (m, 3H), 1.38 (s, 9H), 1.15-1.30 (m, 2H).

g) 2-(1-(3-Bromo-4-(piperidin-4-ylmethoxy)phenyl)-2,2-difluoro-2-(phenyl-sulfonyl)ethyl)isoindoline

Treatment of tert-butyl 4-((2-bromo-4-(2,2-difluoro-1-(isoindolin-2-yl)-2-(phenylsulfonyl)ethyl)phenoxy)methyl)piperidine-1-carboxylate (3.0 g, 4.3 mmol, 1.0 eq.) in DCM (20 mL) with trifluoroacetic acid (4.9 g, 43.4 mmol, 10.0 eq.) afforded the title compound as a brown solid. Yield 3.0 g (crude). MS (ESI) m/z [M+1]⁺: 591.05.

h) 2-(1-(3-Bromo-4-((1-(pyrimidin-2-ylmethyl)piperidin-4-yl)methoxy)phenyl)-2,2-difluoro-2-(phenylsulfonyl)ethyl)isoindoline

To a solution of 2-(1-(3-bromo-4-(piperidin-4-ylmethoxy)phenyl)-2,2-difluoro-2-(phenylsulfonyl)ethyl)isoindoline (3.0 g, 5.07 mmol, 1.0 eq.) in THF (30.0 mL) and H₂O (10.0 mL), were added pyrimidin-2-ylmethyl methanesulfonate (1.9 g, 10.2 mmol, 2.0 eq.) and Et₃N (5.1 g, 7 mL, 51.0 mmol, 10.0 eq.) at 25° C. The reaction mixture was stirred at 25° C. for 24 h. After completion of the reaction, the reaction mixture was quenched with ice cold water and extracted (3 times) with ethyl acetate. The combined organic layer was washed with brine, dried over anhydrous sodium sulphate, filtered and concentrated under reduced pressure. This crude product was purified by combi-flash chromatography using 0-50% ethyl acetate in heptane as an eluent afforded the title compound as an off white solid. Yield 1.3 g, 38%. MS (ESI) m/z [M+1]⁺: 683.16. ¹H NMR (400 MHz, DMSO-d₆): δ 8.76 (d, 2H), 7.85 (d, 2H), 7.70-7.92 (m, 2H), 7.46-7.55 (m, 2H), 7.43-7.45 (m, 1H), 7.38 (t, 1H), 7.09-7.18 (m, 5H), 5.10-5.19 (m, 1H), 3.90-4.00 (m, 2H), 3.85-3.89 (m, 2H), 3.70-3.80 (m, 2H), 3.68 (bs, 2H), 2.88-2.92 (m, 2H), 2.08-2.15 (m, 2H), 1.68-1.75 (m, 3H), 1.30-1.40 (m, 2H).

i) 2-(1-(3-Bromo-4-((1-(pyrimidin-2-ylmethyl)piperidin-4-yl)methoxy)phenyl)-2,2-difluoroethyl)isoindoline

To a solution of 2-(1-(3-bromo-4-((1-(pyrimidin-2-ylmethyl)piperidin-4-yl)-methoxy)phenyl)-2,2-difluoro-2-(phenylsulfonyl)ethyl)isoindoline (0.5 g, 0.73 mmol, 1.0 eq.) in DMF (10 mL) at 0° C., acetic acid (0.88 g, 0.837 mL, 14.6 mmol, 20.0 eq.) and sodium acetate (1.20 g, 14.6 mmol, 20.0 eq.) were added portionwise. Then magnesium turning (0.366 g, 14.6 mmol, 20.0 eq.) was added and the reaction mass was stirred for 16 h at 25° C. After completion of the reaction, the mixture was quenched with ice cold water and extracted (3 times) with 10% methanol in ethyl acetate. The combined organic layer was dried over anhydrous sodium sulphate, filtered and concentrated under reduced pressure. This crude product was purified by combi-flash chromatography using 0-10% methanol in ethyl acetate as an eluent. The desired fractions were concentrated under reduced pressure to afford the title compound as a brown sticky solid. Yield 0.13 g, 33%. MS (ESI) m/z [M+1]⁺: 543.14. ¹H NMR (400 MHz, DMSO-d₆): δ 8.77 (d, 2H), 7.63-7.69 (m, 1H), 7.44-7.52 (m, 2H), 7.15-7.25 (m, 4H), 7.11-7.13 (m, 1H), 6.47 (t, 1H), 4.03-4.05 (m, 1H), 3.88-3.91 (m, 4H), 3.79-3.82 (m, 2H), 3.65-3.69 (m, 2H), 2.91-2.94 (m, 2H), 2.11-2.16 (m, 2H), 1.70-1.80 (m, 3H), 1.35-1.38 (m, 2H). MS (ESI) m/z [M+1]⁺: 543.14.

j) 2-(2,2-Difluoro-1-(3-(methylsulfonyl)-4-((1-(pyrimidin-2-ylmethyl)piperidin-4-yl)methoxy)phenyl)ethyl)isoindoline (Compound 22)

Treatment of 2-(1-(3-bromo-4-((1-(pyrimidin-2-ylmethyl)piperidin-4-yl)-methoxy)phenyl)-2,2-difluoroethyl)isoindoline (0.14 g, 0.25 mmol 1.0 eq.) with sodium methanesulfinate (0.13 g, 1.29 mmol, 5.0 eq.) in the presence of potassium carbonate (0.03 g, 10.25 mmol, 1.0 eq.), copper(I) iodide (0.048 g, 0.25 mmol, 1.0 eq.) and 2-picolinic acid (0.061 g, 0.5 mmol, 2.0 eq.) in DMSO (4.0 mL) at 110° C. for 16 h followed by preparative HPLC purification using method A (rt: 13.38 min) afforded the title compound as a white solid. Yield 0.012 g. MS (ESI) m/z [M+1]⁺: 543.2. ¹H NMR (400 MHz, DMSO-d₆): δ 10.05 (bs,1H), 8.95 (d, 2H), 7.90 (s, 1H), 7.75 (d, 1H), 7.61 (t, 1H), 7.37 (d, 1H), 7.18-7.24 (m, 4H), 6.48 (t, 1H), 4.64 (bs, 2H), 4.24 (bs, 1H), 4.12 (d, 2H), 3.63-3.94 (m, 4H), 3.39 (bs, 1H), 3.30 (s, 3H), 3.16-3.21 (m, 2H), 1.98-2.15 (m, 4H), 1.69-1.78 (m, 2H).

### Example 23

2-((5-(1,3,4-Oxadiazol-2-yl)isoindolin-2-yl)methyl)-5-(piperidin-4-ylmethoxy)-4H-pyran-4-one (Compound 23a),

2-((5-(1,3,4-Oxadiazol-2-yl)isoindolin-2-yl)methyl)-5-((1-(pyrimidin-2-yl-methyl) piperidin-4-yl)methoxy)-4H-pyran-4-one (Compound 23b) and

2-((5-(1,3,4-Oxadiazol-2-yl)-2H-isoindol-2-yl)methyl)-5-((1-(pyrimidin-2-yl-methyl)piperidin-4-yl)methoxy)-4H-pyran-4-one (Compound 23c)

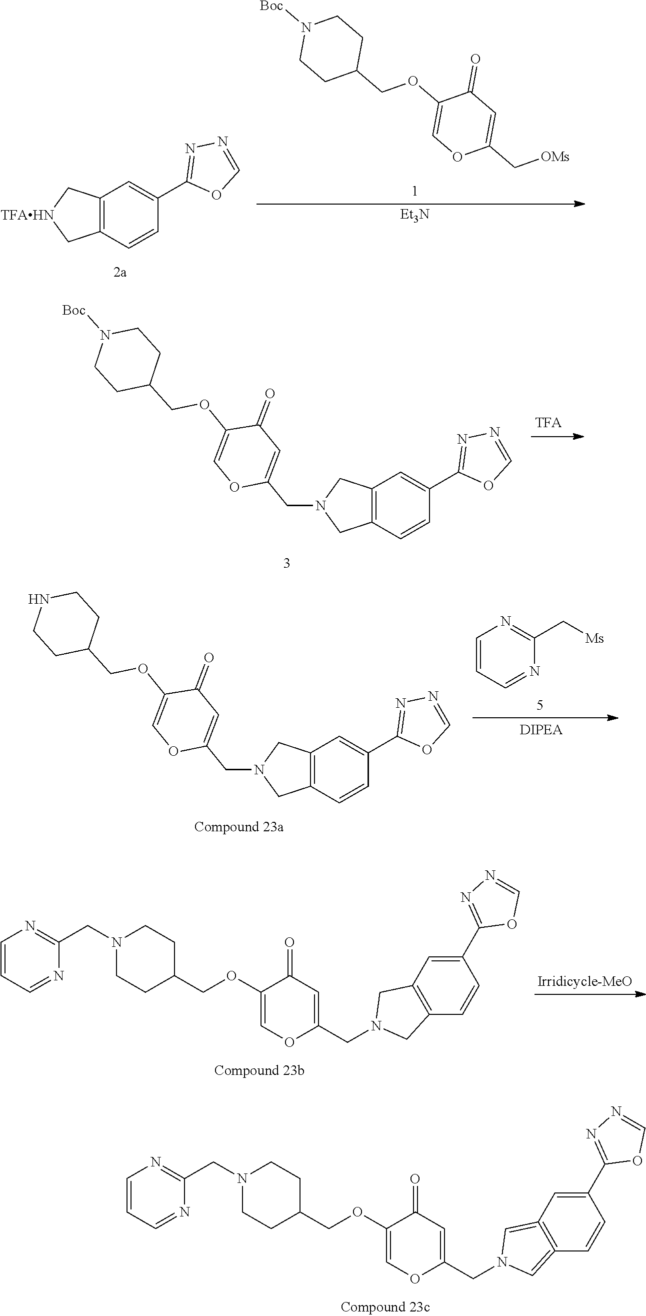

a) tert-Butyl 4-(((6-((5-(1,3,4-oxadiazol-2-yl)isoindolin-2-yl)methyl)-4-oxo-4H-pyran-3-yl)oxy)methyl)piperidine-1-carboxylate

Treatment of 2-(isoindolin-5-yl)-1,3,4-oxadiazole 2,2,2-trifluoroethan-1-one salt (1.0 g, 3.52 mmol, 1.0 eq.) with tert-butyl 4-(((6-(((methylsulfonyl)oxy)methyl)-4-oxo-4H-pyran-3-yl)oxy)methyl)piperidine-1-carboxylate (1.47 g, 3.52 mmol, 1.0 eq.) in the presence of Et₃N (1.78 g, 2.4 mL, 17.6 mmol, 5.0 eq.) in THF (10 mL), and water (10 mL) at 65° C. for 4 h afforded the title compound as a brown liquid. Yield 1.0 g, 33%. MS (ESI) m/z [M+1]⁺: 509.25.

b) 2-((5-(1,3,4-Oxadiazol-2-yl)isoindolin-2-yl)methyl)-5-(piperidin-4-ylmethoxy)-4H-pyran-4-one (Compound 23a)

Treatment of tert-butyl 4-(((6-((5-(1,3,4-oxadiazol-2-yl)isoindolin-2-yl)methyl)-4-oxo-4H-pyran-3-yl)oxy)methyl)piperidine-1-carboxylate (1.0 g, 1.96 mmol, 1.0 eq.) with TFA (5 mL) in DCM (10 mL) at 0° C. for 1 h afforded the title compound as a brown sticky solid. Yield 1.0 g (crude). MS (ESI) m/z [M+1]+: 409.28.

c) 2-((5-(1,3,4-Oxadiazol-2-yl)isoindolin-2-yl)methyl)-5-((1-(pyrimidin-2-yl-methyl) piperidin-4-yl)methoxy)-4H-pyran-4-one (Compound 23b)

Treatment of 2-((5-(1,3,4-oxadiazol-2-yl)isoindolin-2-yl)methyl)-5-(piperidin-4-ylmethoxy)-4H-pyran-4-one (1.0 g, 2.4 mmol, 1.0 eq.) with pyrimidin-2-ylmethyl methanesulfonate (0.46 g, 2.4 mmol, 1.0 eq.) in the presence of DIPEA (1.6 g, 2.1 mL, 12 mmol, 5.0 eq.) in CH₃CN (10 mL) at 65° C. for 2 h afforded the title compound as a colourless liquid. Yield 0.40 g. MS (ESI) m/z [M+1]⁺: 501.41. ¹H NMR (400 MHz, DMSO-d₆): δ 9.32 (s, 1H), 8.71-8.77 (m, 2H), 8.13 (s, 1H), 7.88-7.91 (m, 2H), 7.48 (d, 1H), 7.35-7.45 (m, 1H), 6.39 (s, 1H), 4.07 (s, 4H), 3.81 (s, 2H), 3.60-3.75 (m, 4H), 2.85-3.00 (m, 2H), 2.00-2.20 (m, 2H), 1.60-180 (m, 3H), 1.20-135 (m, 2H).

d) 2-((5-(1,3,4-Oxadiazol-2-yl)-2H-isoindol-2-yl)methyl)-5-((1-(pyrimidin-2-ylmethyl) piperidin-4-yl)methoxy)-4H-pyran-4-one (Compound 23c)

To a solution of 2-((5-(1,3,4-oxadiazol-2-yl)isoindolin-2-yl)methyl)-5-((1-(pyri-midin-2-ylmethyl)piperidin-4-yl)methoxy)-4H-pyran-4-one (0.4 g, 0.8 mmol, 1.0 eq.) in trifluoroethanol (6 mL) under N₂ atmosphere, chloro[5-methoxy-2-[1-[(4-methoxy-phenyl)imino-N]ethyl]phenyl-C][(1,2,3,4,5-eta)-1,2,3,4,5-pentamethyl-2,4-cyclopenta dien-1-yl]iridium (0.049 g, 0.08 mmol, 0.1 eq.) was added at 25° C. followed by heating at 110° C. for 48 h under dark conditions. The mixture was filtered under dark conditions and the filtrate was concentrated under vacuum. This crude product was purified by preparative HPLC using method C (rt; 13.79 min, column: X select hexyl phenyl) to afford the title compound as a brown solid. Yield 0.04 g. MS (ESI) m/z [M+1]⁺: 499.26. ¹H NMR (400 MHz, DMSO-d₆): δ 9.25 (s, 1H), 8.75 (d, 2H), 8.27 (s, 1H), 8.11 (s, 1H), 7.67 (d, 2H), 7.49 (s, 1H), 7.45 (d, 1H), 7.37 (t, 1H), 6.29 (s, 1H), 5.48 (s, 2H), 3.61-3.66 (m, 4H), 2.87 (d, 2H), 2.08 (t, 2H), 1.64 (d, 3H), 1.20-123 (m, 2H).

### Example 24

5-((1-(Methylsulfonyl)piperidin-4-yl)methoxy)-2-((S*)-1-((1aR*,7bR*)-1,1a,3,7b-tetrahydro-2H-cyclopropa[c]isoquinolin-2-yl)ethyl)-4H-pyran-4-one (Compound 24a) and

5-((1-(Methylsulfonyl)piperidin-4-yl)methoxy)-2-((R*)-1-((1aR*,7bR*)-1,1a,3,7b-tetrahydro-2H-cyclopropa[c]isoquinolin-2-yl)ethyl)-4H-pyran-4-one (Compound 24b)

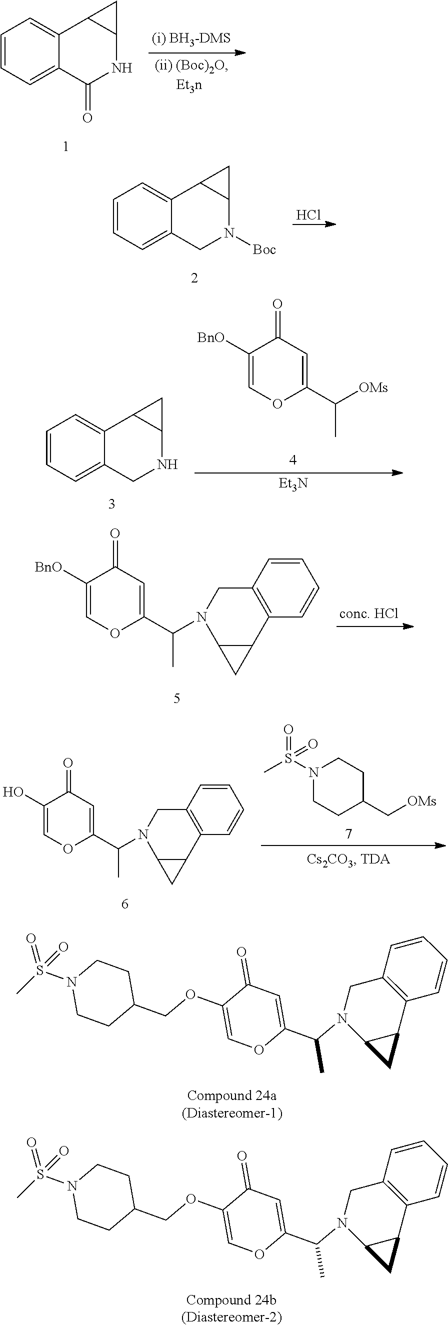

a) tert-Butyl 1,1a,3,7b-tetrahydro-2H-cyclopropa[c]isoquinoline-2-carboxylate

To a solution of 1,1a,2,7b-tetrahydro-3H-cyclopropa[c]isoquinolin-3-one (0.85 g, 5.34 mmol, 1.0 eq.) in THF (20 mL), borane-DMS (6.08 g, 7.60 mL, 80.1 mmol, 15.0 eq.) was added at 0° C. followed by stirring at 60° C. for 16 h. The mixture was cooled to 0° C., quenched with methanol, then refluxed at 60° C. for 1 h and concentrated with high vacuum to get colorless liquid. This crude product was taken in DCM (10 mL). Et₃N (1.62 g, 2.23 mL, 16.02 mmol, 3.0 eq.) and boc-anhydride (2.33 g, 2.45 mL, 10.6 mmol, 2.0 eq.) were added at 0° C. followed by stirring at 25° C. for 16 h. After completion of the reaction, the reaction mixture was quenched with ice cold water and extracted (3 times) with EtOAc. The combined organic layer was washed with brine, dried over anhydrous sodium sulphate, filtered and concentrated under reduced pressure. The crude product was purified by combi-flash chromatography using 0-10% ethyl acetate in hexanes as an eluent. The desired fractions were concentrated under reduced pressure to afford tert-butyl 1,1a,3,7b-tetrahydro-2H-cyclopropa[c]isoquinoline-2-carboxylate as a colorless sticky solid. Yield 0.40 g, 39%. MS (ESI) m/z [M+1−56]⁺: 190.11. ¹H NMR (400 MHz, DMSO-d₆): δ 7.36 (d, 1H), 7.19 (t, 1H), 7.12 (t, 1H), 7.05-7.10 (m, 1H), 4.27-4.56 (m, 2H), 3.30-3.40 (m, 1H), 2.09-2.15 (m, 1H), 1.47 (s, 9H), 1.20-1.30 (m, 1H), 0.69-0.83 (m, 1H).

b) 1a,2,3,7b-Tetrahydro-1H-cyclopropa[c]isoquinoline

Treatment of tert-butyl 1,1a,3,7b-tetrahydro-2H-cyclopropa[c]isoquinoline-2-carboxylate (0.40 g, 1.63 mmol, 1.0 eq.) with HCl in EtOAc (5.0 mL, 1.0 M solution) in EtOAc (2 mL) for 12 h afforded the title compound as a white solid. Yield 0.20 g (crude). MS (ESI) m/z [M+1]⁺: 146.02. ¹H NMR (400 MHz, DMSO-d₆): δ 10.31 (bs, 1H), 9.40-9.70 (bs, 1H), 7.45 (d, 1H), 7.26-7.31 (m, 1H), 7.19-7.23 (m, 2H), 4.01-4.23 (m, 2H), 3.40-3.50 (m, 1H), 2.26-2.32 (m, 1H), 1.52-1.57 (m, 1H), 1.10-1.25 (m, 1H).

c) 5-(Benzyloxy)-2-(1-(1,1a,3,7b-tetrahydro-2Hcyclopropa[c]isoquinolin-2-yl)ethyl)-4H-pyran-4-one

Treatment of 1a,2,3,7b-tetrahydro-1H-cyclopropa[c]isoquinoline hydrochloride (0.50 g, 3.44 mmol, 1.0 eq.) with 1-(5-(benzyloxy)-4-oxo-4H-pyran-2-yl)ethyl methane-sulfonate (0.54 g, 1.65 mmol, 0.6 eq.) in the presence of Et₃N (1.74 g, 2.3 mL, 17.2 mmol, 5.0 eq.) in THF and H₂O (1:1, 10.0 mL) at 25° C. for 24 h followed by column chromatography afforded the title compound as a colorless liquid as mixture of diastereomers. Yield 0.40 g, 31%. MS (ESI) m/z [M+1]⁺: 374.27. ¹H NMR (400 MHz, DMSO-d₆): δ 8.15, 8.18 (2 s, 1H), 7.30-7.40 (m, 5H), 7.23 (t, 1H), 7.08-7.12 (m, 1H), 6.96-7.04 (m, 2H), 6.34, 6.38 (2 s, 1H), 4.90 (d, 2H), 3.80-3.85 (m, 1H), 3.50-3.66 (m, 2H), 2.81-2.89 (m, 1H), 1.86-1.91 (m, 1H), 1.39-1.42 (m, 3H), 0.91-0.98 (m, 1H), 0.71-0.77 (m, 1H).

d) 5-Hydroxy-2-(1-(1,1a,3,7b-tetrahydro-2H-cyclopropa[c]isoquinolin-2-yl)-ethyl)-4H-pyran-4-one

Treatment of 5-(benzyloxy)-2-(1-(1,1a,3,7b-tetrahydro-2H-cyclopropa[c]iso-quinolin-2-yl)ethyl)-4H-pyran-4-one (0.40 g, 1.07 mmol, 1.0 mmol) in concentrated HCl (5 mL) for 16 h afforded the title compound as a colorless liquid as mixture of diastereomers. Yield 0.30 g (crude). MS (ESI) m/z [M+1]⁺: 284.19. ¹H NMR (400 MHz, DMSO-d₆): δ 9.04 (bs, 1H), 7.97, 8.01 (2 s, 1H), 7.21-7.44 (m, 2H), 6.92-7.11 (m, 2H), 6.35, 6.39 (2 s, 1H), 3.80-3.88 (m, 1H), 3.49-3.62 (m, 1H), 3.32-3.39 (m, 1H), 2.80-2.88 (m, 1H), 1.88-198 (m, 1H), 1.40-1.42 (m, 3H), 0.90-0.95 (m, 1H), 0.71-0.85 (m, 1H). MS (ESI) m/z [M+1]⁺: 284.19.

e) 5-((1-(Methylsulfonyl)piperidin-4-yl)methoxy)-2-((S*)-1-((1aR*,7bR*)-1,1a,3,7b-tetrahydro-2H-cyclopropa[c]isoquinolin-2-yl)ethyl)-4H-pyran-4-one (Compound 24a, diastereomer-1) and

5-((1-(Methylsulfonyl)piperidin-4-yl)methoxy)-2-((R*)-1-((1aR*,7bR*)-1,1a,3,7b-tetrahydro-2H-cyclopropa[c]isoquinolin-2-yl)ethyl)-4H-pyran-4-one (Compound 24b, diastereomer-2)

Treatment of 5-hydroxy-2-(1-(1,1a,3,7b-tetrahydro-2H-cyclopropa[c]isoquinolin-2-yl)ethyl)-4H-pyran-4-one (0.3 g, 1.06 mmol, 1.0 eq.) with (1-(methylsulfonyl)piperidin-4-yl)methyl methanesulfonate (0.23 g, 1.06 mmol, 1.0 eq.) in the presence of cesium carbonate (0.86 g, 2.65 mmol, 2.5 eq.) and tris(2-(2-methoxyethoxy)ethyl)amine (0.03 g, 0.11 mmol, 0.1 eq.) in DMSO (5 mL) at 60° C. for 5 h afforded mixture of diastereomers. This mixture was purified by preparative HPLC using method C (rt: 11.99 min for diastereomer-1, rt: 12.80 min for diastereomer-2) to give two corresponding diastereomeres as white solids. Yield 0.03 g for diastereomer-1 and 0.04 g for diastereomer-2.

Diastereomer-1: MS (ESI) m/z [M+1]⁺: 459.15. ¹H NMR (400 MHz, DMSO-d₆): δ 8.02 (s,1H),7.22 (d,1H), 7.08 (t, 1H), 6.96-7.05 (m, 2H), 6.31 (s, 1H), 3.83 (d, 1H), 3.66 (d, 2H), 3.50-3.62 (m, 4H), 2.87-2.89 (m, 1H), 2.85 (s, 3H), 2.66-2.74 (m, 2H), 1.81-1.91-(m, 4H), 1.41 (d, 3H), 1.25-1.31 (m, 2H), 0.92-0.96 (m, 1H), 0.69-0.75 (m, 1H).

Diastereomer-2: MS (ESI) m/z [M+1]⁺: 459.15. ¹H NMR (400 MHz, DMSO-d₆): δ 8.02 (s,1H), 7.24 (d,1H), 7.10 (t, 1H), 6.99-7.06 (m, 2H), 6.36 (s, 1H), 3.78 (d, 1H), 3.69 (d, 2H), 3.50-3.62 (m, 4H), 2.85 (s, 3H), 2.66-2.82 (m, 3H), 1.81-1.91 (m, 4H), 1.39 (d, 3H), 1.25-1.31 (m, 2H), 0.94-0.98 (m, 1H), 0.74-0.80 (m, 1H).

### Example 25

2-(3-(Ethylsulfonyl)-4-((1-(methylsulfonyl)piperidin-4-yl)methoxy)benzyl)iso-indoline (Compound 25)

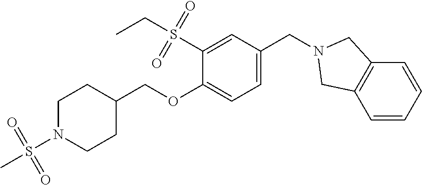 [SMILES: CCS(=O)(=O)c1cc(CN2Cc3ccccc3C2)ccc1OCC1CCN(S(C)(=O)=O)CC1]

a) Methyl 3-iodo-4-((1-(methylsulfonyl)piperidin-4-yl)methoxy)benzoate

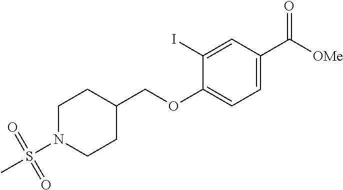 [SMILES: COC(=O)c1ccc(OCC2CCN(S(C)(=O)=O)CC2)c(I)c1]

To a mixture of methyl 4-hydroxy-3-iodobenzoate (0.278 g, 1.00 mmol) and 4-(methanesulphonyloxymethyl)-1-methanesulphonylpiperidine (0.298 g, 1.10 mmol) in dry DMSO (3.0 ml) was added potassium carbonate (0.304 g, 2.20 mmol). The mixture was stirred at 100° C. until the reaction was completed. Water was added to the cooled reaction mixture and stirred at RT overnight. The precipitated product was filtered, washed with water and dried under vacuum to afford the title compound (0.41 g). ¹H NMR (400 MHz, DMSO-d₆): δ 8.29 (d, 1H), 7.96 (dd, 1H), 7.11 (d, 1H), 4.05 (d, 2H), 3.83 (s, 3H), 3.57-3.66 (m, 2H), 2.87 (s, 3H), 2.77 (td, 2H), 1.87-1.99 (m, 3H), 1.40-1.54 (m, 2H).

b) Methyl 3-(ethylsulfonyl)-4-((1-(methylsulfonyl)piperidin-4-yl)methoxy)-benzoate

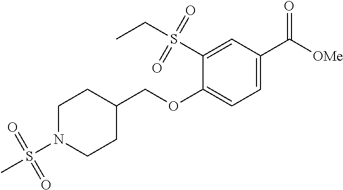 [SMILES: CCS(=O)(=O)c1cc(C(=O)OC)ccc1OCC1CCN(S(C)(=O)=O)CC1]

A mixture of methyl 3-iodo-4-((1-(methylsulfonyl)piperidin-4-yl)methoxy)-benzoate (0.41 g, 0.904 mol), sodium ethanesulfinate (0.273 g, 2.352 mmol), Copper (I) iodide (0.172 g, 0.904 mmol) and L-proline (0.104 g, 0.904 mmol) in dry DMSO (4.5 ml) was stirred at 100° C. for 6 h. Then, more sodium ethanesulfinate (0.273 g, 2.352 mmol) was added and stirring was continued at 100° C. for 4 h. The mixture was diluted with water and extracted with EtOAc (2×). Combined organic phases were washed with water and brine, dried over anhydrous Na₂SO₄, filtered and evaporated to obtain crude product. Purification by flash column chromatography afforded the title compound (0.19 g). ¹H NMR (400 MHz, CDCl₃): δ 8.62 (d, 1H), 8.28 (dd, 1H), 7.07 (d, 1H), 4.08 (d, 2H), 3.87-3.95 (m, 2H), 3.93 (s, 3H), 3.32 (q, 2H), 2.81 (s, 3H), 2.74 (td, 2H), 1.98-2.12 (m, 3H), 1.48-1.62 (m, 2H), 1.25 (t, 3H).

c) 3-(Ethylsulfonyl)-4-((1-(methylsulfonyl)piperidin-4-yl)methoxy)phenyl)-methanol

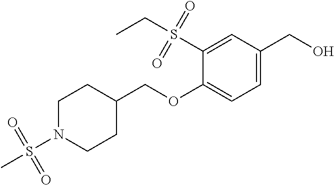 [SMILES: CCS(=O)(=O)c1cc(CO)ccc1OCC1CCN(S(C)(=O)=O)CC1]

To a cooled (0-5° C.) mixture of methyl 3-(ethylsulfonyl)-4-((1-(methylsulfonyl)-piperidin-4-yl)methoxy)benzoate (0.19 g, 0.453 mmol) in dry THF (2 ml) was added lithium borohydride (2 M in THF, 0.6 ml, 1.20 mmol). The mixture was stirred at RT for 3.5 h and then at 60° C. until the reaction was completed. The mixture was cooled to 0-5° C., treated with dry acetone (2 ml), stirred at RT and the evaporated to dryness. To the residue was added water and saturated NH₄Cl-solution and then the mixture was extracted with EtOAc (2×). Combined organic phases were washed with water and brine, dried over anhydrous Na₂SO₄, filtered and evaporated to obtain the title compound (0.17 g). ¹H NMR (400 MHz, CDCl₃): δ 7.93 (d, 1H), 7.62 (dd, 1H), 7.02 (d, 1H), 4.70 (d, 2H), 4.01 (d, 2H), 3.85-3.93 (m, 2H), 3.33 (q, 2H), 2.80 (s, 3H), 2.73 (td, 2H), 1.97-2.06 (m, 3H), 1.89-1.95 (m, 1H), 1.46-1.59 (m, 2H), 1.26 (t, 3H).

d) 4-((4-(Chloromethyl)-2-(ethylsulfonyl)phenoxy)methyl)-1-(methylsulfonyl)-piperidine

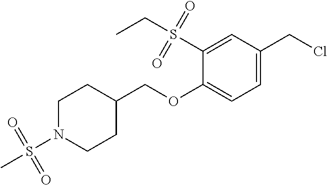 [SMILES: CCS(=O)(=O)c1cc(CCl)ccc1OCC1CCN(S(C)(=O)=O)CC1]

To a mixture of (3-(ethylsulfonyl)-4-((1-(methylsulfonyl)piperidin-4-yl)methoxy)phenyl)methanol (0.17 g, 0.434 mmol) in dry DCM (3 ml) containing catalytic amount of DMF was added thionyl chloride (0.063 ml, 0.868 mmol). The mixture was stirred at RT until the reaction was completed. Solvents were evaporated, dry DCM was added and the evaporation repeated. Drying under vacuum afforded the title compound (0.148 g). ¹H NMR (400 MHz, CDCl₃): δ 7.97 (d, 1H), 7.62 (dd, 1H), 7.01 (d, 1H), 4.58 (s, 2H), 4.01 (d, 2H), 3.86-3.94 (m, 2H), 3.33 (q, 2H), 2.81 (s, 3H), 2.74 (td, 2H), 1.97-2.09 (m, 3H), 1.47-1.60 (m, 2H), 1.26 (t, 3H).

e) 2-(3-(Ethylsulfonyl)-4-((1-(methylsulfonyl)piperidin-4-yl)methoxy)benzyl)-isoindoline (Compound 25)

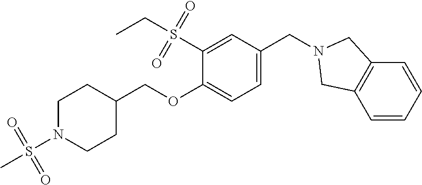 [SMILES: CCS(=O)(=O)c1cc(CN2Cc3ccccc3C2)ccc1OCC1CCN(S(C)(=O)=O)CC1]

A mixture of 4-((4-(chloromethyl)-2-(ethylsulfonyl)phenoxy)methyl)-1-(methyl-sulfonyl)piperidine (0.148 g, 0.361 mmol), isoindoline hydrochloride (0.062 g, 0.397 mmol) and N,N-diisopropylamine (0.138 ml, 0.794 mmol) in dry DMSO (1.5 ml) was stirred at 60° C. until the reaction was completed. Water was added to the cooled mixture followed by was stirring at RT for 30 min. The precipitated product was filtered, washed with water and dried under vacuum to afford the crude pruct. Purification with reverse phase flash chromatography afforded the title compound (0.061 g). ¹H NMR (400 MHz, DMSO-d₆): δ 7.79 (d, 1H), 7.67 (dd, 1H), 7.27 (d, 1H), 7.15-7.25 (m, 4H), 4.07 (d, 2H), 3.88 (s, 2H), 3.83 (s, 4H), 3.55-3.65 (m, 2H), 3.37 (q, 2H), 2.87 (s, 3H), 2.71-2.81 (m, 2H), 1.84-2.01 (m, 3H), 1.34-1.48 (m, 2H), 1.09 (t, 3H). MS: m/z 493.8 (M+H)⁺.

### Example 26

### 2-(3-(Cyclopropylsulfonyl)-4-((1-(methylsulfonyl)piperidin-4-yl)methoxy)-benzyl)isoindoline (Compound 26)

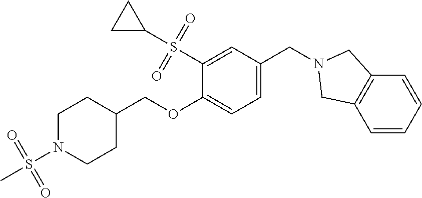 [SMILES: CS(=O)(=O)N1CCC(COc2ccc(CN3Cc4ccccc4C3)cc2S(=O)(=O)C2CC2)CC1]

a) Methyl 3-(cyclopropylsulfonyl)-4-((1-(methylsulfonyl)piperidin-4-yl)methoxy)benzoate

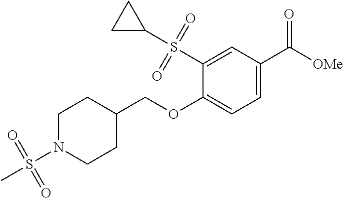 [SMILES: COC(=O)c1ccc(OCC2CCN(S(C)(=O)=O)CC2)c(S(=O)(=O)C2CC2)c1]

The compound was prepared according to the procedure of Example 25(b) starting from 3-iodo-4-((1-(methylsulfonyl)piperidin-4-yl)methoxy)benzoate (0.50 g, 1.103 mmol), sodium cyclopropanesulfinate (0.567 g, 4.425 mmol), copper(I) iodide (0.127 g, 1.103 mmol) and L-Proline (0.210 g, 1.103 mmol) in dry DMSO (5.0 ml). Purification by column chromatography afforded the title compound (0.25 g). ¹H NMR (400 MHz, CDCl₃): δ 8.53 (d, 1H), 8.26 (dd, 1H), 7.08 (d, 1H), 4.10 (d, 2H), 3.86-3.95 (m, 2H), 3.92 (s, 3H), 2.81-2.88 (m, 1H), 2.81 (s, 3H), 2.75 (td, 2H), 2.01-2.15 (m, 3H), 1.48-1.62 (m, 2H), 1.28-1.35 (m, 2H), 0.98-1.05 (m, 2H).

b) (3-(Cyclopropylsulfonyl)-4-((1-(methylsulfonyl)piperidin-4-yl)methoxy)-phenyl)methanol

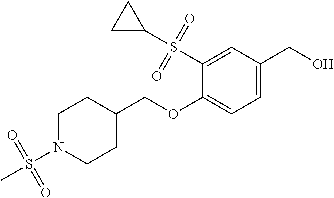 [SMILES: CS(=O)(=O)N1CCC(COc2ccc(CO)cc2S(=O)(=O)C2CC2)CC1]

The compound was prepared according to the procedure of Example 25(c) starting from methyl 3-(cyclopropylsulfonyl)-4-((1-(methylsulfonyl)piperidin-4-yl)-methoxy)benzoate (0.25 g, 0.579 mmol) and lithium borohydride (2M in THF, 0.9 ml, 1.80 mmol) in dry THF (3.0 ml)at 60° C. Purification by reverse phase flash chromatography afforded the title compound (0.0814 g). ¹H NMR (400 MHz, CDCl₃): δ 7.84 (d, 1H), 7.59 (dd, 1H), 7.03 (d, 1H), 4.68 (d, 2H), 4.03 (d, 2H), 3.85-3.93 (m, 2H), 2.84-2.92 (m, 1H), 2.80 (s, 3H), 2.74 (td, 2H), 2.00-2.12 (m, 3H), 1.86 (t, 1H), 1.46-1.60 (m, 2H), 1.26-1.32 (m, 2H), 0.96-1.03 (m, 2H).

c) 4-((4-(Chloromethyl)-2-(cyclopropylsulfonyl)phenoxy)methyl)-1-(methyl-sulfonyl)piperidine

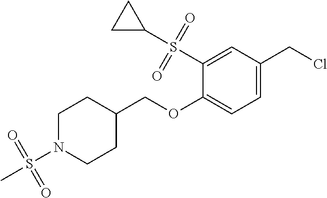 [SMILES: CS(=O)(=O)N1CCC(COc2ccc(CCl)cc2S(=O)(=O)C2CC2)CC1]

The compound was prepared according to the procedure of Example 25(d) starting from (3-(cyclopropylsulfonyl)-4-((1-(methylsulfonyl)piperidin-4-yl)methoxy)-phenyl)methanol (0.81 g, 0.201 mmol) and thionyl chloride (0.03 ml, 0.411 mmol) in dry DCM (1.5 ml). Yield 0.081 g. ¹H NMR (600 MHz, DMSO-d₆): δ 7.80 (d, 1H), 7.74 (d, 1H), 7.32 (d, 1H), 4.83 (s, 2H), 4.11 (d, 2H), 3.57-3.64 (m, 2H), 2.96-3.02 (m, 1H), 2.87 (s, 3H9, 2.72-2.80 (m, 2H), 1.89-2.04 (m, 3H), 1.36-1.45 (m, 2H), 1.00-1.10 (m, 4H).

d) 2-(3-(Cyclopropylsulfonyl)-4-((1-(methylsulfonyl)piperidin-4-yl)methoxy)-benzyl)isoindoline (Compound 26)

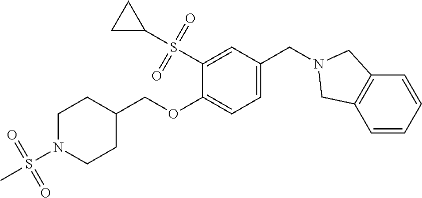 [SMILES: CS(=O)(=O)N1CCC(COc2ccc(CN3Cc4ccccc4C3)cc2S(=O)(=O)C2CC2)CC1]

The compound was prepared according to the procedure of Example 25(e) starting from 4-((4-(chloromethyl)-2-(cyclopropylsulfonyl)phenoxy)methyl)-1-(methyl-sulfonyl)piperidine (0.081 g, 0.192 mmol), isoindoline (0.024 ml, 0.211 mmol) and DIPEA (0.074 ml, 0.422 mmol) in dry DMSO (1.0 ml). Raw product was triturated with diethylether-methanol and dried under vacuum to afford the title compound (0.023 g).

¹H NMR (400 MHz, DMSO-d₆): δ 7.72 (d, 1H), 7.65 (dd, 1H), 7.28 (d, 1H), 7.15-7.25 (m, 4H), 4.09 (d, 2H), 3.87 (s, 2H), 3.83 (d, 4H), 3.57-3.66 (m, 2H), 2.94-3.03 (m, 1H), 2.87 (s, 3H), 2.77 (td, 2H), 1.89-2.05 (m, 3H9, 1.34-1.48 (m, 2H), 1.00-1.07 (m, 4H).

MS: m/z 505.8 (M+H)⁺.

### Example 27

2-(3-(Ethylsulfonyl)-4-((1-(methylsulfonyl)piperidin-4-yl)methoxy)benzyl)-5-(trifluoromethyl)isoindoline (Compound 27)

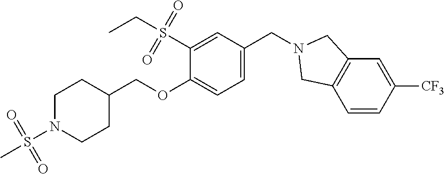 [SMILES: CCS(=O)(=O)c1cc(CN2Cc3ccc(C(F)(F)F)cc3C2)ccc1OCC1CCN(S(C)(=O)=O)CC1]

The compound was prepared according to the procedure of Example 25(e) starting from 4-((4-(chloromethyl)-2-(ethylsulfonyl)phenoxy)methyl)-1-(methylsulfonyl)piperidine (0.143 g, 0.35 mmol), 5-(trifluoromethyl)isoindoline hydrochloride (0.086, 0.385 mmol) and DIPEA (0.134 ml, 0.77 mmol) in dry DMSO (1.5 ml). Purification by reverse phase flash chromatography afforded the title compound (0.0313 g). ¹H NMR (400 MHz, CDCl₃): δ 7.95 (d, 1H), 7.65 (dd, 1H), 7.47 (d, 1H), 7.43 (s, 1H), 7.29 (s, 1H), 7.01 (d, 1H), 4.01 (d, 2H), 3.95 (s, 4H9, 3.51-3.93 (m, 4H), 3.35 (q, 2H), 2.81 (s, 3H), 2.68-2.79 (m, 2H), 1.96-2.11 (m, 3H), 1.47-1.61 (m, 2H), 1.26 (t, 3H). MS: m/z 561.5 (M+H)⁺.

### Example 28

2-(3-(Butylsulfonyl)-4-((1-(methylsulfonyl)piperidin-4-yl)methoxy)benzyl)iso-indoline (Compound 28)

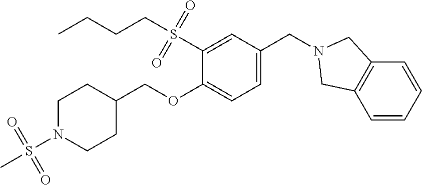 [SMILES: CCCCS(=O)(=O)c1cc(CN2Cc3ccccc3C2)ccc1OCC1CCN(S(C)(=O)=O)CC1]

a) Butyl 3-(butylsulfonyl)-4-fluorobenzoate

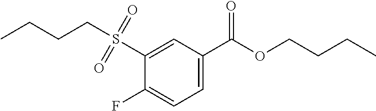 [SMILES: CCCCOC(=O)c1ccc(F)c(S(=O)(=O)CCCC)c1]

A mixture of 4-fluoro-3-sulfinobenzoic acid (0.15 g, 0.735 mmol), 1-iodobutane (0.25 ml, 2.204 mmol) and potassium carbonate (0.305 g, 2.204 mmol) in dry DMF (1.0 ml) was stirred at 80° C. until the reaction was completed. Cooled reaction mixture was diluted with water and extracted with EtOAc (2×). Combined organic phases were washed with water and brine, dried over anhydrous Na₂SO₄, filtered and evaporated to obtain the crude product. Purification by phase flash chromatography afforded the title compound (0.14 g). ¹H NMR (400 MHz, CDCl₃): δ 8.61 (dd, 1H), 8.34 (ddd, 1H), 7.32 (dd, 1H), 4.36 (t, 2H), 3.28-3.35 (m, 2H), 1.68-1.82 (m, 4H), 1.39-1.52 (m, 4H), 0.99 (t, 3H), 0.92 (t, 3H).

b) Butyl 3-(butylsulfonyl)-4-((1-(methylsulfonyl)piperidin-4-yl)methoxy)benzoate

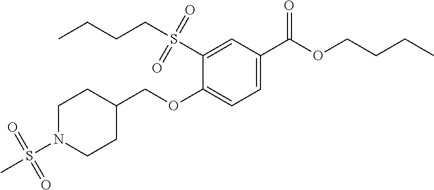 [SMILES: CCCCOC(=O)c1ccc(OCC2CCN(S(C)(=O)=O)CC2)c(S(=O)(=O)CCCC)c1]

To a cooled mixture of sodium hydride (50 wt-% in oil, 0.013 g, 0.553 mmol) in dry DMF (1.5 ml) was added a solution of (1-(methylsulfonyl)piperidin-4-yl)methanol (0.094 g, 0.487 mmol). The mixture was stirred at RT for 15 min after which a solution of butyl 3-(butylsulfonyl)-4-fluorobenzoate (0.14 g, 0.442 mmol) in dry DMF (1.0 ml) was added and stirring at RT was continued until the reaction was completed. Reaction mixture was diluted with water and extracted with EtOAc (2×). Combined organic phases were washed with water and brine, dried over anhydrous Na₂SO₄, filtered and evaporated to obtain crude product. Purification by phase flash chromatography afforded the title compound (0.10 g). ¹H NMR (400 MHz, DMSO-d₆): δ 8.33 (d, 1H), 8.24 (dd, 1H), 7.43 (d, 1H), 4.29 (t, 2H), 4.19 (d, 2H), 3.56-3.65 (m, 2H), 3.37-3.44 (m, 2H), 2.87 (s, 3H), 2.71-2.81 (m, 2H), 1.85-2.04 (m, 3H), 1.65-1.75 (m, 2H), 0.94 (t, 3H), 0.83 (t, 3H).

c) 3-(Butylsulfonyl)-4-((1-(methylsulfonyl)piperidin-4-yl)methoxy)phenyl)-methanol

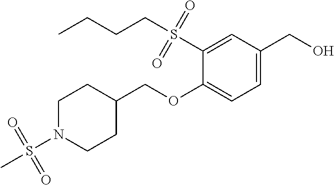 [SMILES: CCCCS(=O)(=O)c1cc(CO)ccc1OCC1CCN(S(C)(=O)=O)CC1]

The compound was prepared according to the procedure of Example 25(c) starting from butyl 3-(butylsulfonyl)-4-((1-(methylsulfonyl)piperidin-4-yl)methoxy)-benzoate (0.10 g, 0.204 mmol) and lithium borohydride (2 M in THF, 0.5 ml, 1.00 mmol) in dry THF (2 ml). Yield of crude product 0.108 g. LC-MS: m/z 420.4 (M+H)⁺.

d) 4-((2-(Butylsulfonyl)-4-(chloromethyl)phenoxy)methyl)-1-(methylsulfonyl)-piperidine

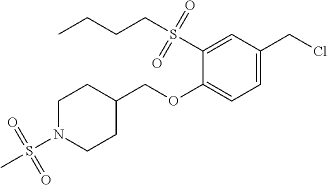 [SMILES: CCCCS(=O)(=O)c1cc(CCl)ccc1OCC1CCN(S(C)(=O)=O)CC1]

The compound was prepared according to the procedure of Example 25(d) starting from (3-(butylsulfonyl)-4-((1-(methylsulfonyl)piperidin-4-yl)methoxy)phenyl)-methanol (0.108 g, 0.206 mmol) and thionyl chloride (0.05 ml, 0.685 mmol) in dry DCM (2.0 ml). Yield 0.081 g. ¹H NMR (400 MHz, CDCl₃): δ 7.97 (d, 1H), 7.62 (dd, 1H), 7.01 (d, 1H), 4.58 (s, 2H), 4.02 (d, 2H), 3.86-3.94 (m, 2H), 3.27-3.34 (m, 2H), 2.81 (s, 3H), 2.74 (td, 2H), 1.97-2.10 (m, 3H), 1.64-1.71 (m, 2H), 1.48-1.60 (m, 2H), 1.36-1.46 (m, 2H), 0.91 (t, 3H).

e) 2-(3-(Butylsulfonyl)-4-((1-(methylsulfonyl)piperidin-4-yl)methoxy)benzyl)isoindoline (Compound 28)

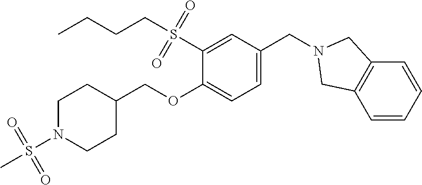 [SMILES: CCCCS(=O)(=O)c1cc(CN2Cc3ccccc3C2)ccc1OCC1CCN(S(C)(=O)=O)CC1]

The compound was prepared according to the procedure of Example 25(e) starting from 4-((2-(butylsulfonyl)-4-(chloromethyl)phenoxy)methyl)-1-(methylsulfonyl)piperidine (0.081 g, 0.185 mmol), isoindoline (0.023 ml, 0.203 mmol) and DIPEA (0.081 ml, 0.462 mmol) in dry DMSO (1.0 ml). Purification by reverse phase flash chromatography afforded the title compound (0.015 g). ¹H NMR (600 MHz, DMSO-d₆): δ 7.79 (d, 1H), 7.67 (dd, 1H), 7.27 (d, 1H), 7.17-7.24 (m, 4H), 4.08 (d, 2H), 3.88 (s, 2H), 3.92 (s, 4H), 3.57-3.63 (m, 2H), 3.35-3.43 (m, 2H), 2.87 (s, 3H), 2.76 (td, 2H), 1.87-1.98 (m, 3H), 1.47-1.54 (m, 2H), 1.38-1.47 (m, 2H), 1.30-1.38 (m, 2H9, 0.83 (t, 3H). MS: m/z 521.9 (M+H)⁺.

### Example 29

2-((1′H-Spiro[cyclopropane-1,4′-isoquinolin]-2′(3′H)-yl)methyl)-5-((1-(methyl-sulfonyl)piperidin-4-yl)methoxy)-4H-pyran-4-one (Compound 29)

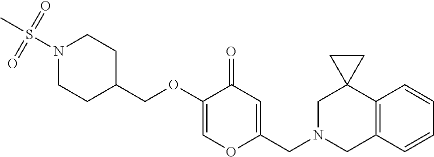 [SMILES: CS(=O)(=O)N1CCC(COc2coc(CN3Cc4ccccc4C4(CC4)C3)cc2=O)CC1]

A mixture of 6′-methoxy-2′,3′-dihydro-1′H-spiro[cyclopropane-1,4′-isoquinoline] hydrochloride (0.050 g, 0.220 mmol), 2-(chloromethyl)-5-((1-(methylsulfonyl)piperidin-4-yl)methoxy)-4H-pyran-4-one (0.074 g, 0.220 mmol) and DIPEA (0.096 ml, 0.550 mmol) in dry DMSO (1.5) was stirred at 60° C. until the reaction was completed. Reaction mixture was diluted with water and extracted with EtOAc (2×). Combined organic phases were washed with water and brine, dried over anhydrous Na₂SO₄, filtered and evaporated to obtain crude product. Purification by reverse phase flash chromatography afforded the title compound (0.087 g). ¹H NMR (400 MHz, CDCl₃): δ 7.60 (s, 1H), 6.92 (d, 1H), 6.66 (dd, 1H), 6.47 (s, 1H), 6.21 (d, 1H), 3.81-3.90 (m, 2H), 3.82 (s, 2H), 3.75 (s, 3H), 3.74 (d, 2H), 3.56 (s, 2H), 2.79 (s, 3H), 2.69 (td, 2H), 2.66 (s, 2H), 1.95-2.08 (m, 3H), 1.36-1.50 (m, 2H), 1.00-1.06 (m, 2H), 0.82-0.89 (m, 2H). MS: m/z 489.9 (M+H)⁺.

### Example 30

tert-Butyl 6-(((6-((3,4-dihydroisoquinolin-2(1H)-yl)methyl)-4-oxo-4H-pyran-3-yl)oxy)methyl)-2-azaspiro[3.3]heptane-2-carboxylate (Compound 30)

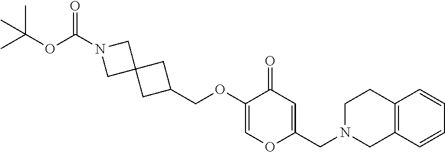 [SMILES: CC(C)(C)OC(=O)N1CC2(CC(COc3coc(CN4CCc5ccccc5C4)cc3=O)C2)C1]

a) tert-Butyl 6-(((methylsulfonyl)oxy)methyl)-2-azaspiro[3.3]heptane-2-carboxylate

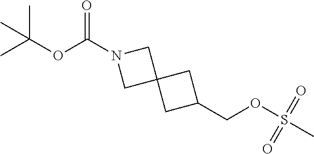 [SMILES: CC(C)(C)OC(=O)N1CC2(CC(COS(C)(=O)=O)C2)C1]

To a cooled (0-5° C.) mixture of tert-butyl 6-(hydroxymethyl)-2-azaspiro[3.3]-heptane-2-carboxylate (0.20 g, 0.880 mmol) and triethylamine (0.25 ml, 1.794 mmol) in dry DCM (4.0 ml) was added methanesulfonyl chloride (0.072 ml, 0.924 mmol) dissolved in dry DCM (2.0 ml). The reaction mixture was stirred at RT overnight and then diluted with DCM and washed with saturated NaHCO₃ solution, water and brine. Organic phase was dried and evaporated to afford the title compound (0.24 g). ¹H NMR (400 MHz, CDCl₃): δ 4.15 (d, 2H), 3.93 (s, 2H), 3.84 (s, 2H), 3.01 (s, 3H), 2.51-2.64 (m, 1H), 2.28-2.37 (m, 2H), 1.99-2.07 (m, 2H), 1.43 (s, 9H).

b) tert-Butyl 6-(((6-((3,4-dihydroisoquinolin-2(1H)-yl)methyl)-4-oxo-4H-pyran-3-yl)oxy)methyl)-2-azaspiro[3.3]heptane-2-carboxylate (Compound 30)

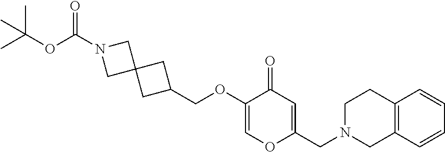 [SMILES: CC(C)(C)OC(=O)N1CC2(CC(COc3coc(CN4CCc5ccccc5C4)cc3=O)C2)C1]

A mixture of tert-butyl 6-(((methylsulfonyl)oxy)methyl)-2-azaspiro[3.3]heptane-2-carboxylate (0.229 g, 0.750 mmol), 2-{[3,4-dihydroisoquinolin-2(1H)-yl]methyl}-5-hydroxy-4H-pyran-4-one (0.193 g, 0.750 mmol) and potassium carbonate (0.228 g, 1.650 mmol) in dry DMSO (4.0 ml) was stirred at 100° C. until the reaction was completed. Cooled reaction mixture was diluted with water and extracted with EtOAc (2×). Combined organic phases were washed with water and brine, dried and evaporated to yield the title compound (0.28 g). Part of the crude product (60 mg) was purified by reverse phase flash chromatography to afford the title compound (0.0255 g). ¹H NMR (400 MHz, CDCl₃): δ 7.60 (s, 1H), 7.08-7.17 (m, 3H), 6.97-7.02 (m, 1H), 6.49 (s, 1H), 3.93 (s, 2H), 3.85 (s, 3H), 3.83 (d, 2H), 3.71 (s, 2H), 3.55 (s, 2H), 2.90-2.97 (m, 2H), 2.80-2.86 (m, 2H), 2.58-2.70 (m, 1H), 2.30-2.39 (m, 2H), 2.00-2.09 (m, 2H), 1.43 (s, 9H). MS: m/z 468.0 (M+H)⁺.

### Example 31

tert-Butyl 6-((6-((3,4-dihydroisoquinolin-2(1H)-yl)methyl)-4-oxo-4H-pyran-3-yl)oxy)-2-azaspiro[3.3]heptane-2-carboxylate (Compound 31)

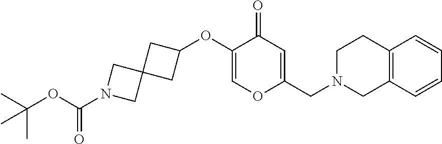 [SMILES: CC(C)(C)OC(=O)N1CC2(CC(Oc3coc(CN4CCc5ccccc5C4)cc3=O)C2)C1]

To a cooled (0-5° C.) mixture of 2-((3,4-dihydroisoquinolin-2(1H)-yl)methyl)-5-hydroxy-4H-pyran-4-one (0.077 g, 0.300 mmol), tert-butyl 6-hydroxy-2-azaspiro[3.3]-heptane-2-carboxylate (0.064 g, 0.300 mmol) and triphenylphosphine (0.118 g, 0.450 mmol) in dry THF (2.5 ml) was added diisopropyl azodicarboxylate (0.089 ml, 0.450 mmol). The mixture was stirred at RT overnight. Reaction mixture was diluted with DCM, washed with water and brine. Organic phase was dried and evaporated. The crude product was purified by reverse phase flash chromatography to afford the title compound (0.039 g). ¹H NMR (600 MHz, DMSO-d₆): δ 7.98 (s, 1H), 7.07-7.16 (m, 3H), 7.03 (d, 1H), 6.37 (s, 1H), 4.37-4.47 (m, 1H), 3.71-3.95 (m, 4H), 3.62 (s, 2H), 3.58 (s, 2H), 2.79-2.87 (m, 2H), 2.70-2.79 (m, 2H), 2.59-2.68 (m, 2H), 2.13-2.22 (m, 2H), 1.36 (s, 9H). MS: m/z 453.8 (M+H)⁺.

The following compounds were prepared according to the procedure described for Compound 31of Example 31 starting from 2-((3,4-dihydroisoquinolin-2(1H)-yl)-methyl)-5-hydroxy-4H-pyran-4-one and another appropriate starting material. The compound number, characterization data and the another starting material are indicated in the table.

| No | Structure and starting material | ¹H NMR / LC-MS |
|---|---|---|
| 32 | 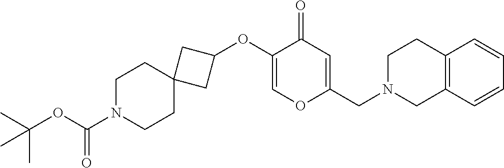 [SMILES: CC(C)(C)OC(=O)N1CCC2(CC1)CC(Oc1coc(CN3CCc4ccccc4C3)cc1=O)C2] | ¹H NMR (600 MHz, DMSO-d₆) δ: 7.96 (s, 1H), 7.06-7.18 (m, 3H), 6.98- 7.06 (m, 1H), 6.37 (s, 1H), 4.49-4.59 (m, 1H), 3.62 (s, 2H), 3.57 (s, 2H), 3.23 (d, 4H), 2.82 (s, 2H), 2.75 (s, 2H), 2.28-2.41 (m, 2H), 1.70-1.83 (m, 2H), 1.46 (s, 4H), 1.38 (s, 9H). LC- MS: m/z 482.4 (M + H). |
| | Starting material: tert-Butyl 2- hydroxy-7-azaspiro [3.5]nonane-7- carboxylate | |
| 33 | 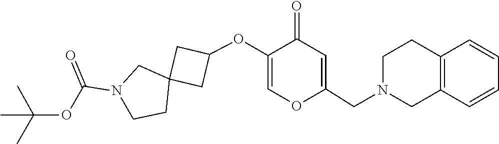 [SMILES: CC(C)(C)OC(=O)N1CCC2(CC(Oc3coc(CN4CCc5ccccc5C4)cc3=O)C2)C1] | ¹H NMR (600 MHz, DMSO-d₆) δ: 7.98-8.02 (m, 1H), 7.06-7.15 (m, 3H), 7.00-7.06 (m, 1H), 6.37 (d, 1H), 4.50- 4.58 (m, 1H), 3.62 (s, 2H), 3.57 (s, 2H), 3.15-3.28 (m, 4H), 2.82 (t, 2H), 2.75 (t, 2H), 2.35-2.47 (m, 2H), 1.95- 2.07 (m, 2H), 1.78-1.88 (m, 2H), 1.35- 1.42 (m, 9H). LC-MS: m/z 468.1 (M + H). |
| | Starting material: tert-Butyl 2- hydroxy-6-azaspiro[3.4]octane-6- carboxylate | |
| 34 | 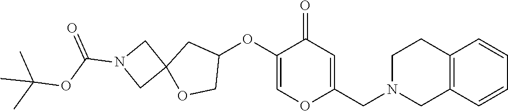 [SMILES: CC(C)(C)OC(=O)N1CC2(CC(Oc3coc(CN4CCc5ccccc5C4)cc3=O)CO2)C1] | ¹H NMR (400 MHz, Chloroform-d₆) δ: 7.66 (s, 1H), 7.09-7.18 (m, 3H), 6.98- 7.03 (m, 1H), 6.51 (s, 1H), 5.24 (br, 1H), 3.92-4.12 (m, 6H), 3.72 (s, 2H), 3.56 (s, 2H), 2.94 (t, 2H), 2.84 (t, 2H), 2.47-2.54 (m, 1H), 2.19 (dd, 1H), 1.44 (s, 9H). LC-MS: m/z 469.9 (M + H). |
| | Starting material: tert-Butyl 7- hydroxy-5-oxa-2-azaspiro[3.4]octane- 2-carboxylate | |
| 35 | 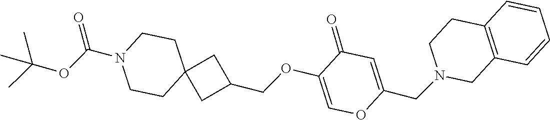 [SMILES: CC(C)(C)OC(=O)N1CCC2(CC1)CC(COc1coc(CN3CCc4ccccc4C3)cc1=O)C2] | ¹H NMR (600 MHz, DMSO-d₆) δ: 8.12 (s, 1H), 7.06-7.15 (m, 2H), 7.03 (d, 1H), 6.37 (s, 1H), 3.79 (d, 2H), 3.61 (s, 2H), 3.57 (s, 2H), 3.25 (br s, 2H), 3.17 (br s, 2H), 2.79-2.85 (m, 2H9, 2.70-2.78 (m, 2H), 2.57-2.67 (m, 1H), 1.85-1.93 (m, 2H), 1.53-1.60 (m, 2H), 1.46-1.53 (m, 2H), 1.39-1.45 (m, 2H), 1.38 (s, 9H). LC-MS: m/z 496.1 (M + H). |
| | Starting material: tert-Butyl 2- (hydroxymethyl)-7-azaspiro [3.5]- nonane-7-carboxylate | |
| 36 | 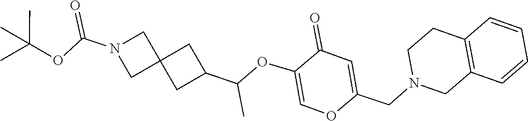 [SMILES: CC(Oc1coc(CN2CCc3ccccc3C2)cc1=O)C1CC2(C1)CN(C(=O)OC(C)(C)C)C2] | ¹H NMR (400 MHz, Chloroform-d₆) δ: |
| | Starting material: tert-Butyl 6-(1- hydroxyethyl)-2-azaspiro [3.3]heptane- 2-carboxylate | 7.65 (s, 1H), 7.08-7.17 (m, 3H), 6.97- 7.03 (m, 1H), 6.47 (s, 1H), 4.32 (quint, 1H), 3.93 (s, 2H), 3.77-3.86 (m, 2H), 3.71 (s, 2H), 3.55 (s, 2H), 2.94 (t, 2H), 2.83 (t, 2H), 2.28-2.41 (m, 1H), 2.15- 2.28 (m, 3H), 1.99-2.07 (m, 1H), 1.42 (s, 9H), 1.10 (d, 3H). LC-MS: m/z 482.0 (M + H). |

### Example 32

tert-Butyl 6-(1-((6-((3,4-dihydroisoquinolin-2(1H)-yl)methyl)-4-oxo-4H-pyran-3-yl)oxy)ethyl)-2-azaspiro[3.3]heptane-2-carboxylate (Compound 36, alternative synthesis)

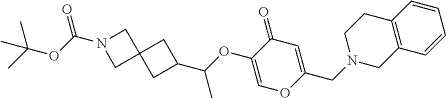 [SMILES: CC(Oc1coc(CN2CCc3ccccc3C2)cc1=O)C1CC2(C1)CN(C(=O)OC(C)(C)C)C2]

a) tert-Butyl 6-(1-((methylsulfonyl)oxy)ethyl)-2-azaspiro[3.3]heptane-2-carboxylate

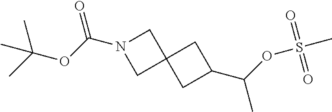 [SMILES: CC(OS(C)(=O)=O)C1CC2(C1)CN(C(=O)OC(C)(C)C)C2]

The compound was prepared according to the procedure of Example 30(a) starting from tert-butyl 6-(1-hydroxyethyl)-2-azaspiro[3.3]heptane-2-carboxylate (0.80 g, 3.31 mmol), triethylamine (0.87 ml, 1.883 mmol) and methanesulfonyl chloride (0.38 ml, 1.481 mmol) in dry DCM (12.5 ml). Yield 1.06 g. ¹H NMR (400 MHz, CDCl₃): δ 4.68 (quint, 1H), 3.93 (s, 2H), 3.78-3.85 (m, 2H), 3.01 (s, 3H), 2.20-2.42 (m, 3H), 2.07-2.16 (m, 1H), 1.94-2.02 (m, 1H), 1.43 (s, 9H(, 1.32 (d, 3H).

b) tert-Butyl 6-(1-((6-((3,4-dihydroisoquinolin-2(1H)-yl)methyl)-4-oxo-4H-pyran-3-yl)oxy)ethyl)-2-azaspiro[3.3]heptane-2-carboxylate

A mixture of 2-((3,4-dihydroisoquinolin-2(1H)-yl)methyl)-5-hydroxy-4H-pyran-4-one (0.854 g, 3.32 mmol), tert-butyl 6-(1-((methylsulfonyl)oxy)ethyl)-2-azaspiro-[3.3]heptane-2-carboxylate (1.06 g, 3.32 mmol), cesium carbonate (2.162 g, 6.64 mmol) and tris(2-(2-methoxyethoxy)ethyl)amine (0.054 g, 0.166 mmol) in dry DMSO (10 ml) was stirred at 60° C. until the reaction was completed. Cooled mixture was diluted with water and extracted with EtOAc (2×). Combined organic phases were washed with water and brine, dried and evaporated. Crude product was purified by flash chromatography to afford the title compound (0.72 g). LC-MS: m/z 482.0 (M+H). ¹H NMR (400 MHz, Chloroform-d) δ: 7.65 (s, 1H), 7.08-7.17 (m, 3H), 6.97-7.03 (m, 1H), 6.47 (s, 1H), 4.32 (quint, 1H), 3.93 (s, 2H), 3.77-3.86 (m, 2H), 3.71 (s, 2H), 3.55 (s, 2H), 2.94 (t, 2H), 2.83 (t, 2H), 2.28-2.41 (m, 1H), 2.15-2.28 (m, 3H), 1.99-2.07 (m, 1H), 1.42 (s, 9H), 1.10 (d, 3H).

### Example 33

tert-Butyl 2-(1-((6-((3,4-dihydroisoquinolin-2(1H)-yl)methyl)-4-oxo-4H-pyran-3-yl)oxy)ethyl)-7-azaspiro[3.5]nonane-7-carboxylate (Compound 37)

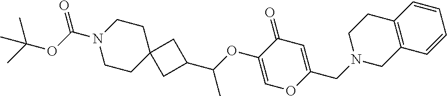 [SMILES: CC(Oc1coc(CN2CCc3ccccc3C2)cc1=O)C1CC2(CCN(C(=O)OC(C)(C)C)CC2)C1]

a) tert-Butyl 2-(1-hydroxyethyl)-7-azaspiro[3.5]nonane-7-carboxylate

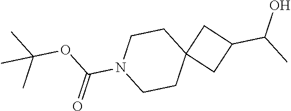 [SMILES: CC(O)C1CC2(CCN(C(=O)OC(C)(C)C)CC2)C1]

The compound was prepared according to the procedure of Intermediate 3(c) starting from tert-butyl 2-acetyl-7-azaspiro[3.5]nonane-7-carboxylate (0.47 g, 1.758 mmol) and sodium borohydride (0.10 g, 2.64 mmol) in dry methanol (5.0 ml). Yield 0.48 g. ¹H NMR (400 MHz, CDCl₃): δ 3.63-3.73 (m, 1H), 3.31-3.40 (m, 2H), 3.22-3.30 (m, 2H), 2.15-2.28 (m, 1H), 1.79-1.94 (m, 2H), 1.53-1.65 (m, 4H), 1.32-1.52 (m, 3H), 1.45 (s, 9H), 1.09 (d, 3H).

b) tert-Butyl 2-(1-((methylsulfonyl)oxy)ethyl)-7-azaspiro[3.5]nonane-7-carboxylate

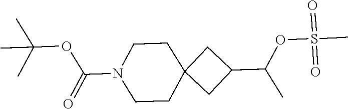 [SMILES: CC(OS(C)(=O)=O)C1CC2(CCN(C(=O)OC(C)(C)C)CC2)C1]

The compound was prepared according to the procedure of Example 32(a) starting from tert-butyl 2-(1-hydroxyethyl)-7-azaspiro[3.5]nonane-7-carboxylate (0.48 g, 1.693 mmol), triethylamine (0.45 ml, 3.23 mmol) and methanesulfonyl chloride (0.20 ml, 2.58 mmol) in dry DCM (7.5 ml). Yield: 0.60 g. ¹H NMR (400 MHz, CDCl₃): δ 4.68-4.77 (m, 1H), 3.19-3.43 (m, 4H), 3.01 (s, 3H), 2.39-2.52 (m, 1H), 1.85-1.99 (m, 2H), 1.71 (dd, 1H), 1.51-1.61 (m, 3H), 1.38-1.48 (m, 2H), 1.45 (s, 9H), 1.33 (d, 3H).

c) tert-Butyl 2-(1-((6-((3,4-dihydroisoquinolin-2(1H)-yl)methyl)-4-oxo-4H-pyran-3-yl)oxy)ethyl)-7-azaspiro[3.5]nonane-7-carboxylate (Compound 37)

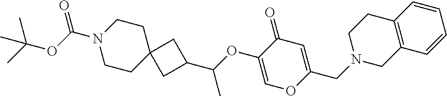 [SMILES: CC(Oc1coc(CN2CCc3ccccc3C2)cc1=O)C1CC2(CCN(C(=O)OC(C)(C)C)CC2)C1]

The compound was prepared according to the procedure of Example 32(b) (Step2a) starting from tert-butyl 2-(1-((methylsulfonyl)oxy)ethyl)-7-azaspiro[3.5]-nonane-7-carboxylate (0.60 g, 1.692 mmol), 2-((3,4-dihydroisoquinolin-2(1H)-yl)-methyl)-5-hydroxy-4H-pyran-4-one (0.435 g, 1.692 mmol), cesium carbonate (1.103 g, 3.38 mmol) and tris(2-(2-methoxyethoxy)ethyl)amine (0.027 g, 0.085 mmol) in dry DMSO (5.0 ml). Yield: 0.30 g. ¹H NMR (400 MHz, DMSO-d₆): δ 8.17 (s, 1H), 7.07-7.13 (m, 3H), 7.01-7.06 (m, 1H), 6.38 (s, 1H), 4.19-4.27 (m, 1H), 3.63 (s, 2H), 3.58 (s, 2H), 3.21-3.29 (m, 2H), 3.11-3.20 (m, 2H), 2.80-2.86 (m, 2H), 2.73-2.79 (m, 2H), 2.35-2.44 (m, 1H), 1.75-1.86 (m, 2H), 1.67 (dd, 1H), 1.45-1.58 (m, 3H), 1.32-1.41 (m, 2H), 1.38 (s, 9H), 1.05 (d, 3H).

### Example 34

5-((2-Azaspiro[3.3]heptan-6-yl)oxy)-2-((3,4-dihydroisoquinolin-2(1H)-yl)-methyl)-4H-pyran-4-one bis-trifluoroacetate (Compound 38)

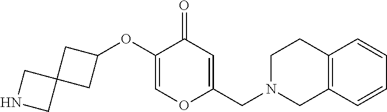 [SMILES: O=c1cc(CN2CCc3ccccc3C2)occ1OC1CC2(CNC2)C1]

To a solution of tert-butyl 6-((6-((3,4-dihydroisoquinolin-2(1H)-yl)methyl)-4-oxo-4H-pyran-3-yl)oxy)-2-azaspiro[3.3]heptane-2-carboxylate (0.30 g, 0.663 mmol) in dry DCM (5.0 ml) was added triluoroacetic acid (0.30 ml, 3.89 mmol). The mixture was stirred at RT overnight. More triluoroacetic acid was added (0.30 ml, 6.89 ml) and stirring was continued at 40° C. until reaction was completed. Solvents were evaporated and the residue was treated with more DCM and evaporation repeated to afford the title compound (0.44 g). ¹H NMR (400 MHz, DMSO-d₆): δ 8.68 (br s, 2H), 8.04 (s, 1H), 7.16-7.33 (m, 4H), 4.45 (quint, 1H), 4.36 (br s, 4H), 3.90-4.05 (m, 4H),3.33-3.62 (br, 2H), 3.00-3.16 (m, 2H), 2.69-2.79 (m, 2H), 2.22-2.31 (m, 2H). MS: m/z 353.3 (M−2TFA+H)⁺.

The following compounds were prepared according to the procedure described for Compound 38 of Example 34. The compound number, characterization data, starting material and possible deviations in reaction conditions (C=concentration of the starting material) are indicated on the table.

| | | ¹H NMR / LC-MS |
|---|---|---|
| No | Structure and starting material | Deviating reaction conditions |
| 39 | 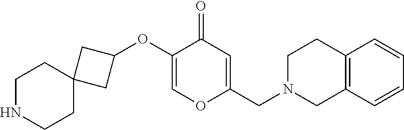 [SMILES: O=c1cc(CN2CCc3ccccc3C2)occ1OC1CC2(CCNCC2)C1] | ¹H NMR (400 MHz, DMSO-d₆): δ 8.41 (br s, 2H), 8.02 (s, 1H), 7.17-7.32 (4H, m), 6.64 (s, 1H), 4.58 (quint, 1H), 4.37 (br s, 4H), 3.34-3.62 (br, 2H), 3.06-3.14 (m, 2H), 2.91-3.06 (m, 3H), 2.36-2.50 (m, 4H), 1.81-1.90 (m, 2H), 1.66-1.75 (m, 4H). MS: m/z 381.5 (M- 2TFA + H)⁺. C: 0.087 M, TFA: 7.23 equiv. |
| | Starting material: tert-Butyl 2-((6- ((3,4-dihydroisoquinolin-2(1H)-yl)- methyl)-4-oxo-4H-pyran-3-yl)oxy)-7- azaspiro[3.5]nonane-7-carboxylate | |
| 40 | 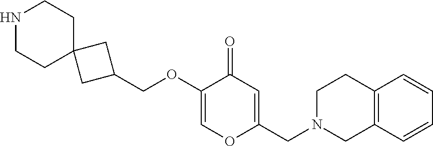 [SMILES: O=c1cc(CN2CCc3ccccc3C2)occ1OCC1CC2(CCNCC2)C1] | ¹H NMR (400 MHz, DMSO-d₆): δ 8.37 (br s, 2H), 8.19 (s, 1H), 7.16-7.31 (m, 4H), 6.63 (s, 1H), 4.35 (br s, 4H), 3.83 (d, 2H), 3.32-3.60 (br, 2H), 3.05- 3.13 (m, 2H), 2.98-3.05 (m, 2H), 2.88- 2.98 (m, 2H), 1.92-2.01 (m, 2H), 1.72- 1.79 (m, 2H), 1.60-1.71 (m, 4H). MS: m/z 395.4 (M-2TFA + H)⁺. C: 0.073 M, TFA: 7.68 equiv. |
| | Starting material: tert-Butyl 2-(((6- ((3,4-dihydroisoquinolin-2(1H)-yl)- methyl)-4-oxo-4H-pyran-3-yl)oxy)- methyl)-7-azaspiro[3.5]-nonane-7- carboxylate | |
| 41 | 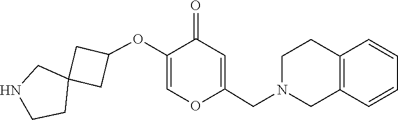 [SMILES: O=c1cc(CN2CCc3ccccc3C2)occ1OC1CC2(CCNC2)C1] | ¹H NMR (400 MHz, DMSO-d₆), 1:1 mixture of diastereomers: δ 8.87 (br s, 1H), 8.78 (br s, 1H), 8.08 (s, 0.5H), 8.05 (s, 0.5H), 7.15-7.32 (m, 4H), 6.64 (s, 1H), 4.55-4.64 (m, 1H), 4.35 (br s, 4H), 3.35-3.59 (br, 2H), 3.11-3.22 (m, 4H), 3.03-3.11 (m, 2H), 2.42-2.60 (m, 3H, obscured by DMSO-d₆), 2.07-2.20 (m, 2H), 1.92-2.02 (m, 2H). MS: m/z 367.4 (M-2TFA + H)⁺. C: 0.088 M, TFA: 12.1 equiv. |
| | Starting material: tert-Butyl 2-((6- ((3,4-dihydroisoquinolin-2(1H)-yl)- methyl)-4-oxo-4H-pyran-3-yl)oxy)-6- azaspiro [3.4]octane-6-carboxylate | |
| 42 | 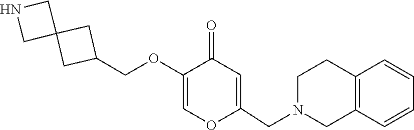 [SMILES: O=c1cc(CN2CCc3ccccc3C2)occ1OCC1CC2(CNC2)C1] | ¹H NMR (400 MHz, DMSO-d₆): δ 8.59 (br s, 2H), 8.20 (s, 1H), 7.17-7.33 (m, 4H), 6.65 (s, 1H), 4.28-4.49 (m, 4H), 3.97 (t, 4H), 3.78 (d, 2H), 3.36- 3.66 (br, 2H), 3.03-3.16 (m, 2H), 2.34- 2.52 (m, 4H), 2.00-2.10 (m, 2H). C: 0.070 M, TFA: 9.49 equiv. |
| | Starting material: tert-Butyl 6-(((6- ((3,4-dihydroisoquinolin-2(1H)-yl)- methyl)-4-oxo-4H-pyran-3-yl)oxy)- methyl)-2-azaspiro[3.3]-heptane-2- carboxylate | |
| 43 | 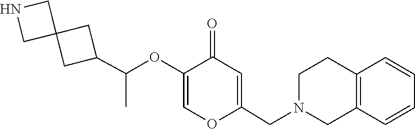 [SMILES: CC(Oc1coc(CN2CCc3ccccc3C2)cc1=O)C1CC2(CNC2)C1] | ¹H NMR (400 MHz, DMSO-d₆): δ 8.60 (br, 2H), 8.26 (s, 1H), 7.14-7.32 (m, 4H), 6.66 (s, 1H), 4.18-4.27 (m, 1H), 3.81-4.01 (m, 5H), 3.41-3.64 (m, 2H), 3.06-3.15 (m, 2H), 2.24-2.32 (m, 3H), 2.13-2.23 (m, 1H), 1.97-2.09 (m, 1H), 1.06 (d, 3H), some signals obscured by DMSO-d₆. MS: m/z 381.6 (M-2TFA + H)⁺. C: 0.132 M, TFA: 9.19 equiv. |
| | Starting material: tert-Butyl 6-(l-((6- ((3,4-dihydroisoquinolin-2(1H)-yl)- methyl)-4-oxo-4H-pyran-3-yl)oxy)- ethyl)-2-azaspiro [3.3]-heptane-2- carboxylate | |
| 44 | 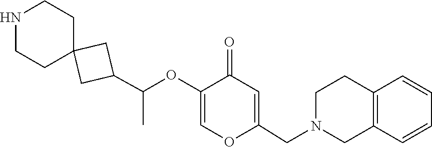 [SMILES: CC(Oc1coc(CN2CCc3ccccc3C2)cc1=O)C1CC2(CCNCC2)C1] | ¹H NMR (400 MHz, DMSO-d₆): δ 8.36 (br s, 2H), 8.25 (s, 1H), 7.15-7.32 (m, 4H), 6.65 (s, 1H), 4.27 (quint, 1H), 3.06-3.14 (m, 2H), 2.97-3.06 (m, 2H), 2.87-2.97 (m, 2H), 2.37-2.49 (m, 3H), 1.83-1.94 (m, 2H), 1.70-1.81 (m, 3H), 1.57-1.68 (m, 3H), 1.08 (d, 3H), some signals obscured by water peak. MS: m/z 409.7 (M-2TFA + H)⁺. C: 0.074 M, TFA: 7.49 equiv. |
| | Starting material: tert-Butyl 2-(l-((6- ((3,4-dihydroisoquinolin-2(1H)-yl)- methyl)-4-oxo-4H-pyran-3-yl)oxy)- ethyl)-7-azaspiro [3.5]nonane-7- carboxylate | |

### Example 35

2-((3,4-Dihydroisoquinolin-2(1H)-yl)methyl)-5-((2-(pyrimidin-2-yl)-2-azaspiro-[3.3]heptan-6-yl)oxy)-4H-pyran-4-one (Compound 45)

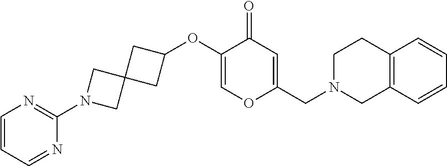 [SMILES: O=c1cc(CN2CCc3ccccc3C2)occ1OC1CC2(C1)CN(c1ncccn1)C2]

To a mixture of 5-((2-azaspiro[3.3]heptan-6-yl)oxy)-2-((3,4-dihydroisoquinolin-2(1H)-yl)methyl)-4H-pyran-4-one bis-trifluoroacetate (0.13 g, 0.224 mmol) and triethylamine (0.125 ml, 0.896 mmol) in dry DMF (1.5 ml) was added 2-bromo-pyrimidine (0.043 g, 0.269 mmol) dissolved in dry DCM (0.5 ml). The mixture was stirred at RT overnight after which it was diluted with DCM, washed with water and brine, dried and evaporated. Crude product was purified by reverse phase flash chromatography to afford the title compound (0.046 g). ¹H NMR (600 MHz, DMSO-d₆): δ 8.25 (d, 2H), 7.95 (s, 1H), 7.01-7.07 (m, 3H), 6.95-6.98 (m, 1H), 6.58 (t, 1H), 6.32 (s, 1H), 4.43 (quint, 1H), 3.98 (s, 2H), 3.93 (s, 2H), 3.55 (s, 2H), 3.51 (s, 2H), 2.76 (t, 2H), 2.62-2.71 (m, 4H), 2.16-2.23 (m, 2H). MS: m/z 431.7 (M+H)⁺.

### Example 36

2-((3,4-Dihydroisoquinolin-2(1H)-yl)methyl)-5-((7-(pyrimidin-2-yl)-7-azaspiro-[3.5]nonan-2-yl)oxy)-4H-pyran-4-one (Compound 46)

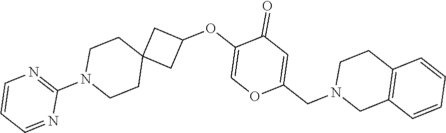 [SMILES: O=c1cc(CN2CCc3ccccc3C2)occ1OC1CC2(CCN(c3ncccn3)CC2)C1]

To a mixture of 5-((7-azaspiro[3.5]nonan-2-yl)oxy)-2-((3,4-dihydroisoquinolin-2(1H)-yl)methyl)-4H-pyran-4-one bis-trifluoroacetate (0.152 g, 0.225 mmol) and triethylamine (0.125 ml, 0.900 mmol) in dry DMF (1.5 ml) was added 2-bromo-pyrimidine (0.043 g, 0.270 mmol) dissolved in dry DMF (0.5 ml). The mixture was stirred at RT overnight after which more of 2-bromopyrimidine (0.020 mg, 0.126 mmol) was added and stirring was continued at 60° C. until the reaction was completed. The cooled mixture was diluted with DCM, washed with water and brine, dried and evaporated. Crude product was purified by reverse phase flash chromatography to afford the title compound (0.023 g). ¹H NMR (400 MHz, CDCl₃): δ 8.28 (d, 2H), 7.44 (s, 1H), 7.08-7.18 (m, 3H), 6.97-7.02 (m, 1H), 6.50)s, 1H), 6.44 (t, 1H), 4.59 (quint, 1H), 3.75-3.80 (m, 2H), 3.68-3.75 (m, 4H), 3.55 (s, 2H), 2.93 (t, 2H), 2.83 (t, 2H), 2.36-2.44 (m, 2H), 2.05-2.13 (m, 2H), 1.60-1.71 (m, 4H). MS: m/z 459.7 (M+H)⁺.

### Example 37

2-((3,4-Dihydroisoquinolin-2(1H)-yl)methyl)-5-((7-(pyrimidin-2-ylmethyl)-7-azaspiro[3.5]nonan-2-yl)oxy)-4H-pyran-4-one (Compound 47)

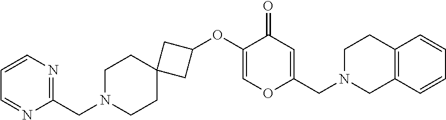 [SMILES: O=c1cc(CN2CCc3ccccc3C2)occ1OC1CC2(CCN(Cc3ncccn3)CC2)C1]

To a mixture of 5-((7-azaspiro[3.5]nonan-2-yl)oxy)-2-((3,4-dihydroisoquinolin-2(1H)-yl)methyl)-4H-pyran-4-one bis-trifluoroacetate (0.152 g, 0.225 mmol) and triethylamine (0.125 ml, 0.900 mmol) in dry DMF (1.5 ml) was added pyrimidin-2-yl-methyl methanesulfonate (0.053 g, 0.281 mmol) dissolved in dry DMF (0.5 ml). The mixture was stirred at RT overnight after which more of pyrimidin-2-ylmethyl methane-sulfonate (0.020 g, 0.106 mmol) was added and stirring was continued at 60° C. until the reaction was completed. The cooled mixture was diluted with DCM, washed with water and brine, dried and evaporated. Crude product was purified by reverse phase flash chromatography to afford the title compound (0.018 g). ¹H NMR (400 MHz, CDCl₃): δ 8.73 (d, 2H), 7.41 (s, 1H), 7.18 (t, 1H), 7.08-7.16 (m, 3H), 6.97-7.02 (m, 1H), 6.49 (s, 1H), 4.50 (quint, 1H), 3.78 (s, 2H), 3.71 (s, 2H), 3.54 (s, 2H), 2.93 (t, 2H), 2.83 (t, 2H), 2.36-2.59 (m, 4H), 2.26-2.36 (m, 2H), 1.96-2.05 (m, 2H), 1.61-1.79 (m, 4H). MS: m/z 473.5 (M+H)⁺.

### Example 38

2-((3,4-Dihydroisoquinolin-2(1H)-yl)methyl)-5-((7-(pyrimidin-2-yl)-7-azaspiro-[3.5]nonan-2-yl)methoxy)-4H-pyran-4-one (Compound 48)

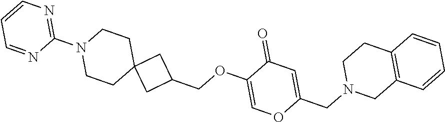 [SMILES: O=c1cc(CN2CCc3ccccc3C2)occ1OCC1CC2(CCN(c3ncccn3)CC2)C1]

The compound was prepared according to the procedure of Example 36 starting from 5-((7-azaspiro[3.5]nonan-2-yl)methoxy)-2-((3,4-dihydroisoquinolin-2(1H)-yl)methyl)-4H-pyran-4-one bis-trifluoroacetate (0.173 g, 0.225 mmol), triethylamine (0.125 ml, 0.897 mmol) and 2-bromopyrimidine (0.075 mg, 0.472 mmol) in dry DMF (2.0 ml). Yield 0.047 g. ¹H NMR (400 MHz, CDCl₃): δ 8.28 (d, 2H), 7.61 (s, 1H), 7.08-7.18 (m, 3H), 6.97-7.02 (m, 1H), 6.49 (s, 1H), 6.42 (t, 1H), 3.90 (d, 2H), 3.73-3.80 (m, 2H), 3.64-3.73 (m, 4H), 3.55 (s, 2H), 2.93 (t, 2h), 2.73-2.87 (m, 3H), 2.02-2.12 (m, 2H), 1.63-1.72 (m, 4H), 1.54-1.62 (m, 2H). MS: m/z 473.5 (M+H)⁺.

### Example 39

2-((3,4-Dihydroisoquinolin-2(1H)-yl)methyl)-5-((6-(pyrimidin-2-yl)-6-azaspiro-[3.4]octan-2-yl)oxy)-4H-pyran-4-one (Compound 49)

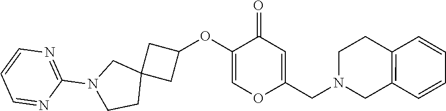 [SMILES: O=c1cc(CN2CCc3ccccc3C2)occ1OC1CC2(CCN(c3ncccn3)C2)C1]

The compound was prepared according to the procedure of Example 36 starting from 5-((6-azaspiro[3.4]octan-2-yl)oxy)-2-((3,4-dihydroisoquinolin-2(1H)-yl)methyl)-4H-pyran-4-one bis-trifluoroacetate (0.149 g, 0.250 mmol), triethylamine (0.14 ml, 1.004 mmol) and 2-bromopyrimidine (0.050 g, 0.313 g) in dry DMF (2.0 ml). Yield: 0.041 g. ¹H NMR (400 MHz, CDCl₃), mixture of diastereomers: δ 8.28-8.34 (m, 2H), 7.40-7.71 (m, 2H), 7.08-7.18 (m, 3H), 6.97-7.03 (m, 1H), 6.44-6.52 (m, 2H), 4.54-4.68 (m, 1H), 3.71 (s, 2H), 3.56-3.65 (m, 4H), 3.55 (s, 2H), 2.93 (t, 2H), 2.83 (t, 2H), 2.43-2.56 (m, 2H), 2.29-2.39 (m, 2H), 2.07 (t, 1H), 2.02 (t, 1H). MS: m/z 445.5 (M+H)⁺.

### Example 40

2-((3,4-Dihydroisoquinolin-2(1H)-yl)methyl)-5-((2-(pyrimidin-2-yl)-2-azaspiro-[3.3]heptan-6-yl)methoxy)-4H-pyran-4-one (Compound 50)

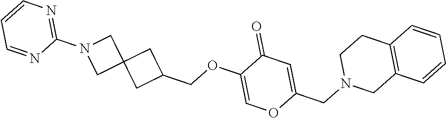 [SMILES: O=c1cc(CN2CCc3ccccc3C2)occ1OCC1CC2(C1)CN(c1ncccn1)C2]

a) Methyl 2-(pyrimidin-2-yl)-2-azaspiro[3.3]heptane-6-carboxylate

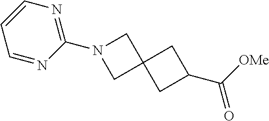 [SMILES: COC(=O)C1CC2(C1)CN(c1ncccn1)C2]

A mixture of methyl 2-azaspiro[3.3]heptane-6-carboxylate trifluoroacetate (0.269 g, 1.00 mmol), 2-chloropyrimidine (0.115 g, 1.00 mmol) and DIPEA (0.383 ml, 2.200 mmol) in dry acetonitrile (2.5 ml) was stirred at 90° C. until the reaction was completed. The cooled mixture was diluted with water and extracted with EtOAc. Organic phase was washed with water and brine, dried and evaporated. Crude product was purified by reverse phase flash chromatography to afford the title compound (0.10 g). ¹H NMR (400 MHz, CDCl₃): δ 8.30 (d, 2H), 6.52 (t, 1H), 4.16 (s, 2H), 4.09 (s, 2H), 3.70 (s, 3H), 3.07 (quint, 1H), 2.45-2.58 (m, 4H).

b) (2-(Pyrimidin-2-yl)-2-azaspiro[3.3]heptan-6-yl)methanol

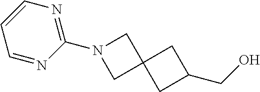 [SMILES: OCC1CC2(C1)CN(c1ncccn1)C2]

To a solution of methyl 2-(pyrimidin-2-yl)-2-azaspiro[3.3]heptane-6-carboxylate (0.20 g, 0.857 mmol) in dry THF (4.5 ml) was added lithium aluminum hydride (2.0 M in THF, 1.3 ml, 1.30 mmol) and the mixture was stirred at RT overnight. The mixture was cooled to 0-5° C., water (50 l), 2 M NaOH (100 μl) and water (150 μl) were added in this order and the mixture was stirred for 1 h at RT. Anhydrous sodium sulphate was added and stirring was continued for 15 min. Mixture was filtered through a plug of Celite. The filter cake was washed with THF and the filtrate was evaporated and dried to afford the title compound (0.18 g). ¹H NMR (400 MHz, CDCl₃/MeOD-d₄): δ 8.28 (d, 2H), 6.52 (t, 1H), 4.15 (s, 2H), 4.05 (s, 2H), 3.57 (d, 2H), 2.29-2.38 (m, 2H), 2.00-2.08 (m, 2H).

c) (2-(Pyrimidin-2-yl)-2-azaspiro[3.3]heptan-6-yl)methyl methanesulfonate

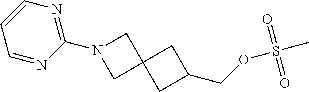 [SMILES: CS(=O)(=O)OCC1CC2(C1)CN(c1ncccn1)C2]

To a cooled (0-5° C.) mixture of (2-(pyrimidin-2-yl)-2-azaspiro[3.3]heptan-6-yl)methanol (0.080 g, 0.390 mmol) and trimethylamine (0.067 ml, 0.477 mmol) in dry DCM (1.5 ml) was added methanesulfonyl chloride (0.033 ml, 0.429 mmol) in dry DCM (0.5 ml). The mixture was stirred at 0-5° C. until the reaction was completed. The mixture was diluted with water and saturated NH₄Cl solution and extracted with DCM. Organic phase was dried and evaporated to affor the title compound (0.063 g). ¹H NMR (400 MHz, CDCl₃): δ 8.30 (d, 2H), 6.53 (t, 1H), 4.20 (d, 2H), 4.16 (s, 2H), 4.07 (s, 2H), 3.04 (s, 3H), 2.62-2.71 (m, 1H), 2.39-2.45 (m, 2H), 2.10-2.16 (m, 2H).

d) 2-((3,4-Dihydroisoquinolin-2(1H)-yl)methyl)-5-((2-(pyrimidin-2-yl)-2-aza-spiro[3.3]heptan-6-yl)methoxy)-4H-pyran-4-one (Compound 50)

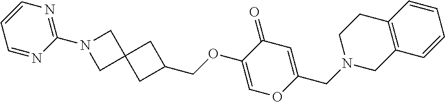 [SMILES: O=c1cc(CN2CCc3ccccc3C2)occ1OCC1CC2(C1)CN(c1ncccn1)C2]

A mixture of (2-(pyrimidin-2-yl)-2-azaspiro[3.3]heptan-6-yl)methyl methane-sulfonate (0.062 g, 0.219 mmol), 2-((3,4-dihydroisoquinolin-2(1H)-yl)methyl)-5-hydroxy-4H-pyran-4-one (0.056 g, 0.219 mmol) ans potassium carbonate (0.067 g, 0.481 mmol) in dry DMSO (1.5 ml) was stirred at 60° C. until the reaction was completed. Cooled mixture was diluted with water and extracted with EtOAc (2×). Combined organic phases were washed with water and brine, dried and evaporated. Crude product was purified by reverse phase flash chromatography to afford the title compound (0.012 g). ¹H NMR (600 MHz, DMSO-d₆): δ 8.32 (d, 2H), 8.15 (s, 1H), 7.08-7.14 (m, 3H), 7.02-7.05 (m, 1H), 6.64 (t, 1H), 6.39 (s, 1H), 4.05 (s, 2H), 3.97 (s, 2H), 3.80 (d, 2H), 3.62 (s, 2H), 3.58 (s, 2H), 2.83 (t, 2H), 2.76 (t, 2H), 2.53-2.60 (m, 1H), 2.31-2.38 (m, 2H), 2.02-2.08 (m, 2H). MS: m/z 445.7 (M+H)⁺.

### Example 41

6-(6-(((6-((3,4-Dihydroisoquinolin-2(1H)-yl)methyl)-4-oxo-4H-pyran-3-yl)oxy)-methyl)-2-azaspiro[3.3]heptan-2-yl)nicotinonitrile (Compound 51)

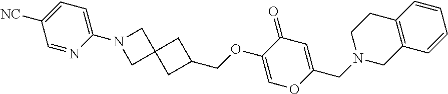 [SMILES: N#Cc1ccc(N2CC3(CC(COc4coc(CN5CCc6ccccc6C5)cc4=O)C3)C2)nc1]

A mixture of 5-((2-azaspiro[3.3]heptan-6-yl)methoxy)-2-((3,4-dihydroiso-quinolin-2(1H)-yl)methyl)-4H-pyran-4-one bis-trifluoroacetate (0.223 g, 0.225 mmol), 5-cyano-2-fluoropyridine (0.048 g, 0.393 mmol) and DIPEA (0.125 ml, 0.720 mmol) in dry DMSO (1.5 ml) was stirred at 100° C. until the reaction was completed. Cooled mixture was diluted with DCM, washed with water and brine, dried and evaporated. Crude product was purified by reverse phase flash chromatography to afford the title compound (0.033 g). ¹H NMR (600 MHz, DMSO-d₆): δ 8.43 (d, 1H), 8.15 (s, 1H), 7.80 (dd, 1H), 7.08-7.17 (m, 3H), 7.01-7-06 (m, 1H), 6.39 (s, 1H), 6.38 (d, 1H), 4.09 (s, 2H), 4.02 (s, 2H), 3.80 (d, 2H), 3.62 (s, 2H9, 3.58 (s, 2H), 2.83 (t, 2H), 2.76 (t, 2H), 2.52-2.62 (m, 1H), 2.33-2.42 (m, 2H), 2.03-2.12 (m, 2H). MS: m/z 469.8 (M+H)⁺.

### Example 42

6-(3-(((6-((3,4-Dihydroisoquinolin-2(1H)-yl)methyl)-4-oxo-4H-pyran-3-yl)oxy)-methyl)azetidin-1-yl)nicotinonitrile (Compound 52)

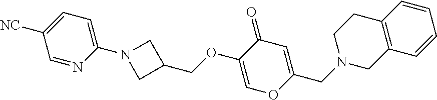 [SMILES: N#Cc1ccc(N2CC(COc3coc(CN4CCc5ccccc5C4)cc3=O)C2)nc1]

a) 6-(3-(Hydroxymethyl)azetidin-1-yl)nicotinonitrile

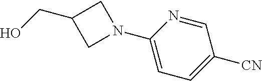 [SMILES: N#Cc1ccc(N2CC(CO)C2)nc1]

A mixture of azetidin-3-ylmethanol hydrochloride (0.124 g, 1.00 mmol), 5-cyano-2-fluoropyridine (0.122 g, 1.00 mmol) and DIPEA (0.35 ml, 2.009 mmol) in dry DMSO (2.0 ml) was stirred at 110° C. until the reaction was completed. Cooled mixture was diluted with DCM, washed with water and brine, dried and evaporated. Crude product was purified by flash chromatography to afford the title compound (0.090 g). ¹H NMR (400 MHz, DMSO-d₆): δ 8.42 (dd, 1H), 7.78 (dd, 1H), 6.40 (dd, 1H), 4.83 (t, 1H), 4.05 (t, 2H), 3.79 (dd, 2H), 3.57 (t, 2H), 2.77-2.89 (m, 1H).

b) (1-(5-Cyanopyridin-2-yl)azetidin-3-yl)methyl methanesulfonate

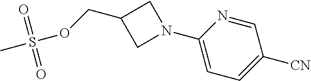 [SMILES: CS(=O)(=O)OCC1CN(c2ccc(C#N)cn2)C1]

The compound was prepared according to the procedure of Example 30 (Step 1) starting from 6-(3-hydroxymethyl)azetidin-1-yl)nicotinonitrile (0.090 g, 0.476 mmol), triethylamine (0.106 ml, 0.761 mmol) and methanesulfonyl chloride (0.055 ml, 0.713 mmol). Yield 0.120 g. ¹H NMR (400 MHz, DMSO-d₆): δ 8.39 (dd, 1H), 7.76 (dd, 1H), 6.39 (dd, 1H), 4.38 (d, 2H), 4.09 (t, 2H), 3.80 (dd, 2H), 3.16 8s, 3H), 3.03-3.14 (m, 1H).

c) 6-(3-(((6-((3,4-Dihydroisoquinolin-2(1H)-yl)methyl)-4-oxo-4H-pyran-3-yl)-oxy)methyl)azetidin-1-yl)nicotinonitrile (Compound 52)

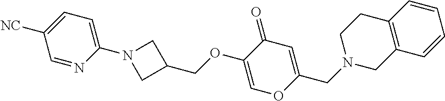 [SMILES: N#Cc1ccc(N2CC(COc3coc(CN4CCc5ccccc5C4)cc3=O)C2)nc1]

A mixture of (1-(5-cyanopyridin-2-yl)azetidin-3-yl)methyl methanesulfonate (0.049 g, 0.183 mmol), 2-((3,4-dihydroisoquinolin-2(1H)-yl)methyl)-5-hydroxy-4H-pyran-4-one (0.047 g, 0.183 mmol), cesium carbonate (0.119 g, 0.367 mmol) and tris(2-(2-methoxyethoxy)ethyl)amine (2.96 mg, 9.17 μmol) in dry DMSO (1.5 ml) was stirred at 60° C. until the reaction was completed. Cooled mixture was diluted with DCM, washed with water and brine, dried and evaporated. Crude product was purified by reverse phase flash chromatography to afford the title compound (0.079 g). ¹H NMR (400 MHz, CDCl₃): δ 8.38 (dd, 1H), 7.71 (s, 1H), 7.57 (dd, 1H), 7.08-7.19 (m, 3H), 6.97-7.02 (m, 1H), 6.51 (s, 1H), 6.23 (dd, 1H), 4.26 (t, 2H), 4.19 (d, 2H), 4.00 (dd, 2H), 3.71 (s, 2H), 3.56 (s, 2H), 3.20-3.31 (m, 1H), 2.93 (t, 2H), 2.83 (t, 2H). MS: m/z 429.8 (M+H)⁺.

### Example 43

2-((3,4-Dihydroisoquinolin-2(1H)-yl)methyl)-5-((7-((1-methyl-1H-pyrazol-5-yl)sulfonyl)-7-azaspiro[3.5]nonan-2-yl)oxy)-4H-pyran-4-one (Compound 53)

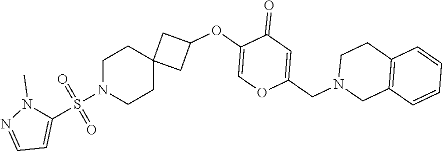 [SMILES: Cn1nccc1S(=O)(=O)N1CCC2(CC1)CC(Oc1coc(CN3CCc4ccccc4C3)cc1=O)C2]

To a mixture of 5-((7-azaspiro[3.5]nonan-2-yl)oxy)-2-((3,4-dihydroisoquinolin-2(1H)-yl)methyl)-4H-pyran-4-one bis-trifluoroacetate (0.114 g, 0.169 mmol), and triethylamine (0.125 ml, 0.897 mmol) in dry DMF (1.5 ml) was added a solution of 1-methyl-1H-pyrazole-5-sulphonyl chloride (0.030 g, 0.169 mmol) in dry DMF (0.5 ml). The mixture was stirred at RT overnight after which it was diluted with DCM, washed with water and brine, dried and evaporated. Crude product was purified by reverse phase flash chromatography to afford the title compound (0.055 g). ¹H NMR (600 MHz, DMSO-d₆): δ 7.93 (s, 1H), 7.63 (d, 1H), 7.06-7.14 (m, 3H), 7.00-7.05 (m, 1H), 6.82 (d, 1H), 6.36 (s, 1H), 4.50 (quint, 1H), 4.01 (s, 3H), 3.61 (s, 2H), 3.56 (s, 2H), 3.03-3.10 (m, 2H), 2.96-3.03 (m, 2H), 2.81 (t, 2H), 2.74 (t, 2H), 2.26-2.33 (m, 2H), 1.70-1.78 (m, 2H), 1.56-1.67 (m, 4H). MS: m/z 526.1 (M+H)⁺.

The following compounds were prepared according to the procedure described for Compound 53 of Example 43. The compound number, characterization data and the starting material are indicated in the table.

| No | Structure and starting material | ¹H NMR / LC-MS |
|---|---|---|
| 54 | 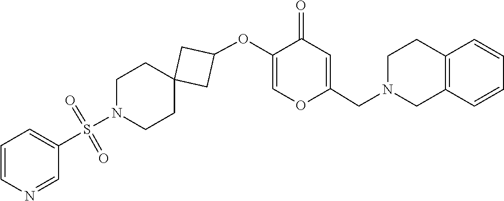 [SMILES: O=c1cc(CN2CCc3ccccc3C2)occ1OC1CC2(CCN(S(=O)(=O)c3cccnc3)CC2)C1] | ¹H NMR (600 MHz, DMSO-d₆): δ 8.90-8.92 (m, 1H), 8.89 (dd, 1H), 8.15-8.18 (m, 1H), 7.90 (s, 1H), 7.67- 7.71 (m, 1H), 7.07-7.14 (m, 3H), 7.00- 7.04 (m, 1H), 6.35 (s, 1H), 4.46 (quint, 1H), 3.60 (s, 2H), 3.55 (s, 2H), 2.92- 2.98 (m, 2H), 2.86-2.92 (m, 2H), 2.81 (t, 2H), 2.73 (t, 2H), 2.20-2.26 (m, 2H), 1.66-1.72 (m, 2H), 1.58-1.64 (m, 4H). MS: m/z 522.9 (M + H)⁺. |
| | Starting material: 5-((7-Azaspiro[3.5]- nonan-2-yl)oxy)-2-((3,4-dihydroiso- quinolin-2(1H)-yl)methyl)-4H-pyran- 4-one bis-trifluoroacetate | |
| 55 | 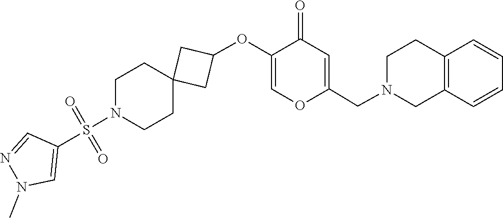 [SMILES: Cn1cc(S(=O)(=O)N2CCC3(CC2)CC(Oc2coc(CN4CCc5ccccc5C4)cc2=O)C3)cn1] | ¹H NMR (400 MHz, CDCl₃): δ 7.73 (s, 1H), 7.70 (s, 1H), 7.41 (s, 1H), 7.06-7.21 (m, 3H), 6.95-7.04 (m, 1H), 6.51 (s, 1H), 6.2-6.8 (br, 1H), 4.50 (quint, 1H), 3.97 (s, 3H), 3.73 (s, 2H), 3.58 (s, 2H), 2.79-3.02 (m, 8H), 2.20 - 2.30 (m, 2H), 1.91-2.03 (m, 2H), 1.65- 1.80 (m, 4H). MS: m/z 526.0 (M- HCOOH + H)⁺. |
| | Starting material: 5-((7-Azaspiro[3.5]- nonan-2-yl)oxy)-2-((3,4-dihydroiso- quinolin-2(1H)-yl)methyl)-4H-pyran- 4-one bis-trifluoroacetate | |
| 56 | 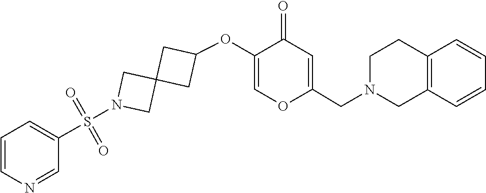 [SMILES: O=c1cc(CN2CCc3ccccc3C2)occ1OC1CC2(C1)CN(S(=O)(=O)c1cccnc1)C2] | ¹H NMR (600 MHz, DMSO-d₆): δ 8.97 (d, 1H), 8.94 (dd, 1H), 8.22-8.26 (m, 1H), 7.90 (s, 1H), 7.74 (dd, 1H), 7.06-7.14 (m, 3H), 6.99-7.04 (m, 1H), 6.34 (s, 1H), 4.31 (quint, 1H), 3.78 (s, 2H), 3.76 (s, 2H), 3.60 (s, 2H), 3.55 (s, 2H), 2.81 (t, 2H), 2.73 (t, 2H), 2.35- 2.42 (m, 2H), 1.99-2.06 (m, 2H). MS: m/z 494.9 (M + H)⁺. |
| | Starting material: 5-((2-Azaspiro[3.3]- heptan-6-yl)oxy)-2-((3,4-dihydroiso- quinolin-2(1H)-yl)methyl)-4H-pyran- 4-one bis-trifluoroacetate | |
| 57 | 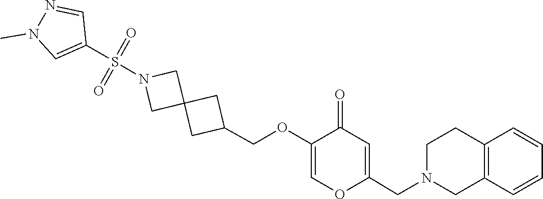 [SMILES: Cn1cc(S(=O)(=O)N2CC3(CC(COc4coc(CN5CCc6ccccc6C5)cc4=O)C3)C2)cn1] | ¹H NMR (400 MHz, CDCl₃): δ 8.11 (br S, 0.7H), 7.83 (s, 1H), 7.79 (s, 1H), 7.60 (s, 1H), 7.06-7.24 (m, 3H), 6.92- 7.06 (m, 1H), 6.51 (s, 1H), 5.80 (br, 1H), 3.99 (s, 3H), 3.65-3.88 (m, 8H), 3.59 (s, 2H), 2.77-3.06 (m, 4H), 2.49- 2.66 (m, 1H), 2.13-2.32 (m, 2H), 1.90- 2.09 (m, 2H). MS: m/z 512.1 (M- HCOOH + H)⁺. |
| | Starting material: 5-((2-Azaspiro[3.3]- heptan-6-yl)methoxy)-2-((3,4-dihydro- isoquinolin-2(1H)-yl)-methyl)-4H- pyran-4-one bis-trifluoroacetate | |
| 58 | 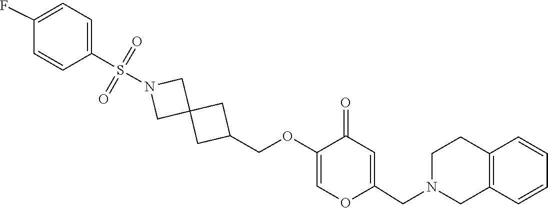 [SMILES: O=c1cc(CN2CCc3ccccc3C2)occ1OCC1CC2(C1)CN(S(=O)(=O)c1ccc(F)cc1)C2] | ¹H NMR (400 MHz, CDCl₃): δ 7.81- 7.89 (m, 2H), 7.57 (s, 1H), 7.20-7.26 (m, 2H), 7.08-7.18 (m, 3H), 6.96-7.02 (m, 1H), 6.47 (s, 1H), 3.75-3.81 (m, 4H), 3.73 (s, 2H), 3.70 (s, 2H), 3.54 (s, 2H), 2.93 (t, 2H), 2.82 (t, 2H), 2.49- 2.62 (m, 1H), 2.13-2.24 (m, 2H), 1.91- 2.03 (m, 2H). MS: m/z 626.2 (M + H)⁺. |
| | Starting material: 5-((2-Azaspiro[3.3]- heptan-6-yl)methoxy)-2-((3,4-dihydro- isoquinolin-2(1H)-yl)-methyl)-4H- pyran-4-one bis-trifluoroacetate | |
| 59 | 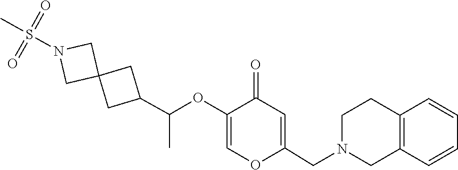 [SMILES: CC(Oc1coc(CN2CCc3ccccc3C2)cc1=O)C1CC2(C1)CN(S(C)(=O)=O)C2] | ¹H NMR (600 MHz, DMSO-d₆): δ 8.17 (s, 1H), 7.08-7.14 (m, 3H), 7.02- 7.06 (m, 1H), 6.39 (s, 1H), 4.20 (quint, 1H), 3.86 (s, 2H), 3.72-3.81 (m, 2H, AB), 3.63 (s, 2H), 3.59 (s, 2H), 2.93 (s, 3H), 2.83 (t, 2H), 2.76 (t, 2H), 2.25-2.33 (m, 1H), 2.18-2.24 (m, 2H), 2.10-2.16 (m, 1H), 1.97-2.03 (m, 1H), 1.04 (d, 3H). MS: m/z 459.9 (M + H)⁺. |
| | Starting material: 5-(l-(2-Azaspiro- [3.3]heptan-6-yl)ethoxy)-2-((3,4- dihydroisoquinolin-2(1H)-yl)-methyl)- 4H-pyran-4-one bis-trifluoroacetate | |
| 60 | 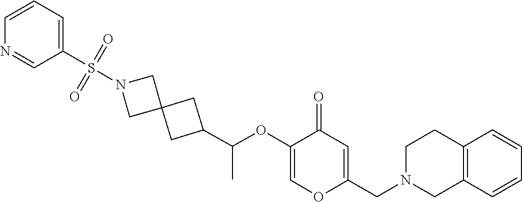 [SMILES: CC(Oc1coc(CN2CCc3ccccc3C2)cc1=O)C1CC2(C1)CN(S(=O)(=O)c1cccnc1)C2] | ¹H NMR (600 MHz, DMSO-d₆): δ 8.97 (d, 1H), 8.93 (dd, 1H), 8.21-8.25 (m, 1H), 8.12 (s, 1H), 7.73 (ddd, 1H), 7.07-7.15 (m, 3H), 7.01-7.06 (m, 1H), 6.37 (s, 1H), 4.10 (quint, 1H), 3.78 (s, 2H), 3.65-3.75 (m, 2H, AB), 3.62 (s, 2H), 3.60 (s, 2H), 2.83 (t, 2H), 2.76 (t, 2H), 2.12-2.21 (m, 1H), 1.88-1.97 (m, 3H), 1.78 (dd, 1H), 0.95 (d, 3H). MS: m/z 523.1 (M + H)⁺. |
| | Starting material: 5-(l-(2-Azaspiro- [3.3]heptan-6-yl)ethoxy)-2-((3,4- dihydroisoquinolin-2(1H)-yl)methyl)- 4H-pyran-4-one bis-trifluoroacetate | |
| 61 | 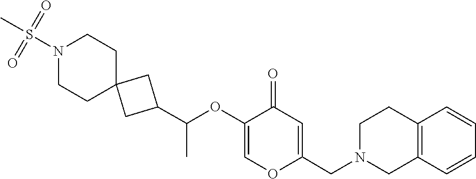 [SMILES: CC(Oc1coc(CN2CCc3ccccc3C2)cc1=O)C1CC2(CCN(S(C)(=O)=O)CC2)C1] | ¹H NMR (400 MHz, DMSO-d₆): δ 8.11 (s, 1H), 7.01-7.08 (m, 3H), 6.95- 6.99 (m, 1H), 6.31 (s, 1H), 4.13-4.20 (m, 1H), 3.56 (s, 2H), 3.52 (s, 2H), 2.95-3.02 (m, 2H), 2.85-2.92 (m, 2H), 2.76 (t, 2H), 2.75 (s, 3H), 2.69 (t, 2H), 2.29-2.38 (m, 1H), 1.72-1.79 (m, 2H), 1.64 (dd, 1H), 1.55-1.60 (m, 2H), 1.49 (dd, 1H), 1.44-1.49 (m, 2H), 0.99 (d, 3H). MS: m/z 488.3 (M + H)⁺. |
| | Starting material: 5-(l-(7-Azaspiro- [3.5]nonan-2-yl)ethoxy)-2-((3,4-di- hydroisoquinolin-2(1H)-yl)methyl)- 4H-pyran-4-one bis-trifluoroacetate | |
| 62 | 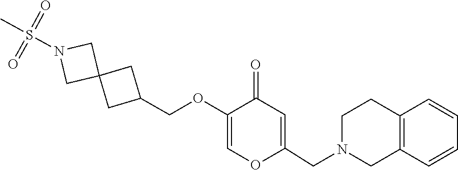 [SMILES: CS(=O)(=O)N1CC2(CC(COc3coc(CN4CCc5ccccc5C4)cc3=O)C2)C1] | ¹H NMR (600 MHz, DMSO-d₆) δ: 8.13 (s, 1H), 7.08-7.14 (m, 3H), 7.02- 7.05 (m, 1H), 6.38 (s, 1H), 3.87 (s, 2H), 3.82 (s, 2H), 3.76 (d, 2H), 3.62 (s, 2H), 3.58 (s, 2H), 2.95 (s, 3H), 2.83 (t, 2H), 2.75 (t, 2H), 2.50-2.56 (m, 1H), 2.27-2.34 (m, 2H), 1.97-2.04 (m, 2H). MS: m/z 446.1 (M + H)⁺. |
| | Starting material: 5-((2-Azaspiro[3.3]- heptan-6-yl)methoxy)-2-((3,4-dihydro- isoquinolin-2(1H)-yl)-methyl)-4H- pyran-4-one bis-trifluoroacetate | |
| 63 | 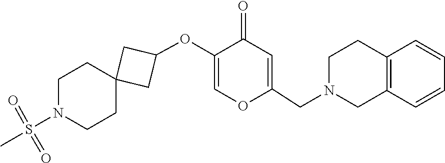 [SMILES: CS(=O)(=O)N1CCC2(CC1)CC(Oc1coc(CN3CCc4ccccc4C3)cc1=O)C2] | ¹H NMR (600 MHz, DMSO-d₆) δ:7.90 (s, 1H), 7.00-7.07 (m, 3H), 6.94-6.98 (m, 1H), 6.31 (s, 1H), 4.48 (quint, 1H), 3.55 (s, 2H), 3.51 (s, 2H), 2.96-3.02 (m, 2H), 2.91-2.96 (m, 2H), 2.76 (s, 3H), 2.75 (t, 2H), 2.68 (t, 2H), 2.26- 2.33 (m, 2H), 1.69-1.75 (m, 2H), 1.52- 1.59 (m, 4H).MS: m/z 460.1 (M + H)⁺. |
| | Starting material: 5-((7-Azaspiro[3.5]- nonan-2-yl)oxy)-2-((3,4-dihydroiso- quinolin-2(1H)-yl)methyl)-4H-pyran- 4-one bis-trifluoroacetate | |

### Example 44

2-(1-(5-Acetylisoindolin-2-yl)ethyl)-5-((1-(methylsulfonyl)piperidin-4-yl)-methoxy)-4H-pyran-4-one (Compound 64)

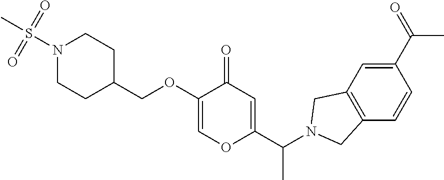 [SMILES: CC(=O)c1ccc2c(c1)CN(C(C)c1cc(=O)c(OCC3CCN(S(C)(=O)=O)CC3)co1)C2]

a) 1-(5-(Benzyloxy)-4-oxo-4H-pyran-2-yl)ethyl methanesulfonate

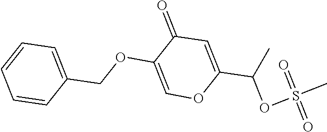 [SMILES: CC(OS(C)(=O)=O)c1cc(=O)c(OCc2ccccc2)co1]

The compound was prepared according to the procedure of Example 30(a) from 5-(benzyloxy)-2-(1-hydroxyethyl)-4H-pyran-4-one (0.246 g, 1.00 mmol), triethylamine (0.25 ml, 1.794 mmol) and methanesulfonyl chloride (0.12 ml, 1.551 mmol) in dry DCM (5.0 ml). Yield: 0.261 g. ¹H NMR (400 MHz, CDCl₃): δ 8.30 (s, 1H), 7.33-7.45 (m, 5H), 6.56 (s, 1H), 5.62 (q, 1H), 4.95 (s, 2H), 2.28 (s, 3H), 1.59 (d, 3H).

b) 2-(1-(5-Acetylisoindolin-2-yl)ethyl)-5-(benzyloxy)-4H-pyran-4-one

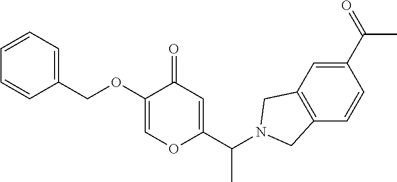 [SMILES: CC(=O)c1ccc2c(c1)CN(C(C)c1cc(=O)c(OCc3ccccc3)co1)C2]

To a solution of 1-(isoindolin-5-yl)ethanone hydrochloride (0.158 g, 0.802 mmol) and DIPEA (0.50 ml, 2.87 mmol) in dry acetonitrile (4.0 ml) was added 1-(5-(benzyloxy)-4-oxo-4H-pyran-2-yl)ethyl methanesulfonate (0.26 g, 0.802 mmol). The mixture was stirred at 90° C. until the reaction was completed. Cooled mixture was diluted with water and extracted with EtOAc (2×). Combined organic phases were washed with water and brine, dried and evaporated. Crude product was purified with flash chromatography affording the title compound (0.085 g). ¹H NMR (400 MHz, CDCl₃): δ 7.83 (dd, 1H), 7.79 (br s, 1H), 7.58 (s, 1H), 7.30-7.44 (m, 5H), 7.27 (d, 1H), 6.46 (s, 1H), 5.08 (s, 2H), 3.93-4.08 (m, 4H), 3.63 (q, 1H), 2.59 (s, 3H), 1.50 (d, 3H).

c) 2-(1-(5-Acetylisoindolin-2-yl)ethyl)-5-hydroxy-4H-pyran-4-one

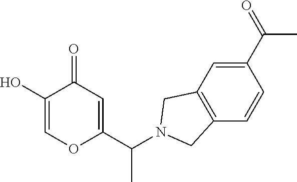 [SMILES: CC(=O)c1ccc2c(c1)CN(C(C)c1cc(=O)c(O)co1)C2]

A mixture of 2-(1-(5-acetylisoindolin-2-yl)ethyl)-5-(benzyloxy)-4H-pyran-4-one (0.085 g, 0.218 mmol) and hydrochloric acid (3 M, 1.0 ml, 3.00 mmol) was stirred at 100° C. until the reaction was completed. Cooled mixture was diluted with water and neutralised with aqueous 2 M NaOH. Mixture was extracted with EtOAc(2×), combined organic phases washed with brine, dried and evaporated to afford the title compound (0.065 g). ¹H NMR (400 MHz, CDCl₃): δ 7.88 (s, 1H), 7.84 (dd, 1H), 7.80 (br s, 1H), 7.28 (d, 1H), 6.53 (s, 1H), 3.97-4.10 (m, 4H), 3.69 (q, 1H), 2.59 (s, 3H), 1.54 (d, 3H).

d) 2-(1-(5-Acetylisoindolin-2-yl)ethyl)-5-((1-(methylsulfonyl)piperidin-4-yl)-methoxy)-4H-pyran-4-one (Compound 64)

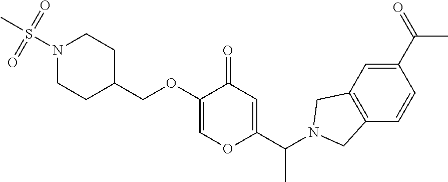 [SMILES: CC(=O)c1ccc2c(c1)CN(C(C)c1cc(=O)c(OCC3CCN(S(C)(=O)=O)CC3)co1)C2]

A mixture of 4-(bromomethyl)1-1(methylsulfonyl)piperidine (0.039, 0.154 mmol), 2-(1-(5-acetylisoindolin-2-yl)ethyl)-5-hydroxy-4H-pyran-4-one (0.040 g, 0.134 mmol) and potassium carbonate (0.041 g, 0.294 mmol) in dry DMSO (1.0 ml) was stirred at 80° C. until the reaction was completed. Cooled mixture was diluted with water and extracted with DCM (2×). Combined organic phases were washed with water and brine, dried and evaporated. Crude product was purified with reverse phase flash chropmatography to afford the title compound (0.015 g). ¹H NMR (400 MHz, DMSO-d₆): δ 8.16 (s, 1H), 7.80-7.85 (m, 2H), 7.38 (d, 1H), 6.40 (s, 1H), 4.00 (d, 2H), 3.89-3.96 (m, 2H), 3.69-3.77 (3H, m), 3.54-3.61 (m, 2H), 2.85 (s, 3H), 2.69-2.76 (m, 2H), 2.55 (s, 3H), 1.89-1.89 (m, 3H), 1.41 (d, 3H), 1.24-1.34 (m, 2H). MS: m/z 476.2 (M+H)⁺.

### Example 45

2-(3-(Methylsulfonyl)-4-((1-(methylsulfonyl)piperidin-4-yl)methoxy)benzyl)-isoindoline (Compound 65)

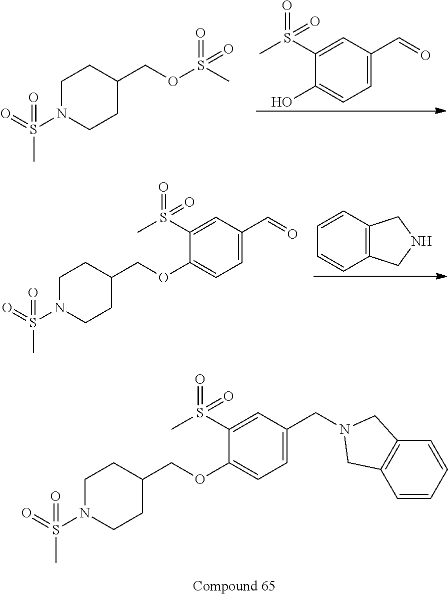

To a stirring solution of isoindoline (0.05 ml, 0.45 mmol) and acetic acid, glacial (0.05 ml, 0.91 mmol), in DCM (20 ml) was added 3-(methylsulfonyl)-4-((1-(methyl-sulfonyl)piperidin-4-yl)methoxy)benzaldehyde (0.17 g, 0.45 mmol). After 30 min, the reaction mixture was cooled to 0° C. followed by addition of sodium triacetoxy boro-hydride (0.14 g, 0.68 mmol). The mixture was stirred overnight at RT and quenched with water (10 ml). The product was extracted with EtOAc. The combined extracts were washed with water, dried with Na₂SO₄ filtered and evaporated. The crude product was purified by column chromatography to afford the titled compound. LC-MS: m/z 479.4 (M+H)⁺. ¹H NMR (Chloroform-d) δ: 7.95 (d, 1H), 7.90 (br d, 1H), 7.19-7.26 (m, 4H), 7.07 (d, 1H), 4.13 (s, 4H), 4.03-4.07 (m, 4H), 3.88-3.94 (m, 2H), 3.21 (s, 3H), 2.81 (s, 3H), 2.70-2.80 (m, 2H), 2.03 (br m, 2H), 1.50-1.62 (m, 3H).

The following compounds were prepared according to the procedure described for Compound 65 of Example 45. The compound number, the characterization data, starting materials and possible deviations in reaction conditions (solvent, reaction temperature, reaction time, purification method), if any, are indicated on the table.

Purification methods used:
A=Crystallization
B=Column chromatography
C=Precipitation in aqueous media
D=Semipreparative HPLC
E=Trituration
F=Salt formation

| | | Deviating reaction conditions / |
|---|---|---|
| No | Structure and starting material | ¹H NMR (400 MHz) / LC-MS |
| 66 | 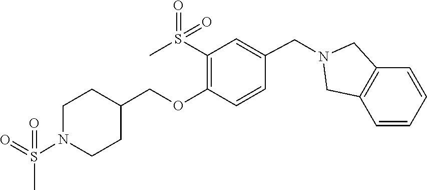 [SMILES: CS(=O)(=O)c1cc(CN2Cc3ccccc3C2)ccc1OCC1CCN(S(C)(=O)=O)CC1] | Conditions: DMF, 90° C., B. ¹H NMR (Chloroform-d) δ: 7.95 (d, 1H), 7.90 (br d, 1H), 7.19-7.26 (m, 4H), 7.07 (d, 1H), 4.13 (s, 4H), 4.03- 4.07 (m, 4H), 3.88-3.94 (m, 2H), 3.21 (s, 3H), 2.81 (s, 3H), 2.70-2.80 (m, 2H), 2.03 (br m, 2H), 1.50-1.62 (m, 3H). LCMS: m/z 479.4 (M + H)⁺. |
| | Starting material: 3-(Methylsulfonyl)- 4-((1-(methylsulfonyl)piperidin-4- yl)methoxy)benzaldehyde and Isoindoline | |
| 67 | 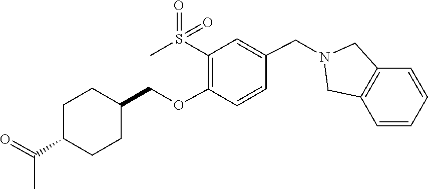 [SMILES: CC(=O)[C@H]1CC[C@H](COc2ccc(CN3Cc4ccccc4C3)cc2S(C)(=O)=O)CC1] | Conditions: DMF, 90° C., B. ¹H NMR (Chloroform-d) δ: 7.96 (d, 1H), 7.69 (m, 1H), 7.17-7.23 (m, 4H), 6.99-7.03 (m, 1H), 3.98 (d, 2H), 3.97 (s, 4H), 3.94 (s, 2H), 3.47 (d, 1H), 3.23 (s, 3H), 2.30-2.42 (m, 1H), 2.17 (s, 3H), 1.82-2.10 (m, 4H), 1.15-1.51 (m, 4H). LC-MS: m/z 442.7 (M + H)⁺. |
| | Starting material: 4-(((lr,4r)-4-Acetyl- cyclohexyl)methoxy)-3-(methyl- sulfonyl)benzaldehyde and Isoindole | |
| 68 | 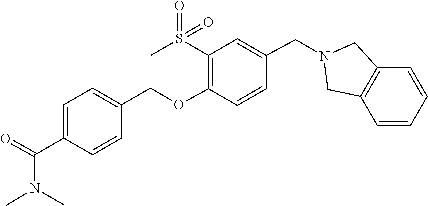 [SMILES: CN(C)C(=O)c1ccc(COc2ccc(CN3Cc4ccccc4C3)cc2S(C)(=O)=O)cc1] | Conditions: DMF, 90° C., B. ¹H NMR (Chloroform-d) δ: 8.00 (d, 1H), 7.68 (m, 1H), 7.45-7.58 (m, 4H), 7.17-7.20 (m, 4H), 7.08 (d, 1H), 5.28 (s, 2H), 3.96 (s, 4H), 3.93 (s, 2H), 3.22 (s, 3H), 3.12 (br s, 3H), 2.99 (br s, 3H). LC-MS: m/z 465.9 (M + H)⁺. |
| | Starting material: 4-((4-Formyl-2- (methylsulfonyl)phenoxy)methyl)- N,N-dimethylbenzamide and Isoindole | |
| 69 | 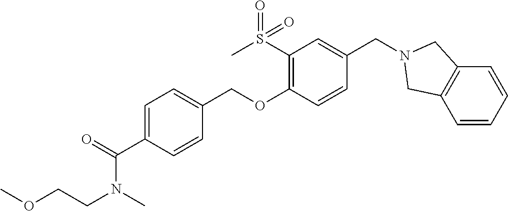 [SMILES: COCCN(C)C(=O)c1ccc(COc2ccc(CN3Cc4ccccc4C3)cc2S(C)(=O)=O)cc1] | Conditions: DMF, 90° C., B. ¹H NMR (Chloroform-d) δ: 8.01 (d, 1H), 7.66 (m, 1H), 7.56 (br d, 2H), 7.46-7.50 (m, 2H), 7.18 (s, 3H), 7.17- 7.20 (m, 1H), 7.07 (d, 1H), 5.28 (s, 2H), 3.92 (s, 4H), 3.90 (s, 2H), 3.66- 3.77 (m, 2H), 3.38-3.50 (m, 3H), 3.22 (s, 3H), 3.03-3.16 (m, 3H), 1.55 (s, 6H). LCMS: m/z 509.9 (M + H)⁺. |
| | Starting material: 4-((4-Formyl-2- (methylsulfonyl)phenoxy)methyl)-N- (2-methoxyethyl)-N-methylbenzamide and Isoindole | |
| 70 | 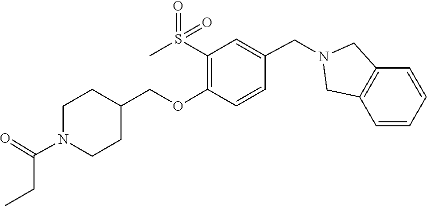 [SMILES: CCC(=O)N1CCC(COc2ccc(CN3Cc4ccccc4C3)cc2S(C)(=O)=O)CC1] | Conditions: DMSO, 80° C., B. ¹H NMR (Chloroform-d) δ: 7.97 (d, 1H), 7.71 (m, 1H), 7.16-7.23 (m, 4H), 7.02 (d, 1H), 4.74 (br d, 1H), 4.03- 4.08 (m, 1H), 3.99 (s, 4H), 3.95 (s, 2H), 3.21 (s, 3H), 3.08 (brm, 1H), 2.56-2.67 (m, 3H), 2.37 (m, 2H), 2.11- 2.23 (m, 1H), 2.05 (br d, 1H), 1.88 (br d, 1H), 1.26-1.43 (m, 2H), 1.16 (m, 3H). LC-MS: m/z 457.7 (M + H)⁺. |
| | Starting material: 3-(Methylsulfonyl)- 4-((1-propionylpiperidin-4-yl)- methoxy)benzaldehyde and Isoindole | |

The following compounds were prepared according to the procedure described Example 17(e) starting from 4-(Isoindolin-2-yl-methyl)-2-(methylsulfonyl)phenol or a derivative thereof and another appropriate starting material. The compound number, the characterization data, starting materials and possible deviations in reaction conditions (solvent, reaction temperature, reaction time, purification method), if any, are indicated on the table.

Purification methods used:
A=Crystallization
B=Column chromatography
C=Precipitation in aqueous media
D=Semipreparative HPLC
E=Trituration
F=Salt formation

| | | Deviating reaction conditions / |
|---|---|---|
| No | Structure and starting material | ¹H NMR (400 MHz) / LC-MS |
| 71 | 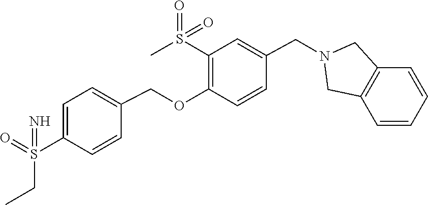 [SMILES: CCS(=N)(=O)c1ccc(COc2ccc(CN3Cc4ccccc4C3)cc2S(C)(=O)=O)cc1] | Conditions: DMF, 90° C., B. ¹H NMR (Chloroform-d) δ: 8.00-8.05 (m, 3H), 7.73 (d, 2H), 7.69 (m, 1H), 7.19 (s, 4H), 7.06 (d, 1H), 5.35 (s, 2H), 3.93 (s, 4H), 3.92 (d, 2H), 3.18- 3.26 (m, 5H), 1.28 (m, 3H). LC-MS: m/z 485.3 (M + H)⁺. |
| | Starting material: l-(Chloromethyl)-4- (ethylsulfonimidoyl)benzene | |
| 72 | 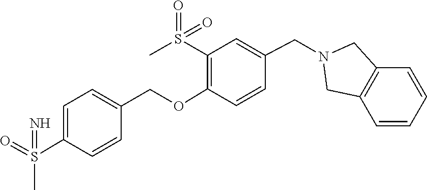 [SMILES: CS(=N)(=O)c1ccc(COc2ccc(CN3Cc4ccccc4C3)cc2S(C)(=O)=O)cc1] | Conditions: DMF, 90° C., B. ¹H NMR (Chloroform-d) δ: 8.05-8.10 (m, 2H), 8.03 (d, 1H), 7.72-7.77 (m, 2H), 7.69 (m, 1H), 7.17-7.21 (m, 4H), 7.07 (d, 1H), 5.34 (s, 2H), 3.93 (s, 4H), 3.91 (s, 2H), 3.23 (s, 3H), 3.13 (d, 3H), 2.70-2.72 (m, 1H). LCMS: m/z 471.4 (M + H)⁺. |
| | Starting material: l-(Chloromethyl)-4- (S-methylsulfonimidoyl)benzene | |
| 73 | 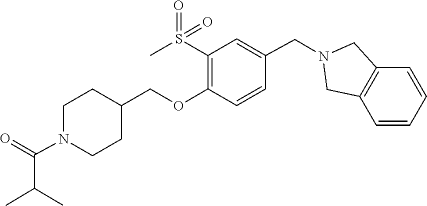 [SMILES: CC(C)C(=O)N1CCC(COc2ccc(CN3Cc4ccccc4C3)cc2S(C)(=O)=O)CC1] | Conditions: DMSO, 80° C., B. ¹H NMR (Chloroform-d) δ: 7.61 (s, 1H), 7.22 (d, 4H), 6.52 (s, 1H), 4.68 (br d, 1H), 4.53 (s, 1H), 4.08 (s, 4H), 3.81 (s, 2H), 3.65-3.78 (m, 2H), 3.00- 3.10 (m, 1H), 2.54-2.65 (m, 1H), 2.61 (m, 1H), 2.30-2.35 (m, 2H), 2.06-2.14 (m, 1H), 2.01 (br d, 1H), 1.84 (br d, 1H), 1.61-1.70 (m, 2H), 1.50 (m, 6H). LC-MS: m/z 471.3 (M + H)⁺. |
| | Starting material: (1-Isobutyrylpiperi- din-4-yl)methyl-4-methylbenzene- sulfonate | |
| 74 | 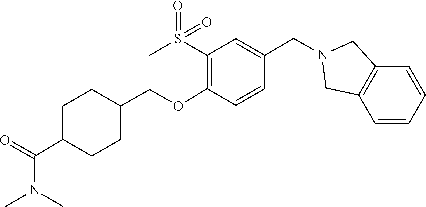 [SMILES: CN(C)C(=O)C1CCC(COc2ccc(CN3Cc4ccccc4C3)cc2S(C)(=O)=O)CC1] | Conditions: DMSO, 80° C., B. ¹H NMR (Chloroform-d) δ: 7.60 (s, 1H), 7.18-7.23 (m, 4H), 6.49 (s, 1H), 4.54-4.61 (m, 1H), 4.35 (br d, 1H), 4.04 (s, 4H), 3.77 (s, 2H), 3.73 (m, 2H), 3.03-3.15 (m, 1H), 2.72 (brm, 1H), 2.11-2.23 (m, 1H), 1.97 (brm, 2H), 1.83 (m, 3H), 1.22-1.40 (m, 2H). LC-MS: m/z 471.7 (M + H)⁺. |
| | Starting material: (4-(Dimethylcarba- moyl)cyclohexyl)methyl 4-methyl- benzenesulfonate | |
| 75 | 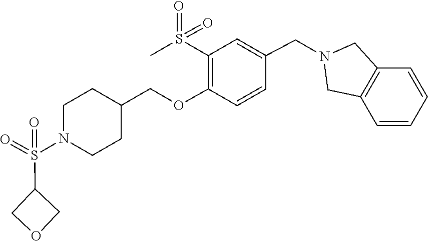 [SMILES: CS(=O)(=O)c1cc(CN2Cc3ccccc3C2)ccc1OCC1CCN(S(=O)(=O)C2COC2)CC1] | Conditions: DMSO, 80° C., B. ¹H NMR (Chloroform-d) δ: 7.97 (d, 1H), 7.68 (br d, 1H), 7.17-7.26 (m, 4H), 6.98-7.03 (m, 1H), 4.92-4.98 (m, 2H), 4.88 (m, 2H), 4.42 (m, 1H), 4.02 (d, 2H), 3.93 (s, 4H), 3.91 (s, 2H), 3.20 (s, 3H), 2.81-2.90 (m, 2H), 2.07 (s, 1H), 2.01 (br d, 2H), 1.42-1.67 (m, 4H). LC-MS: m/z 521.7 (M + H)⁺. |
| | Starting material: 4-(Bromomethyl)-l- (oxetan-3-ylsulfonyl) piperidine | |
| 76 | 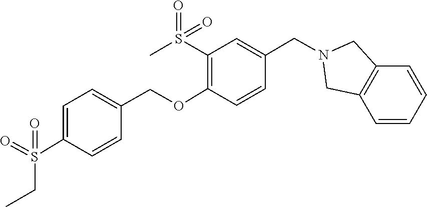 [SMILES: CCS(=O)(=O)c1ccc(COc2ccc(CN3Cc4ccccc4C3)cc2S(C)(=O)=O)cc1] | Conditions: DMSO, 80° C., B. ¹H NMR (Chloroform-d) δ: 8.03 (d, 1H), 7.97 (d, 2H), 7.76 (d, 2H), 7.71 (m, 1H), 7.17-7.21 (m, 4H), 7.07 (d, 1H), 5.35 (s, 2H), 3.96 (s, 4H), 3.94 (s, 2H), 3.23 (s, 3H), 3.14 (m, 2H), 1.30 (m, 3H). LC-MS: m/z 486.32 (M + H)⁺. |
| | Starting material: l-(Bromomethyl)-4- (ethylsulfonyl)benzene | |
| 77 | 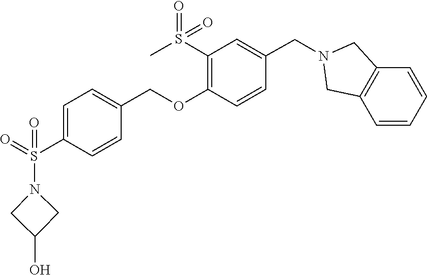 [SMILES: CS(=O)(=O)c1cc(CN2Cc3ccccc3C2)ccc1OCc1ccc(S(=O)(=O)N2CC(O)C2)cc1] | Conditions: DMSO, 80° C., B. ¹H NMR (Chloroform-d) δ: 7.97 (d, 1H), 7.86 (d, 2H), 7.75 (d, 2H), 7.62 (m, 1H), 7.18-7.22 (m, 4H), 6.99 (d, 1H), 5.42-5.44 (m, 1H), 5.43 (s, 1H), 4.19 (m, 1H), 3.94 (br d, 2H), 3.92 (s, 4H), 3.91 (br s, 2H), 3.39 (m, 2H), 3.26 (s, 3H). LC-MS: m/z 529.8 (M + H)⁺. |
| | Starting material: l-((4-(Bromo- methyl)phenyl)sulfonyl)azetidin-3-ol | |
| 78 | 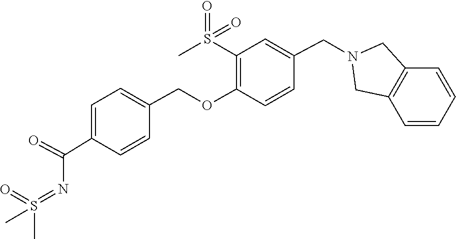 [SMILES: CS(=O)(=O)c1cc(CN2Cc3ccccc3C2)ccc1OCc1ccc(C(=O)N=S(C)(C)=O)cc1] | Conditions: DMSO, 80° C., B. ¹H NMR (Chloroform-d) δ: 8.14-8.18 (m, 2H), 8.00 (d, 1H), 7.65 (m, 1H), 7.56 (d, 2H), 7.16-7.20 (m, 4H), 7.06 (d, 1H), 5.30 (s, 2H), 3.92 (s, 4H), 3.89 (s, 2H), 3.39 (s, 6H), 3.21 (s, 3H). LC-MS: m/z 513.3 (M + H)⁺. |
| | Starting material: 4-Chloromethyl-N- dimethyl(oxo)-A6-sulfaneylidene)- benzamide | |
| 79 | 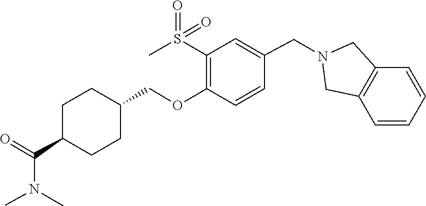 [SMILES: CN(C)C(=O)[C@H]1CC[C@H](COc2ccc(CN3Cc4ccccc4C3)cc2S(C)(=O)=O)CC1] | Conditions: DMSO, 80° C., B. ¹H NMR (DMSO-d₆, 600 MHz) δ 7.81 (br s, 1H), 7.65 (br s, 1H), 7.27 (br s, 1H), 7.1-7.2 (m, 4H), 3.99 (br s, 2H), 3.87 (br s, 2H), 3.83 (br s, 3H), 3.36 (br s, 2H), 3.27 (br s, 3H), 3.02 (br s, 3H), 2.81 (br s, 3H), 2.5-2.6 (m, 3H), 1.93 (br s, 2H), 1.83 (br s, 1H), 1.74 (br s, 2H), 1.41 (br s, 2H), 1.20 (br s, 2H). LC-MS: m/z 471.6 (M + H)⁺. |
| | Starting material: ((1r,4r)-4- (Dimethyl- carbamoyl)cyclohexyl)methyl 4- methylbenzenesulfonate | |
| 80 | 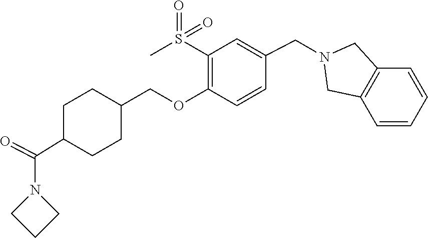 [SMILES: CS(=O)(=O)c1cc(CN2Cc3ccccc3C2)ccc1OCC1CCC(C(=O)N2CCC2)CC1] | Conditions: DMSO, 80° C., B. ¹H NMR (DMSO-d₆, 600 MHz) δ 7.81 (br s, 1H), 7.65 (br s, 1H), 7.27 (br s, 1H), 7.1-7.2 (m, 4H), 3.99 (br s, 2H), 3.87 (br s, 2H), 3.84 (m, 4H), 3.83 (br s, 3H), 3.36 (br s, 2H), 3.27 (br s, 3H), 2.5-2.6 (m, 3H), 2.23 (m, 2 H), 1.93 (br s, 2H), 1.83 (br s, 1H), 1.74 (br s, 2H), 1.41 (br s, 2H), 1.20 (br s, 2H). LC-MS: m/z 483.7 (M + H)⁺. |
| | Starting material: (4-(Azetidine-l- carbonyl)cyclohexyl)methyl 4-methyl- benzenesulfonate | |
| 81 | 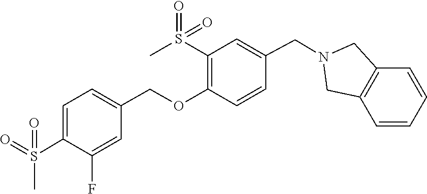 [SMILES: CS(=O)(=O)c1ccc(COc2ccc(CN3Cc4ccccc4C3)cc2S(C)(=O)=O)cc1F] | ¹H NMR (Chloroform-d) δ: 7.99-8.05 (m, 2H), 7.71 (m, 1H), 7.54 (d, 1H), 7.48-7.52 (m, 1H), 7.18-7.21 (m, 4H), 7.04 (d, 1H), 5.33 (s, 2H), 3.96 (s, 4H), 3.94 (s, 2H), 3.25 (s, 3H), 3.23 (s, 3H). LC-MS: m/z 490.7 (M + H)⁺. |
| | Starting material: 4-(Bromomethyl)-2- fluoro-1-(methylsulfonyl)benzene | |
| 82 | 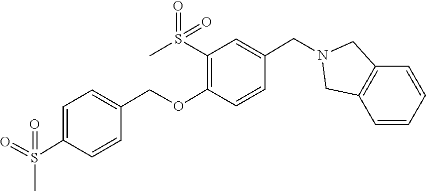 [SMILES: CS(=O)(=O)c1ccc(COc2ccc(CN3Cc4ccccc4C3)cc2S(C)(=O)=O)cc1] | Conditions: DMSO, 80° C., B. ¹H NMR (Chloroform-d) δ: 8.02 (d, 1H), 7.99 (d, 2H), 7.77 (d, 2H), 7.68 (m, 1H), 7.16-7.20 (m, 4H), 7.07 (d, 1H), 5.35 (s, 2H), 3.92 (s, 4H), 3.91 (s, 2H), 3.22 (s, 3H), 3.07 (s, 3H). LC-MS: m/z 472.1.0 (M + H)⁺. |
| | Starting material: 4-Methylsulfonyl- benzyl bromide | |
| 83 | 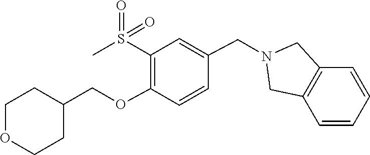 [SMILES: CS(=O)(=O)c1cc(CN2Cc3ccccc3C2)ccc1OCC1CCOCC1] | Conditions: DMSO, 80° C., B. ¹H NMR (Chloroform-d) δ: 7.96 (br s, 1H), 7.72 (br s, 1H), 7.21 (br s, 4H), 7.04 (br d, 1H), 3.90-4.12 (m, 10H), 3.47 (brm, 2H), 3.23 (s, 3H), 2.19 (br s, 1H), 1.83 (br d, 2H), 1.45-1.59 (m, 2H). LC-MS: m/z 402.1 (M + H)⁺. |
| | Starting material: 4-(Bromomethyl)- tetrahydro-2H-pyran | |
| 84 | 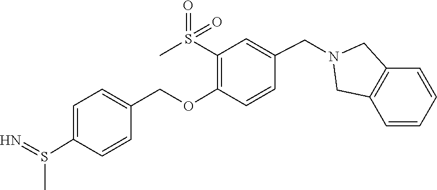 [SMILES: CS(=N)c1ccc(COc2ccc(CN3Cc4ccccc4C3)cc2S(C)(=O)=O)cc1] | Conditions: DMSO, 80° C., B. ¹H NMR (Chloroform-d) δ: 8.25 (s, 1H), 8.01 (s, 1H), 7.82 (br d, 1H), 7.71 (s, 4H), 7.21-7.32 (m, 4H), 7.07-7.21 (m, 1H), 5.34 (s, 2H), 4.23 (s, 4H), 4.16 (s, 2H), 3.22 (s, 3H), 2.76 (s, 3H). LC-MS: m/z 455.1 (M + H)⁺. |
| | Starting material: l-(Chloromethyl)-4- (S-methylsulfinimidoyl)benzene | |
| 85 | 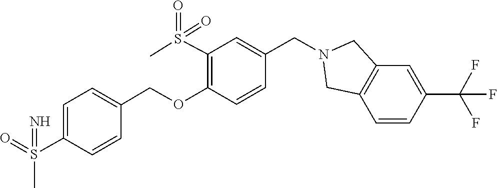 [SMILES: CS(=N)(=O)c1ccc(COc2ccc(CN3Cc4ccc(C(F)(F)F)cc4C3)cc2S(C)(=O)=O)cc1] | Conditions: DMSO, 80° C., B. ¹H NMR (Chloroform-d) δ: 8.05-8.11 (m, 2H), 8.03 (d, 1H), 7.74 (d, 2H), 7.67 (m, 1H), 7.47 (d, 1H), 7.43 (s, 1H), 7.27-7.31 (m, 1H), 7.07 (d, 1H), 5.35 (s, 2H), 3.96 (s, 4H), 3.92 (s, 2H), 3.23 (s, 3H), 3.13 (s, 3H), 2.71 (br s, 1H). LC-MS: m/z 539.6 (M + H)⁺. |
| | Starting material: l-(Chloromethyl)-4- (S-methylsulfonimidoyl)benzene | |
| 86 | 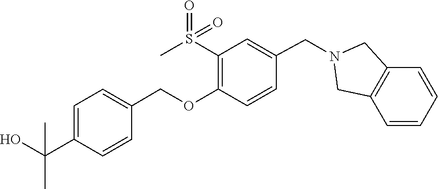 [SMILES: CC(C)(O)c1ccc(COc2ccc(CN3Cc4ccccc4C3)cc2S(C)(=O)=O)cc1] | Conditions: DMSO, 80° C., B. ¹H NMR (Chloroform-d) δ: 8.00 (d, 1H), 7.66 (m, 1H), 7.47-7.56 (m, 4H), 7.16-7.21 (m, 4H), 7.09 (d, 1H), 5.25 (s, 2H), 3.92 (s, 4H), 3.90 (s, 2H), 3.22 (s, 3H), 1.60 (s, 6H). LC-MS: m/z 452.4 (M + H)⁺. |
| | Starting material: 2-(4-(bromomethyl)- phenyl)propan-2-ol | |
| 87 | 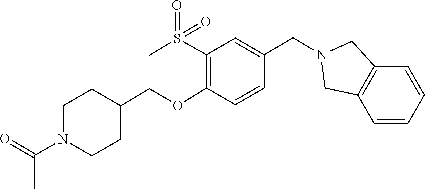 [SMILES: CC(=O)N1CCC(COc2ccc(CN3Cc4ccccc4C3)cc2S(C)(=O)=O)CC1] | Conditions: DMSO, 80° C., B. ¹H NMR (Chloroform-d) δ: 7.97 (d, 1H), 7.67 (m, 1H), 7.16-7.26 (m, 4H), 7.01 (d, 1H), 4.68-4.75 (m, 1H), 4.02- 4.09 (m, 1H), 3.95 (m, 1H), 3.92 (s, 4H), 3.90 (s, 2H), 3.88 (br s, 1H), 3.21 (s, 3H), 3.07-3.16 (m, 1H), 2.62 (m, 1H), 2.13-2.25 (m, 1H), 2.11 (s, 3H), 2.06 (br d, 1H), 1.88 (br d, 1H), 1.26- 1.44 (m, 2H). LC-MS: m/z 443.5 (M + H)⁺. |
| | Starting material: l-(4- (Chloromethyl)piperidin-1- yl)ethanone | |

### Example 46

2-((1′H-Spiro[cyclopropane-1,4′-isoquinolin]-2′(3′H)-yl)methyl)-5-((1-tosyl-piperidin-4-yl)methoxy)-4H-pyran-4-one (Compound 88)

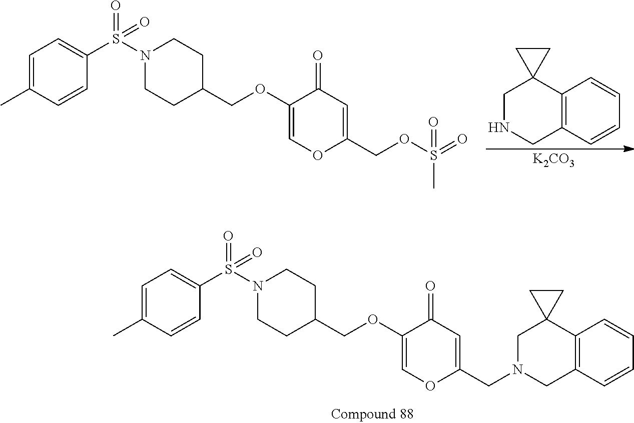

To a solution of (4-oxo-5-((1-tosylpiperidin-4-yl)methoxy)-4H-pyran-2-yl)methyl methanesulfonate (0.10 g, 0.21 mmol) in 2 ml of DMF was added K₂CO₃ (0.088 g, 0.634 mmol) and 2′,3′-dihydro-1′H-spiro[cyclopropane-1,4′-isoquinoline] hydrochloride (0.042 g, 0.21 mmol). The resulting mixture was stirred at 80° C. for 2 h. After completion of the reaction, the reaction mixture was quenched with water and extracted with EtOAc. The combined organic layer was washed with brine, dried over anhydrous sodium sulphate and concentrated under reduced pressure to give the crude product. This crude product was purified by reverse phase chromatography to afford the title compound. LC-MS: m/z 535.4 (M+H)⁺. ¹H NMR (400 MHz, Chloroform-d) δ: 0.78-0.94 (m, 2H), 0.97-1.09 (m, 2H), 1.21 (t, 1H) 1.40 (qd, 2H) 1.72-1.86 (m, 1H), 1.91 (br d, 2H) 2.26 (td, 2H), 2.44 (s, 3H), 2.70 (s, 2H), 3.42-3.55 (m, 1H), 3.59 (s, 2H), 3.67 (d, 2H), 3.78-3.92 (m, 4H), 6.47 (s, 1H), 6.67 (d, 1H), 6.98 (d, 1H), 7.08 (td, 1H), 7.12-7.17 (m, 1H), 7.27-7.32 (m, 1H), 7.34 (s, 1H), 7.55-7.68 (m, 3H).

The following compound was prepared according to the procedure described

Example 1. The compound number, the characterization data, starting materials and possible deviations in reaction conditions (solvent, reaction temperature, reaction time, purification method), if any, are indicated on the table.

| | | Deviating reaction conditions / |
|---|---|---|
| No | Structure and starting material | ¹H NMR (400 MHz) / LC-MS |
| 89 | 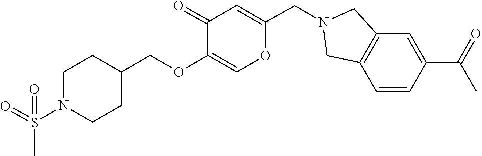 [SMILES: CC(=O)c1ccc2c(c1)CN(Cc1cc(=O)c(OCC3CCN(S(C)(=O)=O)CC3)co1)C2] | Conditions: DMF, 80° C., B. ¹H NMR (400 MHz, Chloroform-d) δ: 0.00 (s, 1H), 1.43 (br dd, 2H), 2.00 (br dd, 3H), 2.60 (s, 3H), 2.69 (td, 2H), 2.79 (s, 3H), 3.69-3.90 (m, 6H), 4.08 (s, 4H), 6.49 (s, 1H), 7.29 (d, 1H), 7.61 (s, 1H), 7.81 (s, 1H), 7.84 (dd, 1H). LC-MS: m/z 460.5 (M + H)+. |
| | Starting materials: 1-(Isoindolin-5-yl)- ethanone HC1 and 2-(Chloromethyl)- 5-((1-(methylsulfonyl)piperidin-4- yl)methoxy)-4H-pyran-4-one | |

### Example 47

(E)-2-((5-(1-(Hydroxyimino)ethyl)isoindolin-2-yl)methyl)-5-((1-(methylsulfonyl)piperidin-4-yl)methoxy)-4H-pyran-4-one (Compound 90)

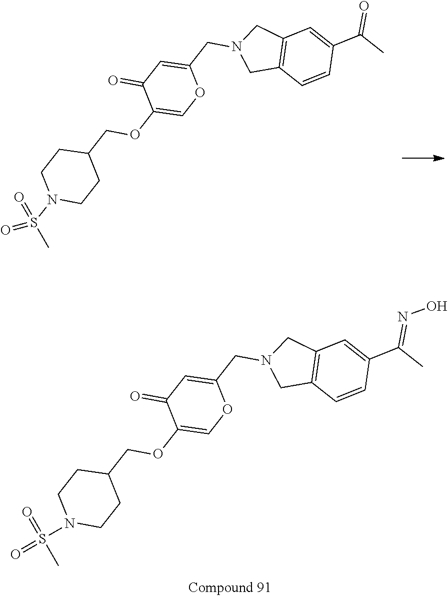 [SMILES: CC(=O)c1ccc2c(c1)CN(Cc1cc(=O)c(OCC3CCN(S(C)(=O)=O)CC3)co1)C2 ; C/C(=N\O)c1ccc2c(c1)CN(Cc1cc(=O)c(OCC3CCN(S(C)(=O)=O)CC3)co1)C2]

To a solution of (E)-2-((5-(1-(hydroxyimino)ethyl)isoindolin-2-yl)methyl)-5-((1-(methylsulfonyl)piperidin-4-yl)methoxy)-4H-pyran-4-one (0.2 g, 0.43 mmol) in EtOH (10 mL) were added sodium acetate (0.160 g, 1.9 mmol) and hydroxylamine hydro-chloride (0.075 g, 1.08 mmol) at 25° C. The mixture was stirred at 70° C. for 2 h. After completion of the reaction, the reaction mixture was quenched with water and extracted with EtOAc. The combined organic layer was washed with brine, dried over anhydrous sodium sulphate and concentrated under reduced pressure. The resulting crude product was purified by column chromatography to give 53.2 mg of the title compound. LC-MS: m/z 476.3 (M+H)⁺. ¹H NMR (600 MHz, DMSO-d₆) δ: 11.12 (s, 1H), 8.14 (s, 1H), 7.52 (s, 1H), 7.49 (d, 1H), 7.24 (d, 1H), 6.40 (s, 1H), 3.96 (br d, 4H), 3.79 (s, 2H), 3.72 (d, 2H), 3.58 (br d, 3H), 2.80-2.87 (m, 3H), 2.72 (br t, 2H), 2.14 (s, 3H), 2.09 (d, 1H), 1.84 (br d, 3H), 1.29 (br dd, 2H).

### Example 48

(E)-2-((5-(1-(Methoxyimino)ethyl)isoindolin-2-yl)methyl)-5-((1-(methylsulfonyl)piperidin-4-yl)methoxy)-4H-pyran-4-one (Compound 91)

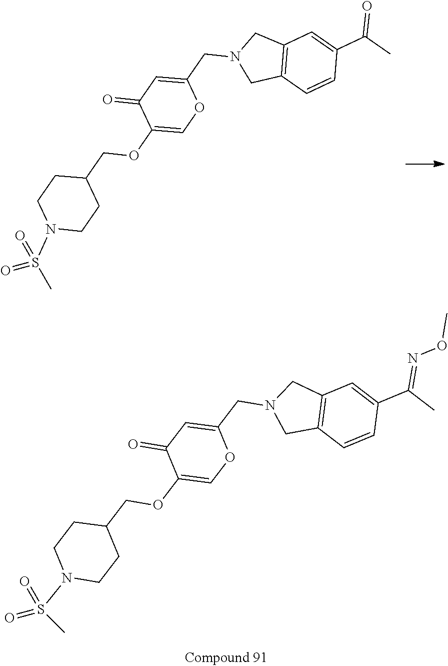 [SMILES: CC(=O)c1ccc2c(c1)CN(Cc1cc(=O)c(OCC3CCN(S(C)(=O)=O)CC3)co1)C2 ; CO/N=C(\C)c1ccc2c(c1)CN(Cc1cc(=O)c(OCC3CCN(S(C)(=O)=O)CC3)co1)C2]

To a solution of (E)-2-((5-(1-(hydroxyimino)ethyl)isoindolin-2-yl)methyl)-5-((1-(methylsulfonyl)piperidin-4-yl)methoxy)-4H-pyran-4-one (0.2 g, 0.43 mmol) in EtOH (10 mL) were added sodium acetate (0.160 g, 1.9 mmol) and methoxyamine HCl (0.091 g, 1.08 mmol) at 25° C. The mixture was stirred at 70° C. for 2 h. After completion of the reaction, the reaction mixture was quenched with water and extracted with EtOAc. The combined organic layer was washed with brine, dried over anhydrous sodium sulphate and concentrated under reduced pressure. The resulting crude product was purified by column chromatography to give 22.5 mg of the title compound. LC-MS: m/z 490.6 (M+H)⁺. ¹H NMR (600 MHz, DMSO-d₆) δ: 8.14 (s, 2H), 7.48-7.55 (m, 4H), 7.26 (d, 3H), 6.39 (s, 2H), 3.96 (br d, 9H), 3.90 (s, 6H), 3.79 (s, 5H), 3.69-3.75 (m, 6H), 3.58 (br d, 5H), 2.85 (s, 7H), 2.72 (br t, 5H), 2.16 (s, 6H), 2.11 (s, 1H), 1.84 (br d, 7H), 1.24-1.34 (m, 5H).

### Example 49

1-(4-((4-(Isoindolin-2-ylmethyl)-2-(methylsulfonyl)phenoxy)methyl)phenyl)-ethanol (Compound 92b)

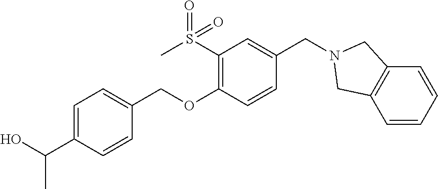 [SMILES: CC(O)c1ccc(COc2ccc(CN3Cc4ccccc4C3)cc2S(C)(=O)=O)cc1]

a) 4-(Chloromethyl)-2-(methylsulfonyl)phenol

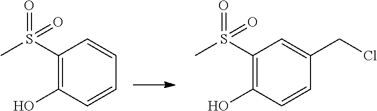

To a solution of paraformaldehyde (1.25 g, 41.8 mmol) and concentrated HCl (14.1 ml, 465 mmol) was added 2-(methylsulfonyl)benzenol (4.0 g, 23.2 mmol). The reaction mixture was stirred at RT for 48 h. The product was filtered, washed with 0.5% Na₂CO₃ (5 ml) and water (20 ml) and dried to give 3.5 g of the title compound. LC-MS: m/z 221.6 (M+H)⁺.

b) 4-(Isoindolin-2-ylmethyl)-2-(methylsulfonyl)phenol

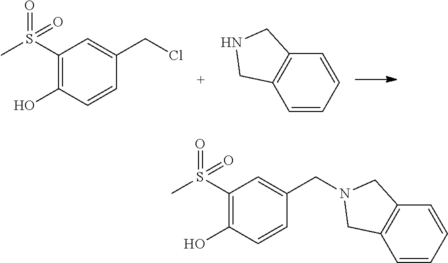 [SMILES: CS(=O)(=O)c1cc(CCl)ccc1O ; c1ccc2c(c1)CNC2 ; CS(=O)(=O)c1cc(CN2Cc3ccccc3C2)ccc1O]

To a solution of 4-(chloromethyl)-2-(methylsulfonyl)phenol (2.5 g, 11.33 mmol) in THF (15 ml) was added isoindoline hydrochloride (2.11 g, 13.59 mmol) and DIPEA (7.9 ml g, 45.30 mmol). The mixture was stirred at RT for 4 h. The mixture was evaporated, quenched with water (20 ml) and extracted with EtOAc. The organic layer was washed with water, dried with anhydrous Na₂SO₄, filtered and concentrated under reduced pressure. Column chromatography (heptane/ethyl acetate) afforded 2.0 g of the titled compound. LC-MS: m/z 304.3 (M+H)⁺.

c) 1-(4-((4-(Isoindolin-2-ylmethyl)-2-(methylsulfonyl)phenoxy)methyl)phenyl)-ethanone (Compound 92a)

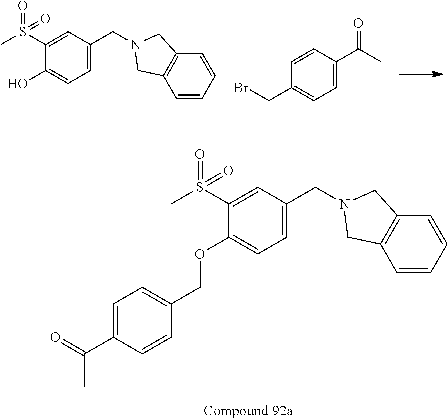

To a solution of 4-(isoindolin-2-ylmethyl)-2-(methylsulfonyl)phenol (0.30 g, 0.99 mmol) in DMF (5 ml) were added 1-[4-(Bromomethyl)phenyl]ethanone (0.21 g, 0.99 mmol) and K₂CO₃ (0.30 g, 2.16 mmol). The reaction mixture was heated at 100° C. for 1 h. The mixture was cooled to RT, water (10 ml) was added and the product was extracted with EtOAc. The combined extracts were washed with water, dried with Na₂SO₄ filtered and evaporated. The crude product was purified by column chromatography to afford 0.05 g of the title compound. LC-MS: m/z 436.53 (M+H)⁺. ¹H NMR (Chloroform-d, 400 MHz) δ 8.0-8.0 (m, 3H), 7.6-7.7 (m, 3H), 7.2-7.2 (m, 4H), 7.06 (d, 1H, J=8.6 Hz), 5.32 (s, 2H), 3.9-3.9 (m, 6H), 3.2-3.2 (m, 3H), 2.6-2.6 (m, 3H)

d) 1-(4-((4-(Isoindolin-2-ylmethyl)-2-(methylsulfonyl)phenoxy)methyl)phenyl)-ethanol (Compound 92b)

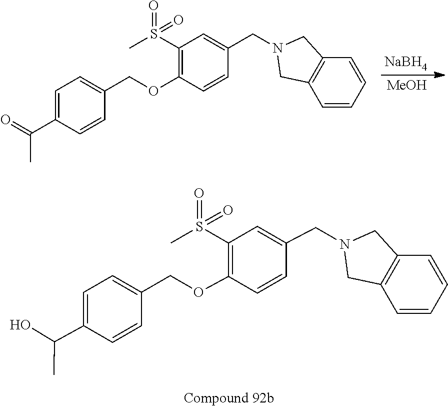

To a solution of 1-(4-((4-(isoindolin-2-ylmethyl)-2-(methylsulfonyl)phenoxy)-methyl)phenyl)ethanone (0.05 g, 0.11 mmol) in MeOH (5 mL) was added at 0° C. NaBH₄ (5.32 mg, 0.14 mmol). The mixture was stirred at 0° C. for 10 min and at RT for 2 h, and then quenched with ice cold water and extracted with DCM. The combined organic layer was washed with water, dried over anhydrous sodium sulphate, filtered and concentrated under reduced pressure. The obtained crude product was purified by column chromatography to afford 5.5 mg of the title product. LC-MS: m/z 439.7 (M+H)⁺. ¹H NMR (Chloroform-d) δ: 8.00 (d, 1H), 7.66 (m, 1H), 7.48-7.53 (m, 2H), 7.40-7.46 (m, 2H), 7.18 (s, 4H), 7.09 (d, 1H), 5.25 (s, 2H), 4.94 (m, 1H), 3.93 (s, 4H), 3.90 (s, 2H), 3.22 (s, 3H), 1.52 (d, 3H).

The following compounds were prepared according to the procedure described in Example 49. The compound number, the characterization data and starting material is indicated on the table.

| No | Structure and starting material | ¹H NMR / LC-MS |
|---|---|---|
| 93 | 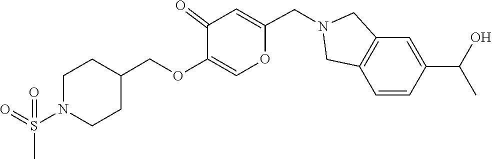 [SMILES: CC(O)c1ccc2c(c1)CN(Cc1cc(=O)c(OCC3CCN(S(C)(=O)=O)CC3)co1)C2] | ¹H NMR (400 MHz, Chloroform-d) δ: -0.0007 (s, 1H) 1.4802 (d, 4H), 1.9306-2.1623 (m, 10H), 2.6924 (td, 2H), 2.7899 (s, 3H), 3.7039-3.8083 (m, 4H), 3.8083-3.8930 (m, 2H), 4.0351 (s, 4H) 4.8970 (d, 1H), 6.4958 (s, 1H), 7.1532-7.2595 (m, 3H), 7.6073 (s, 1H). LC-MS: m/z 462.2 (M + H)⁺. |
| | Starting material: 2-((5-Acetylisoindo- lin-2-yl)methyl)-5-((1-(methylsulfo- nyl)piperidin-4-yl)methoxy)-4H- pyran-4-one | |
| 94 | 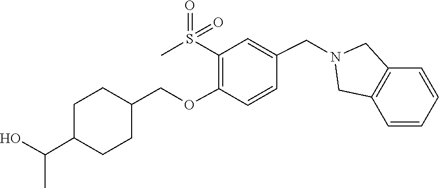 [SMILES: CC(O)C1CCC(COc2ccc(CN3Cc4ccccc4C3)cc2S(C)(=O)=O)CC1] | ¹H NMR (Chloroform-d) δ: 7.96 (d, 1H), 7.70 (br d, 1H), 7.16-7.22 (m, 4H), 7.02 (d, 1H), 3.95-3.98 (m, 6H), 3.93 (s, 2H), 3.46 (d, 1H), 3.24 (s, 3H), 1.72-1.48 (m, 13H). LC-MS: m/z 444.4 (M + H)⁺. |
| | Starting material: l-((lr,4r)-4-((4-(Iso- indolin-2-ylmethyl)-2- (methylsulfonyl)- phenoxy)methyl)cyclohexyl)ethanone | |

The following compounds were made using the preparative HPLC method as defined below. The compound number, the characterization data and starting material is indicated on the table.

Preparative HPLC method:

Instruments: Agilent technologies 1200. Column: Chiralpak IF No: p-41, 5 micron, 20×250 mm; Solvents: solvent A: n-hexane+0.2% DEA, solvent B EtOH+0.2% DEA; Detection wavelength 228 nm; Flow rate 15 mL/min.

| No | Structure and starting material | ¹H NMR/LC-MS |
|---|---|---|
| 95 | 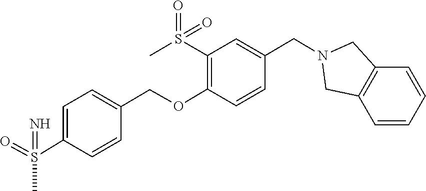 | ¹H NMR (Chloroform-d) δ: 8.05-8.09 (m, 2H), 8.03 (d, 1H), 7.74 (d, 2H), 7.69 (m, 1H), 7.17-7.21 (m, 4H), 7.07 (d, 1H), 5.34 (s, 2H), 3.93 (s, 4H), 3.91 (s, 2H), 3.23 (s, 3H), 3.13 (s, 3H), 2.71 (s, 1H). LC-MS: m/z 471.4 (M + H)⁺. |
| | Starting material: 2-(4-((4-(Methyl- sulfonimidoyl)benzyl)oxy)-3-(methyl- sulfonyl)benzyl)isoindoline | |
| 96 | 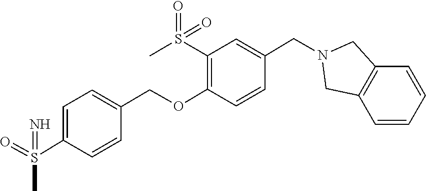 [SMILES: CS(=O)(=O)c1cc(CN2Cc3ccccc3C2)ccc1OCc1ccc([S@@](C)(=N)=O)cc1] | ¹HNMR (Chloroform-d) δ: 8.05-8.10 (m, 2H), 8.03 (d, 1H), 7.74 (d, 2H), 7.69 (m, 1H), 7.66-7.71 (m, 1H), 7.16- 7.21 (m, 4H), 7.07 (d, 1H), 5.34 (s, 2H), 3.93 (s, 4H), 3.91 (s, 2H), 3.23 (s, 3H), 3.13 (s, 3H),2.71 (brs, 1H) LC-MS: m/z 471.4 (M + H)⁺. |
| | Starting material: 2-(4-((4-(Methyl- sulfonimidoyl)benzyl)oxy)-3-(methyl- sulfonyl)benzyl)isoindoline | |
| 97 | 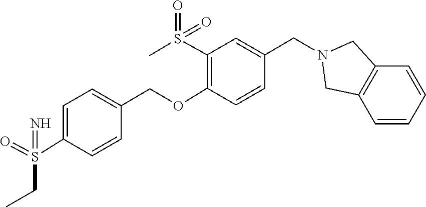 [SMILES: CC[S@](=N)(=O)c1ccc(COc2ccc(CN3Cc4ccccc4C3)cc2S(C)(=O)=O)cc1] | ¹HNMR (Chloroform-d) δ: 8.01-8.05 (m, 3H), 7.73 (d, 3H), 7.20 (m, 4H), 7.08 (d, 1H), 5.35 (s, 2H), 4.00 (s, 4H), 3.97 (s, 2H), 3.23 (s, 3H), 3.17- 3.22 (m, 2H), 1.28 (m, 3H). LC-MS: m/z 485.9 (M + H)⁺. |
| | Starting material: 2-(4-((4-(Ethyl- sulfonimidoyl)benzyl)oxy)-3-(methyl- sulfonyl)benzyl)isoindoline | |
| 98 | 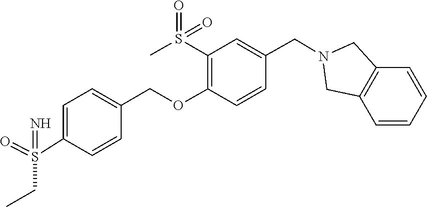 | ¹H NMR (Chloroform-d) δ: 8.01-8.05 (m, 3H), 7.73 (d, 3H), 7.20 (m, 4H), 7.08 (d, 1H), 5.35 (s, 2H), 4.00 (s, 4H), 3.97 (s, 2H), 3.23 (s, 3H), 3.17- 3.22 (m, 2H), 1.28 (m, 3H). LC-MS: m/z 485.9 (M + H)⁺. |
| | Starting material: 2-(4-((4-(Ethyl- sulfonimidoyl)benzyl)oxy)-3-(methyl- sulfonyl)benzyl)isoindoline | |
| 99 | 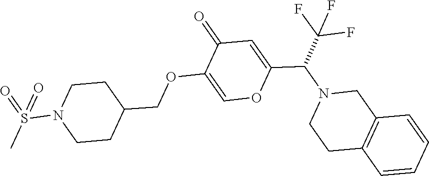 [SMILES: CS(=O)(=O)N1CCC(COc2coc([C@@H](N3CCc4ccccc4C3)C(F)(F)F)cc2=O)CC1] | ¹H NMR (400 MHz, Chloroform-d) δ: 1.42 (br dd, 2H), 1.93-2.07 (m, 3H), 2.65-2.91 (m, 8H), 3.24-3.34 (m, 1H), 3.73 (d, 2H), 3.79-3.91 (m, 4H), 4.25- 4.33 (m, 1H), 6.53 (s, 1H), 7.17-7.24 (m, 2H), 7.26-7.29 (m, 1H), 7.30 (t, 1H), 7.56 (s, 1H). LC-MS: m/z 501.5 (M + H)⁺. |
| | ORM-0042698 Starting material: 2-(l-(3,4-Dihydro- isoquinolin-2(1H)-yl)-2,2,2-trifluoro- ethyl)-5-((l- (methylsulfonyl)piperidin-4- yl)methoxy)-4H-pyran-4-one | |
| 100 | 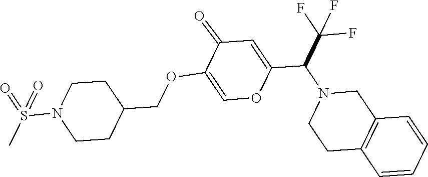 [SMILES: CS(=O)(=O)N1CCC(COc2coc([C@H](N3CCc4ccccc4C3)C(F)(F)F)cc2=O)CC1] | ¹H NMR (400 MHz, Chloroform-d) δ: 0.00 (s, 1H), 1.35-1.48 (m, 2H), 1.93- 2.07 (m, 3H), 2.65-2.91 (m, 8H), 3.25- 3.33 (m, 1H), 3.73 (d, 2H), 3.79-3.91 (m, 4H), 4.25-4.33 (m, 1H), 6.53 (s, 1H) 7.17-7.24 (m, 2H), 7.26-7.29 (m, 1H), 7.30 (t, 1H), 7.57 (s, 1H). LC-MS: m/z 501.5 (M + H)⁺. |
| | ORM-0042699 Starting material: 2-(l-(3,4-Dihydro- isoquinolin-2(1H)-yl)-2,2,2-trifluoro- ethyl)-5-((l- (methylsulfonyl)piperidin-4- yl)methoxy)-4H-pyran-4-one | |

### Example 50

2-((6-(Isoxazol-4-yl)-3,4-dihydroisoquinolin-2(1H)-yl)methyl)-5-((1-(methyl-sulfonyl)piperidin-4-yl)methoxy)-4H-pyran-4-one (Compound 101)

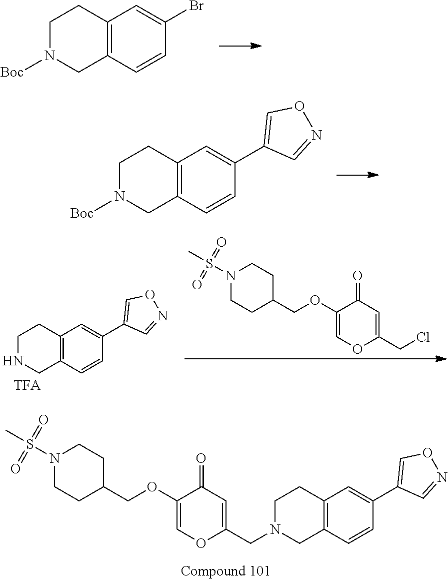

a) tert-Butyl 6-(isoxazol-4-yl)-3,4-dihydroisoquinoline-2(1H)-carboxylate

A sealed reaction vessel was charged with 4-isoxazoleboronic acid pinacol ester (141 mg, 0.721 mmol), tert-butyl 6-bromo-3,4-dihydroisoquinoline-2(1H)-carboxylate (150 mg, 0.480 mmol), bis(triphenylphosphine)palladium(II) dichloride (17 mg, 0.024 mmol), 2 M K₂CO₃ solution in water (0.72 mL, 1.441 mmol) and acetonitrile (2 mL) followed by carefully purging with nitrogen. Vessel was sealed and heated at 80° C. for 5 hours. The reaction was allowed to cool to RT and stirred overnight. Reaction mixture was diluted with EtOAc (5 mL) and water (5 mL). Phases were separated and aqueous phase was extracted with EtOAc (5 mL). Combined organic extracts were washed with brine (5 mL) and the evaporated onto celite. Product was purified with reverse phase chromatography to give 10 mg (7%) of the title compound as beige solid. MS (ESI) m/z [M−tBu+1]⁺: 245.2.

b) 4-(1,2,3,4-Tetrahydroisoquinolin-6-yl)isoxazole, trifluoroacetate

A round bottom flask was charged with tert-butyl 6-(isoxazol-4-yl)-3,4-dihydro-isoquinoline-2(1H)-carboxylate (8 mg, 0.027 mmol), dichloromethane (1 mL) and trifluoroacetic acid (0.5 mL). The mixture was stirred at RT for 3 hours and evaporated to dryness to afford the crude title product which was used as such in a following synthesis step. MS (ESI) m/z [M+1]⁺: 201.2.

c) 2-((6-(Isoxazol-4-yl)-3,4-dihydroisoquinolin-2(1H)-yl)methyl)-5-((1-(methyl-sulfonyl)piperidin-4-yl)methoxy)-4H-pyran-4-one (Compound 101)

Treatment of 4-(1,2,3,4-tetrahydroisoquinolin-6-yl)isoxazole, trifluoroacetate (8 mg, 0.026 mmol) with 2-(chloromethyl)-5-((1-(methylsulfonyl)piperidin-4-yl)methoxy)-4H-pyran-4-one (11 mg, 0.032 mmol) in the presence of DIPEA (0.012 mL, 0.066 mmol) in DMSO (1 mL) at RT for 19 h followed by reverse phase column chromatography afforded 5.7 mg (43%) of the title compound as a white solid. ¹H NMR (400 MHz, CDCl₃) δ: 8.65 (s, 1H), 8.53 (s, 1H), 7.61 (s, 1H), 7.26-7.21 (m, 2H), 7.05 (d, 1H), 6.51(m, 1H), 3.90-3.81 (m, 2H), 3.78-3.69 (m, 4H), 3.57 (s, 2H), 2.97 (t, 2H), 2.86 (t, 2H), 2.79 (s, 3H), 2.69 (dt, 2H), 2.10-1.96 (m, 3H), 1.49-1.36 (m, 2H). MS (ESI) m/z [M+1]⁺: 500.4.

### Example 51

2-((6-(1H-Pyrazol-1-yl)-3,4-dihydroisoquinolin-2(1H)-yl)methyl)-5-((1-(methyl-sulfonyl)piperidin-4-yl)methoxy)-4H-pyran-4-one (Compound 102)

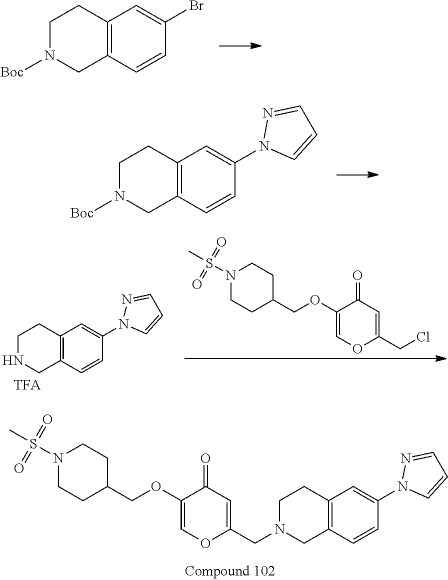

a) tert-Butyl 6-(1H-pyrazol-1-yl)-3,4-dihydroisoquinoline-2(1H)-carboxylate

A sealed reaction vessel was charged with tert-butyl 6-bromo-3,4-dihydroiso-quinoline-2(1H)-carboxylate (150 mg, 0.48 mmol), pyrazole (49 mg, 0.72 mmol), Cs₂CO₃ (313 mg, 0.96 mmol), picolinic acid (12 mg, 0.096 mmol), dimethyl sulfoxide (2 mL) and finally copper(I) iodide (18 mg, 0.096 mmol). The reaction vessel was carefully purged with nitrogen and then heated at 120° C. for total of 12 hours. Mixture was allowed to cool to RT and stirred overnight. Reaction mixture was diluted with EtOAc (10 mL) and water (10 mL), and then filtered trough celite. Phases were separated and aqueous phase was extracted with EtOAc (10 mL). Combined organic extracts were evaporated onto celite. Product was purified with reverse phase chromatography to give 20 mg (14%) of the title product as a semisolid. MS (ESI) m/z [M+1]⁺: 300.3.

b) 6-(1H-Pyrazol-1-yl)-1,2,3,4-tetrahydroisoquinoline, trifluoroacetate

A round bottom flask was charged with tert-butyl 6-(1H-pyrazol-1-yl)-3,4-di-hydroisoquinoline-2(1H)-carboxylate (20 mg, 0.067 mmol), dichloromethane (1 mL) and trifluoroacetic acid (0.5 mL). The mixture was stirred at RT for 1 hour followed by evaporating to dryness to afford crude title product which was used as such in a following synthesis step. MS (ESI) m/z [M+1]⁺: 200.2.

c) 2-((6-(1H-pyrazol-1-yl)-3,4-dihydroisoquinolin-2(1H)-yl)methyl)-5-((1-(methylsulfonyl)piperidin-4-yl)methoxy)-4H-pyran-4-one (Compound 102)

Treatment of 6-(1H-pyrazol-1-yl)-1,2,3,4-tetrahydroisoquinoline, trifluoroacetate (21 mg, 0.067 mmol) with 2-(chloromethyl)-5-((1-(methylsulfonyl)piperidin-4-yl)methoxy)-4H-pyran-4-one (27 mg, 0.080 mmol) in the presence of DIPEA (0.035 mL, 0.20 mmol) in DMSO (1 mL) at 60° C. for 5 h followed by reverse phase column chromatography afforded 11 mg (33%) of the title compound as off-white solid. ¹H NMR (400 MHz, CDCl₃) δ: 7.89 (dd, 1H), 7.71 (d, 1H), 7.61 (s, 1H), 7.49 (d, 1H), 7.43 (dd, 1H), 7.08 (d, 1H), 6.51 (s, 1H), 6.46 (dd, 1H), 3.90-3.81 (m, 2H), 3.77-3.69 (m, 4H), 3.58 (s, 2H), 3.00 (t, 2H), 2.86 (t, 2H), 2.79 (s, 3H), 2.69 (dt, 2H), 2.08-1.95 (m, 3H), 1.50-1.35 (m, 2H). MS (ESI) m/z [M+1]⁺: 499.3.

### Example 52

2-((1′H-Spiro[cyclopropane-1,4′-isoquinolin]-2′(3′H)-yl)methyl)-5-((1-(methyl-sulfonyl)piperidin-4-yl)methoxy)-4H-pyran-4-one (Compound 103)

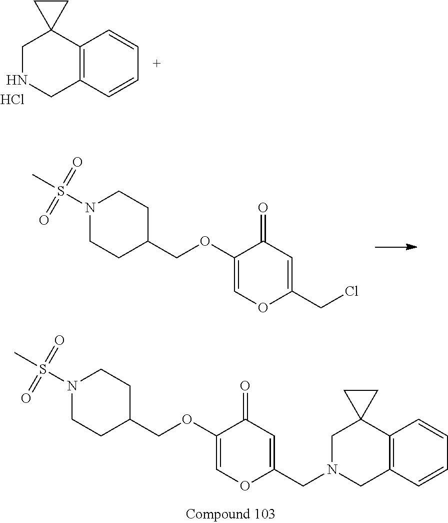 [SMILES: c1ccc2c(c1)CNCC21CC1 ; Cl ; CS(=O)(=O)N1CCC(COc2coc(CCl)cc2=O)CC1 ; CS(=O)(=O)N1CCC(COc2coc(CN3Cc4ccccc4C4(CC4)C3)cc2=O)CC1]

Treatment of 2′,3′-dihydro-1′H-spiro[cyclopropane-1,4′-isoquinoline] hydro-chloride (47 mg, 0.24 mmol) with 2-(chloromethyl)-5-((1-(methylsulfonyl)piperidin-4-yl)methoxy)-4H-pyran-4-one (80 mg, 0.24 mmol) in the presence of DIPEA (0.10 mL, 0.60 mmol) in DMSO (1 mL) at RT for 19 h followed by reverse phase column chromatography afforded 71 mg (65%) of the title compound as off-white solid. ¹H NMR (400 MHz, CDCl₃) δ: 7.60 (s, 1H), 7.14 (dt, 1H), 7.08 (dt, 1H), 7.02-6.96 (m, 1H), 6.68 (dd, 1H), 6.48 (s, 1H), 3.90-3.81 (m, 4H), 3.74 (d, 2H), 3.57 (s, 2H), 2.79 (s, 3H), 2.74-2.64 (m, 4H), 2.07-1.95 (m, 3H), 1.76 (s, 2H), 1.50-1.35 (m, 2H), 1.07-1.00 (m, 2H), 0.90-0.82 (m, 2H). MS (ESI) m/z [M+1]⁺: 459.5.

### Example 53

2-((1′H-Spiro[cyclopentane-1,4′-isoquinolin]-2′(3′H)-yl)methyl)-5-((4-(2-hydroxypropan-2-yl)benzyl)oxy)-4H-pyran-4-one (Compound 104)

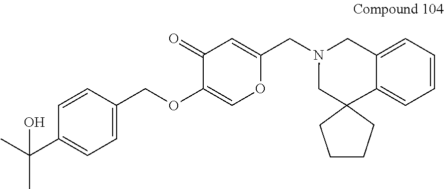 [SMILES: CC(C)(O)c1ccc(COc2coc(CN3Cc4ccccc4C4(CCCC4)C3)cc2=O)cc1]

a) 2-((1′H-Spiro[cyclopentane-1,4′-isoquinoline]-2′(3′H)-yl)methyl)-5-hydroxy-4H-pyran-4-one

To a solution of 2′,3′-dihydro-1′H-spiro[cyclopentane-1,4′-isoquinoline] (0.233 g, 1.246 mmol) and 2-(chloromethyl)-5-hydroxy-4H-pyran-4-one (0.2 g, 1.246 mmol) into a 2-neck flask under nitrogen in DMSO (5 ml) at 0° C. was added DIPEA (0.434 ml, 2.491 mmol) dropwise. The mixture was stirred at 60° C. for 1 h and poured on ice. The aqueos layer was extracted with EtOAc. The organic layers were combined, washed with water and brine, dried with Na₂SO₄, filtered, and evaporated to afford the titled compound. 1H NMR (400 MHz, DMSO-d6) δ: 1.58-1.86 (m, 8H), 2.43-2.48 (m, 2H), 3.53-3.59 (m, 2H), 3.61-3.65 (m, 2H), 6.43 (s, 1H), 6.96-7.01 (m, 1H), 7.04-7.10 (m, 1H), 7.14-7.20 (m, 1H), 7.26-7.31 (m, 1H), 7.96-8.16 (m, 1H), 8.73-9.34 (m, 1H).

b) 2-((1′H-Spiro[cyclopentane-1,4′-isoquinolin]-2′(3′H)-yl)methyl)-5-((4-(2-hydroxypropan-2-yl)benzyl)oxy)-4H-pyran-4-one (Compound 104)

To a solution of [2-((1′H-spiro[cyclopentane-1,4′-isoquinoline]-2′(3′H)-yl)methyl)-5-hydroxy-4H-pyran-4-one (0.22 g, 0.707 mmol) and potassium carbonate (0.293 g, 2.120 mmol) in THF (5 ml) was added 2-(4-(bromomethyl)phenyl)propan-2-ol (0.170 g, 0.742 mmol) in THF (5 ml). The mixture was heated at reflux for 15 min followed by cooling to RT, filtering and evaporating in vacuo. The crude product was purified by reversed phase column chromatography to afford the titled compound. 1H NMR (400 MHz, Chloroform-d) δ: 8.06 (s, 1H), 7.58 (s, 1H), 7.49 (m, 2H), 7.37 (m, 2H), 7.27 (m, 1H), 7.19 (m, 1H), 7.09 (m, 1H), 6.95 (m, 1H), 6.57 (m, 1H), 5.05 (s, 2H), 3.68 (s, 2H), 3.53 (s, 2H), 2.49 (s, 2H), 1.84 (m, 6H), 1.68 (m, 2H), 1.58 (m, 6H). LC-MS: m/z 460.2 (M+H)+.

### Example 54

4-Fluoro-2-(3-(methylsulfonyl)-4-((1-(methylsulfonyl)piperidin-4-yl)methoxy)-benzyl)isoindoline (Compound 105)

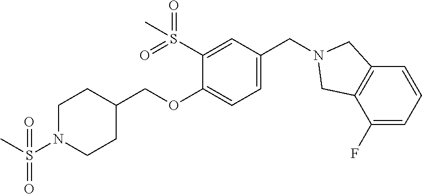 [SMILES: CS(=O)(=O)c1cc(CN2Cc3cccc(F)c3C2)ccc1OCC1CCN(S(C)(=O)=O)CC1]

a) 4-((4-Fluoroisoindolin-2-yl)methyl)-2-(methylsulfonyl)phenol

A mixture of 4-fluoroisoindoline hydrochloride (0.393 g, 1.813 mmol), 4-(chloromethyl)-2-(methylsulfonyl)phenol (0.400 g, 1.813 mmol) and potassium carbonate (0.526 g, 3.81 mmol) in a 2-neck flask under nitrogen in DMSO (3 ml) was stirred at 60° C. for 2 h followed by pouring on ice. The aqueos layer was extracted with EtOAc. The organic layers were combined, washed with water and brine, dried with Na₂SO₄, filtered, and evaporated to afford the title product. LC-MS: m/z 322.3. (M+H)+.

b) 4-Fluoro-2-(3-(methylsulfonyl)-4-((1-(methylsulfonyl)piperidin-4-yl)methoxy)benzyl)isoindoline (Compound 105)

A mixture of (1-(methylsulfonyl)piperidin-4-yl)methyl methanesulfonate (0.150 g, 0.552 mmol), 4-((4-fluoroisoindolin-2-yl)methyl)-2-(methylsulfonyl)phenol (0.178 g, 0.553 mmol) and potassium carbonate (0.115 g, 0.286 mmol) in a 2-neck flask under nitrogen in DMF (2 ml) was stirred at 80° C. for 2.5 h, and poured on ice. The precipitate was filtered, washed with water, dried, and crystallized from 2-propanol to afford the title compound. 1H NMR (400 MHz, DMSO-d6) δ: 7.80-7.84 (m, 1H), 7.63-7.71 (m, 1H), 7.2-7.31 (m, 2H), 7.05-7.11 (m, 1H), 7.00-7.05 (m, 1H), 4.05-4.14 (m, 2H), 3.85-3.96 (m, 6H), 3.57-3.66 (m, 2H), 3.26-3.29 (m, 3H), 2.87 (s, 3H), 2.73-2.81 (m, 2H), 1.89-2.08 (m, 3H), 1.37-1.48 (m, 2H). LC-MS: m/z 497.4. (M+H)+.

### Abbreviations

ACN—Acetonitrile
DCM—Dichloromethane
DEA—Diethanolamine
DIAD—Diisopropyl azodicarboxylate
DIPEA—N,N-diisopropylethylamine
DMA—Dimethylacetamide
DMEDA—N,N′-Dimethylethylenediamine
DMF—N,N-Dimethylformamide
DMS—Dimethyl sulfide
DMSO—Dimethylsulfoxide
EtOAc—Ethyl acetate
EtOH—Ethanol
HMPA—Hexamethylphosphoramide
HPLC—High-performance liquid chromatography
LC-MS—Liquid chromatography-mass spectrometry
LiHMDS—Hexamethyldisilazane lithium salt TAI Lithium bis(trimethylsilyl)amide
MeOH—Methanol
Ms—Methanesulfonyl
RT—Room temperature
rt—Retention time
TEA—Triethylamine
TFA—Trifluoroacetic acid
THF—Tetrahydrofuran
TLC—Thin layer chromatography
Ts—p-Toluenesulfonyl

Experiments

Experiment 1. CYP11A1 Inhibition

The ability of the test compounds to inhibit conversion of cholesterol to pregnenolone and isocaproic acid was measured by modification of isocaproic acid release assay (IARA) described by Ruangwises et al. (Biology of Reproduction 1991; 45(1):143-50) except that human H295R adrenocortical carcinoma cell line was used as source of enzyme and extraction was done with dextran-coated charcoal suspension (Isomaa, V. et al., Endocrinology 1982; 111(3):833-843). The H295R cell line has been shown to express all the key steroidogenic enzymes. To determine the half maximal inhibitory concentration (IC₅₀) of the test compounds on CYP11A1 inhibition, the cells were treated for three days with increasing concentrations of the test compounds in the presence of 3 nM [24,25-3H]-labelled cholesterol (American Radiolabelled Chemicals). The final DMSO concentration was 1%. Cell culture medium was extracted with dextran-coated charcoal suspension and the radiolabelled isocaproic acid was determined by mixing 100 μl of supernatant fraction in 200 μl of scintillation fluid (OptiPhase SuperMix, Perkin Elmer). Radioactivity was measured using a Microbeta scintillation counter (1450 MicroBeta Trilux, Wallac). All the test compounds were studied at 10 concentrations in duplicates.

The compounds of the invention were screened in the above mentioned assay and the IC₅₀ values of the compounds are set forth in Table 1 below wherein “A” refers to an IC₅₀ value of less than 100 nM, “B” refers to IC₅₀ value in range of 101 to 200 nM and “C” refers to IC₅₀ value in range of 201 nM to 2000 nM.

TABLE 1

| Group | Compound No. |
|---|---|
| A | 3, 5, 12, 13b, 14, 15, 16, 17, 27, 28, 65, 66, 67, 68, 69, |
| | 71, 72, 73, 74, 75, 76, 77, 78, 79, 82, 84, 85, 86, 89, |
| | 92b, 96, 97, 98 and 105. |
| B | 1, 2, 4, 6, 7, 8, 10, 13a, 21, 26, 32, 48, 58, 70, 80, 81, |
| | 90, 91, 95, and 104. |
| C | 20, 30, 35, 50, 51, 54, 62, 83, 87, 93, 94, and 103. |

Experiment 2. Formation of Reactive Metabolites

Formation of reactive metabolites was studied as described in Grillo, M., Expert Opin. Drug Metab. Toxicol. (2015), 11(8):1281-1302 by incubating test compounds with recombinant human cytochrome P450 (CYP) CYP3A4 enzyme in the presence of cofactor NADPH and trapping agents glutathione (GSH), potassium cyanide (KCN) and semicarbazide (SCA). Recombinant human CYP3A4 was chosen as the enzyme source because it showed higher metabolite activity towards test compounds than human liver microsomes.

Compounds were dissolved in DMSO and diluted in phosphate buffer (pH 7.4) to obtain the final incubation concentration 1 or 10 μM. Compounds were incubated with one trapping agent (mixture of unlabelled and ¹³C¹⁵N-labelled trapper 1:1) at a time with cofactor NADPH (actual samples) and without NADPH (control samples). Incubation time was 1 hour. Samples at timepoint 0 min (before incubation) and 60 min (after incubation) were analysed using ultra high-pressure liquid chromatography (UHPLC) coupled with high-resolution mass spectrometry (HRMS). Tentative identification of trapped metabolites was based on accurate masses of protonated molecules and product ion spectra of found metabolites and accurate mass differences between unlabelled and labelled trapping agents reacted with reactive metabolites.

The metabolites were classified as “major” if the peak area after incubation was over 10% of parent peak area before incubation. If peak area was between 1% and 10%, metabolites were classified as “minor” and if peak area was less than 1%, metabolite was classified as “trace”. The results are shown in Table 2. Reference compounds A and B represent compounds of prior art (Compound No. 139 and Compound No. 186 of WO 2018/115591, respectively). The results indicate that the compounds of the present invention have lower potential of forming reactive metabolites than reference compounds.

TABLE 2
Formation of reactive metabolites trapped by glutathione (GSH),
potassium cyanide (KCN) and semicarbazide (SCA). Test compound
concentration 10 μM if not indicated otherwise.

| | Number of | Number of | Number of |
|---|---|---|---|
| | SCA trapped | GSH trapped | CN trapped |
| Compound | metabolites | metabolites | metabolites |
| Compound 1 | 2 (trace) | 0 | 0 |
| (ORM-42556) | | | |
| Compound 2 | 3 (trace) | 0 | 0 |
| (ORM-42305) | | | |
| Compound 7 | 1 (trace) | 0 | 0 |
| (ORM-41869) | | | |
| Compound 8 | 1 (minor) + 1 | 1 (trace) | 0 |
| (ORM-41794) | (trace) | | |
| Compound 10 | 2 (trace) | 0 | 1 (trace) |
| (ORM-41754) | | | |
| Compound 17b | 1 (trace) | 0 | 0 |
| (ORM-43020) | | | |
| Compound 24b | 0 | 0 | 2 (trace) |
| (ORM-43758) | | | |
| Compound 30* | 0 | 0 | 0 |
| (ORM-41675) | | | |
| Compound 89 | 1 (trace) | 0 | 0 |
| (ORM-43000) | | | |
| Compound 90 | 1 (trace) | 0 | 0 |
| (ORM-43403) | | | |
| Compound 93 | 3 (trace) | 0 | 0 |
| (ORM-43204) | | | |
| Ref. compound A | 1 (minor) + 5 | 1 (minor) + 1 | 1 (minor) |
| (ORM-31713) | (trace) | (trace) | |
| Ref. compound B | 1 (minor) + 2 | 1 (trace) | 1 (trace) |
| (ORM-32701) | (trace) | | |

*Test compound concentration 1 μM

1. A compound of formula (I) or (II): 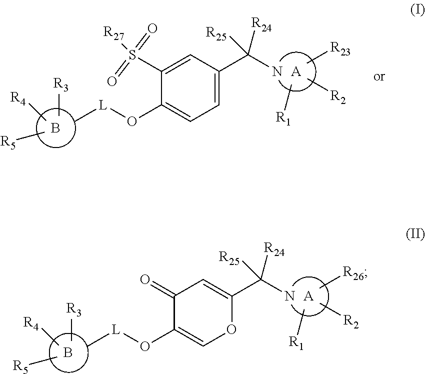
or a pharmaceutically acceptable salt thereof;
wherein:
ring B is a 4-12 membered monocyclic or bicyclic ring or spiro bicyclic ring containing 0-4 heteroatoms independently selected form N, O, or S;
ring A is 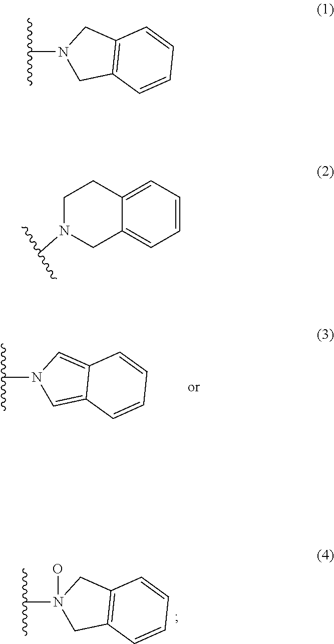
L is absent or is —CH₂—, —CH(CH₃)—, —CH₂—CH₂— or —CH₂—CH₂—CH₂—;
R₁ is hydrogen, C₁₋₇ alkyl, C₁₋₇ alkoxy, halogen, cyano, nitro, halo C₁₋₇ alkyl, or halo C₁₋₇ alkoxy;
R₂ is hydrogen, C₁₋₇ alkyl, halogen, hydroxy, C₁₋₇ alkoxy, halo C₁₋₇ alkyl, or oxo;
or R₁ and R₂ are attached to the same carbon atom and together form, with a carbon atom to which they are attached, a C₃₋₇ cycloalkyl ring;
or R₁ and R₂ together with the carbon atoms to which they are attached form a fused C₃₋₇ cycloalkyl ring;
R₃ is hydrogen, halogen, nitro, cyano, oxo, C₁₋₇ alkyl, C₂₋₇ alkenyl, C₃₋₇ cycloalkyl, hydroxy C₃₋₇ cycloalkyl, C₁₋₇ alkoxy, hydroxy C₁₋₇ alkyl, halo C₁₋₇ alkyl, cyano C₁₋₇ alkyl, C₁₋₇ alkoxy C₁₋₇ alkyl, C₁₋₇ alkylthio, aminocarbonyl C₂₋₇ alkenyl, halo C₁₋₇ alkylthio, C₁₋₇ alkoxycarbonyl C₁₋₇ alkyl, C₁₋₇ alkoxycarbonyl C₂₋₇ alkenyl, ═NSO₂R₂₀,—S(O)—C₁₋₇ alkyl, —S(O)(NR₁₄)(R₂₂), —S(NR₁₅)(C₁₋₇ alkyl), —C(S)NR₁₈R₁₉, -D-C(O)—NR₆R₇, —C(O)R₈, -D-NR₉R₁₀, —SO₂R₁₁, an optionally substituted 3-10 membered carbocyclyl, optionally substituted 3-10 membered carbocyclyl C₁₋₇ alkyl, optionally substituted 4-10 membered heterocyclyl, or optionally substituted 4-10 membered heterocyclyl C₁₋₇ alkyl;
R₄ is hydrogen, halogen, hydroxy, C₁₋₇ alkyl, halo C₁₋₇ alkyl, or oxo;
R₅ is hydrogen, halogen, or C₁₋₇ alkyl;
R₆ is hydrogen, C₁₋₇ alkyl, C₁₋₇ alkoxy C₁₋₇ alkyl, C₂₋₇ alkenyl, C₃₋₇ cycloalkyl, hydroxy C₁₋₇ alkyl, cyano C₁₋₇ alkyl, —C₁₋₇ alkyl-O—C(O)C₁₋₇ alkyl, or optionally substituted 4-10 membered heterocyclyl;
R₈ is hydrogen, C₁₋₇ alkyl, C₂₋₇ alkenyl, C₃₋₇ cycloalkyl, C₁₋₇ alkoxy, halo C₁₋₇ alkyl, C₁₋₇ alkoxy C₁₋₇ alkyl, C₁₋₇ alkylcarbonyl, C₁₋₇ alkoxycarbonyl, —C₁₋₇ alkyl-O—C(O)—C₁₋₇ alkyl, —C₁₋₇ alkyl-SO₂(C₁₋₇ alkyl), —N═S(O)(C₁₋₇ alkyl)(C₁₋₇ alkyl), or optionally substituted 4-10 membered heterocyclyl;
R₉ is hydrogen, C₁₋₇ alkyl, C₃₋₇ cycloalkyl, C₁₋₇ alkylcarbonyl, —SO₂(C₁₋₇ alkyl), or —SO₂(C₃₋₇ cycloalkyl);
R₁₁ is C₁₋₇ alkyl, C₂₋₇ alkenyl, C₃₋₇ cycloalkyl, halo C₁₋₇ alkyl, cyano C₁₋₇ alkyl, C₁₋₇ alkoxy C₁₋₇ alkyl, —NR₁₂R₁₃, optionally substituted 3-10 membered carbocyclyl, or optionally substituted 4-10 membered heterocyclyl;
R₁₂ is hydrogen, C₁₋₇ alkyl, hydroxy C₁₋₇ alkyl, cyano C₁₋₇ alkyl, C₁₋₇ alkoxy, C₁₋₇ alkoxy C₁₋₇ alkyl, or C₁₋₇ alkylcarbonyl;
R₇, R₁₀, R₁₃, R₁₈, and R₁₉ are, independently, hydrogen, C₁₋₇ alkyl, or C₃₋₇ cyclo-alkyl;
R₁₄ is hydrogen, C₁₋₇ alkyl, C₁₋₇ alkylcarbonyl, or —SO₂R₂₁;
R₁₅ is hydrogen, C₁₋₇ alkyl, C₃₋₇ cycloalkyl, C₁₋₇ alkylcarbonyl, or —SO₂R₁₇;
R₁₇ is C₁₋₇ alkyl or an optionally substituted 3-10 membered carbocyclyl;
R₂₀ and R₂₁ are, independently, C₁₋₇ alkyl, C₃₋₇ cycloalkyl, or optionally substituted 3-10 membered carbocyclyl;
R₂₂ is C₁₋₇ alkyl or C₃₋₇ cycloalkyl;
R₂₃ is hydrogen, C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms, or optionally substituted 4-10 membered heterocyclyl;
R₂₄ is hydrogen, C₁₋₇ alkyl, or halo C₁₋₇ alkyl;
R₂₅ is hydrogen or C₁₋₇ alkyl;
R₂₆ is C₁₋₇ alkylcarbonyl, hydroxyimino C₁₋₇ alkyl, C₁₋₇ alkoxyimino C₁₋₇ alkyl, hydroxy C₁₋₇ alkyl optionally substituted by 1-3 halogen atoms, or an optionally substituted 4-10 membered heterocyclyl, with the proviso than when ring B is a spiro bicyclic ring then R₂₆ can also be hydrogen;
R₂₇ is C₁₋₇ alkyl or C₃₋₇ cycloalkyl;
D is absent or is C₁₋₇ alkyl or C₂₋₇ alkenyl;
wherein the optional substitution in each occurrence is selected from 1-3 substituents independently selected from C₁₋₇ alkyl, halogen, hydroxy, cyano, C₁₋₇ alkylcarbonyl, C₁₋₇ alkoxy, C₁₋₇ alkoxy C₁₋₇ alkyl, C₁₋₇ alkoxycarbonyl, or oxo; and
wherein the heterocyclyl group in each occurrence has 1-4 heteroatoms independently selected from N, O, and S.

2. The compound according to claim 1, wherein the compound is represented by formula (I): 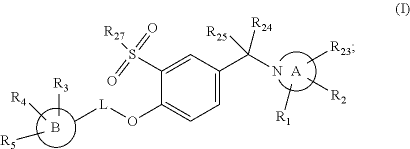
or a pharmaceutically acceptable salt thereof.

3. The compound according to claim 2, wherein the compound is represented by formula (IA): 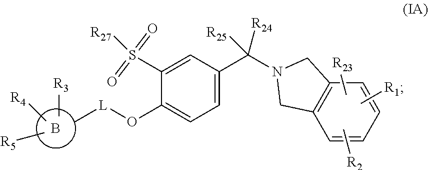
or a pharmaceutically acceptable salt thereof.

4. The compound according to claim 2, wherein the compound is represented by formula (IB): 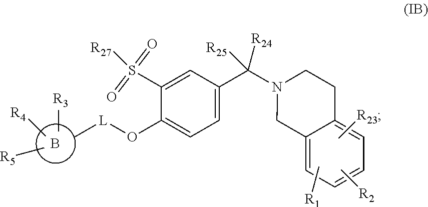
or a pharmaceutically acceptable salt thereof.

5. The compound according to claim 1, wherein the compound is represented by formula (II): 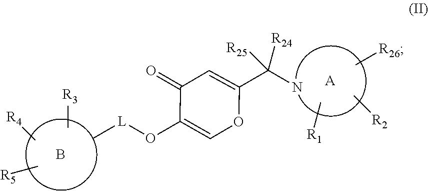
or a pharmaceutically acceptable salt thereof.

6. The compound according to claim 5, wherein the compound is represented by formula (IIA): 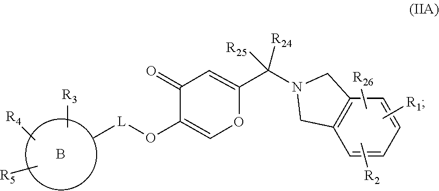
or a pharmaceutically acceptable salt thereof.

7. The compound according to claim 5, wherein the compound is represented by formula (IIB): 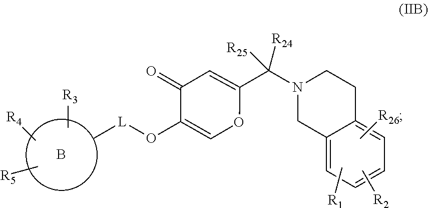
or a pharmaceutically acceptable salt thereof.

8. The compound according to claim 5, wherein the compound is represented by formula (IIC): 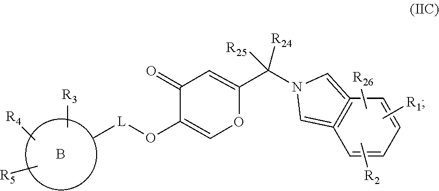
or a pharmaceutically acceptable salt thereof.

9. The compound according to claim 1, wherein L is absent or is —CH₂— or —CH(CH₃)—.

10-12. (canceled)

13. The compound according to claim 1, wherein ring B is 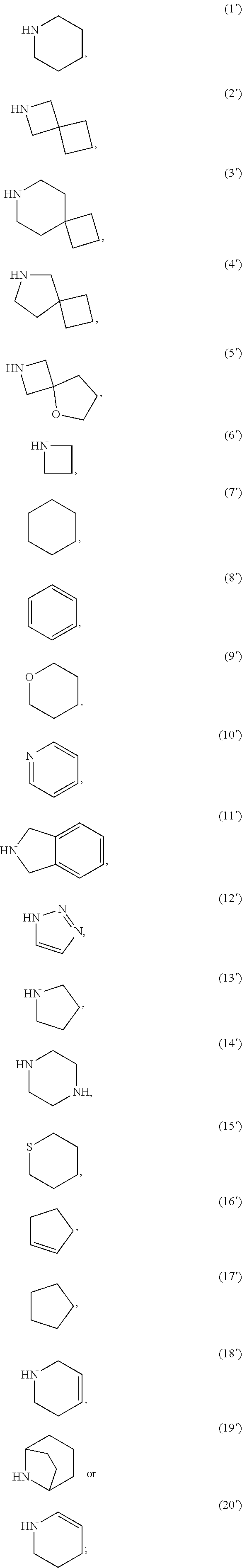
and
R₃, R₄ and R₅ being are attached to the above B rings.

14. The compound according to claim 13, wherein ring B is 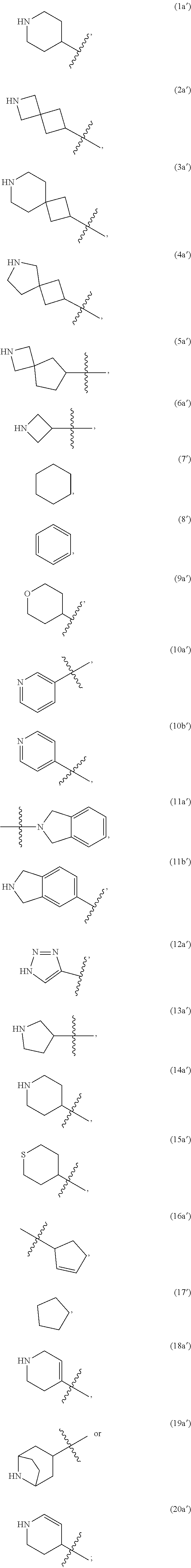
R₃, R₄ and R₅ are attached to the B-rings, and the wavy line denotes the site of attachment to L.

15. The compound according to claim 13, wherein ring B is (1′), (2′), (3′), (4′), (5′), (6′), (7′), (8′), or (9′).

16. The compound according to claim 14, wherein ring B is (1 a′), (2a′), (3a′), (4a′), (5a′), (6a′), (7′), (8′), or (9a′).

17. The compound according to claim 1, wherein R₃ is hydrogen, cyano, C₁₋₇ alkyl, C₂₋₇ alkenyl, hydroxy C₁₋₇ alkyl, C₁₋₇ alkylthio, C₁₋₇ alkoxycarbonyl C₁₋₇ alkyl, —S(O)(NR₁₄)(R₂₂), —S(NR₁₅)(C₁₋₇ alkyl), —C(S)NR₁₈R₁₉, -D-C(O)—NR₆R₇, —C(O)R₈, -D-NR₉R₁₀, —SO₂R₁₁, an optionally substituted 3-10 membered carbocyclyl, optionally substituted 3-10 membered carbocyclyl C₁₋₇ alkyl, optionally substituted 4-10 membered heterocyclyl or an optionally substituted 4-10 membered heterocyclyl C₁₋₇ alkyl; wherein the optional substitution in each occurrence is selected from 1-3 substituents independently selected from C₁₋₇ alkyl, halogen, hydroxy, cyano, C₁₋₇ alkylcarbonyl, C₁₋₇ alkoxy, C₁₋₇ alkoxy C₁₋₇ alkyl, C₁₋₇ alkoxycarbonyl or oxo; and wherein the heterocyclyl group in each occurrence has 1-4 heteroatoms independently selected from N, O, and S.

18. The compound according to claim 17, wherein R₃ is hydrogen, cyano, hydroxy C₁₋₇ alkyl, —S(O)(NR₁₄)(R₂₂), -D-C(O)—NR₆R₇, —C(O)R₈, —SO₂R₁₁, optionally substituted 4-10 membered heterocyclyl or an optionally substituted 4-10 membered heterocyclyl C₁₋₇ alkyl; wherein the optional substitution in each occurrence is selected from 1-3 substituents independently selected from C₁₋₇ alkyl, halogen, hydroxy, cyano, C₁₋₇ alkylcarbonyl and wherein the heterocyclyl group in each occurrence has 1-4 heteroatoms independently selected from N, O, and S.

19. The compound according to claim 1, wherein R₄ and R₅ are hydrogen.

20. The compound according to claim 1, wherein R₁ is hydrogen, C₁₋₇ alkyl, halogen, cyano or halo C₁₋₇ alkyl; R₂ is hydrogen or C₁₋₇ alkyl; or R₁ and R₂ together with the carbon atom to which they are attached form a spiro C₃₋₇ cycloalkyl ring; or R₁ and R₂ together with the carbon atoms to which they are attached form a fused C₃₋₇ cycloalkyl ring.

21. The compound according to claim 1, wherein R₄ is hydrogen or halogen, and R₅ is hydrogen.

22. The compound according to claim 1, wherein ring A is (1) or (2).

23. The compound according to claim 1, wherein R₂₄ is C₁₋₇ alkyl or halo C₁₋₇ alkyl.

24. The compound according to claim 1, wherein the compound is represented by formula (IID): 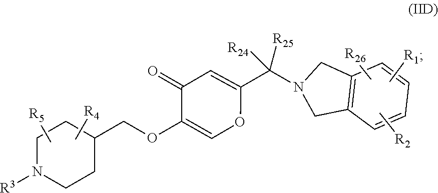
or a pharmaceutically acceptable salt thereof.

25. The compound according to claim 1, wherein the compound is represented by formula (IIE): 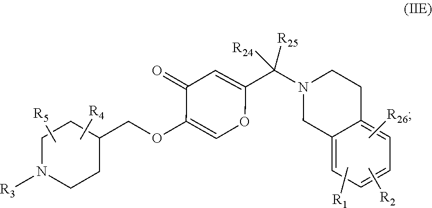
or a pharmaceutically acceptable salt thereof.

26. The compound according to claim 1, wherein R₂₆ is an optionally substituted 4-10 membered heterocyclyl having 1-4 heteroatoms independently selected from N, O, and S.

27. The compound according to claim 1, wherein R₃ is —SO₂R₁₁; R₁, R₂, R₄, and R₅ are hydrogen; and R₁₁ is C₁₋₇ alkyl.

28. The compound according to claim 1, wherein R₃ is an optionally substituted 4-10 membered heterocyclyl C₁₋₇ alkyl; wherein the optional substitution is selected from 1-3 substituents independently selected from C₁₋₇ alkyl, halogen, hydroxy, cyano, C₁₋₇ alkylcarbonyl, and wherein the heterocyclyl group has 1-4 heteroatoms independently selected from N, O, and S; and R₁, R₂, R₄, and R₅ are hydrogen.

29. The compound according to claim 1, wherein R₂₄ is C₁₋₇ alkyl or halo C₁₋₇ alkyl.

30. The compound according to claim 1, wherein the compound is represented by formula (IC): 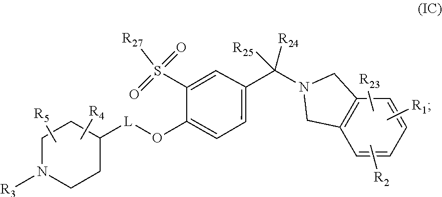
or a pharmaceutically acceptable salt thereof.

31. The compound according to claim 30, wherein R₂₃ is an optionally substituted 4-10 membered heterocyclyl having 1-4 heteroatoms independently selected from N, O, and S.

32. The compound according to claim 30, wherein R₃ is —SO₂R₁₁; R₁, R₂, R₄, and R₅ are hydrogen; and R₁₁ is C₁₋₇ alkyl.

33. The compound according to claim 30, wherein R₃ is an optionally substituted 4-10 membered heterocyclyl C₁₋₇ alkyl; wherein the optional substitution is selected from 1-3 substituents independently selected from C₁₋₇ alkyl, halogen, hydroxy, cyano, C₁₋₇ alkylcarbonyl and wherein the heterocyclyl group has 1-4 heteroatoms independently selected from N, O, and S; and R₁, R₂, R₄, and R₅ are hydrogen.

34. The compound according to claim 30, wherein R₂₄ is C₁₋₇ alkyl or halo C₁₋₇ alkyl.

35. The compound according to claim 16, wherein ring B is (2a′), (3a′), (4a′), (5a′), or (6a′).

36. The compound according to claim 1, wherein
R₃ is hydrogen, —C(O)R₈, —SO₂R₁₁, an optionally substituted 4-10 membered heterocyclyl, or an optionally substituted 4-10 membered heterocyclyl C₁₋₇ alkyl;
R₈ is C₁₋₇ alkoxy;
R₁₁ is C₁₋₇ alkyl, an optionally substituted 4-10 membered heterocyclyl, or an optionally substituted 3-10 membered carbocyclyl;
wherein the optional substitution in each occurrence is selected from 1-3 substituents independently selected from C₁₋₇ alkyl, halogen, hydroxy, cyano, C₁₋₇ alkylcarbonyl, and wherein the heterocyclyl group in each occurrence has 1-4 heteroatoms independently selected from N, O, and S.

37. The compound according to claim 1, wherein the compound is:
2-((5-(1,3,4-Oxadiazol-2-yl)isoindolin-2-yl)methyl)-5-(((methylsulfonyl)piperidin-4-yl)methoxy)-4H-pyran-4-one;
5-((1-(Methylsulfonyl)piperidin-4-yl)methoxy)-2-((5-(oxazol-5-yl)isoindolin-2-yl)methyl)-4H-pyran-4-one;
5-((1-(Methylsulfonyl)piperidin-4-yl)methoxy)-2-((5-(oxazol-2-yl)isoindolin-2-yl)methyl)-4H-pyran-4-one;
2-((5-(Isoxazol-5-yl)isoindolin-2-yl)methyl)-5-((1-(methylsulfonyl)piperidin-4-yl)methoxy)-4H-pyran-4-one;
2-((5-(Isoxazol-3-yl)isoindolin-2-yl)methyl)-5-((1-(methylsulfonyl)piperidin-4-yl)methoxy)-4H-pyran-4-one;
2-((5-(1,3,4-Thiadiazol-2-yl)isoindolin-2-yl)methyl)-5-((1-(methylsulfonyl)piperidin-4-yl)methoxy)-4H-pyran-4-one;
2-((5-(1H-Pyrazol-1-yl)isoindolin-2-yl)methyl)-5-((1-(methylsulfonyl)piperidin-4-yl)methoxy)-4H-pyran-4-one;
2-((5-(1,2,4-Oxadiazol-3-yl)isoindolin-2-yl)methyl)-5-((1-(methylsulfonyl)piperidin-4-yl)methoxy)-4H-pyran-4-one;
5-((1-(Methylsulfonyl)piperidin-4-yl)methoxy)-2-((5-(2,2,2trifluorohydroxyethyl) isoindolin-2-yl)methyl)-4H-pyran-4-one;
2-(2-(3-(Methylsulfonyl)-4-((1-(methylsulfonyl)piperidin-4-yl)methoxy)benzyl)-isoindolin-5-yl)-1,3,4-oxadiazole;
3-(2-(3-(Methylsulfonyl)-4-((1-(methylsulfonyl)piperidin-4-yl)methoxy)benzyl)-isoindolin-5-yl)isoxazole;
5-Bromo-2-(3-(methylsulfonyl)-4-((1-(methylsulfonyl)piperidin-4-yl)methoxy)-benzyl)isoindoline;
2-(3-(Methylsulfonyl)-4-((1-(methylsulfonyl)piperidin-4-yl)methoxy)benzyl)-5-(1H-pyrazol-1-yl)isoindoline;
5-(2-(3-(Methylsulfonyl)-4-((1-(methylsulfonyl)piperidin-4-yl)methoxy)-benzyl)isoindolin-5-yl)oxazole;
2-(2-(3-(Methylsulfonyl)-4-((1-(methylsulfonyl)piperidin-4-yl)methoxy)-benzyl)isoindolin-5-yl)oxazole;
5-(2-(3-(Methylsulfonyl)-4-((1-(methylsulfonyl)piperidin-4-yl)methoxy)benzyl)-isoindolin-5-yl)thiazole;
1-(2-(3-(Methylsulfonyl)-4-((1-(methylsulfonyl)piperidin-4-yl)methoxy)-benzyl)isoindolin-5-yl)ethan-1-one;
3-(Dimethylamino)-1-(2-(3-(methylsulfonyl)-4-((1-(methylsulfonyl)piperidin-4-yl)methoxy)benzyl)isoindolin-5-yl)prop-2-en-1-one;
5-(2-(3-(Methylsulfonyl)-4-((1-(methylsulfonyl)-piperidin-4-yl)methoxy)benzyl)-isoindolin-5-yl)isoxazole;
2-(3-(Methylsulfonyl)-4-((1-(methylsulfonyl)piperidin-4-yl)methoxy)benzyl)-isoindoline-5-carbonitrile;
N—Hydroxy-2-(3-(methylsulfonyl)-4-((1-(methylsulfonyl)piperidin-4-yl)-methoxy)benzyl)isoindoline-5-carboximidamide;
3-(2-(3-(Methylsulfonyl)-4-((1-(methylsulfonyl)piperidin-4-yl)methoxy)benzyl)-isoindolin-5-yl)-1,2,4-oxadiazole;
2-(1-(5-Bromoisoindolin-2-yl)ethyl)-5-((1-(methylsulfonyl)piperidin-4-yl)methoxy)-4H-pyran-4-one;
5-((1-(Methylsulfonyl)piperidin-4-yl)methoxy)-2-(1-(5-(oxazol-2-yl)isoindolin-2-yl)-ethyl)-4H-pyran-4-one;
2-(1-(5-((1-(Methylsulfonyl)piperidin-4-yl)methoxy)-4-oxo-4H-pyran-2-yl)ethyl)-isoindoline-5-carbonitrile;
N—Hydroxy-2-(1-(5-((1-(methylsulfonyl)piperidin-4-yl)methoxy)-4-oxo-4H-pyran-2-yl)ethyl)isoindoline-5-carboximidamide;
2-(1-(5-(1,2,4-Oxadiazol-3-yl)isoindolin-2-yl)ethyl)-5-((1-(methylsulfonyl)piperidin-4-yl)methoxy)-4H-pyran-4-one;
2-(1-(3,4-Dihydroisoquinolin-2(1H)-yl)-2,2,2-trifluoroethyl)-5-((1-(methylsulfonyl)-piperidin-4-yl)methoxy)-4H-pyran-4-one;
5-((1-(Methylsulfonyl)piperidin-4-yl)methoxy)-2-(2,2,2-trifluoro-1-(isoindolin-2-yl)ethyl)-4H-pyran-4-one;
2-(2,2,2-Trifluoro-1-(3-(methylsulfonyl)-4-((1-(methylsulfonyl)piperidin-4-yl)methoxy)phenyl)ethyl)isoindoline;
2-(2,2-Difluoro-1-(3-(methylsulfonyl)-4-((1-(pyrimidin-2-ylmethyl)piperidin-4-yl)-methoxy)phenyl)ethyl)isoindoline;
2-((5-(1,3,4-Oxadiazol-2-yl)isoindolin-2-yl)methyl)-5-(piperidin-4-ylmethoxy)-4H-pyran-4-one;
2-((5-(1,3,4-Oxadiazol-2-yl)isoindolin-2-yl)methyl)-5-((1-(pyrimidin-2-ylmethyl) piperidin-4-yl)methoxy)-4H-pyran-4-one;
2-((5-(1,3,4-Oxadiazol-2-yl)-2H-isoindol-2-yl)methyl)-5-((1-(pyrimidin-2-ylmethyl) piperidin-4-yl)methoxy)-4H-pyran-4-one;
5-((1-(Methylsulfonyl)piperidin-4-yl)methoxy)-2-((S*)-1-((1aR*,7bR*)-1,1a,3,7b-tetrahydro-2H-cyclopropa[c]isoquinolin-2-yl)ethyl)-4H-pyran-4-one;
5-((1-(Methylsulfonyl)piperidin-4-yl)methoxy)-2-((R*)-1-((1aR*,7bR*)-1,1a,3,7b-tetrahydro-2H-cyclopropa[c]isoquinolin-2-yl)ethyl)-4H-pyran-4-one;
2-(3-(Ethylsulfonyl)-4-((1-(methylsulfonyl)piperidin-4-yl)methoxy)benzyl)iso-indoline;
2-(3-(Cyclopropylsulfonyl)-4-((1-(methylsulfonyl)piperidin-4-yl)methoxy)benzyl)-isoindoline;
2-(3-(Ethylsulfonyl)-4-((1-(methylsulfonyl)piperidin-4-yl)methoxy)benzyl)-5-(trifluoromethyl)isoindoline;
2-(3-(Butylsulfonyl)-4-((1-(methylsulfonyl)piperidin-4-yl)methoxy)benzyl)iso-indoline;
2-((1′H-Spiro[cyclopropane-1,4′-isoquinolin]-2′(3′H)-yl)methyl)-5-((1-(methyl-sulfonyl)piperidin-4-yl)methoxy)-4H-pyran-4-one;
tert-Butyl 6-(((6-((3,4-dihydroisoquinolin-2(1H)-yl)methyl)-4-oxo-4H-pyran-3-yl)oxy)methyl)-2-azaspiro[3.3]heptane-2-carboxylate;
tert-Butyl 6-((6-((3,4-dihydroisoquinolin-2(1H)-yl)methyl)-4-oxo-4H-pyran-3-yl)oxy)-2-azaspiro[3.3]heptane-2-carboxylate;
tert-Butyl 2-((6-((3,4-dihydroisoquinolin-2(1H)-yl)methyl)-4-oxo-4H-pyran-3-yl)oxy)-7-azaspiro[3.5]nonane-7-carboxylate;
tert-Butyl 2-((6-((3,4-dihydroisoquinolin-2(1H)-yl)methyl)-4-oxo-4H-pyran-3-yl)oxy)-6-azaspiro[3.4]octane-6-carboxylate;
tert-Butyl 7-((6-((3,4-dihydroisoquinolin-2(1H)-yl)methyl)-4-oxo-4H-pyran-3-yl)oxy)-5-oxa-2-azaspiro[3.4]octane-2-carboxylate;
tert-Butyl 2-(((6-((3,4-dihydroisoquinolin-2(1H)-yl)methyl)-4-oxo-4H-pyran-3-yl)oxy)methyl)-7-azaspiro[3.5]nonane-7-carboxylate;
tert-Butyl 6-(1-((6-((3,4-dihydroisoquinolin-2(1H)-yl)methyl)-4-oxo-4H-pyran-3-yl)oxy)ethyl)-2-azaspiro[3.3]heptane-2-carboxylate;
tert-Butyl 2-(1-((6-((3,4-dihydroisoquinolin-2(1H)-yl)methyl)-4-oxo-4H-pyran-3-yl)-oxy)ethyl)-7-azaspiro[3.5]nonane-7-carboxylate;
5-((2-Azaspiro[3.3]heptan-6-yl)oxy)-2-((3,4-dihydroisoquinolin-2(1H)-yl)methyl)-4H-pyran-4-one bis-trifluoroacetate;
5-((7-Azaspiro[3.5]nonan-2-yl)oxy)-2-((3,4-dihydroisoquinolin-2(1H)-yl)methyl)-4H-pyran-4-one;
5-((7-Azaspiro[3.5]nonan-2-yl)methoxy)-2-((3,4-dihydroisoquinolin-2(1H)-yl)methyl)-4H-pyran-4-one;
5-((6-Azaspiro[3.4]octan-2-yl)oxy)-2-((3,4-dihydroisoquinolin-2(1H)-yl)methyl)-4H-pyran-4-one;
5-((2-Azaspiro[3.3]heptan-6-yl)methoxy)-2-((3,4-dihydroisoquinolin-2(1H)-yl)methyl)-4H-pyran-4-one;
5-(1-(2-Azaspiro[3.3]heptan-6-yl)ethoxy)-2-((3,4-dihydroisoquinolin-2(1H)-yl)methyl)-4H-pyran-4-one;
5-(1-(7-Azaspiro[3.5]nonan-2-yl)ethoxy)-2-((3,4-dihydroisoquinolin-2(1H)-yl)methyl)-4H-pyran-4-one;
2-((3,4-Dihydroisoquinolin-2(1H)-yl)methyl)-5-((2-(pyrimidin-2-yl)-2-azaspiro[3.3]-heptan-6-yl)oxy)-4H-pyran-4-one;
2-((3,4-Dihydroisoquinolin-2(1H)-yl)methyl)-5-((7-(pyrimidin-2-yl)-7-azaspiro[3.5]-nonan-2-yl)oxy)-4H-pyran-4-one;
2-((3,4-Dihydroisoquinolin-2(1H)-yl)methyl)-5-((7-(pyrimidin-2-ylmethyl)-7-azaspiro[3.5]nonan-2-yl)oxy)-4H-pyran-4-one;
2-((3,4-Dihydroisoquinolin-2(1H)-yl)methyl)-5-((7-(pyrimidin-2-yl)-7-azaspiro[3.5]-nonan-2-yl)methoxy)-4H-pyran-4-one;
2-((3,4-Dihydroisoquinolin-2(1H)-yl)methyl)-5-((6-(pyrimidin-2-yl)-6-azaspiro-[3.4]octan-2-yl)oxy)-4H-pyran-4-one;
2-((3,4-Dihydroisoquinolin-2(1H)-yl)methyl)-5-((2-(pyrimidin-2-yl)-2-azaspiro-[3.3]heptan-6-yl)methoxy)-4H-pyran-4-one;
6-(6-(((6-((3,4-Dihydroisoquinolin-2(1H)-yl)methyl)-4-oxo-4H-pyran-3-yl)oxy)-methyl)-2-azaspiro[3.3]heptan-2-yl)nicotinonitrile;
6-(3-(((6-((3,4-Dihydroisoquinolin-2(1H)-yl)methyl)-4-oxo-4H-pyran-3-yl)oxy)-methyl)azetidin-1-yl)nicotinonitrile;
2-((3,4-Dihydroisoquinolin-2(1H)-yl)methyl)-5-((7-((1-methyl-1H-pyrazol-5-yl)sulfonyl)-7-azaspiro[3.5]nonan-2-yl)oxy)-4H-pyran-4-one;
2-((3,4-Dihydroisoquinolin-2(1H)-yl)methyl)-5-((7-(pyridin-3-ylsulfonyl)-7-azaspiro[3.5]nonan-2-yl)oxy)-4H-pyran-4-one;
2-((3,4-Dihydroisoquinolin-2(1H)-yl)methyl)-5-((7-((1-methyl-1H-pyrazol-4-yl)sulfonyl)-7-azaspiro[3.5]nonan-2-yl)oxy)-4H-pyran-4-one;
2-((3,4-Dihydroisoquinolin-2(1H)-yl)methyl)-5-((2-(pyridin-3-ylsulfonyl)-2-azaspiro[3.3]heptan-6-yl)oxy)-4H-pyran-4-one;
2-((3,4-Dihydroisoquinolin-2(1H)-yl)methyl)-5-((2-((1-methyl-1H-pyrazol-4-yl)sulfonyl)-2-azaspiro[3.3]heptan-6-yl)methoxy)-4H-pyran-4-one;
2-((3,4-Dihydroisoquinolin-2(1H)-yl)methyl)-5-((2-((4-fluorophenyl)sulfonyl)-2-azaspiro[3.3]heptan-6-yl)methoxy)-4H-pyran-4-one;
2-((3,4-Dihydroisoquinolin-2(1H)-yl)methyl)-5-(1-(2-(methylsulfonyl)-2-azaspiro[3.3]heptan-6-yl)ethoxy)-4H-pyran-4-one;
2-((3,4-Dihydroisoquinolin-2(1H)-yl)methyl)-5-(1-(2-(pyridin-3-ylsulfonyl)-2-azaspiro[3.3]heptan-6-yl)ethoxy)-4H-pyran-4-one;
2-((3,4-Dihydroisoquinolin-2(1H)-yl)methyl)-5-(1-(7-(methylsulfonyl)-7-azaspiro[3.5]nonan-2-yl)ethoxy)-4H-pyran-4-one;
2-((3,4-Dihydroisoquinolin-2(1H)-yl)methyl)-5-((2-(methylsulfonyl)-2-azaspiro[3.3]heptan-6-yl)methoxy)-4H-pyran-4-one;
2-((3,4-Dihydroisoquinolin-2(1H)-yl)methyl)-5-((7-(methylsulfonyl)-7-azaspiro[3.5]nonan-2-yl)oxy)-4H-pyran-4-one;
2-(1-(5-Acetylisoindolin-2-yl)ethyl)-5-((1-(methylsulfonyl)piperidin-4-yl)methoxy)-4H-pyran-4-one;
2-(3-(Methylsulfonyl)-4-((1-(methylsulfonyl)piperidin-4-yl)methoxy)benzyl)-isoindoline;
2-(3-(Methylsulfonyl)-4-((1-(methylsulfonyl)piperidin-4-yl)methoxy)benzyl)-isoindoline;
1-((1r,4r)-4-((4-(Isoindolin-2-ylmethyl)-2-(methylsulfonyl)phenoxy)methyl)-cyclohexyl)ethan-1-one;
4-((4-(Isoindolin-2-ylmethyl)-2-(methylsulfonyl)phenoxy)methyl)-N,N-dimethylbenzamide;
4-((4-(Isoindolin-2-ylmethyl)-2-(methylsulfonyl)phenoxy)methyl)-N-(2-methoxyethyl)-N-methylbenzamide;
1-(4-((4-Isoindolin-2-ylmethyl)-2-(methylsulfonyl)phenoxy)methyl)piperidin-1-yl)propan-1-one;
Ethyl(imino)(4-((4-(isoindolin-2-ylmethyl)-2-(methylsulfonyl)phenoxy)methyl)-phenyl)-λ⁶-sulfanone;
Imino(4-((4-(isoindolin-2-ylmethyl)-2-(methylsulfonyl)phenoxy)methyl)phenyl)-(methyl)-λ⁶-sulfanone;
1-(4-((4-(Isoindolin-2-ylmethyl)-2-(methylsulfonyl)phenoxy)methyl)piperidin-1-yl)-2-methylpropan-1-one;
4-((4-(Isoindolin-2-ylmethyl)-2-(methylsulfonyl)phenoxy)methyl)-N,N-dimethylcyclohexane-1-carboxamide;
2-(3-(Methylsulfonyl)-4-((1-(oxetan-3-ylsulfonyl)piperidin-4-yl)methoxy)-benzyl)isoindoline;
2-(4-((4-(Ethylsulfonyl)benzyl)oxy)-3-(methylsulfonyl)benzyl)isoindoline;
1-((4-((4-(Isoindolin-2-ylmethyl)-2-(methylsulfonyl)phenoxy)methyl)phenyl)-sulfonyl)azetidin-3-ol;
N-(Dimethyl(oxo)-λ⁶-sulfaneylidene)-4-((4-(isoindolin-2-ylmethyl)-2-(methyl-sulfonyl)phenoxy)methyl)benzamide;
(1r,4r)-4-((4-(Isoindolin-2-ylmethyl)-2-(methylsulfonyl)phenoxy)methyl)-N,N-dimethylcyclohexane-1-carboxamide;
Azetidin-1-yl(4-((4-(isoindolin-2-ylmethyl)-2-(methylsulfonyl)phenoxy)methyl)-cyclohexyl)methanone;
2-(4-((3-Fluoro-4-(methylsulfonyl)benzyl)oxy)-3-(methylsulfonyl)benzyl)isoindoline;
2-(3-(Methylsulfonyl)-4-((4-(methylsulfonyl)benzyl)oxy)benzyl)isoindoline;
2-(3-(Methylsulfonyl)-4-((tetrahydro-2H-pyran-4-yl)methoxy)benzyl)isoindoline;
(4-((4-(Isoindolin-2-ylmethyl)-2-(methylsulfonyl)phenoxy)methyl)phenyl)(methyl)-λ⁴-sulfanimine;
Imino(methyl)(4-((2-(methylsulfonyl)-4-((5-(trifluoromethyl)isoindolin-2-yl)methyl)phenoxy)methyl)phenyl)-λ⁶-sulfanone;
2-(4-((4-(Isoindolin-2-ylmethyl)-2-(methylsulfonyl)phenoxy)methyl)phenyl)propan-2-ol;
1-(4-((4-(Isoindolin-2-ylmethyl)-2-(methylsulfonyl)phenoxy)methyl)piperidin-1-yl)ethan-1-one;
2-((1′H-Spiro[cyclopropane-1,4′-isoquinolin]-2′(3′H)-yl)methyl)-5-((1-tosylpiperidin-4-yl)methoxy)-4H-pyran-4-one;
2-((5-Acetylisoindolin-2-yl)methyl)-5-((1-(methylsulfonyl)piperidin-4-yl)methoxy)-4H-pyran-4-one;
(E)-2-((5-(1-(Hydroxyimino)ethyl)isoindolin-2-yl)methyl)-5-((1-(methylsulfonyl)-piperidin-4-yl)methoxy)-4H-pyran-4-one;
(E)-2-((5-(1-(Methoxyimino)ethyl)isoindolin-2-yl)methyl)-5-((1-(methylsulfonyl)-piperidin-4-yl)methoxy)-4H-pyran-4-one;
1-(4-((4-(Isoindolin-2-ylmethyl)-2-(methylsulfonyl)phenoxy)methyl)phenyl)-ethanone;
1-(4-((4-(Isoindolin-2-ylmethyl)-2-(methylsulfonyl)phenoxy)methyl)phenyl)ethanol;
2-((5-(1-Hydroxyethyl)isoindolin-2-yl)methyl)-5-((1-(methylsulfonyl)piperidin-4-yl)methoxy)-4H-pyran-4-one;
1-(4-((4-(Isoindolin-2-ylmethyl)-2-(methylsulfonyl)phenoxy)methyl)cyclohexyl)-ethan-1-ol;
(R)-Imino(4-((4-(isoindolin-2-ylmethyl)-2-(methylsulfonyl)phenoxy)methyl)phenyl)-(methyl)-λ⁶-sulfanone;
(S)-Imino(4-((4-(isoindolin-2-ylmethyl)-2-(methylsulfonyl)phenoxy)methyl)phenyl)-(methyl)-λ⁶-sulfanone;
(S)-Ethyl(imino)(4-((4-(isoindolin-2-ylmethyl)-2-(methylsulfonyl)phenoxy)methyl)-phenyl)-λ⁶-sulfanone;
(R)-Ethyl(imino)(4-((4-(isoindolin-2-ylmethyl)-2-(methylsulfonyl)phenoxy)methyl)-phenyl)-λ⁶-sulfanone;
(R)-2-(1-(3,4-dihydroisoquinolin-2(1H)-yl)-2,2,2-trifluoroethyl)-5-((1-(methylsulfonyl)piperidin-4-yl)methoxy)-4H-pyran-4-one;
(S)-2-(1-(3,4-dihydroisoquinolin-2(1H)-yl)-2,2,2-trifluoroethyl)-5-((1-(methylsulfonyl)piperidin-4-yl)methoxy)-4H-pyran-4-one;
2-((6-(Isoxazol-4-yl)-3,4-dihydroisoquinolin-2(1H)-yl)methyl)-5-((1-(methyl-sulfonyl)piperidin-4-yl)methoxy)-4H-pyran-4-one;
2-((6-(1H-Pyrazol-1-yl)-3,4-dihydroisoquinolin-2(1H)-yl)methyl)-5-((1-(methyl-sulfonyl)piperidin-4-yl)methoxy)-4H-pyran-4-one;
2-((1′H-Spiro[cyclopropane-1,4′-isoquinolin]-2′(3′H)-yl)methyl)-5-((1-(methyl-sulfonyl)piperidin-4-yl)methoxy)-4H-pyran-4-one;
2-((1′H-Spiro[cyclopentane-1,4′-isoquinolin]-2′(3′H)-yl)methyl)-5-((4-(2-hydroxy-propan-2-yl)benzyl)oxy)-4H-pyran-4-one;
4-Fluoro-2-(3-(methylsulfonyl)-4-((1-(methylsulfonyl)piperidin-4-yl)methoxy)-benzyl)isoindoline; or
tautomers or pharmaceutically acceptable salts thereof.

38. A method for the treatment of a steroid receptor dependent condition or disease comprising administering to a subject in need thereof a therapeutically effective amount of a compound according to claim 1.

39. The method according to claim 38, wherein the steroid receptor dependent disease is cancer.

40. The method according to claim 39, wherein the cancer is prostate cancer.

41. The method according to claim 40, wherein the prostate cancer is castration-resistant prostate cancer (CRPC).

42. The method according to claim 38, wherein the method comprises administering a therapeutically effective amount of a compound of formula (I) in addition to a glucocorticoid and/or a mineralocorticoid and, optionally, one or more anti-cancer agents.

43. The method according to claim 38, wherein the method comprises administering a therapeutically effective amount of a compound of formula (I) in addition to one or more anti-cancer agents selected from the group consisting of:
non-steroidal androgen receptor antagonists;
steroidogenesis inhibitors;
chemotherapeutic agents;
antiestrogens;
epigenetic modulators;
mTOR inhibitors;
AKT inhibitors;
radiopharmaceuticals;
GnRH/LHRH analogues;
PI3K inhibitors; and
CDK4/6 inhibitors.

44. A method according to claim 38, wherein the method comprises administering a therapeutically effective amount of a compound of formula (I) and a therapeutically effective amount of a non-steroidal androgen receptor antagonist.

45-51. (canceled)

52. A pharmaceutical composition comprising a compound according to claim 1 together with a pharmaceutically acceptable carrier.

53. A pharmaceutical combination comprising a compound according to claim 1 and at least one additional active ingredient selected from the list consisting of:
glucocorticoids;
mineralocorticoids;
non-steroidal androgen receptor antagonists;
steroidogenesis inhibitors;
chemotherapeutic agents;
antiestrogens;
epigenetic modulators;
mTOR inhibitors;
AKT inhibitors;
radiopharmaceuticals;
GnRH/LHRH analogues;
PI3K inhibitors; and
CDK4/6 inhibitors; and
wherein the pharmaceutical combination is administered simultaneously, separately, or sequentially.
